SEQUENCE LISTING (50 sequences)

SEQ ID NO: 1 - DNA, 25 bp, synthetic construct
  misc_feature 1..25: note = ESR1
  source 1..25: mol_type = other DNA
ctgaaccgtccgcagctcaagatcc

SEQ ID NO: 2 - DNA, 22 bp, synthetic construct
  misc_feature 1..22: note = ESR1
  source 1..22: mol_type = other DNA
ggatggccctactgcatcagat

SEQ ID NO: 3 - DNA, 21 bp, synthetic construct
  misc_feature 1..21: note = ESR1
  source 1..21: mol_type = other DNA
cttgctgctgtccaggtacac

SEQ ID NO: 4 - DNA, 26 bp, synthetic construct
  misc_feature 1..26: note = PGR
  source 1..26: mol_type = other DNA
catgtggcagatcccacaggagtttg

SEQ ID NO: 5 - DNA, 29 bp, synthetic construct
  misc_feature 1..29: note = PGR
  source 1..29: mol_type = other DNA
cagcggatgaaagaatcatcattctattc

SEQ ID NO: 6 - DNA, 27 bp, synthetic construct
  misc_feature 1..27: note = PGR
  source 1..27: mol_type = other DNA
aggaactcttcttggctaacttgaagc

SEQ ID NO: 7 - DNA, 24 bp, synthetic construct
  misc_feature 1..24: note = HER2
  source 1..24: mol_type = other DNA
cccagccctggtcacctacaacac

SEQ ID NO: 8 - DNA, 19 bp, synthetic construct
  misc_feature 1..19: note = HER2
  source 1..19: mol_type = other DNA
ggcctgcctccacttcaac

SEQ ID NO: 9 - DNA, 19 bp, synthetic construct
  misc_feature 1..19: note = HER2
  source 1..19: mol_type = other DNA
gggacaggcagtcacacag

SEQ ID NO: 10 - DNA, 22 bp, synthetic construct
  misc_feature 1..22: note = ARFIP1
  source 1..22: mol_type = other DNA
gtcgccggtccgactgtcctct

SEQ ID NO: 11 - DNA, 20 bp, synthetic construct
  misc_feature 1..20: note = ARFIP1
  source 1..20: mol_type = other DNA
ggcttcgctgctcttggttc

SEQ ID NO: 12 - DNA, 24 bp, synthetic construct
  misc_feature 1..24: note = ARFIP1
  source 1..24: mol_type = other DNA
agcaactgcagctgtcacaaattc

SEQ ID NO: 13 - DNA, 22 bp, synthetic construct
  misc_feature 1..22: note = COX1
  source 1..22: mol_type = other DNA
attcggcgcatgagctggagtc

SEQ ID NO: 14 - DNA, 25 bp, synthetic construct
  misc_feature 1..25: note = COX1
  source 1..25: mol_type = other DNA
caaaccacaaagacattggaacact

SEQ ID NO: 15 - DNA, 20 bp, synthetic construct
  misc_feature 1..20: note = COX1
  source 1..20: mol_type = other DNA
cagctcggctcgaataagga

SEQ ID NO: 16 - DNA, 21 bp, synthetic construct
  misc_feature 1..21: note = PLIN1
  source 1..21: mol_type = other DNA
tgtccttggcgctagacacca

SEQ ID NO: 17 - DNA, 20 bp, synthetic construct
  misc_feature 1..20: note = PLIN1
  source 1..20: mol_type = other DNA
cggtcactacggactttgtc

SEQ ID NO: 18 - DNA, 24 bp, synthetic construct
  misc_feature 1..24: note = PLIN1
  source 1..24: mol_type = other DNA
accaaggagcttgtgtcgtctaag

SEQ ID NO: 19 - DNA, 26 bp, synthetic construct
  misc_feature 1..26: note = EGFR
  source 1..26: mol_type = other DNA
caccacgtaccagatggatgtgaacc

SEQ ID NO: 20 - DNA, 20 bp, synthetic construct
  misc_feature 1..20: note = EGFR
  source 1..20: mol_type = other DNA
gtgactgctgccacaaccag

SEQ ID NO: 21 - DNA, 32 bp, synthetic construct
  misc_feature 1..32: note = EGFR
  source 1..32: mol_type = other DNA
agctccctctcctgcagcagcctccgcagcgt

SEQ ID NO: 22 - DNA, 20 bp, synthetic construct
  misc_feature 1..20: note = EGFR
  source 1..20: mol_type = other DNA
tctgccgcaaattccgagac

SEQ ID NO: 23 - DNA, 21 bp, synthetic construct
  misc_feature 1..21: note = EGFR
  source 1..21: mol_type = other DNA
ggacacttcttcacgcaggtg

SEQ ID NO: 24 - DNA, 36 bp, synthetic construct
  misc_feature 1..36: note = MMP7
  source 1..36: mol_type = other DNA
ctggaatgttaaactcccgcgtcatagaaataatgc

SEQ ID NO: 25 - DNA, 26 bp, synthetic construct
  misc_feature 1..26: note = MMP7
  source 1..26: mol_type = other DNA
ccaacagtttagaagccaaactcaag

SEQ ID NO: 26 - DNA, 22 bp, synthetic construct
  misc_feature 1..22: note = MMP7
  source 1..22: mol_type = other DNA
gcaacatctggcactccacatc

SEQ ID NO: 27 - DNA, 30 bp, synthetic construct
  misc_feature 1..30: note = TERT
  source 1..30: mol_type = other DNA
ttgaagagggtgcagctgcgggagctgtcg

SEQ ID NO: 28 - DNA, 24 bp, synthetic construct
  misc_feature 1..24: note = P53
  source 1..24: mol_type = other DNA
cccctgcaccagcagctcctacac

SEQ ID NO: 29 - DNA, 37 bp, synthetic construct
  misc_feature 1..37: note = MEDI12
  source 1..37: mol_type = other DNA
cagcagcagcaacagcaacagcagcagcagcagcagc

SEQ ID NO: 30 - DNA, 26 bp, synthetic construct
  misc_feature 1..26: note = RFX2
  source 1..26: mol_type = other DNA
gtaacagtctccacggctacagtctc

SEQ ID NO: 31 - DNA, 20 bp, synthetic construct
  misc_feature 1..20: note = P21
  source 1..20: mol_type = other DNA
acagcggccccggcacccag

SEQ ID NO: 32 - DNA, 23 bp, synthetic construct
  misc_feature 1..23: note = PI3KB
  source 1..23: mol_type = other DNA
catggggcagatgctggagcagt

SEQ ID NO: 33 - DNA, 20 bp, synthetic construct
  misc_feature 1..20: note = SEPT9
  source 1..20: mol_type = other DNA
cccttggtcagcctcctccc

SEQ ID NO: 34 - DNA, 23 bp, synthetic construct
  misc_feature 1..23: note = Sars-Cov-2
  source 1..23: mol_type = other DNA
ccagatgaggatgaagaagaagg

SEQ ID NO: 35 - DNA, 25 bp, synthetic construct
  misc_feature 1..25: note = Sars-Cov-2
  source 1..25: mol_type = other DNA
catggaggaggtgttgcaggagcct

SEQ ID NO: 36 - DNA, 24 bp, synthetic construct
  misc_feature 1..24: note = Sars-Cov-2
  source 1..24: mol_type = other DNA
aagttgatggtgttgatgtagaat

SEQ ID NO: 37 - DNA, 25 bp, synthetic construct
  misc_feature 1..25: note = ESR1-M1
  source 1..25: mol_type = other DNA
ctgaaccgtacgcagctcaagatcc

SEQ ID NO: 38 - DNA, 25 bp, synthetic construct
  misc_feature 1..25: note = ESR1-M2
  source 1..25: mol_type = other DNA
ctgaaccgtacgtagctcaagatcc

SEQ ID NO: 39 - DNA, 25 bp, synthetic construct
  misc_feature 1..25: note = ESR1-M3
  source 1..25: mol_type = other DNA
ctgaaccgtacgtagatcaagatcc

SEQ ID NO: 40 - DNA, 25 bp, synthetic construct
  misc_feature 1..25: note = ESR1-M4
  source 1..25: mol_type = other DNA
ctgaactgtacgtagatcaagatcc

SEQ ID NO: 41 - DNA, 20 bp, synthetic construct
  misc_feature 1..20: note = SEPT9 P1-A
  source 1..20: mol_type = other DNA
cccttggtcagcctccttcc

SEQ ID NO: 42 - DNA, 20 bp, synthetic construct
  misc_feature 1..20: note = SEPT9 P1-B
  source 1..20: mol_type = other DNA
cccttggttagcctcctacc

SEQ ID NO: 43 - DNA, 20 bp, synthetic construct
  misc_feature 1..20: note = SEPT9 P1-C
  source 1..20: mol_type = other DNA
cccttggtcagactactacc

SEQ ID NO: 44 - DNA, 26 bp, synthetic construct
  misc_feature 1..26: note = RFX2 P2-A
  source 1..26: mol_type = other DNA
gtaacagtctccacggctaaagtctc

SEQ ID NO: 45 - DNA, 26 bp, synthetic construct
  misc_feature 1..26: note = RFX2 P2-B
  source 1..26: mol_type = other DNA
gtaacagtctccacggatatagtctc

SEQ ID NO: 46 - DNA, 26 bp, synthetic construct
  misc_feature 1..26: note = RFX2 P2-C
  source 1..26: mol_type = other DNA
gtaacagtcttcaaggctacagtctc

SEQ ID NO: 47 - DNA, 24 bp, synthetic construct
  misc_feature 1..24: note = P53 P3-A
  source 1..24: mol_type = other DNA
cccctgcaccagcagatcataaac

SEQ ID NO: 48 - DNA, 24 bp, synthetic construct
  misc_feature 1..24: note = P53 P3-B
  source 1..24: mol_type = other DNA
accatgaaccagcagctcctacac

SEQ ID NO: 49 - DNA, 24 bp, synthetic construct
  misc_feature 1..24: note = P53 P3-C
  source 1..24: mol_type = other DNA
cccctgcacaagaagctcctacac

SEQ ID NO: 50 - DNA, 49 bp, synthetic construct
  misc_feature 1..49: note = P53
  source 1..49: mol_type = other DNA
cccctgcaccagcagctcctacaccggcggcccctgcaccagccccctc

(19) United States
(12) Patent Application Publication
(10) Pub. No.: US 2024/0301480 A1
(43) Pub. Date: Sep. 12, 2024

(54) METHODS AND COMPOSITIONS FOR ANALYZING NUCLEIC ACIDS AND NUCLEASES IN A BODY FLUID AS INDICATORS OF DISEASE

(71) Applicant: ARNA Genomics US Incorporated, Wellington, FL (US)

(72) Inventors: Anatoliy Melnikov, Wheeling, IL (US); George NIKITIN, Moscow (RU); Egor MELNIKOV, Moscow (RU); Charles R. CANTOR, Dover Plains, NY (US); Takeshi SANO, Newport Coast, CA (US)

(21) Appl. No.: 18/661,301

(22) Filed: May 10, 2024

Related U.S. Application Data
(63) Continuation of application No. PCT/US22/79770, filed on Nov. 11, 2022.
(60) Provisional application No. 63/278,935, filed on Nov. 12, 2021.

(51) Int. Cl. C12Q 1/6851; C12Q 1/37; C12Q 1/44; C12Q 1/6806; C12Q 1/6886; C12Q 1/70

(52) U.S. Cl. CPC C12Q 1/6851; C12Q 1/37; C12Q 1/44; C12Q 1/6806; C12Q 1/6886; C12Q 1/70

(57) ABSTRACT

Provided herein are methods and compositions for analyzing extracellular (cell-free) nucleic acids and/or nucleases in a body fluid, to identify subjects having a disease or condition associated with a change in the amount of cell-free nucleic acids and/or nuclease activity compared to the amount of cell-free nucleic acids and/or nuclease activity in a healthy subject. The methods and compositions provided herein can be used to identify subjects having a disease such as cancer, or a disease caused by a pathogen, for example, a virus, such as SARS-CoV-2.

### RELATED APPLICATIONS

This application is a continuation of International PCT application No. PCT/US22/79770, on Nov. 11, 2022, published as International PCT publication No. WO 2023/086970, on May 19, 2023, entitled “METHODS AND COMPOSITIONS FOR ANALYZING NUCLEIC ACIDS AND NUCLEASES IN A BODY FLUID AS INDICATORS OF DISEASE,” to Applicant ARNA Genomics US Incorporated, and inventors Anatoliy Melnikov, George Nikitin, Egor Melnikov, Charles R. Cantor, and Takeshi Sano. PCT application No. PCT/US22/79770 claims the benefit of priority to U.S. provisional application Ser. No. 63/278,935, filed Nov. 12, 2021, entitled “METHODS AND COMPOSITIONS FOR ANALYZING NUCLEIC ACIDS AND NUCLEASES IN A BODY FLUID AS INDICATORS OF DISEASE,” to Applicant ARNA Genomics US Incorporated, and inventors Anatoliy Melnikov, George Nikitin, Egor Melnikov, and Charles R. Cantor.

This application claims benefit of priority to U.S. provisional application Ser. No. 63/278,935, filed Nov. 12, 2021, entitled “METHODS AND COMPOSITIONS FOR ANALYZING NUCLEIC ACIDS AND NUCLEASES IN A BODY FLUID AS INDICATORS OF DISEASE,” to Applicant ARNA Genomics US Incorporated, and inventors Anatoliy Melnikov, George Nikitin, Egor Melnikov, and Charles R. Cantor.

The subject matter of these applications is incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on May 9, 2024, is 63,904 bytes in size, and is titled 6003SEQ001.xml.

### FIELD OF THE INVENTION

Provided are methods and compositions for analyzing extracellular (cell-free) nucleic acids and/or nucleases in a body fluid, to identify subjects having a disease or condition associated with a change in the amount of cell-free nucleic acids and/or nuclease activity compared to the amount of the cell-free nucleic acids and/or nuclease activity in a healthy subject.

### BACKGROUND

Cell-free DNA (cfDNA or extracellular DNA) is composed of DNA fragments that originate from cell death and circulate in peripheral blood and other body fluids. High concentrations of cfDNA can be indicative of certain clinical conditions such as cancer, trauma, burns, myocardial infarction, acute trauma, inflammation, autoimmune diseases, stroke, sepsis, infection by pathogens, and other illnesses.

For cancers, tumor biopsy is considered the gold standard for diagnosis. Biopsy, however, is an invasive procedure limited only to certain locations and not always feasible in clinical practice. Imaging often cannot provide enough information on tumor character, given its inherent biological heterogeneity, to direct further treatment. Longitudinal or simultaneous multi-site testing is not feasible due to the clinical complications associated with seriate tissue sampling and its effects on patients' quality of life.

To avoid painful procedures such as tissue biopsy, liquid biopsy can represent a convenient and efficient tool. Blood or other body fluid-based circulating biomarkers, including circulating tumor cells (CTCs), cell-free nucleic acids, exosomes and nucleases, have been studied as ‘liquid biopsies’, i.e., biomarkers that can overcome the drawbacks of invasive tissue biopsies. Liquid biopsies to date have had limitations due to, e.g., heterogeneity in samples leading to poor reproducibility and/or compromised specificity and sensitivity. Improved methods that provide higher diagnostic specificity and sensitivity are needed.

### SUMMARY

Provided herein are methods of analyzing the amount of cell-free nucleic acid or cell-free nuclease activity in a body as indicative of the presence or absence of a disease or condition. The methods include the addition of several reagents as described below and elsewhere herein. References to concentrations of the reagents, such as nucleic acids (e.g., primers and nucleic acid probes), cofactors, detergents, enzymes such as proteinase K and the like, unless noted otherwise, indicates final concentrations of these reagents in the reactions.

Provided herein is a method, termed “Method 1” herein, for analyzing cell-free nucleic acids in a body fluid from a subject as indicative of the presence or absence of a disease or condition in the subject, which includes: a) subjecting a sample of body fluid previously obtained from the subject to an amplification reaction, where i) the sample contains cell-free nucleic acid, and cell-free nucleic acid derived from at least one target gene is specifically amplified, and ii) the cell-free nucleic acid derived from the target gene is present in an amount that is about or less than 5, 4, 3, 2, or 1 molecule(s) or molecule(s) per unit volume, in an analogous control or reference sample, and is present in the sample from the subject with the disease or condition in an amount that is greater than the amount in the control or reference sample by 2 or more molecules or molecules per unit volume; and b) quantitating the amplified nucleic acid from a), where i) if the amount of amplified nucleic acid different from a threshold level, depending upon the disease, disorder, or condition, and generally at or above a threshold level, the subject is identified as having the disease or condition; and ii) if the amount of amplified nucleic acid is below a threshold level, the subject is identified as not having the disease or condition.

In some examples, the control or reference sample is an analogous sample from a normal subject or a subject who does not have the disease or condition. In examples, the cell-free nucleic acid derived from the target gene is present in an amount that is 1 molecule or less, or 1 molecule or less per unit volume, in the control or reference sample and is present in the sample from the subject with the disease or condition in an amount that is 3 molecules or more, or 3 molecules or more per unit volume. In some examples, the difference between the number of cell-free nucleic acid molecules derived from the target gene that are present in the control or reference sample and the number of cell-free nucleic acid molecules derived from the target gene that are present in the sample from the subject with the disease or condition is about 2, 3, 4, 5, 6, 7, 8, 9 or 10 molecules, or 2, 3, 4, 5, 6, 7, 8, 9 or 10 molecules per unit volume. In some examples, the difference between the number of cell-free nucleic acid molecules derived from the target gene that are present in the control or reference sample and the number of cell-free nucleic acid molecules derived from the target gene that are present in the sample from the subject with the disease or condition is about 2, 3, 4 or 5 molecules, or 2, 3, 4 or 5 molecules per unit volume. In examples of Method 1, the cell-free nucleic acid derived from at least one target gene contains at least one zinc finger binding site (or motif).

In some examples of Method 1, provided is a method for analyzing cell-free nucleic acids in a body fluid from a subject as indicative of the presence or absence of a disease or condition in the subject, which includes: a) subjecting a sample of body fluid previously obtained from the subject to a nucleic acid amplification reaction, where the sample contains cell-free nucleic acid and where cell-free nucleic acid derived from at least one target gene and containing at least one zinc finger binding site is specifically amplified; and b) quantitating the amplified nucleic acid from a), where: i) if the amount of amplified nucleic acid is at or above a threshold level, the subject is identified as having the disease or condition; and ii) if the amount of amplified nucleic acid is below a threshold level, the subject is identified as not having the disease or condition.

In any of the examples of Method 1 provided herein, in some examples, the threshold level can be determined based on a level that is measured in at least one subject known to have the disease or condition and/or is based on a level that is measured in at least one control or reference sample; or the threshold level can be determined based on a level that is the mean or the median of levels measured in more than one subject known to have the disease or condition and/or the mean or the median of levels measured in more than one control or reference sample. In some examples, the control or reference sample is an analogous sample from a normal subject or a subject who does not have the disease or condition. In some examples, the cell-free nucleic acid derived from the target gene is present in an amount that is about or less than 5, 4, 3, 2, or 1 molecule(s) or molecule(s) per unit volume in an analogous control or reference sample, and is present in the sample from the subject with the disease or condition in an amount that is greater than the amount in the control or reference sample by 2 or more molecules or molecules per unit volume. In some examples, the cell-free nucleic acid derived from the target gene is present in an amount that is 1 molecule or less or 1 molecule or less per unit volume in the control or reference sample and is present in the sample from the subject with the disease or condition in an amount that is 3 molecules or more or 3 molecules or more per unit volume. In examples, the difference between the number of cell-free nucleic acid molecules derived from the target gene that are present in the control or reference sample and the number of cell-free nucleic acid molecules derived from the target gene that are present in the sample from the subject with the disease or condition is about 2, 3, 4, 5, 6, 7, 8, 9 or 10 molecules, or 2, 3, 4, 5, 6, 7, 8, 9 or 10 molecules per unit volume. In some examples, the difference between the number of cell-free nucleic acid molecules derived from the target gene that are present in the control or reference sample and the number of cell-free nucleic acid molecules derived from the target gene that are present in the sample from the subject with the disease or condition is about 2, 3, 4 or 5 molecules, or 2, 3, 4 or 5 molecules per unit volume.

In any of the methods provided herein, including Method 1 and certain combination methods that include quantitating amplified nucleic acid as provided herein, the quantitating can be performed by any method known to those of skill in the art including, but not limited to, gel electrophoresis, Picogreen assay, UV spectrometry, real-time quantitative PCR (real-time (RT)-qPCR), droplet digital PCR (ddPCR), BEAMing or TAm-Seq. In some examples, the quantitating is performed by measuring a signal that is proportional to or inversely proportional to the amount of the amplification product.

In examples of the methods provided herein, including Method 1 and certain combination methods that include quantitating amplified nucleic acid as provided herein, the quantitating is by real-time (RT)-qPCR. In some examples, the quantitating of amplified cell-free nucleic acid derived from at least one target gene includes using a nucleic acid probe that hybridizes to the cell-free nucleic acid derived from at least one target gene. In some examples, the quantitating is performed by measuring a cycle threshold (Ct) value and if the Ct value is at or below a threshold level, the subject is identified as having the disease or condition, and if the Ct value is above a threshold level, the subject is identified as not having the disease or condition. The Ct value can be determined by measuring a signal that is proportional to the amount of the amplification product. In some examples, the Ct value is measured as the amplification cycle number at which a signal above a background level is detected.

In examples, the Ct value is measured as the amplification cycle number where exponential increase of a signal associated with the amplification product begins, e.g., the first amplification cycle number where exponential increase of the signal begins. An algorithm for measuring the first amplification cycle number where exponential increase of a signal associated with the amplification product begins is provided herein: for example, the Ct value can be measured by: a) detecting the baseline angle of the S-curve and adjusting the curve by linear approximation so that the baseline obtained during the early PCR cycles is flat; b) analyzing a change in signal (ΔF), starting from the last cycle at the end of the real-time-qPCR reaction and moving backwards until a cycle number is reached where the value of ΔF changes from a positive value to zero or a negative value; and c) designating the cycle number where the value of ΔF changes from a positive value to zero or a negative value as the Ct value. In examples, the early PCR cycles are between about 10 cycles or 10-30 cycles, inclusive, 30 cycles of the real-time-qPCR reaction, or between about 20 cycles to about 30 cycles, or 20-30 cycles, inclusive, of the real-time-qPCR reaction. In examples, the last cycle at the end of the real-time-qPCR reaction is between about 40 cycles to about 50 cycles, or 40-50 cycles, inclusive, of the real-time-qPCR reaction, or the last cycle at the end of the real-time-qPCR reaction is at least about 44, 45, 46, 47, 48, 49, up to and including 50 cycles of the real-time-qPCR reaction. In some examples, the last cycle at the end of the real-time-qPCR reaction is 46 cycles of the real-time-qPCR reaction. In some examples, the algorithm further includes linear approximation to levels of the signal such that the approximated Ct is defined as a point between two cycles where the linearly approximated signal crosses the background (Signal_{baseline}+σₙₒᵢₛₑ) level. In examples, the Ct value is determined using the algorithm provided herein. In examples: i) if the Ct value is at or below 34, the subject is identified as having the disease or condition; and ii) if the Ct value is above 34, the subject is identified as not having the disease or condition.

In any of the methods provided herein, including Method 1 and certain combination methods that include quantitating amplified nucleic acid as provided herein, the signal that is measured as indicative of the amount of amplified nucleic acid can be fluorescence. In any of the methods provided herein, including Method 1 and certain combination methods that include quantitating amplified nucleic acid as provided herein, in some examples, the cell-free nucleic acid is not extracted or isolated from the body fluid for analysis. In examples, the volume of body fluid analyzed is between about 1 μL to about 1 mL, or 1 μL to 1 mL, inclusive. In some examples, the volume of body fluid analyzed is 100 μL or less. In examples, the volume of body fluid analyzed is between about 5 μL to about 50 μL, or 5 μL to 50 μL, inclusive. In examples, the volume of body fluid analyzed is between about 2 μL to about 25 μL, or between about 1 μL to about 20 μL, or 2 μL to 20 μL, inclusive. In some examples, the volume of body fluid analyzed is about or at least about or at 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL, up to and including 30 μL.

In any of the methods provided herein, including Method 1 and certain combination methods that include quantitating amplified nucleic acid as provided herein, in some examples, the cell-free nucleic acid in the sample of body fluid is treated with a proteinase prior to analysis. In some examples, the proteinase is proteinase K. In examples, the concentration of proteinase is between about 1 mg/mL to about 10 mg/mL, or 1 mg/mL to 10 mL, inclusive, or at least about 1.5 mg/mL, 2.0 mg/mL, 2.5 mg/mL, 3.0 mg/mL, 3.5 mg/mL, 4.0 mg/mL, 4.5 mg/mL, up to 5 mg/mL.

In any of the methods provided herein, including Method 1 and certain combination methods that include quantitating amplified nucleic acid as provided herein, in some examples, the disease or condition is cancer, a non-cancerous proliferative disorder or an infectious disease caused by a pathogen. In examples, the disease or condition is cancer or a non-cancerous proliferative disorder. In some examples, the cancer is selected from among an early-stage cancer, a late-stage cancer, a metastatic cancer, and an undifferentiated cancer. In examples, the cancer or non-cancerous proliferative disorder is selected from among ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, chordoma, chromophobe renal cell carcinoma, clear cell carcinoma, colon cancer, colorectal cancer, cutaneous benign fibrous histiocytoma, fibroadenoma, fibrocystic disease, desmoplastic small round cell tumor, ependymoma, Ewing's tumor, extraskeletal myxoid chondrosarcoma, fibrogenesis imperfecta ossium, fibrous dysplasia of the bone, gallbladder cancer, bile duct cancer, gastric cancer, synovial tissue cancer, gestational trophoblastic disease, germ cell tumor, glioblastoma, hematological malignancy, hepatocellular carcinoma, islet cell tumor, Kaposi's Sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, myelodysplastic syndrome, neuroblastoma, neuroendocrine tumors, papillary thyroid carcinoma, parathyroid tumor, pediatric cancer, peripheral nerve sheath tumor, pheochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, squamous cell cancer of the head and neck (SCCHN), stomach cancer, synovial tissue cancer, synovial sarcoma, testicular cancer, thymic carcinoma, thymoma, thyroid cancer, thyroid metastatic cancer, uterine cancer, hepatoma, glioma, retinoblastoma, myeloma, lymphoma, and leukemia. In some examples, the cancer or non-cancerous proliferative disorder is of the breast; in examples, the cancer or non-cancerous proliferative disorder is selected from among invasive ductal carcinoma breast cancer, luminal A breast cancer, luminal B breast cancer, HER2-enriched breast cancer, triple negative breast cancer, fibroadenoma and fibrocystic disease.

In any of the methods provided herein, including Method 1 and certain combination methods that include quantitating amplified nucleic acid as provided herein, the body fluid is selected from among one or more of whole blood, urine, plasma, serum, cerebrospinal fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In some examples, the body fluid is plasma. In examples, the volume of plasma analyzed is between about 1 μL to about 1 mL or 1 μL to 1 mL, inclusive. In some examples, the volume of plasma analyzed is 100 μL or less. In examples, the volume of plasma analyzed is between about 5 μL to about 50 μL. In some examples, the volume of plasma analyzed is between about 2 μL to about 25 μL. In examples, the volume of plasma analyzed is between about 1 μL to about 20 μL. In some examples, the volume of plasma analyzed is about or at least about 1 ptL, 2.5 ptL, 3 ptL, 3.5 ptL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL, up to 30 μL.

In any of the methods provided herein, including Method 1 and certain combination methods that include quantitating amplified nucleic acid as provided herein, in some examples, the target gene of interest and/or the cell-free nucleic acid derived from the target gene of interest contains at least one zinc finger binding site. In examples, the zinc finger binding site contains a sequence of (CNN)x or (GNN)x repeats, where: a) x is the number of CNN or GNN repeats, and the number is at least 2; and b) N is A, G, C, or T. In some examples, x is 3 or more repeats. In examples, x is 3, 4, 5, 6, 7, 8, 9, 10, 11, 12, 13, 14, 15, 16 or 17 or more repeats. In some examples, x is between about 3 to about 10, 11, 12, 13, 14, 15, 16 or 17 repeats. In some examples, x is at least 3, 4, 5, 6, 7, 8, 9, 10, 11, 12, 13, 14, 15, 16 or 17 repeats. In any of the methods provided herein, including Method 1 and certain combination methods that include quantitating amplified nucleic acid as provided herein, in some examples, such as when quantitating by qPCR, the nucleic acid probe contains at least one zinc finger binding site. In examples, the nucleic acid probe contains a sequence of (CNN)x or (GNN)x repeats, where: a) x is the number of CNN or GNN repeats, and the number is at least 2; and b) N is A, G, C, or T. In some examples, x is 3 or more repeats. In examples, x is 3, 4, 5, 6, 7, 8, 9, 10, 11, 12, 13, 14, 15, 16 or 17 or more repeats. In some examples, x is between about 3 to about 10, or 3 to 10 repeats, inclusive, or, for example, 11, 12, 13, 14, 15, 16 or 17 repeats. In examples, x is at least 3, 4, 5, 6, 7, 8, 9, 10, 11, 12, 13, 14, 15, 16 or 17 repeats.

In any of the methods provided herein, including Method 1 and certain combination methods that include quantitating amplified nucleic acid as provided herein, in some examples, at least one target gene includes at least 2, 3, 4, 5, 6, 7, 8, 9 or 10 or more zinc finger binding sites. In any of the methods provided herein, including Method 1 and certain combination methods that include quantitating amplified nucleic acid as provided herein, in some examples, the target gene(s) is/are selected from among ESR1 (estrogen receptor 1), PGR (progesterone receptor), HER2 (human epidermal growth factor receptor 2), ARF (ADP ribosylation factors) family of proteins and regulators, COX1 (cyclooxygenase 1), COX11 (cytochrome c oxidase, mitochondrial), PLIN (perilipin) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9).

In any of the methods provided herein, including Method 1 and certain combination methods that include quantitating amplified nucleic acid as provided herein, in some examples, the target gene(s) is/are selected from among ESR1, PGR, HER2, ARFIP1, COX1, PLIN1, EGFR and MMP7. In some examples, the quantitating is by real-time-qPCR and the forward and reverse primer pairs, or the forward primer alone or the reverse primer alone, are selected, including in any permutation or combination, from among one or more of the groups of primers pairs having the following sequences (i)-(viii): (i) for the ESR1 gene, SEQ ID NO:2, or a sequence that has at least 95%, 96%, 97%, 98%, 99% or more identity with SEQ ID NO:2 and SEQ ID NO:3, or a sequence that has at least 95%, 96%, 97%, 98%, 99% or more identity with SEQ ID NO:3, (ii) for the PGR gene, SEQ ID NO:5, or a sequence that has at least 95%, 96%, 97%, 98%, 99% or more identity with SEQ ID NO:5 and SEQ ID NO:6, or a sequence that has at least 95%, 96%, 97%, 98%, 99% or more identity with SEQ ID NO:6, (iii) for the HER2 gene, SEQ ID NO:8, or a sequence that has at least 95%, 96%, 97%, 98%, 99% or more identity with SEQ ID NO:8 and SEQ ID NO:9, or a sequence that has at least 95%, 96%, 97%, 98%, 99% or more identity with SEQ ID NO:9, (iv) for the ARFIP1 gene, SEQ ID NO:11, or a sequence that has at least 95%, 96%, 97%, 98%, 99% or more identity with SEQ ID NO:11 and SEQ ID NO:12, or a sequence that has at least 95%, 96%, 97%, 98%, 99% or more identity with SEQ ID NO:12, (v) for the COX1 gene, SEQ ID NO:14, or a sequence that has at least 95%, 96%, 97%, 98%, 99% or more identity with SEQ ID NO:14 and SEQ ID NO:15, or a sequence that has at least 95%, 96%, 97%, 98%, 99% or more identity with SEQ ID NO:15, (vi) for the PLIN1 gene, SEQ ID NO:17, or a sequence that has at least 95%, 96%, 97%, 98%, 99% or more identity with SEQ ID NO:17 and SEQ ID NO:18, or a sequence that has at least 95%, 96%, 97%, 98%, 99% or more identity with SEQ ID NO:18, (vii) for the EGFR gene, SEQ ID NO:22, or a sequence that has at least 95%, 96%, 97%, 98%, 99% or more identity with SEQ ID NO:22 and SEQ ID NO:23, or a sequence that has at least 95%, 96%, 97%, 98%, 99% or more identity with SEQ ID NO:23; and (viii) for the MMP7 gene, SEQ ID NO:25, or a sequence that has at least 95%, 96%, 97%, 98%, 99% or more identity with SEQ ID NO:25 and SEQ ID NO:26, or a sequence that has at least 95%, 96%, 97%, 98%, 99% or more identity with SEQ ID NO:26; or the primer pairs, or the forward primer alone or the reverse primer alone, are selected, including in any permutation or combination, from among one or more of the primers pairs having the following sequences (i)-(viii): (i) for the ESR1 gene, SEQ ID NO:2 and SEQ ID NO:3, (ii) for the PGR gene, SEQ ID NO:5 and SEQ ID NO:6, (iii) for the HER2 gene, SEQ ID NO:8 and SEQ ID NO:9, (iv) for the ARFIP1 gene, SEQ ID NO:11 and SEQ ID NO:12, (v) for the COX1 gene, SEQ ID NO:14 and SEQ ID NO:15, (vi) for the PLIN1 gene, SEQ ID NO:17 and SEQ ID NO:18, (vii) for the EGFR gene, SEQ ID NO:22 and SEQ ID NO:23; and (viii) for the MMP7 gene, SEQ ID NO:25 and SEQ ID NO:26.

In some examples, the target gene(s) is/are selected from among ESR1, PGR, HER2, ARFIP1, COX1, PLIN1, EGFR and MMP7 and the nucleic acid probe(s) is/are selected, alone or including in any permutation or combination, from among one or more of the probes having the following sequences (i)-(viii): (i) for the ESR1 gene, SEQ ID NO:1, or a sequence that has at least 95%, 96%, 97%, 98%, 99% or more identity with SEQ ID NO:1, (ii) for the PGR gene, SEQ ID NO:4, or a sequence that has at least 95%, 96%, 97%, 98%, 99% or more identity with SEQ ID NO:4, (iii) for the HER2 gene, SEQ ID NO:7, or a sequence that has at least 95%, 96%, 97%, 98%, 99% or more identity with SEQ ID NO:7, (iv) for the ARFIP1 gene, SEQ ID NO:10, or a sequence that has at least 95%, 96%, 97%, 98%, 99% or more identity with SEQ ID NO:10, (v) for the COX1 gene, SEQ ID NO:13, or a sequence that has at least 95%, 96%, 97%, 98%, 99% or more identity with SEQ ID NO:13, (vi) for the PLIN1 gene, SEQ ID NO:16, or a sequence that has at least 95%, 96%, 97%, 98%, 99% or more identity with SEQ ID NO:16, (vii) for the EGFR gene, SEQ ID NO:19, or a sequence that has at least 95%, 96%, 97%, 98%, 99% or more identity with SEQ ID NO:19, SEQ ID NO:20, or a sequence that has at least 95%, 96%, 97%, 98%, 99% or more identity with SEQ ID NO:20 or SEQ ID NO:21, or a sequence that has at least 95%, 96%, 97%, 98%, 99% or more identity with SEQ ID NO:21; and (viii) for the MMP7 gene, SEQ ID NO:24, or a sequence that has at least 95%, 96%, 97%, 98%, 99% or more identity with SEQ ID NO:24; or is/are selected, alone or including in any permutation or combination, from among one or more of the probes having the following sequences (i)-(viii): (i) for the ESR1 gene, SEQ ID NO:1, (ii) for the PGR gene, SEQ ID NO:4, (iii) for the HER2 gene, SEQ ID NO:7, (iv) for the ARFIP1 gene, SEQ ID NO:10, (v) for the COX1 gene, SEQ ID NO:13, (vi) for the PLIN1 gene, SEQ ID NO:16, (vii) for the EGFR gene, SEQ ID NO: 19, SEQ ID NO:20, or SEQ ID NO:21; and (viii) for the MMP7 gene, SEQ ID NO:24.

In any of the methods provided herein, including Method 1 and combination methods that include quantitating amplified nucleic acid as provided herein, in some examples, such as when quantitating by qPCR, the nucleic acid probe is labelled with a detectable label to provide a signal that is proportional to or inversely proportional to the amount of amplification product. In some examples, the label is a chromophore, a chemiluminescent label, a radiolabel or a fluorescent label. In examples, the label is a fluorescent label. In some examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxyfluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,1-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. In some examples, the nucleic acid probe further contains a fluorescence quencher label. In examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ (Black Hole Quencher 1), BHQ-2 (Black Hole Quencher 2), BHQ-3 (Black Hole Quencher 3), DABSYL (dimethylamninoazobenzenesulfonic acid), Iowa Black FQ and Iowa Black RQ. In some examples, the fluorescent label is at the 5′-end of the nucleic acid probe and the fluorescence quencher is at the 3′-end of the nucleic acid probe. In examples, the fluorescent label at the 5′-end of the nucleic acid probe is FAM and the fluorescence quencher at the 3′-end of the nucleic acid probe is a Black Hole Quencher, such as BHQ-0, BHQ-1 (used interchangeably herein with BHQ), BHQ-2 or BHQ-3. In some examples, the Black Hole Quencher at the 3′-end of the nucleic acid probe is BHQ. Suitable pairs of fluorescent “reporter” molecules and fluorescence quenchers are known to those of skill in the art. It is understood that a molecule that is selected to be a fluorescent reporter molecule (e.g., 5-TAMRA) cannot also serve as a fluorescence quenching pair to the same reporter molecule.

In any of the methods provided herein, including Method 1 and certain combination methods that include quantitating amplified nucleic acid as provided herein, in some examples, cell-free nucleic acid derived from at least 2, 3, 4, 5, 6, 7 or 8 or more target genes is analyzed. In some examples, if cell-free nucleic acid derived from at 1, at least 2, or at least half or more of the target genes analyzed is indicative of the presence of the disease or condition, the subject is identified as having the disease or condition.

In any of the methods provided herein, including Method 1 and certain combination methods that include quantitating amplified nucleic acid as provided herein, in some examples, the disease or condition is cancer, and at least one target gene is an oncogene, or a gene associated with cancer.

Also provided herein is a method, termed “Method 2A” herein, for analyzing cell-free nuclease activity in a body fluid from a subject as indicative of the presence or absence of a disease or condition in the subject, by: a) procuring a first aliquot from the body fluid, wherein the body fluid was previously obtained from the subject; b) to the first aliquot, adding Zn²⁺, Mg²⁺ and a nucleic acid probe; c) subjecting b) to reaction conditions under which nuclease activity, if present in the body fluid, is determined by measuring digestion of the nucleic acid probe; d) procuring a second aliquot from the previously obtained body fluid, wherein the second aliquot is of the same or similar amount as the first aliquot; e) to the second aliquot, not adding Zn²⁺ and adding Mg²⁺ and the same nucleic acid probe that is used in b); f) subjecting e) to reaction conditions under which nuclease activity, if present in the body fluid, is determined by measuring digestion of the nucleic acid probe; and g) obtaining a ratio of the nuclease activity determined in c) to the nuclease activity determined in f), where: i) if the ratio is at or above a threshold level, the subject is identified as having the disease or condition; and ii) if the ratio is below a threshold level, the subject is identified as not having the disease or condition. In some examples, the first aliquot and the second aliquot are subjected to reaction conditions for determining nuclease activity sequentially, simultaneously or in any order. In examples, the threshold level is determined based on the ratio that is measured in at least one subject known to have the disease or condition and/or is based on the ratio that is measured in at least one control or reference sample. In some examples, the control or reference sample is an analogous sample from a normal subject or a subject who does not have the disease or condition. In some examples, the threshold level is determined based on a ratio that is the mean or the median of ratios measured in more than one subject known to have the disease or condition and/or the mean or the median of ratios measured in more than one control or reference sample. In examples, the threshold level is determined by obtaining a receiver/relative operating characteristic curve (ROC curve) created by plotting Sensitivity against Specificity and determining an optimal cutoff value that provides a desired Sensitivity and a desired Specificity, wherein the optimal cutoff value is assigned as the threshold level. In some examples, the optimal cutoff value is a ratio at which or at about which the ROC curve plateaus. In examples, the optimal cutoff value is determined by the cutoff value that yields the maximum value of J (Youden's index)=Sensitivity+Specificity−1.

In any of the examples of Method 2A provided herein, including Method 2A and certain combination methods that include measuring nuclease activity as provided herein, in some examples, the threshold level is about or at 1.3; if the ratio is at or greater than 1.3, the subject is identified as having the disease or condition; and if the ratio is less than 1.3, the subject is identified as not having the disease or condition. In some examples, the threshold level for identifying a subject as having the disease or condition is a ratio that is ≥1.2, ≥1.3, ≥1.4 or ≥1.5. In such examples, the threshold level for identifying a subject as not having the disease or condition is a ratio that is <1.2, <1.3, <1.4 or <1.5.

In any of the examples of Method 2A provided herein, including Method 2A and certain combination methods that include measuring nuclease activity as provided herein, in some examples, the Mg²⁺ concentration is between about 3 mM to about 20 mM, or 3 mM to 20 mM, inclusive. In examples, the Mg²⁺ concentration is at least about 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM, up to 18 mM, up to 20 mM. In some examples, the Mg²⁺ concentration is between about 5 mM to about 7 mM or 5 mM to 7 mM, inclusive. In examples, the Mg²⁺ concentration is at least about 6 mM, 6.1 mM, 6.2 mM, 6.3 mM, 6.4 mM, up to 6.5 mM.

In any of the examples of Method 2A provided herein, including Method 2A and certain combination methods that include measuring nuclease activity as provided herein, in some examples, the Zn²⁺ concentration is between about 5 mM to about 15 mM, or 5 mM to 15 mM, inclusive. In examples, the Zn²⁺ concentration is at least about 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, up to 15 mM. In some examples, the Zn²⁺ concentration is between about 6 mM to about 10 mM, or 6 mM to 10 mM, inclusive. In some examples, the Zn²⁺ concentration is between about 6 mM to about 7 mM, or 6 mM to 7 mM, inclusive. In examples, the Zn²⁺ concentration is about 9 mM. It was found that Zn²⁺ concentration can be as low as 1 μM, generally 10 μM to 50 μM, such as 25 μM, in Methods 2 in which zinc is added.

In any of the examples of Method 2A provided herein, including Method 2A and certain combination methods that include measuring nuclease activity as provided herein, in some examples, the nuclease activity is determined by measuring a signal that is proportional to the activity or is inversely proportional to the activity. In examples, the signal is fluorescence.

In any of the examples of Method 2A provided herein, including Method 2A and certain combination methods that include measuring nuclease activity as provided herein, in some examples, the nucleic acid probe contains at least one zinc finger binding site. In examples, the nucleic acid probe contains a sequence of (CNN)x or (GNN)x repeats, where: a) x is the number of CNN or GNN repeats, and the number is at least 2; and b) N is A, G, C, or T. In some examples, x is 3 or more repeats. In examples, x is 3, 4, 5, 6, 7, 8, 9, 10, 11, 12, 13, 14, 15, 16 or 17 or more repeats. In some examples, x is between about 3 to about 10, 11, 12, 13, 14, 15, 16 or 17 repeats, or between 3 to 17 repeats. In some examples, x is at least 3, 4, 5, 6, 7, 8, 9, 10, 11, 12, 13, 14, 15, 16, or 17 repeats.

In any of the examples of Method 2A provided herein, including Method 2A and certain combination methods that include measuring nuclease activity as provided herein, in some examples, the nucleic acid probe contains more than one zinc finger binding site.

In any of the examples of Method 2A provided herein, including Method 2A and certain combination methods that include measuring nuclease activity as provided herein, in some examples, the nucleic acid probe contains a sequence that is derived from a target gene in the subject, or is a sequence that is at least 95%, 96%, 97%, 98%, 99% or more identical to or complementary to a sequence derived from a target gene in the subject.

In any of the examples of Method 2A provided herein, including Method 2A and certain combination methods that include measuring nuclease activity as provided herein, in some examples, the volume of body fluid analyzed is between about 1 μL to about 1 mL. In some examples, the volume of body fluid analyzed is 100 μL or less. In examples, the volume of body fluid analyzed is between about 5 μL to about 50 μL. In some examples, the volume of body fluid analyzed is between about 2 μL to about 25 μL, or between about 1 μL to about 20 μL. In examples, the volume of body fluid analyzed is about or at least about 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL, up to 30 μL.

In any of the examples of Method 2A provided herein, including Method 2A and certain combination methods that include measuring nuclease activity as provided herein, in some examples, the disease or condition is cancer, a non-cancerous proliferative disorder or an infectious disease caused by a pathogen. In some examples, the disease or condition is cancer or a non-cancerous proliferative disorder. In examples, the cancer is selected from among an early-stage cancer, a late-stage cancer, a metastatic cancer, and/or an undifferentiated cancer. In some examples, the cancer or non-cancerous proliferative disorder is selected from among ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, a chordoma, a chromophobe renal cell carcinoma, a clear cell carcinoma, colon cancer, colorectal cancer, cutaneous benign fibrous histiocytoma, fibroadenoma, fibrocystic disease, desmoplastic small round cell tumor, ependymoma, Ewing's tumor, extraskeletal myxoid chondrosarcoma, fibrogenesis imperfecta ossium, fibrous dysplasia of the bone, gallbladder cancer, bile duct cancer, gastric cancer, synovial tissue cancer, gestational trophoblastic disease, germ cell tumor, glioblastoma, hematological malignancy, hepatocellular carcinoma, islet cell tumor, Kaposi's sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, myelodysplastic syndrome, neuroblastoma, neuroendocrine tumors, papillary thyroid carcinoma, parathyroid tumor, pediatric cancer, peripheral nerve sheath tumor, pheochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, head and neck squamous cell cancer (HNSCC), stomach cancer, synovial tissue cancer, synovial sarcoma, testicular cancer, thymic carcinoma, thymoma, a thyroid cancer, thyroid metastatic cancer, uterine cancer, hepatoma, glioma, retinoblastoma, myeloma, lymphoma, and leukemia. In examples, the cancer or non-cancerous proliferative disorder is of the breast. In some examples, the cancer or non-cancerous proliferative disorder is selected from among invasive ductal carcinoma breast cancer, luminal A breast cancer, luminal B breast cancer, HER2-enriched breast cancer, triple negative breast cancer, fibroadenoma and fibrocystic disease. In some examples, the disease or condition is cancer, and the cancer is triple negative breast cancer.

In any of the examples of Method 2A provided herein, including Method 2A and certain combination methods that include measuring nuclease activity as provided herein, in some examples, the body fluid is whole blood, urine, plasma, serum, cerebrospinal fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In examples, the body fluid is plasma.

In any of the examples of Method 2A provided herein, including Method 2A and certain combination methods that include measuring nuclease activity as provided herein, in some examples, the nucleic acid probe sequence is derived from a target gene and the target gene(s) is/are selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). In examples, the target gene(s) is/are selected from among ESR1, PGR, HER2, ARFIP1, COX1, PLIN1, EGFR, MMP7, MED12, RFX2, TERT, P21, P13 KB, SEPT9 and P53. In some examples, the nucleic acid probe(s) is/are selected, alone or including in any permutation or combination, from among one or more of the probes having the following sequences (i)-(xviii): (i) SEQ ID NO:1, or a sequence that has at least 95%, 96%, 97%, 98%, 99% or more identity with SEQ ID NO:1, (ii) SEQ ID NO:4, or a sequence that has at least 95%, 96%, 97%, 98%, 99% or more identity with SEQ ID NO:4, (iii) SEQ ID NO:7, or a sequence that has at least 95%, 96%, 97%, 98%, 99% or more identity with SEQ ID NO:7, (iv) SEQ ID NO:10, or a sequence that has at least 95%, 96%, 97%, 98%, 99% or more identity with SEQ ID NO:10, (v) SEQ ID NO:13, or a sequence that has at least 95%, 96%, 97%, 98%, 99% or more identity with SEQ ID NO:13, (vi) SEQ ID NO:16, or a sequence that has at least 95%, 96%, 97%, 98%, 99% or more identity with SEQ ID NO:16, (vii) SEQ ID NO:19, or a sequence that has at least 95%, 96%, 97%, 98%, 99% or more identity with SEQ ID NO:19, (viii) SEQ ID NO:20, or a sequence that has at least 95%, 96%, 97%, 98%, 99% or more identity with SEQ ID NO:20, (ix) SEQ ID NO:21, or a sequence that has at least 95%, 96%, 97%, 98%, 99% or more identity with SEQ ID NO:21, (x) SEQ ID NO:24, or a sequence that has at least 95%, 96%, 97%, 98%, 99% or more identity with SEQ ID NO:24, (xi) SEQ ID NO:29, or a sequence that has at least 95%, 96%, 97%, 98%, 99% or more identity with SEQ ID NO:29, (xii) SEQ ID NO:30, or a sequence that has at least 95%, 96%, 97%, 98%, 99% or more identity with SEQ ID NO:30, (xiii) SEQ ID NO:27, or a sequence that has at least 95%, 96%, 97%, 98%, 99% or more identity with SEQ ID NO:27, (xiv) SEQ ID NO:31, or a sequence that has at least 95%, 96%, 97%, 98%, 99% or more identity with SEQ ID NO:31, (xv) for the P13 KB gene, SEQ ID NO:32, or a sequence that has at least 95%, 96%, 97%, 98%, 99% or more identity with SEQ ID NO:32, (xvi) SEQ ID NO:33, or a sequence that has at least 95%, 96%, 97%, 98%, 99% or more identity with SEQ ID NO:33; (xvii) SEQ ID NO:28, or a sequence that has at least 95%, 96%, 97%, 98%, 99% or more identity with SEQ ID NO:28; and (xviii) SEQ ID NO:50, or a sequence that has at least 95%, 96%, 97%, 98%, 99% or more identity with SEQ ID NO:50, and/or the nucleic acid probe(s) is/are selected, alone or including in any permutation or combination, from among one or more of the probes having the following sequences (i)-(xviii): (i) SEQ ID NO:1, (ii) SEQ ID NO:4, (iii) SEQ ID NO:7, (iv) SEQ ID NO:10, (v) SEQ ID NO:13, (vi) SEQ ID NO:16, (vii) SEQ ID NO:19, (viii) SEQ ID NO: 20, (ix) SEQ ID NO:21, (x) SEQ ID NO:24, (xi) SEQ ID NO:29, (xii) SEQ ID NO:30, (xiii) SEQ ID NO:27, (xiv) SEQ ID NO:31, (xv) SEQ ID NO:32, (xvi) SEQ ID NO:33; (xvii) SEQ ID NO:28; and (xviii) SEQ ID NO:50.

In examples of Method 2A provided herein, including Method 2A and certain combination methods that include measuring nuclease activity as provided herein, in some examples, the disease or condition is an infection caused by a pathogen. In examples, the pathogen is a virus. In some examples, the virus is SARS-CoV-2. In examples, the nucleic acid probe(s) is/are selected, alone or including in any permutation or combination, from among probes having one or more of the following sequences (i)-(iii): (i) SEQ ID NO:34, or a sequence that has at least 95%, 96%, 97%, 98%, 99% or more identity with SEQ ID NO:34, (ii) SEQ ID NO:35, or a sequence that has at least 95%, 96%, 97%, 98%, 99% or more identity with SEQ ID NO:35, and (iii) SEQ ID NO:36, or a sequence that has at least 95%, 96%, 97%, 98%, 99% or more identity with SEQ ID NO:36 and/or the nucleic acid probe(s) is/are selected, alone or including in any permutation or combination, from among probes having one or more of the following sequences (i)-(iii): (i) SEQ ID NO:34, (ii) SEQ ID NO:35, and (iii) SEQ ID NO:36.

In any of the examples of Method 2A provided herein, including Method 2A and certain combination methods that include measuring nuclease activity as provided herein, in some examples, the nucleic acid probe is labelled with a detectable label to provide a signal that is proportional to or inversely proportional to the amount of amplification product. In examples, the label is a chromophore, a chemiluminescent label, a radiolabel or a fluorescent label. In some examples, the label is a fluorescent label. In examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′,5′-dichloro-2′,7′-dimethoxyfluorescein), 6-JOE SE (6-carboxy-4′5′-dichloro-2′,7′-dimethoxyfluorescein, succinimidyl ester), 6-JOE NHS (6-carboxy-4′5′-dichloro-2′,7′-dimethoxyfluorescein, N-hydroxysuccinimide ester), HEX (hexachloro-6-carboxyfluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. In some examples, the nucleic acid probe further includes a fluorescence quencher label. In examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ (Black Hole Quencher 1), BHQ-2 (Black Hole Quencher 2), BHQ-3 (Black Hole Quencher 3), DABSYL (dimethylaminoazobenzene sulfonic acid), Iowa Black FQ and Iowa Black RQ. In some examples, the fluorescent label is at the 5′-end of the nucleic acid probe and the fluorescence quencher is at the 3′-end of the nucleic acid probe. In examples, the fluorescent label at the 5′-end of the nucleic acid probe is FAM and the fluorescence quencher at the 3′-end of the nucleic acid probe is BHQ. It is understood that a molecule that is selected to be a fluorescent reporter molecule (e.g., 5-TAMRA) cannot also serve as a fluorescence quenching pair to the same reporter molecule.

In any of the examples of Method 2A provided herein, including Method 2A and certain combination methods that include measuring nuclease activity as provided herein, in some examples, at least 2, 3, 4, 5, 6, 7 or 8 or more nucleic acid probes are analyzed. In some examples, the threshold level is determined by determining an optimal cutoff value, and the optimal cutoff value is measured by obtaining an ROC curve for a sum of the ratios of nuclease activities measured for each nucleic acid probe. In some examples, if at least 1, at least 2, or at least half or more of the nucleic acid probes analyzed is/are indicative of the presence of the disease or condition, the subject is identified as having the disease or condition. In examples, the optimal cutoff value is measured by obtaining an ROC curve for a mean or a median of the ratios of nuclease activities measured for each nucleic acid probe.

In any of the examples of Method 2A provided herein, including Method 2A and certain combination methods that include measuring nuclease activity as provided herein, in some examples, the reaction conditions further include adding a reducing agent. In examples, the reducing agent is selected from among TCEP (tris(2-carboxyethyl) phosphine), DTT, DTE, glutathione, N-acetylcysteine, 2-mercaptoethanol, cysteine hydrochloride, sodium thioglycolate, diethyldithiocarbamate, thioglycolic acid and DTBA (dithiobutylamine). In some examples, the reducing agent is DTT. In examples, the reducing agent is added in an amount that increases the difference between the ratio of nuclease activities measured in a subject having the disease or condition and a subject not having the disease or condition. In some examples, the concentration of the reducing agent is between about 0.1 mM to about 2 mM, or 0.1 mM to 2 mM, inclusive. In examples, the concentration of the reducing agent is between about 0.1 mM to about 1 mM or 1.5 mM, or 0.1 mM to 1 or 1.5 mM, inclusive. In some examples, the concentration of the reducing agent is between about 0.1 mM to about 0.6 mM, or 0.1 mM to 0.6 mM, inclusive. In examples, the concentration of the reducing agent is about or equal to at least 0.1 mM, 0.2 mM, 0.3 mM, 0.4 mM, up to 0.5 mM. In some examples, the concentration of the reducing agent is about or equal to 0.4 mM, or 0.3 mM to 0.5 mM. In some examples, the concentration of the reducing agent is about or equal to 0.5 mM.

In any of the examples of Method 2A provided herein, including Method 2A and certain combination methods that include measuring nuclease activity as provided herein, in some examples, the reaction conditions further include adding a detergent. In examples, the detergent is NP40.

Also provided herein is a method, termed “Method 2B” herein, for analyzing cell-free nuclease activity in a body fluid from a subject as indicative of the presence or absence of a disease or condition in the subject by performing the following: a) to a previously obtained sample from the subject, adding Mg²⁺ and a nucleic acid probe containing at least one zinc finger binding motif, b) subjecting a) to reaction conditions under which nuclease activity, if present in the body fluid, is determined by measuring digestion of the nucleic acid probe; c) subjecting a reference or control sample to the same or similar reaction conditions as b) under which nuclease activity, if present in the reference or control sample, is determined by measuring digestion of the nucleic acid probe, or obtaining a predetermined value of nuclease activity from a control or reference sample; and d) obtaining a ratio of the nuclease activity determined in b) to the predetermined nuclease activity or nuclease activity that is determined in c), where: i) if the ratio is at or above a threshold level, the subject is identified as having the disease or condition; and ii) if the ratio is below a threshold level, the subject is identified as not having the disease or condition. In some examples, if both the subject and the reference or control are subjected to reaction conditions under which nuclease activity is measured, the reactions are performed sequentially, simultaneously or in any order. In examples, the threshold level is determined based on the ratio that is measured in at least one subject known to have the disease or condition and/or is based on the ratio that is measured in at least one control or reference sample. In some examples, the control or reference sample is an analogous sample from a normal subject or a subject who does not have the disease or condition. In some examples, the threshold level is determined based on a ratio that is the mean or the median of ratios measured in more than one subject known to have the disease or condition and/or the mean or the median of ratios measured in more than one control or reference sample. In examples, the threshold level is determined by obtaining a receiver/relative operating characteristic curve (ROC curve) created by plotting Sensitivity against Specificity and determining an optimal cutoff value that provides a desired Sensitivity and a desired Specificity, wherein the optimal cutoff value is assigned as the threshold level. In some examples, the optimal cutoff value is a ratio at which or at about which the ROC curve plateaus. In examples, the optimal cutoff value is determined by the cutoff value that yields the maximum value of J (Youden's index)=Sensitivity+Specificity−1.

In any of the examples of Method 2B provided herein, including Method 2B and certain combination methods that include measuring nuclease activity as provided herein, in some examples: the threshold level is a ratio that is 1.0; if the ratio is greater than 1.0, the subject is identified as having the disease or condition; and if the ratio is less than 1.0 or less, the subject is identified as not having the disease or condition. In any of the examples of Method 2B provided herein, including Method 2B and certain combination methods that include measuring nuclease activity as provided herein, in some examples: the threshold level is a ratio that is 1.3; if the ratio is at or greater than 1.3, the subject is identified as having the disease or condition; and if the ratio is less than 1.3, the subject is identified as not having the disease or condition. In some other examples, the threshold level for identifying a subject as having the disease or condition is a ratio that is ≥1.2, ≥1.3, ≥1.4 or ≥1.5. In examples, the threshold level for identifying a subject as not having the disease or condition is a ratio that is 1.0 or less, <1.1, <1.2, <1.3, <1.4 or <1.5.

In any of the examples of Method 2B provided herein, including Method 2B and certain combination methods that include measuring nuclease activity as provided herein, in some examples, the Mg²⁺ concentration is between about 3 mM to about 20 mM, or 3 mM to 20 mM, inclusive. In some examples, the Mg²⁺ concentration is about at least 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM, up to 18 mM or 20 mM. In examples, the Mg²⁺ concentration is between about 5 mM to about 7 mM. In some examples, the Mg²⁺ concentration is about at least 6 mM, 6.1 mM, 6.2 mM, 6.3 mM, 6.4 mM, up to 6.5 mM or 7 mM.

In any of the examples of Method 2B provided herein, including Method 2B and certain combination methods that include measuring nuclease activity as provided herein, in some examples, the nuclease activity is determined by measuring a signal that is proportional to the activity or is inversely proportional to the activity. In examples, the signal is fluorescence.

In any of the examples of Method 2B provided herein, including Method 2B and certain combination methods that include measuring nuclease activity as provided herein, in some examples, the nucleic acid probe contains a sequence of (CNN)x or (GNN)x repeats, where: a) x is the number of CNN or GNN repeats, and the number is at least 2; and b) N is A, G, C, or T. In some examples, x is 3 or more repeats. In examples, x is 3, 4, 5, 6, 7, 8, 9, 10, 11, 12, 13, 14, 15, 16, or 17 or more repeats, or 3 to 17 repeats. In some examples, x is between about 3 to about 10, 11, 12, 13, 14, 15, 16, or 17 repeats. In some examples, x is at least 3, 4, 5, 6, 7, 8, 9, 10, 11, 12, 13, 14, 15, 16, or 17 repeats.

In any of the examples of Method 2B provided herein, including Method 2B and certain combination methods that include measuring nuclease activity as provided herein, in some examples, the nucleic acid probe contains more than one zinc finger binding motif. In any of the examples of Method 2B provided herein, including Method 2B and certain combination methods that include measuring nuclease activity as provided herein, in some examples, the nucleic acid probe includes a sequence that is derived from a target gene in the subject, or is a sequence that is at least 95%, 96%, 97%, 98%, 99% or more identical to or complementary to a sequence derived from a target gene in the subject.

In any of the examples of Method 2B provided herein, including Method 2B and certain combination methods that include measuring nuclease activity as provided herein, in some examples, the volume of body fluid analyzed is between about 1 μL to about 1 mL. In examples, the volume of body fluid analyzed is 100 μL or less. In some examples, the volume of body fluid analyzed is between about 5 μL to about 50 μL. In examples, the volume of body fluid analyzed is between about 2 μL to about 25 μL, or between about 1 μL to about 20 μL. In some examples, the volume of body fluid analyzed is about or at least about 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL or 30 μL.

In any of the examples of Method 2B provided herein, including Method 2B and certain combination methods that include measuring nuclease activity as provided herein, in some examples, in some examples, the disease or condition is cancer, a non-cancerous proliferative disorder or an infectious disease caused by a pathogen. In some examples, the disease or condition is cancer or a non-cancerous proliferative disorder. In examples, the cancer is selected from among an early-stage cancer, a late-stage cancer, a metastatic cancer, and/or an undifferentiated cancer. In some examples, the cancer or non-cancerous proliferative disorder is selected from among ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, a chordoma, a chromophobe renal cell carcinoma, a clear cell carcinoma, colon cancer, colorectal cancer, cutaneous benign fibrous histiocytoma, fibroadenoma, fibrocystic disease, desmoplastic small round cell tumor, ependymoma, Ewing's tumor, extraskeletal myxoid chondrosarcoma, fibrogenesis imperfecta ossium, fibrous dysplasia of the bone, gallbladder cancer, bile duct cancer, gastric cancer, synovial tissue cancer, gestational trophoblastic disease, germ cell tumor, glioblastoma, hematological malignancy, hepatocellular carcinoma, islet cell tumor, Kaposi's Sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, myelodysplastic syndrome, neuroblastoma, neuroendocrine tumors, papillary thyroid carcinoma, parathyroid tumor, pediatric cancer, peripheral nerve sheath tumor, phaeochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, squamous cell cancer of the head and neck (SCCHN), stomach cancer, synovial tissue cancer, synovial sarcoma, testicular cancer, thymic carcinoma, thymoma, a thyroid cancer, thyroid metastatic cancer, uterine cancer, hepatoma, glioma, retinoblastoma, myeloma, lymphoma, and leukemia. In examples, the cancer or non-cancerous proliferative disorder is of the breast. In some examples, the cancer or non-cancerous proliferative disorder is selected from among invasive ductal carcinoma breast cancer, luminal A breast cancer, luminal B breast cancer, HER2-enriched breast cancer, triple negative breast cancer, fibroadenoma, and fibrocystic disease. In examples, the disease or condition is cancer, and the cancer is triple negative breast cancer.

In any of the examples of Method 2B provided herein, including Method 2B and certain combination methods that include measuring nuclease activity as provided herein, in some examples, the body fluid is whole blood, urine, plasma, serum, cerebrospinal fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In some examples, the body fluid is plasma.

In examples of Method 2B provided herein, including Method 2B and certain combination methods that include measuring nuclease activity as provided herein, in some examples, the nucleic acid probe has a sequence derived from that of a target gene that is/are selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). In examples, the target gene(s) is/are selected from among ESR1, PGR, HER2, ARFIP1, COX1, PLIN1, EGFR, MMP7, MED12, RFX2, TERT, P21, P13 KB, SEPT9 and P53.

In any of the examples of Method 2B provided herein, including Method 2B and certain combination methods that include measuring nuclease activity as provided herein, in some examples, the nucleic acid probe(s) is/are selected, alone or including in any permutation or combination, from among from among one or more of the probes having the following sequences (i)-(xviii): (i) SEQ ID NO:1, or a sequence that has at least 95%, 96%, 97%, 98%, 99% or more identity with SEQ ID NO:1, (ii) SEQ ID NO:4, or a sequence that has at least 95%, 96%, 97%, 98%, 99% or more identity with SEQ ID NO:4, (iii) SEQ ID NO:7, or a sequence that has at least 95%, 96%, 97%, 98%, 99% or more identity with SEQ ID NO:7, (iv) SEQ ID NO:10, or a sequence that has at least 95%, 96%, 97%, 98%, 99% or more identity with SEQ ID NO:10, (v) SEQ ID NO:13, or a sequence that has at least 95%, 96%, 97%, 98%, 99% or more identity with SEQ ID NO:13, (vi) SEQ ID NO:16, or a sequence that has at least 95%, 96%, 97%, 98%, 99% or more identity with SEQ ID NO:16, (vii) SEQ ID NO:19, or a sequence that has at least 95%, 96%, 97%, 98%, 99% or more identity with SEQ ID NO:19, (viii) SEQ ID NO:20, or a sequence that has at least 95%, 96%, 97%, 98%, 99% or more identity with SEQ ID NO:20, (ix) SEQ ID NO:21, or a sequence that has at least 95%, 96%, 97%, 98%, 99% or more identity with SEQ ID NO:21, (x) SEQ ID NO:24, or a sequence that has at least 95%, 96%, 97%, 98%, 99% or more identity with SEQ ID NO:24, (xi) SEQ ID NO:29, or a sequence that has at least 95%, 96%, 97%, 98%, 99% or more identity with SEQ ID NO:29, (xii) SEQ ID NO:30, or a sequence that has at least 95%, 96%, 97%, 98%, 99% or more identity with SEQ ID NO:30, (xiii) SEQ ID NO:27, or a sequence that has at least 95%, 96%, 97%, 98%, 99% or more identity with SEQ ID NO:27, (xiv) SEQ ID NO:31, or a sequence that has at least 95%, 96%, 97%, 98%, 99% or more identity with SEQ ID NO:31, (xv) for the P13 KB gene, SEQ ID NO:32, or a sequence that has at least 95%, 96%, 97%, 98%, 99% or more identity with SEQ ID NO:32, (xvi) SEQ ID NO:33, or a sequence that has at least 95%, 96%, 97%, 98%, 99% or more identity with SEQ ID NO:33; (xvii) SEQ ID NO:28, or a sequence that has at least 95%, 96%, 97%, 98%, 99% or more identity with SEQ ID NO:28; and (xviii) SEQ ID NO:50, or a sequence that has at least 95%, 96%, 97%, 98%, 99% or more identity with SEQ ID NO:50; and/or the nucleic acid probe(s) is/are selected, alone or in any permutation or combination, from among one or more of the probes having the following sequences (i)-(xviii): (i) SEQ ID NO:1, (ii) SEQ ID NO:4, (iii) SEQ ID NO:7, (iv) SEQ ID NO:10, (v) SEQ ID NO:13, (vi) SEQ ID NO:16, (vii) SEQ ID NO:19, (viii) SEQ ID NO: 20, (ix) SEQ ID NO:21, (x) SEQ ID NO:24, (xi) SEQ ID NO:29, (xii) SEQ ID NO:30, (xiii) SEQ ID NO:27, (xiv) SEQ ID NO:31, (xv) SEQ ID NO:32, (xvi) SEQ ID NO:33; (xvii) SEQ ID NO:28; and (xviii) SEQ ID NO:50, or a sequence that has at least 95%, 96%, 97%, 98%, 99% or more identity with SEQ ID NO:50.

In any of the examples of Method 2B provided herein, including Method 2B and certain combination methods that include measuring nuclease activity as provided herein, the disease or condition is an infection caused by a pathogen. In some examples, the pathogen is a virus. In examples, the virus is SARS-CoV-2. In some examples, the nucleic acid probe(s) is/are selected, alone or including in any permutation or combination, from among probes having one or more of the following sequences (i)-(iii): (i) SEQ ID NO:34, or a sequence that has at least 95%, 96%, 97%, 98%, 99% or more identity with SEQ ID NO:34, (ii) SEQ ID NO:35, or a sequence that has at least 95%, 96%, 97%, 98%, 99% or more identity with SEQ ID NO:35, and (iii) SEQ ID NO:36, or a sequence that has at least 95%, 96%, 97%, 98%, 99% or more identity with SEQ ID NO:36 and/or the nucleic acid probe(s) is/are selected, alone or including in any permutation or combination, from among probes having one or more of the following sequences (i)-(iii): (i) SEQ ID NO:34, (ii) SEQ ID NO:35, and (iii) SEQ ID NO:36.

In any of the examples of Method 2B provided herein, including Method 2B and certain combination methods that include measuring nuclease activity as provided herein, in examples, the nucleic acid probe is labelled with a detectable label to provide a signal that is proportional to or inversely proportional to the amount of amplification product. In some examples, the label is a chromophore, a chemiluminescent label, a radiolabel or a fluorescent label. In examples, the label is a fluorescent label. In some examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. In examples, the nucleic acid probe further contains a fluorescence quencher label. In some examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ-1 (Black Hole Quencher 1), BHQ-2 (Black Hole Quencher 2), BHQ-3 (Black Hole Quencher 3), DABSYL (dimethylaminoazobenzenesulfonic acid), Iowa Black FQ and Iowa Black RQ. In examples, the fluorescent label is at the 5′-end of the nucleic acid probe and the fluorescence quencher is at the 3′-end of the nucleic acid probe. In some examples, the fluorescent label at the 5′-end of the nucleic acid probe is FAM and the fluorescence quencher at the 3′-end of the nucleic acid probe is BHQ. It is understood that a molecule that is selected to be a fluorescent reporter molecule (e.g., 5-TAMRA) cannot also serve as a fluorescence quenching pair to the same reporter molecule.

In any of the examples of Method 2B provided herein, including Method 2B and certain combination methods that include measuring nuclease activity as provided herein, in some examples, at least 2, 3, 4, 5, 6, 7 or 8 or more nucleic acid probes are analyzed. In examples, the optimal cutoff value is measured by obtaining an ROC curve for a sum of the ratios of nuclease activities measured for each nucleic acid probe. In some examples, if at least 1, at least 2, or at least half or more of the nucleic acid probes analyzed is/are indicative of the presence of the disease or condition, the subject is identified as having the disease or condition. In examples, the optimal cutoff value is measured by obtaining an ROC curve for a mean or a median of the ratios of nuclease activities measured for each nucleic acid probe.

In any of the examples of Method 2B provided herein, including Method 2B and certain combination methods that include measuring nuclease activity as provided herein, the reaction conditions further include adding a reducing agent. In some examples, the reducing agent is selected from among TCEP (tris(2-carboxyethyl) phosphine), DTT, DTE, glutathione, N-acetylcysteine, 2-mercaptoethanol, cysteine hydrochloride, sodium thioglycolate, diethyldithiocarbamate, thioglycolic acid and DTBA (dithiobutylamine). In examples, the reducing agent is DTT. In some examples, the reducing agent is added in an amount that increases the difference between the ratio of nuclease activities measured in a subject having the disease or condition and a subject not having the disease or condition. In examples, the concentration of the reducing agent is between about 0.1 mM to about 2 mM, or 0.1 mM to 2 mM, inclusive. In examples, the concentration of the reducing agent is between about 0.1 mM to about 1 mM or 1.5 mM, or 0.1 mM to 1 or 1.5 mM. In some examples, the concentration of the reducing agent is between about 0.1 mM to about 0.6 mM, or 0.1 mM to 0.6 mM, inclusive. In examples, the concentration of the reducing agent is about or equal to at least 0.1 mM, 0.2 mM, 0.3 mM, 0.4 mM, up to 0.5 mM. In examples, the concentration of the reducing agent is about or equal to 0.4 mM, or 0.3 mM to 5 mM. In examples, the concentration of the reducing agent is about or equal to 0.5 mM.

In any of the examples of Method 2B provided herein, including Method 2B and certain combination methods that include measuring nuclease activity as provided herein, the disease or condition is cancer, and at least one target gene is an oncogene, or any gene associated with cancer.

In any of the examples of Method 2B provided herein, including Method 2B and certain combination methods that include measuring nuclease activity as provided herein, the reaction conditions further include adding Zn²⁺. In examples, the Zn²⁺ concentration is between about 5 mM to about 15 mM, or 5 mM to 15 mM, inclusive. In some examples, the Zn²⁺ concentration is at least about 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, up to 15 mM, inclusive. In some examples, the Zn²⁺ concentration is between about 6 mM to about 10 mM, or 6 mM to 10 mM. In examples, the Zn²⁺ concentration is between about 6 mM to about 7 mM, or 6 mM to 7 mM, inclusive. In some examples, the Zn²⁺ concentration is about 9 mM, or 8 to 10 mM.

Also provided herein are combination methods of analyzing a body fluid from a subject as indicative of the presence or absence of a disease or condition in the subject using more than one modality, where the methods include: a) subjecting a first aliquot of a sample of body fluid previously obtained from the subject to an amplification reaction, where the sample contains cell-free nucleic acid; b) quantitating the amplified nucleic acid from a), and determining whether the amount of amplified nucleic acid is at or above a threshold level, or below a threshold level; c) subjecting a second aliquot of the same sample of body fluid to a reaction to determine an enzymatic activity; d) determining whether the enzymatic activity in c) is at or above a threshold level, or below a threshold level; and e) if either or both of the following conditions are met: i) the amount of amplified nucleic acid is at or above a threshold level, and/or ii) the enzymatic activity is at or above a threshold level, identifying the subject as having the disease or condition. In examples, the enzymatic activity is a kinase, a protease or a nuclease activity. In some examples, the enzymatic activity is a nuclease activity. In some examples, the quantitating in b) is by gel electrophoresis, Picogreen assay, UV spectrometry, real-time quantitative PCR (real-time-qPCR), droplet digital PCR (ddPCR), BEAMing or TAm-Seq. In some examples, in b): the threshold level is determined based on a level that is measured in at least one subject known to have the disease or condition and/or is based on a level that is measured in at least one control or reference sample; or the threshold level is determined based on a level that is the mean or the median of levels measured in more than one subject known to have the disease or condition and/or the mean or the median of levels measured in more than one control or reference sample. In some examples, in d): the threshold level is determined based on the enzymatic activity that is measured in at least one subject known to have the disease or condition and/or is based on the ratio that is measured in at least one control or reference sample. In some examples, the control or reference sample is an analogous sample from a normal subject or a subject who does not have the disease or condition. In examples, the enzymatic activity is determined based on an enzymatic activity that is the mean or the median of ratios measured in more than one subject known to have the disease or condition and/or the mean or the median of ratios measured in more than one control or reference sample. In examples, the threshold level is determined by obtaining a receiver/relative operating characteristic curve (ROC curve) created by plotting Sensitivity against Specificity and determining an optimal cutoff value that provides a desired Sensitivity and a desired Specificity, wherein the optimal cutoff value is assigned as the threshold level. In some examples, the optimal cutoff value is an enzymatic activity at which or at about which the ROC curve plateaus. In examples, the optimal cutoff value is determined by the cutoff value that yields the maximum value of J (Youden's index)=Sensitivity+Specificity−1. In some examples, in d), determining whether the enzymatic activity in c) is at or above a threshold level is performed by obtaining a ratio of two enzymatic activities, where: the two enzymatic activities are measured on the same sample of body fluid under two different conditions; and/or the two enzymatic activities are measured in the sample of body fluid from the subject and in a control or reference sample.

In any of the examples of the combination methods provided herein, in some examples, the quantitating in b) is by real-time-qPCR. In some examples, the quantitating of the amplified cell-free nucleic acid includes using, in the amplification conditions of a), a nucleic acid probe that hybridizes to the cell-free nucleic acid. In examples, quantitating is performed by measuring a Ct value and if the Ct value is at or below a threshold, the subject is identified as having the disease or condition and if the Ct value is above a threshold, the subject is identified as not having the disease or condition. In some examples, the Ct value is determined by measuring a signal that is proportional to the amount of the amplification product. In examples, the Ct value is measured as the amplification cycle number at which a signal above a background level is detected.

In some examples, the Ct value is measured as the amplification cycle number where exponential increase of a signal associated with the amplification product begins. In examples, the Ct value is measured by: a) detecting the baseline angle of the S-curve and adjusting the curve by linear approximation so that the baseline obtained during the early PCR cycles is flat; b) analyzing a change in signal (ΔF), starting from the last cycle at the end of the real-time-qPCR reaction and moving backwards until a cycle number is reached where the value of ΔF changes from a positive value to zero or a negative value; and c) designating the cycle number where the value of ΔF changes from a positive value to zero or a negative value as the Ct value. In some examples, the amplification cycle number where exponential increase of a signal associated with the amplification product begins is determined using the algorithm provided herein. In examples, the early PCR cycles are between about 10 cycles to about 30 cycles of the real-time-qPCR reaction. In some examples, the early PCR cycles are between about 20 cycles to about 30 cycles of the real-time-qPCR reaction. In some examples, the last cycle at the end of the real-time-qPCR reaction is between about 40 cycles to about 50 cycles of the real-time-qPCR reaction. In examples, the last cycle at the end of the real-time-qPCR reaction is about 44, 45, 46, 47, 48, 49 or 50 cycles of the real-time-qPCR reaction. In examples, the last cycle at the end of the real-time-qPCR reaction is 46 cycles of the real-time-qPCR reaction. In examples, the method further includes linear approximation to levels of the signal such that the approximated Ct is defined as a point between two cycles where the linearly approximated signal crosses the background (Signal_{baseline}+σₙₒᵢₛₑ) level. In examples, i) if the Ct value is at or below 34, the subject is identified as having the disease or condition; and ii) if the Ct value is above 34, the subject is identified as not having the disease or condition.

In any of the examples of the combination methods provided herein, in some examples, the enzymatic activity determined in c) is a nuclease activity. In examples, the nuclease activity is measured by adding a nucleic acid probe in c) and by measuring digestion of the nucleic acid probe. In some examples, determining the nuclease activity and determining the threshold level is performed by a method that includes one or both of method (i) and method (ii): method (i)-(1) procuring a first aliquot from the body fluid, where the body fluid was previously obtained from the subject; (2) to the first aliquot, adding Zn²⁺, Mg²⁺ and a nucleic acid probe; (3) subjecting (2) to reaction conditions under which nuclease activity, if present in the body fluid, is determined by measuring digestion of the nucleic acid probe; (4) procuring a second aliquot from the previously obtained body fluid, wherein the second aliquot is of the same or similar amount as the first aliquot; (5) to the second aliquot, not adding Zn²⁺ and adding Mg²⁺ and the same nucleic acid probe that is used in (2); (6) subjecting (5) to reaction conditions under which nuclease activity, if present in the body fluid, is determined by measuring digestion of the nucleic acid probe; and (7) obtaining a ratio of the nuclease activity determined in (3) to the nuclease activity determined in (6), where if the ratio is at or above a threshold level, the subject is identified as having the disease or condition; and if the ratio is below a threshold level, the subject is identified as not having the disease or condition; and/or method (ii) (A) to a previously obtained sample from the subject, adding Mg²⁺ and a nucleic acid probe; (B) subjecting (A) to reaction conditions under which nuclease activity, if present in the body fluid, is determined by measuring digestion of the nucleic acid probe; (C) subjecting a reference or control sample to the same or similar reaction conditions as b) under which nuclease activity, if present in the reference or control sample, is determined by measuring digestion of the nucleic acid probe, or obtaining a predetermined value of nuclease activity from a control or reference sample; and (D) obtaining a ratio of the nuclease activity determined in (B) to the predetermined nuclease activity or nuclease activity that is determined in (C), where if the ratio is at or above a threshold level, the subject is identified as having the disease or condition; and if the ratio is at or below a threshold level, the subject is identified as not having the disease or condition. In examples, the control or reference sample is an analogous sample from a normal subject or a subject who does not have the disease or condition. In some examples, in e), if either or both of the following conditions are met: i) the amount of amplified nucleic acid is at or above a threshold level, and/or ii) the ratio determined by method (i) and/or the ratio determined by method (ii) is at or above a threshold level, the subject is identified as having the disease or condition. In some examples, the methods further include, in method (ii), adding Zn²⁺ in (A) and (C).

In any of the examples of the combination methods provided herein, in some examples, the cell-free nucleic acid is derived from at least one target gene, and the cell-free nucleic acid derived from the target gene is specifically amplified. In examples, the cell-free nucleic acid derived from the at least one target gene contains at least one zinc finger binding motif. In examples, the zinc finger binding motif contains a sequence of (CNN)x or (GNN)x repeats, where: a) x is the number of CNN or GNN repeats, and the number is at least 2; and b) N is A, G, C, or T. In some examples, x is 3 or more repeats. In examples, x is 3, 4, 5, 6, 7, 8, 9, 10, 11, 12, 13, 14, 15, 16 or 17 or more repeats. In some examples, x is between about 3 to about 10, 11, 12, 13, 14, 15, 16 or 17 repeats. In examples, x is at least 3, 4, 5, 6, 7, 8, 9, 10, 11, 12, 13, 14, 15, 16 or 17 repeats.

In examples of the combination methods provided herein, in some examples, the nucleic acid probe contains a sequence that is derived from a target gene in the subject, or is a sequence that is at least 95%, 96%, 97%, 98%, 99% or more identical to or complementary to a sequence derived from a target gene in the subject. In examples, the nucleic acid probe contains at least one zinc finger binding motif. In some examples, the nucleic acid probe contains a sequence of (CNN)x or (GNN)x repeats, where: a) x is the number of CNN or GNN repeats, and the number is at least 2; and b) N is A, G, C, or T. In examples, x is 3 or more repeats. In some examples, x is 3, 4, 5, 6, 7, 8, 9, 10, 11, 12, 13, 14, 15, 16 or 17 or more repeats. In examples, x is between about 3 to about 10, 11, 12, 13, 14, 15, 16 or 17 repeats. In some examples, x is at least 3, 4, 5, 6, 7, 8, 9, 10, 11, 12, 13, 14, 15, 16 or 17 repeats.

In examples of the combination methods provided herein, in some examples, at least one target gene from which the nucleic acid sequence is derived contains at least 2, 3, 4, 5, 6, 7, 8, 9 or 10 or more zinc finger binding motifs. In examples, cell-free nucleic acid derived from at least 2, 3, 4, 5, 6, 7 or 8 or more target genes is analyzed. In some examples, if cell-free nucleic acid derived from at least 1, at least 2, or at least half or more of the target genes analyzed is indicative of the presence of the disease or condition as determined by b), the subject is identified as having met condition i).

In examples of the combination methods provided herein, in some examples, at least 2, 3, 4, 5, 6, 7 or 8 or more nucleic acid probes are analyzed. In some examples, if at least 1, at least 2, or at least half or more of the nucleic acid probes analyzed is/are indicative of the presence of the disease or condition, the subject is identified as having the disease or condition.

In examples of the combination methods provided herein, in some examples, at least one nucleic acid probe in a) and one nucleic acid probe in c) have the same sequence. In examples of any of the combination methods provided herein, in some examples, in e), condition i) is not met and condition ii) is met.

Also provided herein are combination methods of analyzing a body fluid from a subject as indicative of the presence or absence of a disease or condition in the subject using more than one modality, where the methods include performing at least two of the methods selected from among any of the examples of Method 1, Method 2A and Method 2B and determining the amount of nucleic acid and/or nuclease activity, where if the amount of nucleic acid and/or nuclease activity measured in the body fluid by at least one of Method 1, Method 2A and Method 2B, relative to a threshold level, is indicative of the presence of a disease or condition in the subject, the subject is identified as having the disease or condition. In some examples, if the amount of nucleic acid and/or nuclease activity measured in the body fluid by at least two of the methods selected from among Method 1, Method 2A and Method 2B, relative to the threshold level, is indicative of the presence of a disease or condition in the subject, the subject is identified as having the disease or condition. In examples, if the amount of nucleic acid measured in the body fluid by Method 1, relative to the threshold level, is indicative of the absence of a disease or condition in the subject; and if the amount of nuclease activity measured in the body fluid by Method 2A and/or Method 2B, relative to the threshold level, is indicative of the presence of a disease or condition in the subject, the subject is identified as having the disease or condition.

In examples of the combination methods provided herein, the nucleic acid probe contains a sequence that is derived from a target gene in the subject and the target gene(s) is/are selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). In some examples, the target gene(s) is/are selected from among ESR1, PGR, HER2, ARFIP1, COX1, PLIN1, EGFR and MMP7. In examples, the nucleic acid probe(s) is/are selected, alone or including in any permutation or combination, from among one or more of the probes having the following sequences (i)-(xviii): (i) SEQ ID NO:1, or a sequence that has at least 95%, 96%, 97%, 98%, 99% or more identity with SEQ ID NO:1, (ii) SEQ ID NO:4, or a sequence that has at least 95%, 96%, 97%, 98%, 99% or more identity with SEQ ID NO:4, (iii) SEQ ID NO:7, or a sequence that has at least 95%, 96%, 97%, 98%, 99% or more identity with SEQ ID NO:7, (iv) SEQ ID NO:10, or a sequence that has at least 95%, 96%, 97%, 98%, 99% or more identity with SEQ ID NO:10, (v) SEQ ID NO:13, or a sequence that has at least 95%, 96%, 97%, 98%, 99% or more identity with SEQ ID NO:13, (vi) SEQ ID NO:16, or a sequence that has at least 95%, 96%, 97%, 98%, 99% or more identity with SEQ ID NO:16, (vii) SEQ ID NO:19, or a sequence that has at least 95%, 96%, 97%, 98%, 99% or more identity with SEQ ID NO:19, (viii) SEQ ID NO:20, or a sequence that has at least 95%, 96%, 97%, 98%, 99% or more identity with SEQ ID NO:20, (ix) SEQ ID NO:21, or a sequence that has at least 95%, 96%, 97%, 98%, 99% or more identity with SEQ ID NO:21, (x) SEQ ID NO:24, or a sequence that has at least 95%, 96%, 97%, 98%, 99% or more identity with SEQ ID NO:24, (xi) SEQ ID NO:29, or a sequence that has at least 95%, 96%, 97%, 98%, 99% or more identity with SEQ ID NO:29, (xii) SEQ ID NO:30, or a sequence that has at least 95%, 96%, 97%, 98%, 99% or more identity with SEQ ID NO:30, (xiii) SEQ ID NO:27, or a sequence that has at least 95%, 96%, 97%, 98%, 99% or more identity with SEQ ID NO:27, (xiv) SEQ ID NO:31, or a sequence that has at least 95%, 96%, 97%, 98%, 99% or more identity with SEQ ID NO:31, (xv) for the P13 KB gene, SEQ ID NO:32, or a sequence that has at least 95%, 96%, 97%, 98%, 99% or more identity with SEQ ID NO:32, (xvi) SEQ ID NO:33, or a sequence that has at least 95%, 96%, 97%, 98%, 99% or more identity with SEQ ID NO:33; (xvii) SEQ ID NO:28, or a sequence that has at least 95%, 96%, 97%, 98%, 99% or more identity with SEQ ID NO:28; and (xviii) SEQ ID NO:50, or a sequence that has at least 95%, 96%, 97%, 98%, 99% or more identity with SEQ ID NO:50, and/or the nucleic acid probe(s) is/are selected, alone or including in any permutation or combination, from among one or more of the probes having the following sequences (i)-(xviii): (i) SEQ ID NO:1, (ii) SEQ ID NO:4, (iii) SEQ ID NO:7, (iv) SEQ ID NO:10, (v) SEQ ID NO:13, (vi) SEQ ID NO:16, (vii) SEQ ID NO:19, (viii) SEQ ID NO:20, (ix) SEQ ID NO:21, (x) SEQ ID NO:24, (xi) SEQ ID NO:29, (xii) SEQ ID NO:30, (xiii) SEQ ID NO:27, (xiv) SEQ ID NO:31, (xv) SEQ ID NO:32, (xvi) SEQ ID NO:33; (xvii) SEQ ID NO:28; and (xviii) SEQ ID NO:50. In examples, the nucleic acid probe is labelled with a detectable label to provide a signal that is proportional to or inversely proportional to the amount of amplification product. In examples, the label is a chromophore, a chemiluminescent label, a radiolabel or a fluorescent label. In some examples, the label is a fluorescent label. In examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0³′ ]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. In some examples, the nucleic acid probe further contains a fluorescence quencher label. In examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ-1 (Black Hole Quencher 1), BHQ-2 (Black Hole Quencher 2), BHQ-3 (Black Hole Quencher 3), DABSYL (dimethylaminoazobenzenesulfonic acid), Iowa Black FQ and Iowa Black RQ. In examples, the fluorescent label is at the 5′-end of the nucleic acid probe and the fluorescence quencher is at the 3′-end of the nucleic acid probe. In some examples, the fluorescent label at the 5′-end of the nucleic acid probe is FAM and the fluorescence quencher at the 3′-end of the nucleic acid probe is BHQ. It is understood that a molecule that is selected to be a fluorescent reporter molecule (e.g., 5-TAMRA) cannot also serve as a fluorescence quenching pair to the same reporter molecule.

In any of the examples of the combination methods provided herein, in some examples, the disease or condition is cancer. In examples, the cancer is selected from among ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, a chordoma, a chromophobe renal cell carcinoma, a clear cell carcinoma, colon cancer, colorectal cancer, desmoplastic small round cell tumor, ependymoma, Ewing's tumor, extraskeletal myxoid chondrosarcoma, gallbladder cancer, bile duct cancer, gastric cancer, synovial tissue cancer, germ cell tumor, glioblastoma, hematological malignancy, hepatocellular carcinoma, islet cell tumor, Kaposi's Sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, neuroblastoma, neuroendocrine tumors, papillary thyroid carcinoma, parathyroid tumor, pediatric cancer, peripheral nerve sheath tumor, phaeochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, head and neck squamous cell cancer (HNSCC), stomach cancer, synovial tissue cancer, synovial sarcoma, testicular cancer, thymic carcinoma, thymoma, a thyroid cancer, thyroid metastatic cancer, uterine cancer, hepatoma, glioma, retinoblastoma, myeloma, lymphoma, and leukemia. In some examples, the cancer is breast cancer. In examples, the breast cancer is selected from among invasive ductal carcinoma breast cancer, luminal A breast cancer, luminal B breast cancer, HER2-enriched breast cancer and triple negative breast cancer. In some examples, the cancer is triple negative breast cancer.

In any examples of the combination methods provided herein, in some examples, the body fluid is whole blood, urine, plasma, serum, cerebrospinal fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In some examples, the body fluid is plasma. In examples, the volume of body fluid analyzed is between about 1 μL to about 1 mL. In some examples, the volume of body fluid analyzed is 100 μL or less. In examples, the volume of body fluid analyzed is between about 5 μL to about 50 μL. In examples, the volume of body fluid analyzed is between about 2 μL to about 25 μL, or between about 1 μL to about 20 μL. In some examples, the volume of body fluid analyzed is about or at least about 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL or 30 μL.

Also provided herein is a method of monitoring the progression of a disease or condition in a subject that includes: (a) performing one or more of any of the method(s) of analysis provided herein (e.g., Method 1, Method 2A, Method 2B, combination methods); and (b) if the subject is identified as having a disease or condition, monitoring whether the disease is progressing or decreasing by continuing to perform one or more of the method(s) of analysis provided herein (e.g., Method 1, Method 2A, Method 2B, combination methods) until the subject is identified as being in remission or as not having a disease or condition. In some examples, treatment of the subject for the disease or condition is determined based on whether the disease has progressed, decreased or is in remission or the disease or condition no longer exists.

Also provided herein is a method of treating a disease or condition in a subject previously identified as having a disease or condition by one or more of any of the method(s) of analysis provided herein (e.g., Method 1, Method 2A, Method 2B, combination methods); or previously identified as having a progressing or decreasing disease or condition by the monitoring methods provided herein, which includes administering a therapeutically effective amount of a treatment to the subject previously identified as having the disease or condition or previously identified as having the progressing or decreasing disease or condition. In examples, the disease or condition is cancer. In some examples, the cancer is selected from among ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, a chordoma, a chromophobe renal cell carcinoma, a clear cell carcinoma, colon cancer, colorectal cancer, desmoplastic small round cell tumor, ependymoma, Ewing's tumor, extraskeletal myxoid chondrosarcoma, gallbladder cancer, bile duct cancer, gastric cancer, synovial tissue cancer, germ cell tumor, glioblastoma, hematological malignancy, hepatocellular carcinoma, islet cell tumor, Kaposi's Sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, neuroblastoma, neuroendocrine tumors, papillary thyroid carcinoma, parathyroid tumor, pediatric cancer, peripheral nerve sheath tumor, phaeochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, squamous cell cancer of the head and neck (SCCHN), stomach cancer, synovial tissue cancer, synovial sarcoma, testicular cancer, thymic carcinoma, thymoma, a thyroid cancer, thyroid metastatic cancer, uterine cancer, hepatoma, glioma, retinoblastoma, myeloma, lymphoma, and leukemia. In some examples, the cancer is breast cancer. In examples, the breast cancer is selected from among invasive ductal carcinoma breast cancer, luminal A breast cancer, luminal B breast cancer, HER2-enriched breast cancer and triple negative breast cancer.

Provided are Methods 1 for detecting the presence or absence of a disease, disorder, or condition in the subject, comprising:
a) analyzing a cell-free nucleic acid sample in a body fluid from a subject to detecting the presence of a target gene at a higher level than in a control or reference sample; and,
b) analyzing cell-free nuclease activity in the body, wherein if a) and b) are indicative of the disease or condition, identifying the subject as having the disease, disorder, or condition.

Also provided are methods for detecting the presence or absence of a disease, disorder, or condition in a subject, comprising:
a) analyzing a cell-free nucleic acid sample in a body fluid from a subject to detecting the presence of a target gene at a higher level than in a control or reference sample, which indicates the presence of a disease, disorder, or condition in the subject; and,
b) analyzing cell-free nuclease activity in the body fluid, such as by a method provided herein, to assess whether such levels ae indicative of the disease, disorder, or condition. If a) and b) are indicative of the disease, disorder, or condition, identifying the subject as having the disease, disorder, or condition.

In embodiments of Methods 1, the methods can comprise analyzing a reference gene in parallel to a target gene(s). For example, a plurality or multiple reference genes can be analyzed in parallel to a target gene(s). Exemplary of a reference gene (for all methods) are single copy genes, such as, for example, glyceraldehyde-3-phosphate dehydrogenase, β-actin, and other single-copy genes. In selecting or choosing a reference gene, the nucleotide composition of a target segment(s) of a reference gene(s) can be selected to be similar to that for a target gene(s). Similar genes generally have GC content that is at least ±25% or ±20% or more of the target gene.

Reference probes as described above, can be used in the Methods 2 and combination of Methods 1 and 2. The reference probe can be analyzed in parallel to a probe(s) for the discrimination of different subject groups. Multiple reference probes can be analyzed in parallel to a probe(s) for the discrimination of different subject groups. The reference probe can be selected or chosen to have a nucleotide composition of a reference probe(s) that is similar to that for a probe(s) for the discrimination of different subject groups. For all embodiments of the methods herein a similar reference probe generally has at least ±25%, ±20%, ±15%, ±10%, of the GC content of the target or segment of the target gene.

For all methods herein, the detection/quantification of PCR products can be performed by any method known to those of skill in the art, including, but not limited to, digital PCR, mass-spectrometry-mediated detection of amplicons, single-molecule real time sequencing, and detection of amplicons by fluorometry with fluorometers. Detection of amplicons by fluorometry with fluorometers can be effected by methods employing fluorescence plate readers and/or fluorescence spectrophotometers.

For Methods 2, the detection/quantification of intact and digested probes can be performed by methods selected from among mass-spectrometry-mediated detection of probes, single-molecule real time sequencing, and detection of probes by fluorometry with fluorometers.

For Methods 1 and combinations of Methods 1 and 2, a plurality of target genes can be analyzed simultaneously. Analysis can be effected by real-time quantitative PCR with the detection/quantification of each amplicon species by the corresponding TaqMan probe. A plurality, such as 2, 3, 4, or more target genes, such as 4, can be analyzed simultaneously. For example, a reference gene is analyzed by real-time quantitative PCR concurrently with the analysis of up to three target genes; and/or multiple reference genes can be analyzed by real-time quantitative PCR concurrently with the analysis of a target gene(s). The number of reference genes that can be analyzed concurrently with the analysis of a target gene(s) is, for example, 1, 2, or 3.

For Methods 2 and combination methods with Method one, a plurality probe species can be used simultaneously for detection and/or quantification of the changes in fluorescence intensity derived from each probe species. For example, the number of probe species that are used simultaneously can be 2, 3, or 4. A reference probe(s) can be used in parallel to the analysis of probes for a given disease(s). Parallel analysis of a reference probe(s) can be used to assess overall assay performance. Multiple reference probes can be used simultaneously in parallel to the analysis of probes for the discrimination of different subject groups. For example, the number of reference probe species that can be used simultaneously is 2 or 3. For these methods, the nucleotide composition of a reference probe(s) is similar to that for a probe(s) for the discrimination of different subject groups, wherein a similar probe has at least ±25%, 20%, ±15%, ±10%, of the GC content of the target.

Provided are embodiments of Methods 1 for the identification of target genes and their segments in cell-free nucleic acids in a body fluid from a subject as indicative of the presence or absence of a disease or health condition in the subject, comprising:
a) subjecting a sample of body fluid, previously obtained from the subject, to the isolation of cell-free nucleic acid;
b) subjecting the isolated cell-free nucleic acid to amplification to generate large amounts of nucleic acid;
c) subjecting the amplified nucleic acid fragments to hybridization analysis;
d) quantifying individual hybridization events between applied nucleic acid fragments and microarray/chip elements;
e) comparing the amount of individual hybridization events of nucleic acid fragments from more than one subject groups, wherein:
   i) analyzing two subject groups, wherein, one group comprises normal or healthy subjects and the other comprises those with a disease or a health condition; and/or
   ii) analyzing and comparing more than two subject groups;
f) identifying genes and their fragments that yield statistically significant differences between and/or among subject groups; and
g) assessing the identified genes and their fragments for involvement and relevance to a disease or a health condition; and optionally or additionally; h) subjecting the identified genes and their fragments to quantitative analysis for further evaluation of the discrimination between and among subject groups. In accord with these methods, each or any of the steps can be performed as detailed in the disclosure herein. For example, step a) can be effected by linear polyacrylamide coprecipitation; and/or step b) can be effected by whole-genome amplification with DNA polymerase from bacteriophage phi29; and/or in step c) hybridization analysis can effected by microarrays or chips, each containing a large number of nucleic acid fragments (microarray/chip elements), wherein a large number is greater than 50, 100, 200, 300, 1000, several 1000, 10,000, 100,000, 200,000, 500,000, 1 million, or more; and/or in step h) quantitative analysis is effected by one or more of quantitative PCR, digital PCR, mass-spectrometry-mediated detection of PCR products (amplicons), single-molecule real time sequencing, and detection of PCR products by fluorometry with fluorometers. Detection by fluorometry can employ fluorescence plate readers and/or fluorescence spectrophotometers.

In all of the methods provided herein, subjects can be identified by first performing Method 1 and/or Method 2A and/or Method 2B to identify, and, then on subjects identified by the methods as potentially having disease, disorder, or condition, then, performing diagnostic imaging. The methods can be used for routine screening of subjects. The methods can be used for subjects, who have undergone diagnostic imaging, to determine the need for additional tests, including a biopsy, such as biopsy of breast tissue, with pathological evaluation. Hence the methods herein can be used for diagnosis, routine screening, monitoring treatment, and can be combined with other such methods. The methods can be used to monitor subjects for the effectiveness of the treatment the subjects have received, the detection of recurrence of a given cancer, the detection of the development of cancer derived from tumors that are not detectable by diagnostic imaging, and the detection of pre-existing or post-treatment metastasis of cancer.

In general, in practicing Methods 1, a segment in a target gene selected for amplification is highly GC-rich. The segment in a target gene is one that contains a potential site for interaction with or is a potential binding site recognized by one or more zinc finger proteins. Similarly in practicing Methods 2 and combinations with Methods 1, the probes can be highly GC-rich. For example, the probe can comprise a potential site for interaction with or a potential binding site recognized by one or more zinc finger proteins.

Also provided herein are compositions that include one or more nucleic acid molecule(s) that are probe(s) associated with a detectable label, where the nucleic acid molecule(s) is/are selected, including alone or in any permutation or combination, from a sequence selected from among: (i) SEQ ID NO:1, or a sequence that has at least 95%, 96%, 97%, 98%, 99% or more identity with SEQ ID NO:1, (ii) SEQ ID NO:4, or a sequence that has at least 95%, 96%, 97%, 98%, 99% or more identity with SEQ ID NO:4, (iii) SEQ ID NO:7, or a sequence that has at least 95%, 96%, 97%, 98%, 99% or more identity with SEQ ID NO:7, (iv) SEQ ID NO:10, or a sequence that has at least 95%, 96%, 97%, 98%, 99% or more identity with SEQ ID NO:10, (v) SEQ ID NO:13, or a sequence that has at least 95%, 96%, 97%, 98%, 99% or more identity with SEQ ID NO:13, (vi) SEQ ID NO:16, or a sequence that has at least 95%, 96%, 97%, 98%, 99% or more identity with SEQ ID NO:16, (vii) SEQ ID NO:19, or a sequence that has at least 95%, 96%, 97%, 98%, 99% or more identity with SEQ ID NO:19, (viii) SEQ ID NO:20, or a sequence that has at least 95%, 96%, 97%, 98%, 99% or more identity with SEQ ID NO:20, (ix) SEQ ID NO:21, or a sequence that has at least 95%, 96%, 97%, 98%, 99% or more identity with SEQ ID NO:21, (x) SEQ ID NO:24, or a sequence that has at least 95%, 96%, 97%, 98%, 99% or more identity with SEQ ID NO:24, (xi) SEQ ID NO:29, or a sequence that has at least 95%, 96%, 97%, 98%, 99% or more identity with SEQ ID NO:29, (xii) SEQ ID NO:30, or a sequence that has at least 95%, 96%, 97%, 98%, 99% or more identity with SEQ ID NO:30, (xiii) SEQ ID NO:27, or a sequence that has at least 95%, 96%, 97%, 98%, 99% or more identity with SEQ ID NO:27, (xiv) SEQ ID NO:31, or a sequence that has at least 95%, 96%, 97%, 98%, 99% or more identity with SEQ ID NO:31, (xv) for the P13 KB gene, SEQ ID NO:32, or a sequence that has at least 95%, 96%, 97%, 98%, 99% or more identity with SEQ ID NO:32, (xvi) SEQ ID NO:33, or a sequence that has at least 95%, 96%, 97%, 98%, 99% or more identity with SEQ ID NO:33; (xvii) SEQ ID NO:28, or a sequence that has at least 95%, 96%, 97%, 98%, 99% or more identity with SEQ ID NO:28; and (xviii) SEQ ID NO:50, or a sequence that has at least 95%, 96%, 97%, 98%, 99% or more identity with SEQ ID NO:50, and/or are selected, including in any permutation or combination, from a sequence selected from among: (i) SEQ ID NO:1, (ii) SEQ ID NO:4, (iii) SEQ ID NO:7, (iv) SEQ ID NO:10, (v) SEQ ID NO:13, (vi) SEQ ID NO:16, (vii) SEQ ID NO:19, (viii) SEQ ID NO:20, (ix) SEQ ID NO:21, (x) SEQ ID NO:24, (xi) SEQ ID NO:29, (xii) SEQ ID NO:30, (xiii) SEQ ID NO:27, (xiv) SEQ ID NO:31, (xv) SEQ ID NO:32, (xvi) SEQ ID NO:33; (xvii) SEQ ID NO:28; and (xviii) SEQ ID NO:50. In some examples, the detectable label is a chromophore, a chemiluminescent label, a radiolabel or a fluorescent label. In examples, the detectable label is covalently linked to the nucleic acid molecule. In some examples, the label is a fluorescent label. In examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethvl-1-aza-3-azonia-2-boranuidatricyclo[7.3.0.0]dodeca-3,5,7,9,11-pentaen-4-yl)-1-(3-hydroxy-2,5-dioxopyrrolidin-1-yl)-1-oxohexan-2-yl]propenanide), xanthenes, fluoresceins, cyanines, carbocyanines, coumarins and derivatives thereof. In examples, the nucleic acid probe further comprises a fluorescence quencher label. In some examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ-1 (Black Hole Quencher 1), BHQ-2 (Black Hole Quencher 2), BHQ-3 (Black Hole Quencher 3), DABSYL (dimethylaminoazobenzenesulfonic acid), Iowa Black FQ and Iowa Black RQ. It is understood that a molecule that is selected to be a fluorescent reporter molecule (e.g. 5-TAMRA) cannot also serve as a fluorescence quenching pair to the same reporter molecule. In examples, the fluorescent label is at the 5′-end of the nucleic acid probe and the fluorescence quencher is at the 3′-end of the nucleic acid probe. In some examples, the fluorescent label at the 5′-end of the nucleic acid probe is FAM and the fluorescence quencher at the 3′-end of the nucleic acid probe is BHQ.

Also provided herein are kits that contain the compositions provided herein, one or more reagents for performing real-time-qPCR; and optionally, instructions for use. In some examples, the kit further contains at least one primer pair, or a forward primer alone or a reverse primer alone, including in any permutation or combination, selected from among one or more of the following: (i) SEQ ID NO:2, or a sequence that has at least 95%, 96%, 97%, 98%, 99% or more identity with SEQ ID NO:2 and SEQ ID NO:3, or a sequence that has at least 95%, 96%, 97%, 98%, 99% or more identity with SEQ ID NO:3, (ii) SEQ ID NO:5, or a sequence that has at least 95%, 96%, 97%, 98%, 99% or more identity with SEQ ID NO:5 and SEQ ID NO:6, or a sequence that has at least 95%, 96%, 97%, 98%, 99% or more identity with SEQ ID NO:6, (iii) SEQ ID NO:8, or a sequence that has at least 95%, 96%, 97%, 98%, 99% or more identity with SEQ ID NO:8 and SEQ ID NO:9, or a sequence that has at least 95%, 96%, 97%, 98%, 99% or more identity with SEQ ID NO:9, (iv) SEQ ID NO:11, or a sequence that has at least 95%, 96%, 97%, 98%, 99% or more identity with SEQ ID NO:11 and SEQ ID NO:12, or a sequence that has at least 95%, 96%, 97%, 98%, 99% or more identity with SEQ ID NO:12, (v) SEQ ID NO:14, or a sequence that has at least 95%, 96%, 97%, 98%, 99% or more identity with SEQ ID NO:14 and SEQ ID NO:15, or a sequence that has at least 95%, 96%, 97%, 98%, 99% or more identity with SEQ ID NO:15, (vi) SEQ ID NO:17, or a sequence that has at least 95%, 96%, 97%, 98%, 99% or more identity with SEQ ID NO:17 and SEQ ID NO:18, or a sequence that has at least 95%, 96%, 97%, 98%, 99% or more identity with SEQ ID NO:18, (vii) SEQ ID NO:22, or a sequence that has at least 95%, 96%, 97%, 98%, 99% or more identity with SEQ ID NO:22 and SEQ ID NO:23, or a sequence that has at least 95%, 96%, 97%, 98%, 99% or more identity with SEQ ID NO:23; and (viii) SEQ ID NO:25, or a sequence that has at least 95%, 96%, 97%, 98%, 99% or more identity with SEQ ID NO:25 and SEQ ID NO:26, or a sequence that has at least 95%, 96%, 97%, 98%, 99% or more identity with SEQ ID NO:26, and/or at least one primer pair, or a forward primer alone or a reverse primer alone, including in any permutation or combination, selected from among one or more of the following: (i) for the ESR1 gene, SEQ ID NO:2 and SEQ ID NO:3, (ii) for the PGR gene, SEQ ID NO:5 and SEQ ID NO:6, (iii) for the HER2 gene, SEQ ID NO:8 and SEQ ID NO:9, (iv) for the ARFIP1 gene, SEQ ID NO:11 and SEQ ID NO:12, (v) for the COX1 gene, SEQ ID NO:14 and SEQ ID NO:15, (vi) for the PLIN1 gene, SEQ ID NO:17 and SEQ ID NO:18, (vii) for the EGFR gene, SEQ ID NO:22 and SEQ ID NO:23; and (viii) for the MMP7 gene, SEQ ID NO:25 and SEQ ID NO:26.

Also provided herein are kits that include: (a) a nucleic acid molecule containing at least one zinc finger binding motif; (b) a detectable label; (c) one or more reagents for measuring nuclease activity; and (d) optionally, instructions for use. In some examples, the nucleic acid molecule includes at least 3, 4, 5, 6, 7, 8, 9, 10, 11, 12, 13, 14, 15, 16 or 17 or more zinc finger binding motifs. In examples, the nucleic acid molecule contains between about 3 to about 10, 11, 12, 13, 14, 15, 16 or 17 zinc finger binding motifs. In some examples, the one or more reagents include Mg²⁺. In some examples, the one or more reagents include a reducing agent. In examples, the reducing agent is selected from among TCEP (tris(2-carboxyethyl) phosphine), DTT, DTE, glutathione, N-acetylcysteine, 2-mercaptoethanol, cysteine hydrochloride, sodium thioglycolate, diethyldithiocarbamate, thioglycolic acid and DTBA (dithiobutylamine). In some examples, the reducing agent is DTT. In examples, the detectable label is a chromophore, a chemiluminescent label, a radiolabel or a fluorescent label. In examples, the label is covalently linked to the nucleic acid molecule. In some examples, the label is a fluorescent label. In examples, the fluorescent label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.00]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. In examples the kit further includes a fluorescence quencher label. In examples, the fluorescence quencher label is covalently linked to the nucleic acid molecule. In some examples, the fluorescence quencher label is selected from among 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0 (Black Hole Quencher 0), BHQ-1 (Black Hole Quencher 1), BHQ-2 (Black Hole Quencher 2), BHQ-3 (Black Hole Quencher 3), DABSYL (dimethylamninoazobenzenesulfonic acid), Iowa Black FQ and Iowa Black RQ. It is understood by those of skill in the art that a molecule that is selected to be a fluorescent reporter molecule (e.g., 5-TAMRA) cannot also serve as a fluorescence quenching pair to the same reporter molecule. In examples, the fluorescent label is at the 5′-end of the nucleic acid probe and the fluorescence quencher is at the 3′-end of the nucleic acid probe. In some examples, the fluorescent label at the 5′-end of the nucleic acid probe is FAM and the fluorescence quencher at the 3′-end of the nucleic acid probe is BHQ. In some examples, the nucleic acid molecule(s) is/are selected, including alone or in any permutation or combination, from among: (i) SEQ ID NO:1, or a sequence that has at least 95%, 96%, 97%, 98%, 99% or more identity with SEQ ID NO:1, (ii) SEQ ID NO:4, or a sequence that has at least 95%, 96%, 97%, 98%, 99% or more identity with SEQ ID NO:4, (iii) SEQ ID NO:7, or a sequence that has at least 95%, 96%, 97%, 98%, 99% or more identity with SEQ ID NO:7, (iv) SEQ ID NO:10, or a sequence that has at least 95%, 96%, 97%, 98%, 99% or more identity with SEQ ID NO:10, (v) SEQ ID NO:13, or a sequence that has at least 95%, 96%, 97%, 98%, 99% or more identity with SEQ ID NO:13, (vi) SEQ ID NO:16, or a sequence that has at least 95%, 96%, 97%, 98%, 99% or more identity with SEQ ID NO:16, (vii) SEQ ID NO:19, or a sequence that has at least 95%, 96%, 97%, 98%, 99% or more identity with SEQ ID NO:19, (viii) SEQ ID NO:20, or a sequence that has at least 95%, 96%, 97%, 98%, 99% or more identity with SEQ ID NO:20, (ix) SEQ ID NO:21, or a sequence that has at least 95%, 96%, 97%, 98%, 99% or more identity with SEQ ID NO:21, (x) SEQ ID NO:24, or a sequence that has at least 95%, 96%, 97%, 98%, 99% or more identity with SEQ ID NO:24, (xi) SEQ ID NO:29, or a sequence that has at least 95%, 96%, 97%, 98%, 99% or more identity with SEQ ID NO:29, (xii) SEQ ID NO:30, or a sequence that has at least 95%, 96%, 97%, 98%, 99% or more identity with SEQ ID NO:30, (xiii) SEQ ID NO:27, or a sequence that has at least 95%, 96%, 97%, 98%, 99% or more identity with SEQ ID NO:27, (xiv) SEQ ID NO:31, or a sequence that has at least 95%, 96%, 97%, 98%, 99% or more identity with SEQ ID NO:31, (xv) for the P13 KB gene, SEQ ID NO:32, or a sequence that has at least 95%, 96%, 97%, 98%, 99% or more identity with SEQ ID NO:32, (xvi) SEQ ID NO:33, or a sequence that has at least 95%, 96%, 97%, 98%, 99% or more identity with SEQ ID NO:33; (xvii) SEQ ID NO:28, or a sequence that has at least 95%, 96%, 97%, 98%, 99% or more identity with SEQ ID NO:28; and (xviii) SEQ ID NO:50, or a sequence that has at least 95%, 96%, 97%, 98%, 99% or more identity with SEQ ID NO:50; and/or the nucleic acid molecule(s) is/are selected, including alone or in any permutation or combination, from among: (i) SEQ ID NO:1, (ii) SEQ ID NO:4, (iii) SEQ ID NO:7, (iv) SEQ ID NO:10, (v) SEQ ID NO:13, (vi) SEQ ID NO:16, (vii) SEQ ID NO:19, (viii) SEQ ID NO:20, (ix) SEQ ID NO:21, (x) SEQ ID NO:24, (xi) SEQ ID NO:29, (xii) SEQ ID NO:30, (xiii) SEQ ID NO:27, (xiv) SEQ ID NO:31, (xv) SEQ ID NO:32, (xvi) SEQ ID NO:33; (xvii) SEQ ID NO:28; and (xviii) SEQ ID NO: 50.

### BRIEF DESCRIPTION OF THE FIGURES

FIG. 1 is an ROC curve depicting an exemplary assay measuring the ratio of exogenously added zinc-dependent nuclease activity relative to nuclease activity in the absence of added zinc (K-A3 coefficient) as indicative of the presence or absence of cancer, using DNA probes containing zinc finger binding motifs as substrates.

FIG. 2 is an ROC curve depicting an exemplary assay measuring the ratio of nuclease activity in a sample from a subject with cancer relative to the nuclease activity in an analogous sample from a normal or healthy subject (K-A3B coefficient) as indicative of the presence or absence of cancer, using DNA probes containing zinc finger binding motifs as substrates. The K-A3B coefficient values depicted are the sum of individual K-A3B values measured for 3 probes.

FIG. 3 is an ROC curve depicting an exemplary assay measuring the ratio of nuclease activity in a sample from a subject with cancer relative to the nuclease activity in an analogous sample from a normal or healthy subject (K-A3B coefficient) as indicative of the presence or absence of cancer, using DNA probes containing zinc finger binding motifs as substrates. The K-A3B coefficient values depicted are the average value of K-A3B for a single probe.

### DETAILED DESCRIPTION

### Outline

A. Definitions
B. Method of Quantitating Extracellular (Cell-Free) Nucleic Acids for Disease Detection and/or Monitoring Progression (Method 1)
   1. Overview of the Method
   2. Amplification of Cell-Free Nucleic Acids in a Body Fluid
   3. Treatment with a Proteinase
   4. Amplification Methods
   5. Methods of Quantitating Cell-Free Nucleic Acids
      a. Quantitation Methods
      b. Real Time qPCR
      c. Incorporation of reference genes and probes
      d. Multiplexing
      e. Algorithm for Measuring Ct Values
C. Methods of Analyzing Extracellular (Cell-Free) Nuclease Activities for Disease Detection and/or Monitoring Progression (Method 2A and Method 2B)
   1. Overview of the Methods
   2. Cell-Free Nucleases
   3. Measurement of Nuclease Activity
      a. Absence of Added Zinc
      b. Presence of Added Zinc
      c. Determining a Ratio of Cell-free Nuclease Activities
      d. Presence of a Redox Reagent
      e. Reference Probes
      f. Multiplexing
D. Combination Methods
E. Determination of a Threshold Level
F. Target Genes
G. Zinc Finger Proteins
H. Zinc Finger Binding Motifs
I. Methods of Monitoring and/or Treating a Disease or Condition
J. Types of Diseases and Conditions
   1. Cancer or Non-Cancerous Proliferative Conditions
      Breast Cancer
   2. Pathogenic Infections
      SARS-CoV-2
K. Compositions, Combinations and Kits
L. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the terms “marker” and “biomarker” are used interchangeably herein and generally refer to a measurable substance in a tissue or body fluid that is identified to be indicative of, for example, a disease, such as cancer or infection by a pathogen, such as a virus. In the methods provided herein, biomarkers, such as the amount of extracellular nucleic acid or nuclease activity are found to be indicative of subjects having cancer or other disease conditions, such as infection by a pathogen, e.g., a virus such as SARS-Cov-2.

As used herein, “extracellular” is referred to or used interchangeably herein with “cell-free” or “cell-free circulating” and refers to a source, such as a body fluid, that contains substantially no cells. The extracellular source can contain, for example, nucleic acids such as DNA and RNA (extracellular or cell-free or cell-free circulating nucleic acids), such as the cell-free nucleic acids analyzed by the methods provided herein, or nucleases (extracellular or cell-free or cell-free circulating nucleases), such as the cell-free nucleases analyzed by the methods provided herein. Extracellular sources, such as body fluids, which contain nucleic acids and/or nucleases, often include no detectable cells but can contain cellular elements or cellular remnants that are either associated or not associated with the nucleic acids and/or nucleases. In the methods provided herein, the extracellular nucleic acid or nucleases can be extracted from the body fluid prior to analyzing the amount of extracellular nucleic acid and/or nuclease activity, according to the methods provided herein, or can be analyzed directly in the body fluid without prior extraction.

As used herein, “biological sample” or “sample” are used interchangeably and refers to a portion of a body fluid or body tissue obtained from a living or viral source or other source of macromolecules and biomolecules that includes any cell type or tissue of a subject from which nucleic acid, protein, or other macromolecule, can be obtained. The biological sample can be a sample obtained directly from a biological source, or from a biological source that is processed prior to obtaining the sample. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals, including biopsied tumor samples.

As used herein, a “sample previously obtained from a subject” refers to samples obtained from subjects prior to analysis by the methods provided herein.

As used herein, “aliquot” refers to a portion of a sample that is used for a single analysis under a set of reaction conditions for performing a method selected from among the methods provided herein. More than one aliquot of a sample can be used to perform either the same method in duplicate, triplicate, quadruplicate or higher numbers. For example, for a given sample of body fluid, aliquots of similar or identical volume or other equivalent can be subjected to identical or similar analyses of the same sample simultaneously, intermittently or sequentially in any order as duplicate, triplicate, quadruple or higher numbers of analyses to determine the same parameter, e.g., the amount of cell-free nucleic acid derived from or associated with a target gene of interest, in the sample. Aliquots of a sample also can be used to perform analyses of the same sample simultaneously, intermittently or sequentially in any order, by subjecting each aliquot to a different set of reaction conditions for performing a method selected from among the methods provided herein. For example, the nuclease activity in two aliquots of the same sample can be measured under two sets of conditions: in one aliquot, the nuclease activity can be measured in the presence of exogenously added zinc (Zn²⁺) and in the other aliquot, the nuclease activity can be measured in the absence of exogenously added zinc (Zn²⁺). As another example, two aliquots of the same sample can each be subjected to reaction conditions that measure a different parameter, e.g., one aliquot can be used to determine the amount of cell-free nucleic acid derived from or associated with a target gene of interest in the sample and another aliquot can be used to measure cell-free nuclease activity in the sample.

As used herein, a “body fluid” refers to any liquid substance extracted, excreted, or secreted from an organism or a tissue of an organism. The body fluid often includes no detectable cells but can contain cellular elements or cellular remnants that are either associated or not associated with the nucleic acids and/or nucleases. Non-limiting examples of body fluids for detecting extracellular nucleic acid and/or nuclease activity include whole blood, blood plasma, blood serum, saliva, sputum, sweat, tears, urine, synovial fluid, amniotic fluid, and cerebrospinal fluid.

As used herein, “Method 1” refers to a method of analyzing cell-free nucleic acids in a body fluid from a subject as indicative of the presence or absence of a disease or condition in the subject by specifically amplifying cell-free nucleic acid in the body fluid that is derived from at least one target gene, quantitating the amount of amplified nucleic acid and, i) if the amount of amplified nucleic acid is at or above a threshold level, identifying the subject is having the disease or condition; and ii) if the amount of amplified nucleic acid is below a threshold level, the subject is identified as not having the disease or condition. In some examples, the cell-free nucleic acid that is derived from the target gene can be present in a normal or control or reference or healthy or normal body fluid sample in an amount that is as little as one molecule, or one molecule per unit volume, and the cell-free nucleic acid that is derived from the target gene can be present in the body fluid of a subject with a disease or condition in an amount that is as little as three molecules, or three molecules per unit volume, or differs from the amount in the normal or control or reference or healthy body fluid sample by as little as two molecules, or two molecules per unit volume. In examples, the target gene contains at least one zinc finger protein binding motif, where motif is a sequence with which zinc fingers can interact and/or bind.

It is shown herein, in some examples, that the specific amplification of cell-free nucleic acid derived from certain target genes, such as those containing at least one zinc finger protein binding motif and/or those that are present in amounts in subjects having a disease or condition that differ from (generally, are greater than) amounts that are present in control or reference samples by a small amount, such as between about 2 molecules or 2 molecules per unit volume to about 10 molecules or 10 molecules per unit volume, when analyzed by Method 1, results in the ability to correctly identify subjects as having a disease or condition with a high level of sensitivity (e.g., 95%-100% sensitivity) and, additionally, good specificity, i.e., the ability to correctly identify subjects as not having a disease or condition (e.g., at least 60% or more specificity, up to 100% specificity). The threshold level generally is determined as a level that provides high sensitivity without compromising the specificity by identifying too many false positives (subjects who do not have the disease or condition being incorrectly identified as having the disease or condition).

As used herein, a reference gene or segment thereof that has a “similar” sequence to a target gene refers to reference gene or segment that has a GC content that is at least ±25%, ±20%, ±15%, ±10%, of the GC content of the target gene or corresponding segment of the target gene.

As used herein, healthy subjects, used interchangeably with normal subjects, refers to subjects that do not have the disease, disorder, or condition that is being detected and/or have levels of the marker within the range of those who do not have the particular disease, disorder, or condition.

As used herein, “Method 2” refers to a method of analyzing cell-free nuclease activity in a body fluid from a subject as indicative of the presence or absence of a disease or condition in the subject. In these methods provided herein, the cell-free nuclease activity can be assessed as a ratio of nuclease activities in one of two ways: In “Method 2A,” a body fluid sample from a subject is exposed to two sets of reaction conditions: one in which the cell-free nuclease activity is measured in the presence of exogenously added zinc (Zn²⁺) and magnesium (Mg²⁺), and another in which the cell-free nuclease activity is measured in the presence of exogenously added magnesium alone, with no exogenously added zinc. If the ratio (K-A3) of the nuclease activity in the presence of exogenously added zinc relative to the nuclease activity in the absence of exogenously added zinc is at or above a threshold level, the subject is identified as having the disease or condition and if this ratio is below a threshold level, the subject is identified as not having the disease or condition. In “Method 2B,” a body fluid sample from the subject being tested for the presence or absence of a disease or condition is exposed to reaction conditions in which the cell-free nuclease activity of the sample is measured. A reference or control sample is subjected to the same or similar reaction conditions for measuring the nuclease activity, or a predetermined value of nuclease activity from a control or reference sample is obtained. If the ratio (K-A3B) of the nuclease activity measured in the subject being tested relative to the nuclease activity of the control or reference sample is at or above a threshold level, the subject is identified as having the disease or condition and if this ratio is below a threshold level, the subject is identified as not having the disease or condition. The reactions to measure nuclease activity in the sample from the subject and in the control or reference sample can both be performed in the presence of exogenously added magnesium alone, with no exogenously added zinc, or can both be performed in the presence of both exogenously added zinc and magnesium.

In “Method 2A” and in “Method 2B,” the nuclease activities are measured by adding an exogenous nucleic acid probe as substrate. In some examples, the nucleic acid probe contains one or more zinc finger protein binding sequences or motifs. Zinc finger binding sites tend to be highly GC-rich, and target segments in the probes herein are highly GC-rich. Hence, the appearance of sites that appear to be zinc finger binding sequences or motifs. For purposes herein, they are referred to as zinc finger binding sites or motifs, but it is understood that actual binding of zinc fingers is not required for practice of the methods.

In the methods provided herein, it is described and demonstrated that the assessment of nuclease activity that is modulated by the effects of zinc, e.g., exogenously added zinc and/or the presence of a nucleic acid probe that contains at least one zinc binding motif, results in the ability to correctly identify subjects as having a disease or condition with a high level of sensitivity (e.g., 95%-100% sensitivity) and, additionally, good specificity, i.e., the ability to correctly identify subjects as not having a disease or condition (e.g., at least 60% or more specificity, up to 100% specificity). The threshold level generally is determined as a level that provides high sensitivity without compromising the specificity by identifying too many false positives (subjects who do not have the disease or condition being incorrectly identified as having the disease or condition).

As used herein, “target gene” in the context of Method 1 means a gene or fragments of the gene that are specifically amplified from the cell-free nucleic acid present in the body fluid. Cell-free nucleic acid that is “derived from” a target gene, as used herein, refers to cell-free nucleic acid that has the target gene sequence or a fragment thereof. In the context of Method 1, a nucleic acid probe that is derived from a target gene refers to a probe for quantitating amplified target gene-specific cell-free nucleic acid, whose sequence specifically hybridizes to the sequence of a target gene, or a fragment thereof, or a complement thereof.

As used herein, “specifically hybridizes” refers to annealing, by complementary base-pairing, of a nucleic acid molecule (e.g., an oligonucleotide, such as a primer or a probe) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically, the two molecules are selected such that they will hybridize under conditions of high stringency.

As used herein in the context of Method 2A and Method 2B, a nucleic acid probe that is “derived from” a target gene refers to a probe for measuring nuclease activity whose sequence is 95%, 96%, 97%, 98%, 99% or more up to 100% identical to the sequence of a target gene, or a fragment thereof, or a complement thereof.

As used herein, a target gene can be any gene of interest, all of which or fragments of which are known to be present or are identified as being present in cell-free nucleic acid (e.g., for Method 1) or are known to contain sequences which, when used as substrates for nucleases (e.g., for Method 2A and Method 2B), modulate the activity of the nuclease in a manner that is zinc-dependent, e.g., the nuclease activity is influenced by the introduction of exogenous zinc and/or by using nucleic acid probes that contain at least one zinc finger protein binding site or motif. In some examples, the target gene is selected based on its presence (or the presence of fragments thereof) in cell-free nucleic acid that is amplified (e.g., by whole genome amplification) in an amount that is at least 5, 6, 7, 8, 9, or 10 or more fold abundant than its presence in a control or reference sample amplified under the same conditions. In some examples, the target gene is connected by a causative link to the disease or condition whose presence or absence is being analyzed such as, for example, an oncogene in cancer. In some examples, the target gene is associated with the disease or condition whose presence or absence is being analyzed, e.g., the target gene is over-expressed, under-expressed, mutated, has insertions or deletions, is hypermethylated or hypomethylated in a subject having the disease or condition when compared to a control or reference subject.

As used herein, the term “specifically amplified” means that a cell-free nucleic acid of interest, such as cell-free nucleic acid derived from a target gene, is amplified using primers that specifically hybridize to sequences of the cell-free nucleic acid derived from the target gene, i.e., the amplified product flanked by the two primers can be identified as a specific product of amplification of the target gene or a fragment or complement thereof.

As used herein, “each reaction” means a single container, such as a tube, well or other container, that contains all of the components to perform a single reaction according to the methods provided herein. For example, a single reaction can contain a sample of body fluid, such as plasma, and reagents for performing an amplification reaction, including primers and/or nucleic acid probes for amplifying and/or quantitating cell-free nucleic acid derived from at least one target gene. Cell-free nucleic acid derived from more than one target gene can be quantitated in the same reaction, i.e., the same container, e.g., by using distinct detectable labels for the nucleic acid probes used to quantitate cell-free nucleic acid derived from each distinct target gene, or cell-free nucleic acid derived from each distinct target gene can be analyzed in a separate reaction, i.e., container. In addition, in some examples, the same reaction can be performed, in duplicate, triplicate, quadruplicate or higher numbers, i.e., the same reaction can be repeated in 2, 3, 4, 5, 6, 7, 8, 9, or 10 or more containers and each container is a separate reaction. Similarly, for example, a single reaction can contain a sample of body fluid, such as plasma, and reagents for performing a reaction to determine nuclease activity, including adding at least one nucleic acid probe as a substrate for determining nuclease activity by digestion of the probe. More than one nucleic acid probe (different nucleic acid sequences, e.g.) can be used to determine nuclease activity of the sample through multiple determinations of activity in the same reaction i.e., the same container, e.g., by using distinct detectable labels for cell-free nucleic acid derived from each distinct target gene, or each nucleic acid probe can be analyzed in a separate reaction, i.e., container. In addition, in some examples, the same reaction can be performed, in duplicate, triplicate, quadruplicate or higher numbers, i.e., the same reaction can be repeated in 2, 3, 4, 5, 6, 7, 8, 9, or 10 or more containers and each container is a separate reaction.

The terms “assessing” and “analyzing” are used interchangeably herein and are intended to include quantitative and qualitative determination in the sense of obtaining an absolute value of the amount of cell-free nucleic acid derived from a target gene, or the activity of a protein, such as an enzyme, e.g., a kinase, protease or nuclease, that is present in the sample, and also of obtaining an index, ratio, percentage, visual or other value indicative of the level of the activity. Assessment can be direct or indirect. For example, the chemical species actually detected need not of course be the product itself but can for example be a derivative thereof or some further substance. For example, detection of amplified nucleic acid, or of a product of cleavage by a nuclease can be a detectable moiety such as a fluorescent moiety. The terms “quantitate,” “quantitating,” “quantify,” “quantifying” and the like are used interchangeably herein to refer to a determination of an amount, such as the amount of an enzyme activity in a body fluid sample or the amount of cell-free nucleic acid derived from a target gene n a body fluid sample.

As used herein, “threshold level” generally means a predetermined level or amount that differentiates between subjects having a disease or condition and subjects not having a disease or condition. The threshold level can be based on a scoring function that is empirical or is based on prior knowledge regarding assessment of the parameter and/or assays used to assess the parameter, e.g., determination of an enzymatic activity or determination of the amount of cell-free nucleic acid. For example, the threshold level can empirically be determined based on the mean or median of a parameter in subjects having a disease or condition, when compared to the same parameter measured in one or more normal or control or reference sample(s), as measured by one skilled in the art. It is understood that the particular predetermined criteria for setting a threshold level of a parameter for the methods herein are dependent on the assay that is used to measure the parameter, e.g., quantitation of cell-free nucleic acid or an enzymatic activity, such as a nuclease activity. It also is understood that in methods involving comparisons to a predetermined level or amount, or to a control or reference sample, that the references are made with the same type of sample and using the same assay.

For example, when Method 1 is performed, in some examples, the quantitation of cell-free nucleic acid derived from one or more target genes is by real-time quantitative PCR (real-time-qPCR) in samples from one or more subjects having a disease or condition and control or reference samples assayed under the same or analogous conditions. The amount of amplified nucleic acid detected by real-time-qPCR can, in some examples, be quantitated using one or more nucleic acid probes that specifically hybridize to cell-free nucleic acid derived from one or more target genes. Quantitation can be performed by measuring a Ct (cycle threshold) value where if the Ct value is at or below a threshold level, the subject is identified as having the disease or condition and if the Ct value is above a threshold level, the subject is identified as not having the disease or condition. The Ct value can, in some examples, be determined by measuring a signal, such as a fluorescent signal, that is proportional to the amount of the amplification product. In some examples, the Ct value is measured as the amplification cycle number at which a signal above a background level is detected. In some examples, the Ct value is measured as the amplification cycle number where exponential increase of a signal associated with the amplification product begins. In examples, the amplification cycle number where exponential increase of a signal associated with the amplification product begins can be identified using the algorithm provided herein.

The Ct values obtained, e.g., for: (1) a single sample of body fluid using more than one nucleic acid probe, or (2) multiple samples of body fluid from subjects having a disease or condition or not having a disease or condition using the same nucleic acid probe, can be analyzed as a mean or a median of multiple values of Ct, thereby empirically assigning a threshold Ct level at or below which the subject is identified as having the disease or condition and above which the subject is identified as not having a disease or condition. In some examples, a Box Plot analysis can reveal Ct values that are grouped together for subjects known to be normal or reference or control subjects, Ct values that are grouped together for subjects known to have a disease or condition being tested for, and outlier values. The Box Plot analysis can be used to determine the Sensitivity and Specificity of the method for different threshold levels of Ct. In examples, the endpoint signal measurement (e.g., fluorescence measured at the last PCR cycle performed during real-time-qPCR) can be included in the scoring function (in addition to mean or median Ct values).

For example, when Methods 2A and 2B are performed, in some examples, the quantitation of nuclease activity measured in a sample of body fluid using one or more nucleic acid probes as substrates can be performed by measuring a signal, such as a fluorescent signal, that is proportional to the amount of nuclease activity (i.e., the more the amount of nucleic acid probe that is digested, the greater the amount of measured fluorescence). In some examples, the fluorescence value is measured as one or more of the following: end point fluorescence of the reaction minus the starting point fluorescence of the reaction, end point fluorescence of the reaction divided by the starting point fluorescence of the reaction, initial slope of fluorescence as a function of time, or initial slope of fluorescence as a function of time divided by fluorescence at the starting point of the reaction.

The resulting fluorescence measurement(s) can be assessed as a ratio of nuclease activities in one of two ways: In “Method 2A,” the ratio (K-A3) of the nuclease activity (as determined by the amount of fluorescence) in the presence of exogenously added zinc relative to the nuclease activity (as determined by the amount of fluorescence) in the absence of exogenously added zinc and, in “Method 2B,” the ratio (K-A3B) of the nuclease activity measured in the subject relative to the nuclease activity of the control or reference sample. The ratios obtained, e.g., for: (1) a single sample of body fluid using more than one nucleic acid probe, or (2) multiple samples of body fluid from subjects having a disease or condition or not having a disease or condition using the same nucleic acid probe, can be analyzed as a mean or a median of multiple values of K-A3 or K-A3B, thereby empirically assigning a threshold ratio at or above which the subject is identified as having the disease or condition and below which the subject is identified as not having a disease or condition. In some examples, a Box Plot analysis can reveal K-A3 or K-A3B values that are grouped together for subjects known to be normal or reference or control subjects, K-A3 or K-A3B values that are grouped together for subjects known to have a disease or condition being tested for, and outlier values. The Box Plot analysis can be used to determine the Sensitivity and Specificity of the method for different threshold levels of K-A3 or K-A3B.

The Sensitivity and Specificity determined (e.g., based on Box Plot analysis) for different threshold levels of Ct, K-A3 and/or K-A3B values, measured as described above and elsewhere herein, can be used to obtain a receiver/relative operating characteristic curve (ROC curve) created by plotting the sensitivity (rate of correctly identifying a subject as having a disease or condition) against specificity (rate of correctly identifying a subject as not having a disease or condition), using various cutoffs. The threshold level typically is selected at the point where a ROC curve plateaus or “at about which” (e.g., within about 1%, 2%, 3%, 4%, 5%, 6%, 7%, 8%, 9% or 10%) of a point at which the ROC curve plateaus, although any point on the ROC curve that provides high sensitivity without significantly compromising specificity can be selected, or that provides the desired combination of sensitivity and specificity can be selected. In some examples, the threshold level can be based on the selection of a value that provides the optimum differentiation between subjects having a disease or condition and subjects not having a disease or condition. In examples, the optimal threshold level can be determined by the cutoff yielding the maximum value of J (Youden's index)=Sensitivity+Specificity−1, and represents the level of biomarker (e.g., amount of cell-free nucleic acid or cell-free nuclease activity) that is optimal in its differentiating ability when equal weight is given to sensitivity and specificity.

As used herein, a receiver operating characteristics curve (ROC curve) is a plot of the true positive rate, generally on they-axis (TPR, or Sensitivity, i.e., the rate of correctly identifying a subject as having a disease or condition) against the false positive rate, generally on the x-axis (FPR, which is =1−Specificity, the rate of correctly identifying a subject as not having a disease or condition). The area under the curve (AUC) represents the degree or measure of separability, i.e., capability of distinguishing between subjects having a disease or condition and subjects not having a disease or condition. In general, a good model means that the AUC has a value that is as close to 1 as possible, i.e., there is a good measure of separability between the class of subjects having a disease or condition and the class of subjects not having a disease or condition. When the AUC is 0.5, it means that the model has no ability to distinguish between the class of subjects having a disease or condition and the class of subjects not having a disease or condition, and AUC below 0.5 and approaching 0 means that, more often than not, subjects having a disease or condition are identified as not having a disease or condition, and vice versa.

As used herein, the terms “quantitative PCR (qPCR)” and “real-time qPCR (real-time-qPCR),” used interchangeably herein, refer to a reaction in which amplification of a nucleic acid of interest, such as cell-free nucleic acid or cell-free nucleic acid derived from a target gene, is monitored during a PCR amplification reaction (i.e., in real-time). This monitoring can be used quantitatively (quantitative real-time PCR) or semi-quantitatively (i.e., above/below a certain amount of nucleic acid molecules; semi-quantitative real-time PCR).

Methods for performing qPCR are known to those of skill in the art and include, for example, the use of non-specific fluorescent dyes that intercalate with double-stranded DNA, such as SYBR Green I (N′,N′-dimethyl-N-[4-[E-(3-methyl-1,3-benzothiazol-2-ylidene)methyl]-1-phenylquinolin-1-ium-2-yl]-N-propylpropane-1,3-diamine), and sequence-specific nucleic acid probes labelled with a fluorescent reporter, which generally allows detection after hybridization of the probe with its complementary sequence. For non-specific detection, for example, a DNA-binding dye such as SYBR Green I binds to all double-stranded (ds) DNA during PCR. An increase in DNA product during PCR therefore leads to an increase in fluorescence intensity measured at each cycle.

For specific detection, fluorescent reporter molecule labelled nucleic acid probes specifically hybridize to and detect nucleic acid containing the sequence complementary to the probe. Accordingly, use of the reporter molecule labelled probe increases the specificity of detection of a nucleic acid of interest, such as cell-free nucleic acid derived from a target gene, in the presence of cell-free nucleic acids. Using different types of labels, fluorescent reporter molecules can be used to monitor amplification of more than one nucleic acid sequence of interest in the same reaction. In some examples, the nucleic acid probe is labelled with a fluorescent reporter at one end and a quencher of fluorescence at the other end of the probe. The close proximity of the reporter to the quencher prevents detection of its fluorescence. During PCR, the probe is broken down by the 5′ to 3′ exonuclease activity of the polymerase, which breaks the reporter-quencher proximity and thus allows unquenched emission of fluorescence, which can be detected upon excitation with a laser. An increase in the product targeted by the reporter probe at each PCR cycle causes a proportional increase in fluorescence due to the breakdown of the probe and release of the reporter.

As used herein, “TaqMan probes,” which are well known to those of skill in the art and are commercially available from a variety of sources, are hydrolysis probes that increase specificity of quantitative PCR by relying on the 5′-3′ exonuclease activity of Taq polymerase to cleave a dual-labeled probe during hybridization to the complementary target sequence and fluorophore-based detection so that the resulting fluorescence signal permits quantitative measurements of the accumulation of the product during the exponential stages of the PCR.

As used herein, a Ct value refers to a threshold signal that reflects a statistically significant increase over the baseline signal, e.g., the first PCR cycle at which a statistically significant increase in the signal, such as fluorescence, is seen. Quantitation of amplified nucleic acid by qPCR can, in some examples, be measured as a “Ct value.” The threshold also can be set to any desired value that distinguishes the amplification signal from the background, such as 5, 6, 7, 8, 9, or 10 or more times the standard deviation of the fluorescence value of the baseline. In some examples, the Ct value means the cycle number at which exponential increase of the signal, such as fluorescence, begins; in examples, an algorithm provided herein can be used to determine the Ct value at which exponential increase of the signal begins. In some examples, the Ct value also can be used to calculate the initial DNA copy number, because the Ct value is inversely related to the starting amount of target. As the template nucleic acid amount decreases, the cycle number at which significant amplification is seen increases.

As used herein, a “normal,” “reference,” “control” or “healthy” sample is used interchangeably and refers to a sample of body fluid obtained or previously obtained from a subject who has not been diagnosed as having the disease or condition or who does not exhibit symptoms of the disease or condition. The reference or control sample generally is analogous to the sample being tested, with the exception of not having the disease or condition being tested for, but is obtained from a different subject. The control or reference subject can be a subject or a population of subjects that is normal (i.e., does not have a disease or condition and/or does not exhibit symptoms of the disease or condition), or a subject or a population of subjects that has a disease but does not have the type of disease or condition that the subject being tested has or is suspected of having.

As used herein, “same or analogous” or “same or similar” means that reaction conditions that are identical or otherwise provide an equivalent environment, but for one or a few variables. For example, in Method 2A, “same or similar” can be used in the context of a sample of body fluid, such as plasma, from a subject being analyzed under two reaction conditions: one set of conditions in which the nuclease activity is measured in the presence of Mg²⁺ alone as cofactor and another set of conditions in which the same sample of body fluid from the same subject is analyzed for nuclease activity under conditions that are identical or otherwise provide an equivalent environment, but for the added presence of Zn²⁺ as a cofactor (in addition to Mg²⁺) For example, in Method 2B, “same or similar” can be used in the context of two samples being tested for cell-free nuclease activity: one being a sample of body fluid, such as plasma, from a subject being analyzed for the presence or absence of a disease or condition and the other sample being a control or reference sample of the same body fluid, e.g., plasma, being subjected to reaction conditions that are identical or otherwise provide an equivalent environment.

The term “cofactor,” as used herein, refers to a substance that either is needed for an activity, such as an enzyme activity, or that modulates the activity, e.g., increases or decreases the activity. The modulation can be direct, e.g., the cofactor, such as Zn²⁺ or Mg²⁺, directly interacts with the source of the activity, such as an enzyme, or the modulation can be indirect, e.g., a cofactor such as Zn²⁺ modulates the activity of a zinc finger protein and/or binding of a zinc finger protein to a nucleic acid, such as a nucleic acid probe, which in turn modulates the activity of, e.g., a nuclease for which the nucleic acid probe is a substrate.

As used herein, a “label” or “detectable label” refers to a compound or composition that binds to or is otherwise associated with a nucleic acid, such as a nucleic acid probe, to generate a labeled polynucleotide. The label can be detectable by itself (e.g., radioisotope labels or fluorescent labels) or can catalyze chemical alteration of a substrate compound or composition, which is detectable such as, e.g., in some examples of the methods provided herein, the label is a fluorescent “reporter” molecule, such as a fluorophore, which, prior to the amplification reaction (Method 1) or nuclease digestion (Method 2A and Method 2B), is in the vicinity of a fluorescence quencher molecule, generally present on the same nucleic acid probe as the fluorescent reporter molecule. As described elsewhere herein, during amplification (Method 1) or nuclease digestion (Method 2A and Method 2B), the fluorescent reporter molecule is removed from the vicinity of the fluorescence quencher molecule and generates a fluorescent signal that is proportional to the amount of amplification (Method 1) or nuclease digestion (Method 2A and Method 2B). Non-limiting examples of labels include chromophores, radiolabels, chemiluminescent compounds and fluorophores.

As used herein, “chromophore” refers to a compound or composition, such as a complex containing more than one compound, that is colored and is detectable as an optical signal.

As used herein, “chemiluminescent compound” refers to a compound or composition that emits light (e.g., visible light or infrared light) as a result of a chemical reaction.

As used herein, “fluorophore,” “fluorescent compound” or “fluorescent label,” as used interchangeably herein, refers to a compound or composition that emits light when it absorbs light or other electromagnetic radiation. Examples of such labels are provided herein; any detectable label suitable for labeling nucleic acids and providing a signal that is dependent on activity associated with the nucleic acid, such as amplification or digestion by one or more nucleases, is contemplated for use in the methods herein. Such labels, such as labels for use in amplification methods such as real-time-qPCR and/or for measuring enzymatic activity, such as nuclease activity, are well known to those of skill in the art.

In some examples, when a fluorophore is used as a detectable label in the methods provided herein, a fluorescence quencher can be used to quench the fluorescence until the requisite reaction, such as amplification or nucleic acid digestion, releases the fluorophore (also referred to herein as a fluorescence “reporter” molecule) and the resulting fluorescent signal is a measure of the amount of amplification or of enzymatic activity, such as nuclease activity. A “fluorescence quencher,” as used herein, refers to a compound or composition which when placed in proximity to a fluorescent compound or composition, quenches the fluorescence. For example, the nucleic acid probes used in the methods provided herein can be labeled with a fluorescent reporter molecule and a fluorescence quencher, and their proximity on the nucleic acid probe adjusted such that the reporter molecule does not fluoresce in the presence of the fluorescence quencher. An increase in fluorescence occurs when the event being monitored in a reaction takes place, such as amplification of cell-free nucleic acids or digestion of the nucleic acid probe, due to a change, e.g., increase in the distance between the fluorescent reporter molecule and the fluorescence quencher.

It is understood that the detectable labels, including fluorescence quenchers, contemplated for use in the methods provided herein can be covalently linked to the nucleic acid probes or can be associated with the probes through non-covalent interactions non-limiting examples of which include protein-ligand interactions (e.g., biotin-streptavidin interaction-mediated labeling), Van der Waals interactions, hydrogen bonding, ionic interactions, electrostatic interactions and/or hydrophilic or hydrophobic interactions. Thus, as used herein, reference to a detectable label, such as a fluorescent label, being “at” the 5′-end or the 3′-end of a nucleic acid probe means that the label can be covalently or non-covalently linked at the 5′ or 3′ end.

As used herein, “reducing agent” means an element or compound that loses or “donates” an electron to an electron recipient in a redox chemical reaction, whereby the reducing agent becomes oxidized, and the electron recipient is in a reduced state. In some examples of the methods provided herein, a reducing agent is added. In examples, the reducing agent is a thiol (having the “SH” functionality) reducing agent, such as glutathione or 2-mercaptoethanol. In some examples, the reducing agent is a dithio (having the “S-S” functionality) reducing agent, such as dithiothreitol (DTT).

As used herein, the term “detergent” is used interchangeably with the term “surfactant” or “surface acting agent.” Surfactants typically are organic compounds that are amphiphilic, i.e., containing both hydrophobic groups (“tails”) and hydrophilic groups (“heads”), which render surfactants soluble in both organic solvents and water. A surfactant can be classified by the presence of formally charged groups in its head. A non-ionic surfactant has no charge groups in its head, whereas an ionic surfactant carries a net charge in its head. A zwitterionic surfactant contains a head with two oppositely charged groups. Some examples of common surfactants include: Anionic (based on sulfate, sulfonate or carboxylate anions): perfluorooctanoate (PFOA or PFO), perfluorooctane sulfonate (PFOS), sodium polyanethol sulfonate (SPS), sodium dodecyl sulfate (SDS), ammonium lauryl sulfate, and other alkyl sulfate salts, sodium laureth sulfate (also known as sodium lauryl ether sulfate, or SLES), alkyl benzene sulfonate; cationic (based on quaternary ammonium cations): cetyl trimethylammonium bromide (CTAB) a.k.a. hexadecyl trimethyl ammonium bromide, and other alkyltrimethylammonium salts, cetylpyridinium chloride (CPC), polyethoxylated tallow amine (POEA), benzalkonium chloride (BAC), benzethonium chloride (BZT); Zwitterionic (amphoteric): dodecyl betaine; cocamidopropyl betaine; coco ampho glycinate; nonionic: alkyl poly(ethylene oxide), alkylphenol poly(ethylene oxide), copolymers of poly(ethylene oxide) and poly(propylene oxide) (commercially known as Poloxamers or Poloxamines), alkyl polyglucosides, including octyl glucoside, decyl maltoside, fatty alcohols (e.g., cetyl alcohol and oleyl alcohol), cocamide MWA, cocamide DEA, polysorbates (e.g., Tween® 20 or Tween® 80, etc.), poloxamer 188 (sold as PLURONICS® such as PLURONIC® F68), TETRONICS®, polysorbate 20, polysorbate 80, PEG 400, PEG 3000, Triton® X-100, SPAN®, MYRJ®, BRIJ@, CREMOPHOR®, polypropylene glycols, Triton detergents, dodecyl dimethylamine oxide and NP40 (an ethoxylated nonylphenol for non-ionic surfactants, also known as nonyl phenoxypolyethoxylethanol).

As used herein, the terms “procuring” and “obtaining” are used interchangeably and can refer to samples (e.g., of a body fluid) that are obtained directly from a subject or can refer to samples that previously were obtained from a subject or can refer to obtaining portions of a sample obtained directly from a subject or previously obtained from a subject.

As used herein, the term “nuclease,” refers to enzymes that can catalyze the hydrolysis of nucleic acids by cleaving the phosphodiester bonds between nucleotide subunits of the nucleic acids. Nucleases, as used herein, can be present in a body fluid, such as plasma and includes exonucleases, which digest nucleotides beginning from the 5′ or 3′ end of a nucleic acid fragment, and endonucleases, which cleave phosphodiester bonds within the nucleic acid molecule. Examples of nucleases are Bal 31, which is a double-stranded exonuclease commonly used for producing deletion sets, exonuclease I and exonuclease III for 3′-5′ exonuclease activity, Dnase I, which is an endonuclease used for splitting single-stranded and double-stranded DNA molecules, and nuclease S1 capable of degrading both single-stranded DNA and RNA molecules.

As used herein, the term “protease,” used interchangeably with “proteinase” herein, refers to an enzyme that can hydrolyze proteins into smaller polypeptides or amino acids. In some examples of Method 1 or other methods that include nucleic acid amplification as provided herein (e.g., combination methods), the sample of body fluid is treated with a proteinase prior to subject the cell-free nucleic acid in the body fluid to an amplification reaction. In examples, the proteinase is proteinase K.

As used herein, the term “kinase” refers to an enzyme that catalyzes the transfer of a phosphate (e.g., from ATP) to a substrate of interest.

As used herein, the term “sensitivity,” refers to a measure of the ability of an assay, test or method to correctly identify a subject having a disease or condition as having the disease or condition.

As used herein, the term “specificity” refers to a measure of the ability of an assay, test or method to correctly identify a subject not having a disease or condition as not having the disease or condition.

As used herein, “cutoff ratio” means a threshold level that refers to a ratio of nuclease activities below which the subject is identified as normal or healthy, i.e., no detectable disease such as cancer, and at or above which the subject is identified as having a disease such as cancer. The cutoff ratio typically is selected at the point where a ROC curve plateaus, although any point on the ROC curve that provides high sensitivity without significantly compromising specificity can be selected. For example, in the ROC curve in Example 3, at a cutoff ratio of 1.3, the Sensitivity was found to be 94% (95% CI [86%, 98%]) and the Specificity was found to be 81%. If the cutoff ratio of 1.2 is selected, the Sensitivity increases to around 97% but the Specificity decreases to about 76-77%.

For a given subject, in the methods provided herein, the cutoff ratio can be measured as a ratio of two nuclease activities in the subject: zinc-dependent nuclease activity to magnesium-dependent nuclease activity (nuclease activity measured in the presence of Zn²⁺+Mg²⁺/nuclease activity measured in the presence of Mg²⁺ alone; Method 2A, ratio K-A3) or the cutoff can be measured as a ratio between the nuclease activities of two subjects: nuclease activity measured in the presence of Mg²⁺ of a subject being tested for the presence or absence of a disease or condition, such as cancer relative to (divided by)/nuclease activity measured in the presence of Mg²⁺ of a reference subject identified as having no detectable disease or condition, or identified as not having the type of disease or condition that the subject being tested has (Method 2B, ratio K-A3B). In examples of the methods provided herein, the nuclease activity being measured is a zinc finger protein-dependent nuclease activity.

As used herein, the term “zinc finger protein-dependent nuclease activity” or “zinc finger-dependent nuclease activity” refers to a nuclease activity that is measured using substrates that are nucleic acid probes, e.g., DNA, containing sequences that are motifs to which zinc finger proteins can bind (zinc finger binding sequence or motifs). Zinc finger-dependent nuclease activity also can, in some examples, refer to a nuclease activity that is measured in the presence of exogenous zinc. It is found herein that extracellular (cell-free) nuclease activity in a body fluid that is measured using nucleic acid probes containing at least one zinc finger binding sequences or motifs or sites and/or measured in the presence of exogenous zinc can be used to distinguish between fluid samples from healthy subjects and fluid samples from subjects having a disease, such as cancer or SARS-CoV-2. Without being bound by theory, the data indicate that the nuclease(s) whose activity is/are being determined can be zinc finger proteins, and/or the nuclease activity can be modulated by the presence, absence and/or amount of zinc finger proteins in the body fluid. It is known that zinc finger binding sequences or motifs are GC rich as are the target segments in Methods and the probes in Methods 2.

As used herein, “zinc finger protein” means a protein containing at least one domain which, when bound to zinc, forms a fold that is finger shaped. The fold can bind to nucleic acids, such as DNA and RNA. In general, a zinc finger in a zinc finger protein is a protein motif of between about 27 amino acids to about 35 amino acids, such as about 30 amino acids, characterized by the coordination of one or more zinc ions (Zn²⁺) that can stabilize the fold and bind to a zinc finger binding motif in DNA or RNA.

As used herein, a “Zinc Hand” contains several zinc fingers, e.g., from between about 2 to about 20 or more fingers, such as about 3, 4, 5, 6, 7, 8, 9, 10, 11, 12, 13, 14, 15, 16 or 17 or more zinc fingers.

As used herein, “zinc finger binding motif” or “zinc finger protein binding motif” or “zinc finger binding site or sequence” refers to any two or three-dimensional feature of a nucleotide segment to which a zinc finger protein or derivative polypeptide potentially can bind with specificity. For purposes herein, reference to a zinc finger binding motif, site or sequence, describes the sequence; it does not necessarily require that a zinc finger bind to the sequence or motif. Included within this definition are nucleotide sequences as well as the three-dimensional aspects of the DNA double helix, such as, but are not limited to, the major and minor grooves and the face of the helix. The motif typically is any sequence of suitable length to which the zinc finger polypeptide potentially can bind or interact. For example, nucleic acid molecules containing sequence motifs that include “GC Box” elements, such as the “GGGCGG” consensus, can be a binding site for zinc finger proteins. In some examples, a zinc finger in a protein can bind to triplet base pairs in the “GC Box,” such as the base pairs formed by a GGG motif, or the base pairs formed by the GCG motif. A three-finger zinc finger polypeptide binds to a motif typically having about 9 to about 14 base pairs. Generally, to ensure specificity in a genome the size of a human, the recognition sequence is at least about 16 base pairs. Zinc finger binding motifs of any specificity are contemplated and provided herein. The zinc finger binding motif can be any sequence designed empirically or to which the zinc finger protein or derivative thereof potentially can bind. The motif, sequence or site, can occur in any DNA or RNA sequence, including regulatory sequences, exons, introns, or any non-coding sequence. These are GC-rich sequences; the targets in Method 1 and probes in Methods 2 are GC-rich. Reference to a zinc binding motif or sequence or site refers to the potential for a zinc finger to bind or interact.

In some examples, a zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats. A “Zinc Neck” refers to an oligonucleotide sequence that is a target for binding a Zinc Hand.

As used herein, “subject” and “patient” are used interchangeably in instances in which the subject is a human patient and has a disease or condition being analyzed by the methods provided herein. Reference herein to a “patient” means a human subject exhibiting symptoms of a disease or disorder. Subject refers to a candidate, for example a candidate with cancer, evaluated for whether a cancer is present or whether no detectable cancer is present. The term “subject” also can refer to animals, including mammals, other than human beings, such as gorillas and monkeys; rodents, such as mice and rats; fowl, such as chickens; ruminants, such as goats, cows, deer, sheep; pigs and other animals.

As used herein, a polypeptide “domain” is a part of a polypeptide (a sequence of three or more, generally 5, 10 or more amino acids) that is structurally and/or functionally distinguishable or definable. An exemplary polypeptide domain is a part of the polypeptide that can form an independently folded structure within a polypeptide made up of one or more structural motifs (e.g., a zinc finger domain formed by binding to zinc) and/or that is recognized by a particular functional activity, such as enzymatic activity, dimerization or DNA-binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, “specific binding” refers to binding partners that bind with a binding affinity Kₐ of typically at least about 10⁷l/mol, 10⁸l/mol or more. Generally, it refers to binding partners that selectively and specifically bind. For purposes herein, the antibodies are raised against a neo-epitope, specifically and selectively bind to the polypeptide that contains the neo-epitope compared to a longer polypeptide or shorter polypeptide that does not contain the neo-epitope because it is hidden or not present by virtue of the different conformation.

As used herein, “activity” refers to a functional activity or activities of a polypeptide or portion thereof associated with a full-length (complete) protein. For example, active fragments of a polypeptide can exhibit an activity of a full-length protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, “specific activity” refers to Units of activity per mg protein.

As used herein, “nucleic acids” include DNA, RNA and analogs thereof, including peptide nucleic acids (PNA) and mixtures thereof. Nucleic acids can be single or double-stranded. When referring to nucleic acid probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally, a probe or primer contains at least about 14, 15, 16, 17, 18, 19, 20, 21, 22, 23, 24, 25, 26, 27, 28, 29 or 30 or more contiguous nucleotides of sequence complementary to or identical to a gene of interest. Probes and primers can be between about 10, 20 or 30 bases long to about 50, 100 or more bases long. In some examples, the probes and primers can be between about 10 bases long to about 50 bases long, such as about or at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases long.

As used herein, a “DNA construct” is a single- or double-stranded, linear or circular DNA molecule that contains segments of DNA combined and juxtaposed and/or including components in a manner for a particular purpose, and generally in a manner not found in nature. DNA constructs exist as a result of human manipulation, and include clones and other copies of manipulated molecules.

As used herein, a “nucleic acid segment” or “nucleic acid fragment” is a portion of a larger nucleic acid molecule, e.g., DNA, having specified attributes such as, e.g., a target gene sequence.

As used herein, the term “polynucleotide” means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term “nucleotides” is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus, all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physicochemical properties of the residues involved. Alignments can be global (alignment of the compared sequences over the entire length of the sequences and including all residues) or local (the alignment of a portion of the sequences that includes only the most similar region or regions).

As used herein, identity (of sequences) is its art-recognized meaning. “Identity” per se has an art-recognized meaning and can be calculated using published techniques (See, e.g. Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carrillo, H. & Lipton, D., SIAM J Applied Math 48:1073 (1988)).

As used herein, “homologous” (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein, the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Nat. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Mol Biol 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, WI) and the University of Wisconsin Genetics Computer Group (UWG) “Gap” program (Madison WI). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986)Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

As used herein, the term “identity” or “homology,” thus, represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an “aligned” sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, quantitative PCR (qPCR), Real Time (RT)-qPCR, Reverse Transcriptase-qPCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g., by PCR) and a 3′ (downstream) primer that hybridizes with the complement of the 3′ end of the sequence to be amplified.

As used herein, the term “probe,” used interchangeably herein with “nucleic acid probe,” in the context of performing real-time-qPCR (real-time qPCR) to quantitate desired target nucleic acid sequence(s) of interest in samples from healthy subjects and samples from subjects having a disease such as cancer (Method 1 or other methods that include nucleic acid amplification as provided herein, e.g., combination methods), generally refers to a nucleic acid molecule that specifically hybridizes to a sequence within the amplification product of interest. The probe generally is labeled, and the amount of measured label is indicative of the quantity of amplification product that is generated. A probe for quantitating amplified target nucleic acid sequences can also be a molecule other than a nucleic acid molecule, e.g., a dye that fluoresces when it binds to the double-stranded amplification product, where the amount of fluorescence is proportional to the amount of amplification product.

When used in the context of a probe for measuring nuclease activity (Method 2A and Method 2B), the term “probe” or “nucleic acid probe” means a nucleic acid molecule that contains a label which, when digested by a nuclease, provides a detectable signal that is proportional to the amount of nuclease activity.

In some examples, the nucleic acid probes for use in the methods provided herein (e.g., Method 1 or other methods that include nucleic acid amplification as provided herein, e.g., combination methods, Method 2A, Method 2B) are nucleic acid molecules, such as DNA, that are labelled with a fluorescent reporter molecule, e.g., FAM, at one end and a fluorescence quencher, e.g., BHQ, at the other end. When the probe is intact, fluorescence of the reporter molecule is quenched due to proximity of the quencher molecule. During amplification (Method 1 or other methods that include performing nucleic acid amplification as provided herein, e.g., combination methods), the 5′→3′ exonuclease activity of the polymerase releases the fluorescence label, thereby increasing the fluorescence in the reaction in a manner that is proportional to the amount of amplified product generated. During nuclease digestion (Method 2A and Method 2B), nuclease activity releases the fluorescent the reporter molecule, so it no longer is proximal to the quencher molecule, thereby increasing the fluorescence in the reaction in a manner that is proportional to the nuclease activity.

In some examples, the nucleic acid probe molecule contains at least one zinc finger protein binding motif. In some examples, the zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats. In the methods provided herein, in some examples, it is found that when the probe contains at least two (CNN)x or (GNN)x repeats, generally at least 3 or more (CNN)x or (GNN)x repeats, or a number between about 3 to about 4, 5, 6, 7, 8, 9, 10, 11, 12, 13, 14, 15, 16, 17, 18, 19 or 20 (CNN)x or (GNN)x repeats, the difference between the nuclease activities measured in a body fluid sample from a subject having a disease, such as cancer, and a body fluid sample from a healthy subject or a subject not having the same disease, permits identifying a subject as having or not having the disease with high sensitivity and/or specificity.

As used herein, “substantially identical” to a product means sufficiently similar so that the property of interest is sufficiently unchanged so that the substantially identical product can be used in place of the product.

As used herein, it is understood that the terms “substantially identical” or “similar” varies with the context as understood by those skilled in the relevant art.

As used herein, modification is in reference to modification of a sequence of amino acids of a polypeptide or a sequence of nucleotides in a nucleic acid molecule and includes deletions, insertions, and replacements (e.g., substitutions), and transpositions, of amino acids and nucleotides, respectively. Modified primer and probe nucleic acid sequences exhibiting 90%, 91%, 92%, 93%, 94%, 95%, 96%, 97%, 98%, 99% or more sequence identity, or between about 9% to about 99% or more sequence identity to the primer and probe nucleic acids provided herein can be used in the methods provided herein.

As used herein, the term “promoter” means a portion of a gene containing DNA sequences that provide for the binding of RNA polymerase and initiation of transcription. Promoter sequences are commonly, but not always, found in the 5′ non-coding region of genes.

As used herein, an “isolated” or “purified” polynucleotide or nucleic acid is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties and/or biological activities, such as the ability to hybridize to a corresponding complementary sequence or, when a zinc finger binding motif is present, the ability to bind to a zinc finger protein.

As used herein, “synthetic” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by “recombinant means” or using “recombinant DNA methods” means the use of the well-known methods of molecular biology for expressing proteins encoded by cloned DNA.

As used herein, “vector” (or plasmid) refers to discrete elements that are used to introduce a heterologous nucleic acid into cells for either expression or replication thereof. The vectors typically remain episomal but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “equivalent”, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide.

As used herein, “modulate” and “modulation” or “alter” refer to a change of an activity of a molecule, such as a protein or antibody. Exemplary activities include, but are not limited to, biological activities, such as enzymatic activity, e.g., nuclease activity. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, a “composition” refers to any mixture. It can be a solution, suspension, liquid, powder, paste, aqueous, non-aqueous or any combination thereof.

As used herein, a “combination” refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a “kit” refers to a combination of components, such as a combination of the compositions herein and another item for a purpose including, but not limited to, reconstitution, activation, and instruments/devices for delivery, administration, diagnosis, and assessment of a biological activity or property. Kits optionally include instructions for use.

As used herein, “disease or condition” refers to a pathological condition in an organism resulting from cause or condition including, but not limited to, infections, acquired conditions, genetic conditions, and characterized by identifiable symptoms. Exemplary diseases and disorders of interest herein are cancers and infectious diseases caused by pathogens such as viruses, including corona viruses such as SARS-Cov-2.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, an “article of manufacture” is a product that is made and sold. As used throughout this application, the term is intended to encompass, for Method 1 or other methods that include nucleic acid amplification as provided herein, e.g., combination methods, primers and/or probes that target one or more genes of interest in cell-free nucleic acid of a body fluid, and, optionally, reagents for performing real-time quantitative PCR (real-time-qPCR), contained in articles of packaging. For Method 2A and Method 2B, the term is intended to encompass nucleic probes containing one or more zinc finger binding motifs and, optionally, reagents for measuring cell-free nuclease activities in a body fluid (e.g., Zn²⁺ and Mg²⁺ for Method 2A; Mg²⁺ for Method 2B).

As used herein, “fluid” refers to any composition that can flow. Fluids thus encompass compositions that are in the form of semi-solids, pastes, solutions, aqueous mixtures, gels, lotions, creams and other such compositions.

As used herein, a “cellular extract” or “lysate” refers to a preparation or fraction which is made from a lysed or disrupted cell.

As used herein, “animal” includes any animal, such as, but are not limited to primates including humans, gorillas and monkeys; rodents, such as mice and rats; fowl, such as chickens; ruminants, such as goats, cows, deer, sheep, pigs and other animals. Non-human animals exclude humans as the contemplated animal.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to the addition of a nucleic acid probe to a sample can include one or a plurality of probes being added to the sample.

As used herein, ranges and amounts can be expressed as “about” a particular value or range. “About” also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. METHOD OF QUANTITATING EXTRACELLULAR (CELL-FREE) NUCLEIC ACIDS FOR DISEASE DETECTION AND/OR MONITORING PROGRESSION (METHOD 1)

### 1. Overview of the Method

Provided herein is a method of analyzing cell-free nucleic acids in a body fluid from a subject as indicative of the presence or absence of a disease or condition in the subject by specifically amplifying cell-free nucleic acid in the body fluid that is derived from at least one target gene, as described elsewhere herein, quantitating the amount of amplified nucleic acid and, i) if the amount of amplified nucleic acid is at or above a threshold level, identifying the subject is having the disease or condition; and ii) if the amount of amplified nucleic acid is below a threshold level, the subject is identified as not having the disease or condition.

It is shown herein that in this method, termed “Method 1,” in some examples, a target gene or cell-free nucleic acid derived from the target gene can be quantitated when present in an amount that is as little as one copy or one molecule, or one copy or one molecule per unit volume. In some examples, the difference between the amount of the target gene or cell-free nucleic acid derived from the target gene in the body fluid of a control or reference sample and the amount of the target gene or cell-free nucleic acid derived from the target gene in a control or reference sample and the amount of the target gene or cell-free nucleic acid derived from the target gene in the body fluid of a sample having a disease or condition control can be as little as two copies or two molecules, or two copies or two molecules per unit volume.

It further is shown herein that the specific amplification of cell-free nucleic acid derived from certain target genes, including those that can be present in amounts in subjects having a disease or condition that differ from (generally, are greater than) amounts that are present in control or reference samples by between about 2 molecules or 2 molecules per unit volume to about 10 molecules or 10 molecules per unit volume, when analyzed by Method 1, provide the ability to correctly identify subjects as having a disease or condition with a high level of sensitivity (e.g., 95%-100% sensitivity) and, additionally, good specificity, i.e., the ability to correctly identify subjects as not having a disease or condition (e.g., at least 60% or more specificity, up to 100% specificity). The threshold level generally is determined as a level that provides high sensitivity (rate of correctly identifying a subject as having a disease or condition) without compromising the specificity by identifying too many false positives (subjects who do not have the disease or condition being incorrectly identified as having the disease or condition), i.e., the threshold level provides the optimum differentiation between subjects having a disease or condition and subjects not having a disease or condition.

In some examples, cell-free nucleic acid derived from more than one target gene, such as 2, 3, 4, 5, 6, 7, 8, 9, 10 or more target genes in the same sample of body fluid from a subject can be analyzed by Method 1 or other methods that include nucleic acid amplification as provided herein, e.g., combination methods, and if the level of the amount of amplified cell-free nucleic acid derived from at least 1, at least 2, at least 3, at least 4, at least 5, at least a quarter, a third, half, two thirds, three quarters or more, up to all of the target genes assessed is indicative of the presence of a disease or condition, the subject is identified as having the disease or condition. In some examples, if amplified cell-free nucleic acid derived from a majority of the target genes assessed (e.g., greater than 50%) show a level of the amount of amplified cell-free nucleic acid that is indicative of the presence of a disease or condition, the subject is identified as having the disease or condition.

In some examples, cell-free nucleic acid derived from more than one target gene, such as 2, 3, 4, 5, 6, 7, 8, 9, 10 or more target genes in the same sample of body fluid from a subject can be analyzed by Method 1 or other methods that include nucleic acid amplification as provided herein, e.g., combination methods, and if the level of the amount of amplified cell-free nucleic acid derived from at least 1, at least 2, at least 3, at least 4, at least 5, at least a quarter, a third, half, two thirds, three quarters or more, up to all of the target genes assessed is indicative of the absence of a disease or condition, the subject is identified as not having the disease or condition. In some examples, if amplified cell-free nucleic acid derived from a majority of the target genes assessed (e.g., greater than 50%) show a level of the amount of amplified cell-free nucleic acid that is indicative of the absence of a disease or condition, the subject is identified as not having the disease or condition.

### 2. Amplification of Cell-Free Nucleic Acids in a Body Fluid

The presence of circulating cell-free nucleic acids was first described in 1948. Their origin is not fully understood, with several possible mechanisms proposed (Jung et al., Clinica Chimica Acta., 411:1611-1624 (2010); Schwarzenbach et al., Nature Reviews Cancer, 11:426 (2011). They are heterogeneous in size and composition, can include nucleic acids such as, but not limited to, DNA, RNA and microRNA, and can be detected in a variety of body fluids including, but not limited to, whole blood, serum, plasma, urine, cerebrospinal fluid, pleural fluid, and saliva. Electrophoresis assays demonstrated that most cell-free DNA fragments range from between 180 and 200 base pairs (bp) and often are associated with histone proteins that form the nucleosome, suggesting that apoptotic cells can be a source of cell-free DNA (Diehl et al., Nat. Med. 2008; 14:985-990 (2008); Mouliere et al., PLoS ONE 6:e23418 (2011)).

Cell-free nucleic acid is present in healthy people in varying, usually small, amounts, e.g., between 1 and 10 ng ml-1 in plasma (Mouliere et al., Mol. Oncol. 8: 927-941 (2014); Mouliere et al., PLoS ONE 6:e23418 (2011)). Increased levels of cell-free DNA were first reported in 1977, in the serum of patients with cancer (Leon et al., Cancer Res., 37:646-650 (1977)). In the years since, an increase in total cell-free nucleic acid has been observed not only in cancer but also in several diseases or conditions including, but not limited to, stroke, trauma, including acute trauma, myocardial infarction, cerebral infarction, exercise, transplantation, inflammation, autoimmune diseases and other diseases that increase inflammation such as systemic lupus erythematosus, arthritis, and hepatitis, burns, sepsis, infection by pathogens, brain injury and other diseases or conditions.

In healthy individuals, cell-free nucleic acids appear to arise from apoptosis, but it also is possible that healthy cells, e.g., leukocytes and haematopoietic cells, actively release DNA fragments (Cai et al., Trends Genet., 31:564-75 (2015); Jung et al., Clinica Chimica Acta., 411:1611-1624 (2010); Lehmann-Werman et al., Proc. Natl Acad Sci. USA, 113:E1826-E1834 (2016); Sun et al., Proc. Natl Acad Sci. USA, 112:E5503-E5512 (2015); Lui et al., Clin. Chem., 48:421-427 (2002)). In cancers, e.g., certain tumors, cell-free nucleic acids can originate from both normal cells and cancerous cells and can be released by a combination of mechanisms, e.g., apoptosis, necrosis and macrophage phagocytosis (Choi et al., Immunology, 115:55-62 (2005)).

Cell-free nucleic acid, e.g., cell-free DNA, is rapidly cleared from circulation, with an estimated half-life of less than several hours, as low as 16 minutes to 2.5 hours; it therefore can be used as a real-time biomarker (Schwarzenbach et al., Nature Reviews Cancer, 11:426 (2011); Diehl et al., Nat. Med 14, 985-990 (2008); To et al., Clin. Cancer Res., 9:3254-3259 (2003); Lo et al., Am. J. Hum. Genet., 64:218-224 (1999); Yao et al., Gene, 590(1):142-148 (2016)). Other studies indicate that cell-free nucleic acid can be cleared from the circulation via nuclease action (Lo et al., Am. J. Hum. Genet., 64:218-224 (1999); Tamkovich et al., Ann. NY Acad Sci., 1075: 191-196 (2006)), renal excretion into the urine (Botezatu et al., Clin. Chem., 46: 1078-1084 (2000); Reckamp et al., J. Thorac. Oncol., 11:1690-1700 (2016); Tsui, et al., PLoS ONE, 7:e48319 (2012)) and uptake by the liver and spleen, followed by degradation by macrophages (Diehl et al., Proc. Natl Acad Sci. USA, 102:16368-16373 (2005); Chused et al., Clin. Exp. Immunol., 12:465-476 (1972)).

In some examples of Method 1 or other methods that include nucleic acid amplification as provided herein, e.g., combination methods provided herein, the amount of amplified cell-free nucleic acid derived from a target gene, as assessed in a sample of body fluid from a subject having a disease or condition, can be distinguished from a control or reference sample of body fluid not having the disease or condition, when the copy number or number of molecules of the target gene or cell-free nucleic acid derived from the target gene, or the copy number or number of molecules per unit volume of the target gene or cell-free nucleic acid derived from the target gene, differs between the subject having the disease or condition and the control or reference sample, prior to amplification, by 10 or less copies, molecules, copies per unit volume or molecules per unit volume, such as between about 1 copy, molecule, copy per unit volume or molecule per unit volume to about 10 copies, molecules, copies per unit volume or molecules per unit volume, e.g., about 1, 2, 3, 4, 5, 6, 7, 8, 9 or 10 copies, molecules, copies per unit volume or molecules per unit volume. For example, the difference between the amount of cell-free DNA in a sample having a disease or condition and a control or reference sample, prior to amplification, can be about or at least 2, 3, 4, 5, 6, 7, 8, 9, 10 or more copies, molecules, copies per unit volume or molecules per unit volume. In examples, the amount of cell-free nucleic acid, or the amount of cell-free nucleic acid per unit volume, in the subject having the disease or condition is greater than the amount of cell-free nucleic acid, or the amount of cell-free nucleic acid per unit volume, in a control or reference sample. In some examples, the amount of cell-free nucleic acid derived from a target gene, or the amount of cell-free nucleic acid per unit volume of a cell-free nucleic acid derived from a target gene, in the subject having the disease or condition, is greater than the amount of cell-free nucleic acid derived from a target gene, or the amount of cell-free nucleic acid per unit volume of a cell-free nucleic acid derived from a target gene, in a control or reference sample.

The target gene from which the cell-free nucleic acid to be amplified is derived, as described elsewhere herein, can be any gene that is identified as causing the disease or condition (e.g., an oncogene, when the disease or condition is cancer) or can be any gene that is associated with the disease or condition, such as having a mutation, being over-expressed or under-expressed, or being hypermethylated or hypomethylated when the disease or condition is present, compared to when the disease or condition is not present. In some examples, the target gene and/or cell-free nucleic acid derived from the target gene, when subjected to amplification conditions, e.g., whole genome amplification, for an amount of time sufficient to visualize the amplified product, e.g., on a chip, such as an amount of time sufficient to produce at least about 10 μg, 15, μg 20 μg, 25 μg, 30 μg, 35 μg, 40 μg, 45 μg, 50 μg or more amplified target gene and/or cell-free nucleic acid derived from the target gene, is detected in an amount that is at least 5-fold greater than the amount that is detected upon similar amplification of a control or reference sample, such as between about 5-fold to about 6-fold, 7-fold, 8-fold, 9-fold or 10-fold or more greater. In some examples, the target gene contains at least 1, 2, 3, 4, 5, 6, 7, 8, 9, 10, 11, 12, 13, 14, 15, 16, 17, 18, 19 or 20 or more zinc finger binding motifs. In some examples, the primers and/or the nucleic acid probe used to specifically amplify cell-free nucleic acid derived from the target gene contains at least 1, 2, 3, 4, 5, 6, 7, 8, 9, 10, 11, 12, 13, 14, 15, 16, 17, 18, 19 or 20 or more zinc finger binding motifs. In any of the examples of Method 1 or other methods that include nucleic acid amplification as provided herein, e.g., combination methods provided herein, in some examples, the disease or condition is cancer.

In some examples, the cell-free nucleic acid in a body fluid sample can be extracted prior to amplification using methods known to those of skill in the art including, but not limited to, affinity column-based, magnetic bead-based, polymer-based and phenol-chloroform-based methods, or by filtration or by preparative gel electrophoresis. In examples, the sample of the body fluid can directly be analyzed (subjected to conditions for specific amplification of cell-free nucleic acid derived from a target gene), without prior extraction of the nucleic acid from the sample. In some examples, the sample of body fluid is treated with a preservation agent, such as glycerol or other equivalent glycol or glycerol-Tris (e.g., 80% glycerol in Tris, pH 7.5) in an amount that is at least about or about 10%, 15%, 20%, 25%, 30%, 35%, 40%, 45% or 50% or more by volume relative to the volume of body fluid, or in an amount that is at least about a tenth, a quarter, a third, half, two-thirds, three quarters or more by volume, up to an equal volume relative to the volume of body fluid.

The volume of body fluid that is analyzed in each amplification reaction can be between about 1 μL to about 1 mL, such as 100 μL or less, between about 5 μL to about 50 μL, between about 2 μL to about 25 μL, or between about 1 μL to about 20 μL, or between about 1 μL to about 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL or 10 μL. In some examples, the volume of body fluid analyzed is about or at least about 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL or 30 μL.

The total volume of each amplification reaction, in some examples, can be between about 15 μL to about 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL or 100 μL or more, such as at least about 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 100 μL or 1 mL or more. In any of the examples of Method 1 or other methods that include nucleic acid amplification as provided herein, e.g., combination methods provided herein, the body fluid sample can be selected from among whole blood, serum, plasma, urine, cerebrospinal fluid, pleural fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In some examples, the body fluid is plasma.

### 3. Treatment with a Proteinase

In examples of Method 1 or other methods that include nucleic acid amplification as provided herein, e.g., combination methods provided herein, the sample of body fluid is treated with a proteinase, e.g., to protect the cell-free nucleic acid from modification or degradation, e.g., by nucleases and other proteins. In examples of the method, treatment with a proteinase can permit direct amplification of the cell-free nucleic acid in the sample, without prior extraction. Proteinases are one of the largest families of hydrolytic enzymes, which catalyze the hydrolysis of peptide bonds and thus are involved in protein turnover and several other physiological pathways throughout all life forms (Gurumallesh et al., Review Int. J. Biol. Macromol., 128:254-67 (2019); Nduwimana et al., Ann. Biol. Clin. (Paris), 53(5):251-64 (1995)). Based on the cleavage site, proteinases can be classified into endopeptidases, which cut internal peptide bonds, and exopeptidases, which exhibit their catalytic activity near the amino- or carboxy-termini of their substrates. In addition, there are six types of proteinases that are classified based on their catalytic mechanism:
Aspartic proteases: use an aspartatic acid in their catalytic mechanism.
Glutamic proteases: use a glutamatic acid in their mechanism.
Metalloproteases: use a metal ion to catalyze the reaction.
Cysteine proteases: use a cysteine as a nucleophile.
Threonine proteases: use a threonine in the catalytic mechanism.
Asparagine peptide lyases: use an asparagine for an elimination reaction.
Serine proteases: use a serine as a nucleophile.

Any proteinase such as those described above is contemplated for use in Method 1 or other methods that include nucleic acid amplification as provided herein, e.g., combination methods provided herein. A number of proteinases are commercially available and readily recognized by those of skill in the art as being suitable for use in examples of Method 1 or other methods that include nucleic acid amplification as provided herein, e.g., combination methods as provided herein. For example, several alkaline serine proteases (EC 3.4.21) and genes encoding such enzymes have been isolated from eukaryotic organisms, including yeast and fungi. Serine proteases are proteases characterized by the presence of a reactive serine residue in a catalytic triad. A catalytic triad is a group of three amino acids at the enzyme active site that are involved in catalysis. The catalytic triad allows the transfer of protons into and out of the active site. An example of a catalytic triad for the serine proteinase, chymotrypsin, is Ser 195, His 57, and Asp 102. The catalytic triad serine proteases usually contain serine, aspartic acid, and histidine, although some serine proteases can have a different combination of residues in the catalytic triad, such as Ser-His-Glu, Ser-Lys/His, or His-Ser-His. All serine proteases, however, have serine as the nucleophile and the same or similar catalytic mechanism.

In general, mature serine proteases have a molecular mass around 25 to 30 kDa (Rao et al., Microbiol. Mol. Biol. Rev., 62:597-635 (1998)) are generally active at neutral or alkaline pH, with an optimum between pH 7.0 and 11.0, and have broad substrate specificity. This group can include enzymes that are active and stable at pH 9.0 to pH 11.0 or even at pH 10.0 to 12.5 (Shimogaki et al., Agric. Biol. Chem., 55:2251-8 (1991)) with an isoelectric point of about pH 9.0. Alkaline serine proteases represent the largest subgroup of commercially used serine proteases, and their molecular masses range between 15 and 35 kDa. The temperature optima of the natural serine proteases are around 15-60° C., depending on the ecological niche of the producing organism (Rao et al., Microbiol. Mol. Biol. Rev., 62:597-635 (1998)). Some proteases for commercial use however are active at moderate temperatures and at a neutral or slightly acidic pH value. Examples of proteases suitable for use in Method 1 or other methods that include nucleic acid amplification as provided herein, e.g., combination methods provided herein include trypsin, chymotrypsin, subtilisin, elastase and those described in Skowron et al., Microb. Cell Fact. 19:135 (2020) and Huang et al., Appl. Microbiol. Biotechnol., 99:9635-9649 (2015), the contents of which are incorporated expressly by reference herein.

In some examples of Method 1 or other methods that include nucleic acid amplification as provided herein, e.g., combination methods provided herein, the proteinase is proteinase K. Proteinase K is a subtilisin-related endopeptidase that is a serine protease. Proteinase K was isolated from the mold Parengyodontium album (previously known as Tritirachium album), and its name originated from its catalytic activity using keratin as a substrate. Proteinase K has five cysteine residues, four of which form two disulfide bonds (Cys 34-Cys 124 and Cys 179-Cys 248, respectively) and one of which (Cys 73) lies below one of the catalytic triads. Proteinase K often binds two calcium ions for full enzymatic activity and has the catalytic triad Ser 224, His 69, and Asp 39. The substrate recognition sites are two peptide chains, amino acids 99-104 and amino acids 132-136.

Those of skill in the art understand the amount of proteinase needed for optimal protection of cell-free nucleic acids in a body fluid sample from degradation or other modification. In some examples, the concentration of the proteinase in each amplification reaction is between about 1 mg/mL to about 10 mg/mL. In examples, the concentration of proteinase is about or at least about 1.5 mg/mL, 2.0 mg/mL, 2.5 mg/mL, 3.0 mg/mL, 3.5 mg/mL, 4.0 mg/mL, 4.5 mg/mL or 5 mg/mL.

### 4. Amplification Methods

The cell-free nucleic acids in a body fluid can be amplified by any of several methods known to those of skill in the art, using primers designed to amplify cell-free nucleic acid derived from at least one target gene of interest. Methods of designing primers for amplifying a target gene of interest, or cell-free nucleic acid derived from a target gene of interest, and methods of quantitating the amplified nucleic acid(s), are known to those of skill in the art. In some examples, the target gene(s) can be selected as provided herein. In examples, the amplified nucleic acid can be quantitated and/or the threshold level for determining whether a disease or condition is present or absent in a subject can be determined using the analytical methods, such as adjusting the amplification S-curve and/or using the algorithm as provided herein.

Any suitable amplification technique can be utilized. Amplification methods can include, but are not limited to, polymerase chain reaction (PCR); ligation amplification (or ligase chain reaction (LCR)); amplification methods based on the use of Q-beta replicase or template-dependent polymerase (see, e.g., U.S. Patent Publication Number US2005/0287592); helicase-dependent isothermal amplification (Vincent et al., EMBO reports 5 (8):795-800 (2004)); strand displacement amplification (SDA); thermophilic SDA nucleic acid sequence based amplification (3SR or NASBA), and transcription-associated amplification (TAA).

Non-limiting examples of PCR amplification methods include, but are not limited to, standard PCR, AFLP-PCR, allele-specific PCR, Alu-PCR, asymmetric PCR, colony PCR, hot start PCR, inverse PCR (IPCR), in situ PCR (ISH), intersequence-specific PCR (ISSR-PCR), long PCR, multiplex PCR, nested PCR, quantitative PCR (qPCR), reverse transcriptase PCR (real-time-PCR), reverse transcriptase quantitative PCR (real-time-qPCR), TAQMAN qPCR, real-time PCR, single cell PCR, solid phase PCR, combinations thereof, and the like. Reagents and hardware for conducting PCR are commercially available.

An exemplary generalized description of an amplification process is as follows. Primers and template, e.g., cell-free nucleic acid derived from a target gene of interest, are contacted. The primers can specifically hybridize to the cell-free nucleic acid derived from the target gene of interest, flanking the region of the cell-free nucleic acid to be amplified. A reaction mixture containing components necessary for enzymatic functionality is added to the primer-cell-free nucleic acid hybrid, and amplification can occur under suitable conditions. Components of an amplification reaction can include, but are not limited to, e.g., primers (e.g., individual primers, primer pairs, a plurality of primer pairs, and the like) a polynucleotide template (e.g., cell-free nucleic acid derived from one or more target genes), a polymerase, nucleotides, dNTPs and the like. In some examples, non-naturally occurring nucleotides or nucleotide analogs, such as analogs containing a detectable label (e.g., fluorescent, chemiluminescent or colorimetric label) can be used, or one or more of the primers or a nucleic acid probe can be labeled. Any suitable polymerase can be selected, which can include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

PCR reaction conditions can be dependent upon primer sequences, template abundance, and the desired amount of amplification, and therefore, any suitable PCR protocol can be selected and those of skill in the art understand how such conditions can be adjusted. PCR typically can be performed as an automated process with a thermostable enzyme. In this process, the temperature of the reaction mixture is cycled through a denaturing step, a primer-annealing step, and an extension reaction step automatically. Some PCR protocols also include an activation step and a final extension step. Machines specifically adapted for this purpose are commercially available. A non-limiting example of a PCR protocol that may be suitable for embodiments described herein is as follows: treating the sample at 95° C. for 2 minutes; repeating 40 cycles of 95° C. for 15 seconds and 60° C. for 30 seconds. Additional examples of suitable PCR protocols are provided, e.g., in Examples 1 and 2. A completed PCR reaction optionally can be maintained at 4° C. until further action is desired. Multiple cycles frequently are performed using a commercially available thermal cycler. Suitable isothermal amplification processes also can be applied, in some examples.

The primers used for amplification of cell-free nucleic acid derived from a target gene can allow for specific determination, in a body fluid such as plasma or other body fluids known to those of skill in the art and described herein, of the amount of cell-free nucleic acid derived from the target gene. A primer can be a naturally occurring nucleic acid fragment or can be a synthetic sequence. The term “specific” or “specificity,” as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the terms “anneal” and “hybridize” refer to the formation of a stable complex between two molecules.

A primer nucleic acid can be designed and/or synthesized using suitable processes known to those of skill in the art and can be of any length suitable for hybridizing to a nucleotide sequence of interest. Primers can be designed based on a complementary nucleotide sequence of interest; such methods are known to those of skill in the art. In examples, a primer can be about 10 to about 100 nucleotides, about 10 to about 70 nucleotides, about 10 to about 50 nucleotides, about 15 to about 30 nucleotides, or about 15, 16, 17, 18, 19, 20, 21, 22, 23, 24, 25, 26, 27, 28, 29 or 30 nucleotides in length, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer can include naturally occurring and/or non-naturally occurring nucleotides (e.g., labeled nucleotides), or a mixture thereof. Primers suitable for use in amplification methods as described herein can be synthesized and labeled using known techniques. Primers can be chemically synthesized according to the solid phase phosphoramidite triester method first described by Beaucage and Caruthers, Tetrahedron Letts., 22:1859-1862 (1981), using an automated synthesizer, as described in Needham-VanDevanter et al., Nucleic Acids Res. 12:6159-6168 (1984). Purification of primers can be effected by native acrylamide gel electrophoresis or by anion-exchange high-performance liquid chromatography (HPLC), for example, as described in Pearson and Regnier, J. Chrom., 255:137-149 (1983).

In some examples, a primer can be a polynucleotide where one or more nucleotide positions contain a nonstandard nucleotide and/or a degenerate nucleotide. A nonstandard nucleotide may be, for example, a non-natural base, a modified base, or a universal base. A universal base is a base capable of indiscriminately base pairing with each of the four standard nucleotide bases: A, C, G and T. Universal bases that can be incorporated into a primer herein include, but are not limited to, inosine, deoxyinosine, 2′-deoxyinosine (dI, dInosine), nitroindole, 5-nitroindole, and 3-nitropyrrole (e.g., 5′ nitroindole, deoxyinosine, deoxynebularine). A degenerate nucleotide typically refers to a mixture of nucleotides at a given position and can be represented by a letter other than A, T, G or C. For example, a degenerate nucleotide can be represented by R (A or G), Y (C or T), S (G or C), W (A or T), K (G or T), M (A or C), B (C or G or T), D (A or G or T), H (A or C or T), V (A or C or G), or N (any base), for example. Such symbols for degenerate nucleotides are part of the International Union of Pure and Applied Chemistry (IUPAC) standard nomenclature for nucleotide base sequence names and represent degenerate or nonstandard nucleotides that can bind multiple nucleotides. For example, an “M” in a primer or probe would include a mixture of A and C at that position, and thus could bind to either T or G in a complementary DNA strand. An “N” in a primer or probe would include a mixture of A, T, G and C at that position, and thus could bind to any nucleotide at that position in the complementary DNA strand.

All or a portion of a primer sequence can be complementary or substantially complementary to cell-free nucleic acid derived from a target gene of interest, such as 95%, 96%, 97%, 98%, 99% or more, up to 100% complementary to cell-free nucleic acid derived from a target gene of interest. The stringency of the hybridization conditions can be altered to tolerate varying amounts of sequence mismatch. Included are cell-free nucleic acid derived from a target gene, and primer sequences, that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more up to 100% complementarity. The template cell-free nucleic acid derived from the target gene of interest can be about 50 to about 500 or more nucleotides, about 75 to about 300 or more nucleotides, about 100 to about 400 or more nucleotides, about 150 to about 300 or more nucleotides, about 160, 165, 170, 175, 180 nucleotides to about 200, 225, 250 or 300 or more nucleotides in length or at least or about 100, 105, 110, 115, 120, 125, 130, 140, 150, 175, 200, 225, 250, 275, 300, 325, 350, 375, 400, 425, 450, 475 or 500 or more nucleotides in length.

In some examples, the amount of each primer (forward and reverse primer) added to each reaction in Method 1 or other methods that include nucleic acid amplification as provided herein, e.g., combination methods provided herein can be from at least about or at 0.01 μM to about 2 μM, such as between about 0.05 μM to about 1.5 μM or between about 0.1 μM to about 1 μM. In some examples, the amount of primer added to each reaction in Method 1 or other methods that include nucleic acid amplification as provided herein, e.g., combination methods provided herein can be about or at least about 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM or 1 μM.

In some examples, the target gene from which the cell-free nucleic acid is derived is selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). Exemplary primers for detecting certain target genes include:
(i) for the ESR1 gene, SEQ ID NO:2 and SEQ ID NO:3,
(ii) for the PGR gene, SEQ ID NO:5 and SEQ ID NO:6,
(iii) for the HER2 gene, SEQ ID NO:8 and SEQ ID NO:9,
(iv) for the ARFIP1 gene, SEQ ID NO:11 and SEQ ID NO:12,
(v) for the COX1 gene, SEQ ID NO:14 and SEQ ID NO:15,
(vi) for the PLIN1 gene, SEQ ID NO:17 and SEQ ID NO:18,
(vii) for the EGFR gene, SEQ ID NO:22 and SEQ ID NO:23; and
(viii) for the MMP7 gene, SEQ ID NO:25 and SEQ ID NO:26, and primer pairs wherein, for each primer pair, one primer (forward or reverse) is selected from among those listed above and the other primer (reverse or forward, respectively) is selected from among those listed below, or both primers in the primer pairs have sequences selected from among:
(i) for the ESR1 gene, SEQ ID NO:2, or a sequence that has at least 95%, 96%, 97%, 98%, 99% or more up to 100% identity with SEQ ID NO:2 and SEQ ID NO:3, or a sequence that has at least 95%, 96%, 97%, 98%, 99% or more up to 100% identity with SEQ ID NO:3,
(ii) for the PGR gene, SEQ ID NO:5, or a sequence that has at least 95%, 96%, 97%, 98%, 99% or more up to 100% identity with SEQ ID NO:5 and SEQ ID NO:6, or a sequence that has at least 95%, 96%, 97%, 98%, 99% or more up to 100% identity with SEQ ID NO:6,
(iii) for the HER2 gene, SEQ ID NO:8, or a sequence that has at least 95%, 96%, 97%, 98%, 99% or more up to 100% identity with SEQ ID NO:8 and SEQ ID NO:9, or a sequence that has at least 95%, 96%, 97%, 98%, 99% or more up to 100% identity with SEQ ID NO:9,
(iv) for the ARFIP1 gene, SEQ ID NO:11, or a sequence that has at least 95%, 96%, 97%, 98%, 99% or more identity with SEQ ID NO:11 and SEQ ID NO:12, or a sequence that has at least 95%, 96%, 97%, 98%, 99% or more identity with SEQ ID NO:12,
(v) for the COX1 gene, SEQ ID NO:14, or a sequence that has at least 95%, 96%, 97%, 98%, 99% or more identity with SEQ ID NO:14 and SEQ ID NO:15, or a sequence that has at least 95%, 96%, 97%, 98%, 99% or more identity with SEQ ID NO:15,
(vi) for the PLIN1 gene, SEQ ID NO:17, or a sequence that has at least 95%, 96%, 97%, 98%, 99% or more identity with SEQ ID NO:17 and SEQ ID NO:18, or a sequence that has at least 95%, 96%, 97%, 98%, 99% or more identity with SEQ ID NO:18,
(vii) for the EGFR gene, SEQ ID NO:22, or a sequence that has at least 95%, 96%, 97%, 98%, 99% or more identity with SEQ ID NO:22 and SEQ ID NO:23, or a sequence that has at least 95%, 96%, 97%, 98%, 99% or more identity with SEQ ID NO:23; and
(viii) for the MMP7 gene, SEQ ID NO:25, or a sequence that has at least 95%, 96%, 97%, 98%, 99% or more identity with SEQ ID NO:25 and SEQ ID NO:26, or a sequence that has at least 95%, 96%, 97%, 98%, 99% or more identity with SEQ ID NO:26. It is understood that in some examples, any permutation or any combination of more than one of the sets of primers provided herein can be used in the methods and/or included in the compositions, combinations, kits and articles of manufacture provided herein, e.g., to quantitate cell-free nucleic acid derived from more than one target gene.

### 5. Methods of Quantitating Cell-Free Nucleic Acids

a. Quantitation Methods

Any of the amplification methods described above can be adapted to include quantitation. Methods of quantitating cell-free nucleic acid are known to those of skill in the art and can include, for example, those based on fluorometry such as the Picogreen assay or on ultraviolet spectrometry such as the Nanodrop. In some examples, cell-free nucleic acid derived amplification products can be quantitated using electrophoresis. Any suitable electrophoresis method, whereby amplified nucleic acids are separated by size, can be used in conjunction with the methods provided herein, which include, but are not limited to, standard electrophoretic techniques and specialized electrophoretic techniques, such as, for example capillary electrophoresis (e.g., Capillary Zone Electrophoresis (CZE), also known as free-solution CE (FSCE), Capillary Isoelectric Focusing (CIEF), Isotachophoresis (ITP), Electrokinetic Chromatography (EKC), Micellar Electrokinetic Capillary Chromatography (MECC OR MEKC), Micro Emulsion Electrokinetic Chromatography (MEEKC), Non-Aqueous Capillary Electrophoresis (NACE), and Capillary Electrochromatography (CEC)).

A non-limiting standard electrophoresis example can be as follows. After running an amplified nucleic acid sample in an agarose or polyacrylamide gel, the gel can be labeled (e.g., stained) with ethidium bromide or other DNA binding dye (see, Sambrook and Russell, Molecular Cloning: A Laboratory Manual 3d ed., (2001)). The presence of a band of the same size as the standard control is an indication of the presence of a target gene or cell-free nucleic acid derived from the target gene, the amount of which may then be compared to the control based on the intensity of the band, thus detecting and quantifying the target sequence of interest.

A variety of other quantitation methods are available. For example, digital PCR takes advantage of nucleic acid (e.g., DNA, cDNA or RNA) amplification on a single molecule level and can offer a sensitive method for absolute quantification of low copy number nucleic acid (Sykes et al., Biotechniques, 13:444-449 (1992); Vogelstein et al., Proc. Natl. Acad. Sci. USA, 96:9236-9241 (1999)). Systems for digital amplification and analysis of nucleic acids are available (e.g., Fluidigm® Corporation). One common digital PCR (dPCR) method is the droplet digital PCR (ddPCR), characterized by the discretization and amplification of each DNA template in single emulsion droplets (Wang et al., Water Resour. Res. 200). The separation of DNA molecules in a large number of compartments through a microfluidic system can permit high throughput analyses; by creating individual reaction chambers, the cross-contamination between neighboring compartments can be avoided to achieve more accurate quantification of targets in each sample (Gevensleben et al., Clin. Cancer Res., 19:3276-84 (2013); Pekin et al., D, Lab. Chip, 2011; 11:2156 (2011)). “Beads, Emulsion, Amplification, Magnetics digital PCR” (BEAMing) is an alternate approach that combines emulsion PCR with magnetic beads and flow cytometry for the detection and quantification of target nucleic acid. After the amplification step, each droplet contains a bead that is coated with thousands of copies of the single DNA molecule. Then, the beads are magnetically recovered and analyzed within minutes using flow cytometry or optical scanning instruments (Board et al., Breast Cancer Res. Treat., 120:461-467 (2010); Diehl et al., Nat. Methods, 3:551-559 (2006); Board et al., Ann. N. Y. Acad Sci., 1137:98-107 (2008)).

Other PCR-based quantitation techniques include real-time quantitative PCR (RT Q-PCR)-based techniques, such as the Intplex Q-PCR method, the amplification-refractory mutation system (ARMS), the Peptide Nucleic Acid (PNA) or the Locked Nucleic Acid (LNA) clamping PCR, pyrophosphorolysis-activated polymerization (PAP/biPAP), co-amplification at lower denaturation temperature (COLD PCR) and differential strand at critical temperature (DISSECT). The Intplex assay is a Q-PCR-based method where nucleic acid, such as cell-free nucleic acid, can be measured in terms of concentration and presence/absence of mutations through a multi-marker analysis of short fragments (Mouliere et al., Mol. Oncol., 8:927-941 (2014); Mouliere et al., Transl. Oncol., 6:319-IN8 (2013); Thierry et al., Nat. Med, 20:430-435 (2014)).

Next Generation Sequencing (NGS) approaches and/or a combination of PCR and NGS approaches also can be used including, but not limited to, synchronous coefficient of drag alteration (SCODA) has been developed (Thompson et al., Clin. Cancer Res., 22:5772-5782 (2016); Kidess et al., Oncotarget 2015; 6:2549-2561 (2015)). Other approaches of PCR-based targeted deep sequencing include tagged-amplicon deep sequencing (TamSeq) (Forshew et al., Sci. Transl. Med 4:136ra68 (2012)) and the Safe Sequencing System (SafeSeqS) (Bettegowda et al., 224ra24-224ra24, Sci. Transl. Med., 6 (2014); Fox et al., Next Gener. Seq. Appl., 1:1-4 (2014); Kinde et al., Proc. Natl. Acad Sci., 108:9530-9535 (2011); Tie et al., 346ra92-346ra92, Sci. Transl. Med, 8 (2016)). Cancer Personalized Profiling by deep Sequencing (CAPP-Seq) can detect and quantify circulating cell-free nucleic acid, such as circulating tumor DNA (ctDNA), with high specificity and low detection limits (Chen et al., Sci. Rep., 2016; 6:31985 (2016)). Another sensitive method, called targeted error correction sequencing (TEC-Seq), can detect low-abundance sequences and/or sequence alterations using NGS (Tie et al., 346ra92-346ra92, Sci. Transl. Med., 8 (2016)).

b. Real Time qPCR

In examples of Method 1 or other methods that include nucleic acid amplification as provided herein, e.g., combination methods provided herein, the cell-free nucleic acid is quantified using quantitative PCR (qPCR), also referred to herein as real-time qPCR or real-time-qPCR. A reverse transcriptase quantitative PCR approach also can be used, e.g., using cell-free RNA to generate a corresponding cDNA which is then quantified. In real-time-qPCR, the amplification of cell-free nucleic acid derived from a target gene of interest can be monitored during a PCR reaction (i.e., in real-time). This method can be used quantitatively to identify whether a subject has or does not have a disease or condition, e.g., by measuring the actual amount, such as by determining the actual Ct value (quantitative real-time PCR) or semi-quantitatively, e.g., by detecting by manual, automated or semi-automated methods, whether the Ct value is above or below a predetermined threshold level. (i.e., semi-quantitative real-time PCR). A detectable label that produces a signal that is associated with the progression of amplification, e.g., a directly proportional signal or an inversely proportional signal or other such related signal, can be used to quantitate the amount of amplified nucleic acid.

Examples of detectable labels include, but are not limited to, radiolabels, chromophores, chemiluminescent moieties, bioluminescent moieties, fluorescent moieties and metals. In examples, the detection method can include combinations of signaling moieties such as one or more of fluorescence, chemiluminescence, radionuclide or protein/ligand interactions that generates multimodal signaling.

Exemplary chemiluminescent materials include any selected from among oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

Exemplary chromophores include, but are not limited to, 3,3′-diaminobenzidine (DAB); 3-amino-9-ethyl carbazole (AEC); Fast Red; FD&C Yellow 5 (Tartrazine); Malachite Green Carbinol hydrochloride; Crocein Scarlet 7B (Dark Red); Erloglaucine (Dark Blue); Crystal Violet (Dark Purple); Bromophenol Blue; Cobalt(II) Chloride Hexahydrate (Red); Basic Violet 3; Acid Blue 9; Acid Red 71; FD&C Blue 1 (Brilliant Blue FCF); FD&C Red 3 (Erythrozine); and FD&C Red 40 (Allura Red AC).

Exemplary fluorophores include, but are not limited to, di-8-ANEPPS, di-4-ANEPPS, a carbocyanine dye (e.g., DiO, DiL), a PKH dye (exemplary of which are PKH-26 and PKH-67), Dylight488, Brilliant Violet, Pacific Blue, Chrome Orange, Brilliant Blue 515, phycoerythrin (PE), rhodamine, fluorescein, FITC, PE-Cy5.5, PE-Cy7, APC, Alexa647, APC-Alexa700 and APC-Alexa750, Oregon Green®, derivatives of rhodamine (e.g., Texas Red and tetrarhodimine isothiocynate (TRITC)), AMCA, Alexa Fluor®, Li-COR®, CyDyes® or DyLight® Fluors); tdTomato, mCherry, mPlum, Neptune, TagRFP, mKate2, TurboRFP and TurboFP635 (Katushka).

Examples of non-specific dyes that emit a fluorescent signal when bound to double-stranded DNA (e.g., by intercalation) include, but are not limited to, ethidium bromide, thiazole orange, oxazole yellow, BOXTO (4-[6-(benzoxazole-2-yl-(3-methyl-)-2,3-dihydro-(benzo-1,3-thiazole)-2-methylidene)]-1-methyl-quinolinium chloride) and its positive divalent derivative BOXTO-PRO (4-[(3-methyl-6-(benzoxazole-2-yl)-2,3-dihydro-(benzo-1,3-thiazole)-2-methylidene)]-1-(3-trimethylammonium-propyl)-quinolinium dibromide), SYBR GREEN I (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR GOLD [2-[N-(3-dimethylaminopropyl)-N-propylamino]-4-[2,3-dihydro-3-methyl-(benzo-1,3-thiazol-2-yl)-methylidene]-1-phenyl-quinolinium], YoYo-1 [1²(2)Z,16(17²)Z]-1³,7,7,11,11,17³-Hexamethyl-13H,173H-7,11-diaza-3¹λ⁵15¹λ⁵-3(4,1),15(1,4)-diquinolina-1,17(2)-bis([1,3]benzoxazola)heptadecaphane-1²(2),16(17²)-diene-7,11-diium-3¹,15¹-bis(ylium) tetraiodide), Yo-Pro-1 (trimethyl-[3-[4-[(Z)-(3-methyl-1,3-benzoxazol-2-ylidene)methyl]quinolin-1-ium-1-yl]propyl]azanium;diiodide), BEBO (4-[(3-methyl-6-(benzothiazol-2-yl)-2,3-dihydro-(benzo-1,3-thiazole)-2-methylidene)]-1-methyl-pyridinium iodide) and others such as those described, for example, in PCT Publication No. WO 2002/090443 A1 and in U.S. Pat. No. 7,378,240 B2. Many non-specific detection labels, both proprietary and non-proprietary, are readily available and known to those of skill in the art for use in real-time-qPCR.

Fluorescent “reporter” labels for labelling primers or nucleic acid probes, e.g., for use in qPCR also are known to those of skill in the art and can include, for example, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. Many equivalent detection labels, both proprietary and non-proprietary, are readily available and known to those of skill in the art for use in real-time-qPCR.

Fluorescence quencher labels for labelling primers or nucleic acid probes, e.g., for use in qPCR also are known to those of skill in the art and include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, **BHQ-0 (Black Hole Quencher 0), having the following structure (shown as a label at the 3′-phosphate (P) end of a nucleic acid molecule (“Oligo”)):

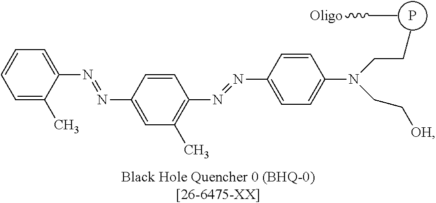

BHQ-1 (Black Hole Quencher 1) having the following structure (shown as a label at the 3′-phosphate (P) end of a nucleic acid molecule (“Oligo”)):

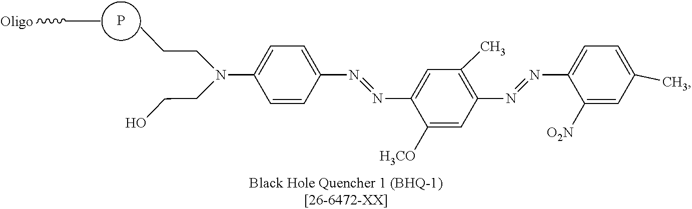

BHQ-2 (Black Hole Quencher 2) having the following structure (shown as a label at the 3′-phosphate (P) end of a nucleic acid molecule (“Oligo”)):

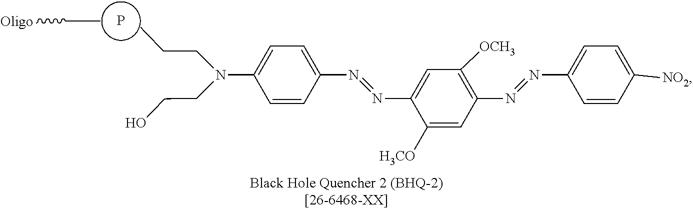

BHQ-3 (Black Hole Quencher 3), having the following structure (shown as a label at the 3′-phosphate (P) end of a nucleic acid molecule (“Oligo”)):

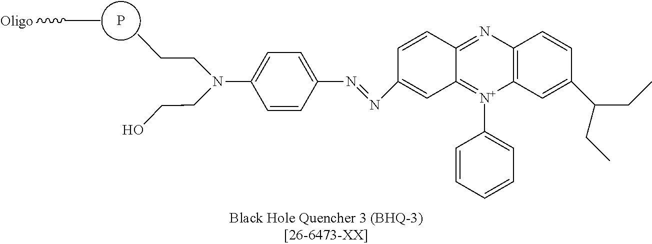

**The structures of BHQ-0, BHQ-1, BHQ-2 and BHQ-3 were obtained from Gene Link™; Catalog Numbers for each of the compounds are provided beneath each structure

DABSYL (dimethylaminoazobenzenesulfonic acid), Iowa Black FQ/Iowa Black RQ and related fluorescence quenchers such as those described in U.S. Pat. Nos. 7,439,341, 7,803,536, 7,476,735, 7,605,243, 7,645,872, 8,030,460, 8,084,588, 8,114,979, 8,258,276 and 8,916,345, the contents of which are incorporated expressly by reference herein. Many equivalent fluorescence quencher labels, both proprietary and non-proprietary, are readily available and known to those of skill in the art for use in real-time-qPCR.

The selection of suitable fluorescence reporter molecule/fluorescence quencher molecule pairs for obtaining an optimal increase in measurable fluorescence when the reporter and quencher molecules no longer are in proximity is also information that readily is available to those of skill in the art.

Methods for quantitation by qPCR include use of a non-specific detectable label, e.g., a free label in solution that binds to double-stranded DNA, such as, for example, by intercalation, or by sequence-specific quantitation using nucleic acid probes labelled with a reporter molecule (e.g., labelling through covalent, non-covalent or other interactions, such as protein-ligand interactions (e.g., biotin-streptavidin complex formation)); sequence-specific quantitation generally permits detection and/or quantitation of the amplification product after hybridization of the nucleic acid probe with its complementary sequence. Quantitative PCR methods typically are performed in a thermal cycler with the capacity to illuminate each reaction and measure a signal emitted by the label as amplification of the cell-free nucleic acid progresses in real-time.

For example, for non-specific detection, a DNA-binding dye or other label that binds to double-stranded DNA generated during PCR is added to the reaction. The label emits a strong signal when bound to double-stranded DNA (in a non-sequence specific manner). An increase in the (double-stranded) DNA product generated during PCR amplification therefore leads to an increase in the signal intensity that is measured at each cycle. Prior to amplification, when the amount of cell-free nucleic acid in the sample of body fluid is small, the dye is mostly free in solution and produces a weak signal. The reaction can be run in a real-time PCR instrument, and, after each cycle, the intensity of the signal can be measured using a detector for detecting the chromophore, chemiluminescent, fluorescent, radiolabel or other reporter signal. In some examples, cell-free nucleic acid species derived from more than one target gene can be measured in the same reaction container, such as a tube or well, using distinct detectable labels for each cell-free nucleic acid species.

Examples of non-specific dyes that emit a fluorescent signal when bound to double-stranded DNA (e.g., by intercalation) include, but are not limited to, ethidium bromide, thiazole orange, oxazole yellow, BOXTO (4-[6-(benzoxazole-2-yl-(3-methyl-)-2,3-dihydro-(benzo-1,3-thiazole)-2-methylidene)]-1-methyl-quinolinium chloride) and its positive divalent derivative BOXTO-PRO (4-[(3-methyl-6-(benzoxazole-2-yl)-2,3-dihydro-(benzo-1,3-thiazole)-2-methylidene)]-1-(3-trimethylammonium-propyl)-quinolinium dibromide), SYBR GREEN I (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR GOLD [2-[N-(3-dimethylaminopropyl)-N-propylamino]-4-[2,3-dihydro-3-methyl-(benzo-1,3-thiazol-2-yl)-methylidene]-1-phenyl-quinolinium], YoYo-1 [1²(2)Z,16(17²)Z]-1³,7,7,11,11,17³-Hexamethyl-13H,173H-7,11-diaza-3¹λ⁵,15¹λ⁵-3(4,1),15(1,4)-diquinolina-1,17(2)-bis([1,3]benzoxazola)heptadecaphane-1²(2),16(17²)-diene-7,11-diium-3¹,15¹-bis(ylium) tetraiodide), Yo-Pro-1 (trimethyl-[3-[4-[(Z)-(3-methyl-1,3-benzoxazol-2-ylidene)methyl]quinolin-1-ium-1-yl]propyl]azanium;diiodide), BEBO (4-[(3-methyl-6-(benzothiazol-2-yl)-2,3-dihydro-(benzo-1,3-thiazole)-2-methylidene)]-1-methyl-pyridinium iodide) and others such as those described, for example, in PCT Publication No. WO 2002/090443 A1 and in U.S. Pat. No. 7,378,240 B2. Many equivalent non-specific detection labels, both proprietary and non-proprietary, are readily available and known to those of skill in the art for use in real-time-qPCR.

For specific detection, for example, nucleic acid probes associated with a label that can provide a detectable “reporter” signal, such as by covalent linkage or non-covalent interactions, which can emit a signal that is dependent on the nucleic acid probe binding/hybridizing to a complementary sequence in the cell-free nucleic acid, thereby monitoring specific amplification of the complementary sequence to which it binds. Accordingly, use of the nucleic acid probe containing the reporter signal increases specificity, and permits detection of a target nucleic acid of interest, such as cell-free nucleic acid derived from a target gene of interest, even when other double stranded DNA or other nucleic acid is present. Any labels that can provide a detectable signal indicative of the amount of amplified cell-free nucleic acid, such as those known to those of skill in the art and/or described herein, can be used. Using distinct labels, more than one nucleic acid probe can be used in the same reaction to analyze the amount of cell-free nucleic acid derived from more than one target gene of interest and/or more than one region of a target gene of interest.

This assay can be performed by using other methods/techniques for the detection/quantification of PCR products (amplicons) without the change of the assay principle and with no or minimal routine modification of assay procedures. These methods include, but are not limited to, digital PCR, mass-spectrometry-mediated detection of amplicons, single-molecule real time sequencing, detection of amplicons by fluorometry with fluorometers, including fluorescence plate readers and fluorescence spectrophotometers, and any other such methods known to those of skill in the art.

In some examples of Method 1 or other methods that include nucleic acid amplification as provided herein, e.g., combination methods provided herein, a nucleic acid probe is labelled with a fluorescent reporter molecule and a fluorescence quencher molecule. Examples of fluorescent reporter molecules for nucleic acid include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. Many equivalent detection labels, both proprietary and non-proprietary, are readily available and known to those of skill in the art for use in real-time-qPCR.

Fluorescence quencher labels for labelling nucleic acid probes include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0, BHQ-1, BHQ-2, BHQ-3, DABSYL (dimethylaminoazobenzenesulfonic acid), Iowa Black FQ/Iowa Black RQ and related fluorescence quenchers such as those described in U.S. Pat. Nos. 7,439,341, 7,803,536, 7,476,735, 7,605,243, 7,645,872, 8,030,460, 8,084,588, 8,114,979, 8,258,276 and 8,916,345, the contents of which are incorporated expressly by reference herein. Many equivalent fluorescence quencher labels, both proprietary and non-proprietary, are readily available and known to those of skill in the art for use in real-time-qPCR.

In examples, the fluorescent reporter molecule is at the 5′-end of the nucleic acid probe and the fluorescence quencher molecule is at the 3′-end of the nucleic acid probe. In examples, the fluorescent reporter molecule is FAM and the fluorescence quencher molecule is BHQ-1. For specific detection of cell-free nucleic acid derived from a target gene of interest using real-time-qPCR, before the PCR amplification reaction is initiated, the close proximity of the reporter to the quencher prevents detection of its fluorescence. During PCR, the probe is broken down by the 5′ to 3′ exonuclease activity of the polymerase such as a Taq polymerase, which breaks the reporter-quencher proximity and thus allows unquenched emission of fluorescence, which can be detected by excitation with a laser. An increase in the product targeted by the reporter probe at each PCR cycle therefore causes a proportional increase in fluorescence due to the breakdown of the probe and release of the reporter.

As with primers, nucleic acid probes can be designed and/or synthesized using suitable processes known to those of skill in the art and can be of any length suitable for hybridizing to a nucleotide sequence of interest. Nucleic acid probes can be designed based on a complementary nucleotide sequence of interest; such methods are known to those of skill in the art. In examples, a probe can be about 10 to about 100 nucleotides, about 10 to about 70 nucleotides, about 10 to about 50 nucleotides, about 15 to about 30 nucleotides, generally at least or about 15, 16, 17, 18, 19, 20, 21, 22, 23, 24, 25, 26, 27, 28, 29 or 30 nucleotides in length. A probe can include naturally occurring and/or non-naturally occurring nucleotides (e.g., labeled nucleotides), or a mixture thereof. Probes suitable for use in amplification methods as described herein can be synthesized and labeled using known techniques. The probes can be chemically synthesized according to the solid phase phosphoramidite triester method first described by Beaucage and Caruthers, Tetrahedron Letts., 22:1859-1862 (1981), using an automated synthesizer, as described in Needham-VanDevanter et al., Nucleic Acids Res. 12:6159-6168 (1984). Purification of probes can be effected by native acrylamide gel electrophoresis or by anion-exchange high-performance liquid chromatography (HPLC), for example, as described in Pearson and Regnier, J. Chrom., 255:137-149 (1983).

In some examples, a probe can be a polynucleotide where one or more nucleotide positions contain a nonstandard nucleotide and/or a degenerate nucleotide. A nonstandard nucleotide may be, for example, a non-natural base, a modified base, or a universal base. A universal base is a base capable of indiscriminately base pairing with each of the four standard nucleotide bases: A, C, G and T. Universal bases that can be incorporated into a primer herein include, but are not limited to, inosine, deoxyinosine, 2′-deoxyinosine (dI, dInosine), nitroindole, 5-nitroindole, and 3-nitropyrrole (e.g., 5′ nitroindole, deoxyinosine, deoxynebularine). A degenerate nucleotide typically refers to a mixture of nucleotides at a given position and can be represented by a letter other than A, T, G or C. For example, a degenerate nucleotide can be represented by R (A or G), Y (C or T), S (G or C), W (A or T), K (G or T), M (A or C), B (C or G or T), D (A or G or T), H (A or C or T), V (A or C or G), or N (any base), for example. Such symbols for degenerate nucleotides are part of the International Union of Pure and Applied Chemistry (IUPAC) standard nomenclature for nucleotide base sequence names and represent degenerate or nonstandard nucleotides that can bind multiple nucleotides. For example, an “M” in a primer or probe would include a mixture of A and C at that position, and thus could bind to either T or G in a complementary DNA strand. An “N” in a primer or probe would include a mixture of A, T, G and C at that position, and thus could bind to any nucleotide at that position in the complementary DNA strand.

All or a portion of the probe can be complementary or substantially complementary to cell-free nucleic acid derived from a target gene of interest, such as 95%, 96%, 97%, 98%, 99% or more, up to 100% complementary to cell-free nucleic acid derived from a target gene of interest. The stringency of the hybridization conditions can be altered to tolerate varying amounts of sequence mismatch. Included are cell-free nucleic acid derived from a target gene, and probe sequences, that share 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more up to 100% complementarity. The template cell-free nucleic acid derived from the target gene of interest can be about 50 to about 500 or more nucleotides, about 75 to about 300 or more nucleotides, about 100 to about 400 or more nucleotides, about 150 to about 300 or more nucleotides, about 160, 165, 170, 175, 180 nucleotides to about 200, 225, 250 or 300 or more nucleotides in length or at least or about 100, 105, 110, 115, 120, 125, 150, 175, 200, 225, 250, 275, 300, 325, 350, 375, 400, 425, 450, 475 or 500 or more nucleotides in length.

In some examples, the amount of a nucleic acid probe added to each reaction in Method 1 or other methods that include nucleic acid amplification as provided herein, e.g., combination methods provided herein, can be from at least about or at 0.01 μM to about 2 μM, such as between about 0.05 μM to about 1.5 μM or between about 0.1 μM to about 1 μM. In some examples, the amount of nucleic acid probe added to each reaction in Method 1 or other methods that include nucleic acid amplification as provided herein, e.g., combination methods provided herein can be about or at least about 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM or 1 μM. In some examples, the amount of nucleic acid probe added to each reaction can be between about 0.3 μM to about 0.6 μM, such as at least or about 0.3 μM, 0.35 μM, 0.4 μM, 0.45 μM, 0.5 μM, 0.55 μM or 0.6 μM.

In some examples, the target gene from which the cell-free nucleic acid is derived contains at least one zinc finger binding motif. In examples, the zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats.

In some examples, the nucleic acid probe contains at least one zinc finger binding motif. In examples, the zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats.

In some examples, the target gene from which the cell-free nucleic acid is derived is selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). Exemplary nucleic acid probes for quantitating cell-nucleic acids derived from certain target genes include those selected from among one or more of the following:

SEQ ID NO:1 (complementary to a portion of ESR1), a sequence that has at least 95%, 96%, 97%, 98%, 99% or more identity with SEQ ID NO:1, SEQ ID NO:4 (complementary to PGR), a sequence that has at least 95%, 96%, 97%, 98%, 99% or more identity with SEQ ID NO:4, SEQ ID NO:7 (complementary to a portion of HER2), a sequence that has at least 95%, 96%, 97%, 98%, 99% or more identity with SEQ ID NO:7, SEQ ID NO:10 (complementary to a portion of ARFIP1), a sequence that has at least 95%, 96%, 97%, 98%, 99% or more identity with SEQ ID NO:10, SEQ ID NO:13 (complementary to a portion of COX1), a sequence that has at least 95%, 96%, 97%, 98%, 99% or more identity with SEQ ID NO:13, SEQ ID NO:16 (complementary to a portion of PLIN1), a sequence that has at least 95%, 96%, 97%, 98%, 99% or more identity with SEQ ID NO:16, SEQ ID NO:19 (complementary to a portion of EGFR), a sequence that has at least 95%, 96%, 97%, 98%, 99% or more identity with SEQ ID NO:19, SEQ ID NO:20 (complementary to a portion of EGFR), a sequence that has at least 95%, 96%, 97%, 98%, 99% or more identity with SEQ ID NO:20, SEQ ID NO:21 (complementary to a portion of EGFR), a sequence that has at least 95%, 96%, 97%, 98%, 99% or more identity with SEQ ID NO:21, SEQ ID NO:24 (complementary to a portion of MMP7), a sequence that has at least 95%, 96%, 97%, 98%, 99% or more identity with SEQ ID NO:24, SEQ ID NO:29 (complementary to a portion of MED12), a sequence that has at least 95%, 96%, 97%, 98%, 99% or more identity with SEQ ID NO:29, SEQ ID NO:30 (complementary to a portion of RFX2), a sequence that has at least 95%, 96%, 97%, 98%, 99% or more identity with SEQ ID NO:30, SEQ ID NO:27 (complementary to a portion of TERT), a sequence that has at least 95%, 96%, 97%, 98%, 99% or more identity with SEQ ID NO:27, SEQ ID NO:31 (complementary to a portion of P21), a sequence that has at least 95%, 96%, 97%, 98%, 99% or more identity with SEQ ID NO:31, SEQ ID NO:32 (complementary to a portion of P13 KB), a sequence that has at least 95%, 96%, 97%, 98%, 99% or more identity with SEQ ID NO:32, SEQ ID NO:33 (complementary to a portion of SEPT9), a sequence that has at least 95%, 96%, 97%, 98%, 99% or more identity with SEQ ID NO:33, SEQ ID NO:28 (complementary to a portion of P53), a sequence that has at least 95%, 96%, 97%, 98%, 99% or more identity with SEQ ID NO:28, SEQ ID NO:50 (complementary to a portion of P53), and a sequence that has at least 95%, 96%, 97%, 98%, 99% or more identity with SEQ ID NO:50. It is understood that in some examples, any permutation or any combination of more than one of the nucleic acid probes provided herein, along with any permutation or any combination of more than one of the sets of primers provided herein, can be used in the methods and/or included in the compositions, combinations, kits and articles of manufacture provided herein, e.g., to quantitate cell-free nucleic acid derived from more than one target gene.

c. Incorporation of Reference Genes and Probes

The performance of Method 1 assays can be enhanced by the incorporation of the analysis of one or more reference gene(s) that indicate(s) the concentration of cell-free DNA in each plasma sample and is/are useful for the normalization of assay data against the subject-to-subject variations in the amount of input cell-free DNA. Such reference gene or genes can be analyzed in parallel, for example, in different assay tubes/wells from those for a target gene(s), or simultaneously, for example in the same assay tubes/wells with the use of multi-color detection as described in below. Reference gene include, for example, single-copy genes, such as, but not limited to, the genes for glyceraldehyde-3-phosphate dehydrogenase (GAPDH) and β-actin. Parallel analysis of a reference gene(s) also allows for the assessment of overall assay performance in a given reaction, in addition to the determination of the amount of input cell-free DNA. For example, a probe for a reference gene can have similar nucleotide composition similar to that for a target gene(s) of interest. For example, similar nucleotide composition includes, for example, 85%, 90%, 95%, 98% sequence identity or similar ratio, within 1%, 2%, 3%, 4%, 5%, of GC/AT content. The skilled artisan can select appropriate probes for this purpose.

d. Multiplexing

The performance of Method 1 assay can be enhanced by multiplexing, such as by employing differentially detectable PCR products. Differentially detectable PCR products include, for example, multi-color detection/quantification of PCR products (amplicons), for which a unique fluorophore-quencher pair is used in a TaqMan probe for each target gene. Fluorophore-quencher pairs are available for real-time quantitative PCR applications. By using appropriate fluorophore-quencher pairs in TaqMan probes, each of which is directed to a target gene, up to, for example, four target genes can be detected and quantified simultaneously. For example, the following four fluorophores can be detected with minimal interference: 6-FAM (peak emission wavelength, 517 nm), Cy3 (569 nm), TEX615 (613 nm), and Cy5 (670 nm). Each of four TaqMan probe species is directed to a target gene and has one of the four fluorophores (reporter dyes) at one of the termini with a quencher at the other terminus (Black Hole Quencher-1 for 6-FAM, and Black Hole QuencHER2 for the other three fluorophores). Segments of four target genes are amplified simultaneously with appropriate primers by real-time quantitative PCR, in which each of the four TaqMan probes is used to detect and quantify the amplicon derived from each of the four genes. This allows for simultaneous detection/quantification of the four genes, which can include a reference gene(s) (as described above).

e. Algorithm for Measuring Ct Values

In some examples, when real-time-qPCR is used to quantitate the amplified cell-free nucleic acid in Method 1 or other methods that include nucleic acid amplification as provided herein, e.g., combination methods provided herein, the Ct value can be measured as the first cycle at which exponential amplification begins, using the algorithm provided herein. The real-time-qPCR amplification generates S-curves as output (e.g., amount of fluorescence or other signal on the Y-axis; PCR amplification cycle number on the X-axis) as output. The curves are analyzed to measure “Ct”, the cycle at which the exponential part of the S-curve begins. This Ct value is used to distinguish positive (e.g., having a disease or condition) and negative (e.g., not having a disease or condition) test results. Ct can be derived using automated calculation procedures as follows:

(1) The S-curve is validated for fitting within a predefined band. This step is used to automatically disqualify measurements that lead to curves that are not S-shaped. The band is defined as an array of upper and lower values for each cycle. If the PCR measurement is larger than or lower than the defined band, this measurement is automatically disqualified. The upper bound is defined as a narrow band for early cycles, with linear growth for late cycles.

(2) The S-curve baseline is linearly approximated at lower cycles, e.g., between about 5, 6, 7, 8, 9 or 10 cycles to about 15, 20, 25, 30, 31 or 32 cycles or between about 10, 11, 12, 13, 14 or 15 cycles to about 20, 21, 22, 23, 24 or 25 cycles or between about 10 cycles to about 20 cycles or between about 20, 21, 22, 23, 24 or 25 cycles to about 30, 31 or 32 cycles or between about 20 cycles to about 30 cycles. The linear approximation can be adjusted depending on the correlation between the signal generated by the amplification product (e.g., fluorescence) and the cycle number. The baseline is then subtracted from the S-curve in order to adjust the curve.

(3) The Ct is calculated using an iterative math algorithm that analyzes the S-curve backwards from the last PCR cycle performed (e.g., 36, 37, 38, 39, 40, 41, 42, 43, 44, 45, 46, 47, 48, 49 or 50 or more cycles; in examples of the methods performed herein, the last cycle is Cycle 46) towards the first PCR cycle. At each iteration, the change (delta) between RFU (relative fluorescence units) values for neighboring cycles is calculated and the result is compared to a precalculated baseline level increased by 1 noise standard deviation (⁷). If delta is lower than this level, the Ct is defined at this point.

(4) The accuracy of Ct measurement can further be increased by linear approximation to levels of RFU values (or other signal), so the approximated Ct is defined as a point between two cycles, where the linearly approximated signal crosses the background (RFU_{baseline}+σₙₒᵢₛₑ) level. This linear approximation approach provides more precise information about Ct cycle without excessive overfitting.

(5) In some examples of the methods provided herein, Ct can be calculated for fragments amplified from more than one target gene, e.g., 1, 2, 3, 4, 5, 6, 7, 8, 9, 10 or more target genes, or between about 4 target genes to about 6 target genes, such as 4, 5 or 6 target genes. The final Ct value can be calculated as a mean value of all the measured Ct values, while omitting Ct values that are disqualified. Alternately, a weighting approach can be taken, depending, for example, on the target gene and the nature of its association with a disease or condition. In some examples, rather than measuring a mean Ct value, the Ct value for each target gene is assessed independently. If the majority (e.g., at least 50% or greater than 50%) of the Ct values are indicative of the disease or condition, e.g., cancer, the subject is identified as having the disease or condition. If between about 0% to less than about 50% of the Ct values, such as less than about 50%, 45%, 40%, 35%, 30%, 25%, 20%, 15%, 10%, 5% or less of the Ct values are indicative of the disease or condition, e.g., cancer, the subject is identified as not having the disease or condition.

(6) Samples with final Ct values (or majority of the final Ct values, see (5) above) equal to or lower than a cut-off value are classified as positive for a disease or condition, those Ct values (or majority of the final Ct values, see (5) above) higher than the cut-off value are classified as negative for a disease or condition, and disqualified values are ignored (omitted) from the analysis.

In some examples of the methods provided herein, Endpoint Analysis (measurement of the amount of signal generated in the last PCR cycle performed for a real-time qPCR analysis) can be added as a second parameter to the scoring function (in addition to Ct value).

### C. METHODS OF ANALYZING EXTRACELLULAR (CELL-FREE) NUCLEASE ACTIVITIES FOR DISEASE DETECTION AND/OR MONITORING PROGRESSION (METHOD 2A AND METHOD 2B)

### 1. Overview of the Methods

Provided herein are methods of analyzing a sample of body fluid from a subject for the presence or absence of a disease or condition in the subject by determining the amount of cell-free nuclease activity in the body fluid as indicative of the presence or absence of a disease or condition in the subject. A sample of body fluid that is obtained from a subject or has previously been obtained from a subject is subjected to conditions under which cell-free nuclease activity is assessed as a ratio of nuclease activities in one of two ways:

In “Method 2A,” a body fluid sample from a subject is exposed to two sets of reaction conditions: one in which the cell-free nuclease activity is measured in the presence of exogenously added zinc (Zn²⁺) and magnesium (Mg²⁺), and another in which the cell-free nuclease activity is measured in the presence of exogenously added magnesium alone, with no exogenously added zinc. If the ratio (K-A3) of the nuclease activity in the presence of exogenously added zinc relative to the nuclease activity in the absence of exogenously added zinc is at or above a threshold level, the subject is identified as having the disease or condition, and if this ratio is below a threshold level, the subject is identified as not having the disease or condition.

In “Method 2B,” a body fluid sample from the subject being tested for the presence or absence of a disease or condition is exposed to reaction conditions in which the cell-free nuclease activity of the sample is measured. A reference or control sample is subjected to the same or similar reaction conditions for measuring the nuclease activity, or a predetermined value of nuclease activity from a control or reference sample is obtained. If the ratio (K-A3B; depicted in the equation below as “K_{ARNA-3}”) of the nuclease activity measured in the subject being tested relative to the nuclease activity of the control or reference sample is at or above a threshold level, the subject is identified as having the disease or condition and if this ratio is below a threshold level, the subject is identified as not having the disease or condition. In some examples, the ratio is measured as follows:

$${K}_{ARNA - 3} = \frac{( {ES}_{endpoint} - {ES}_{first point} )}{( {RS}_{endpoint} - {RS}_{first point} )} ,$$

where ES is the “Examined Sample” (from a subject having a disease or condition, e.g., from a cancer patient) and RS is the “Reference Sample” (sample from validated control or reference or healthy subject). The reactions to measure nuclease activity in the sample from the subject and in the control or reference sample can both be performed in the presence of exogenously added magnesium alone, with no exogenously added zinc, or can both be performed in the presence of both exogenously added zinc and magnesium.

In “Method 2A” and “Method 2B,” the nuclease activities are measured by adding an exogenous nucleic acid probe as a substrate. In some examples, the nucleic acid probe contains one or more zinc finger protein binding sites (also referred to as motifs). It is found in the methods provided herein that the assessment of nuclease activity that is modulated by the effects of zinc, e.g., exogenously added zinc and/or the presence of a nucleic acid probe that contains at least one zinc binding motif, results in the ability to correctly identify subjects as having a disease or condition with a high level of sensitivity (e.g., 95%-100% sensitivity) and, additionally, good specificity, i.e., the ability to correctly identify subjects as not having a disease or condition (e.g., at least 60% or more specificity, up to 100% specificity). The threshold level generally is determined as a level that provides high sensitivity without compromising the specificity by identifying too many false positives (subjects who do not have the disease or condition being incorrectly identified as having the disease or condition).

In some examples of Method 2A and/or Method 2B, more than one nucleic acid probe, whose sequences differ from one another by one or more bases, are used as substrate(s) to measure nuclease activity in the same body fluid or sample thereof from the subject, such as 2, 3, 4, 5, 6, 7, 8, 9 or 10 or more different nucleic acid probes, and if the ratio of nuclease activities measured using at least 1, at least 2, at least 3, at least 4, at least 5, at least a quarter, a third, half, two thirds, three quarters or more, up to all of the nucleic acid probes assessed is indicative of the presence of a disease or condition, the subject is identified as having the disease or condition. In some examples of Method 2A and/or Method 2B, more than one nucleic acid probe, whose sequences differ from one another by one or more bases, are used as substrate(s) to measure nuclease activity in the same body fluid or sample thereof from the subject, such as 2, 3, 4, 5, 6, 7, 8, 9 or 10 or more different nucleic acid probes, and if the ratio of nuclease activities measured using at least 1, at least 2, at least 3, at least 4, at least 5, at least a quarter, a third, half, two thirds, three quarters or more, up to all of the nucleic acid probes assessed is indicative of the absence of a disease or condition, the subject is identified as not having the disease or condition. In examples, the mean or the median of the ratio of nuclease activity measured using more than one nucleic acid probe is obtained and if the mean or median of the ratio of nuclease activity levels measured using more than one nucleic acid probe is indicative of the presence or absence of a disease or condition, the subject is identified as either having or not having the disease or condition, respectively.

In some examples, at least one nucleic acid probe is derived from a target gene. In some examples, at least one nucleic acid probe contains at least one zinc finger binding site (motif).

As shown in the examples and discussed below, the Methods 2 assess a combination of reactions; primarily, the digestion of probes by nucleases, and the binding of plasma components to the probes. Thus, for a given disease, disorder, or condition, such as cancer or other health condition, positive results (i.e., higher digestion rates of probes) are not necessarily derived from higher nuclease activities. Instead, positive results can result from greater amounts of the binding of plasma components to probes, which results in the protection of the probes and the reduction of the digestion rate, even when the nuclease activity has no differences between normal subjects and those with a particular disease, disorder, or condition, such as cancer.

In particular, in Methods 2 assays, the changes in fluorescence intensity, derived from probe molecules (substrates), are monitored. Each probe molecule has a fluorophore and a dark quencher at its termini, so that fluorescence emission is suppressed by fluorescence resonance energy transfer between the fluorophore and the dark quencher at varying degrees that are dependent on the nucleotide sequence and the conformation of probe molecules. These assays involve two independent, but interactive events. The primary event, which induces the changes in probe-derived fluorescence emission, is the digestion (cleavage) of probe molecules, which results in the increase in fluorescence intensity due to the cancellation of fluorescence resonance energy transfer. Other events also affect the properties of fluorescence emission from probe molecules. One such event is the binding of plasma components, primarily plasma proteins, to probe molecules. Binding of a plasma component(s) to probes induces the following changes of probe-derived fluorescence emission: (i) Binding of a plasma component(s) to (or near) the fluorophore on the probe reduces fluorescence emission by the shielding of the fluorophore; (ii) binding of a plasma component(s) to (or near) the fluorophore can make the distance between the fluorophore and the dark quencher larger, resulting in the increase of fluorescence emission; (iii) binding of a plasma component(s) to (or near) the dark quencher increases fluorescence emission by the shielding of the dark quencher; and (iv) binding of a plasma component(s) to probe molecules induces their conformational changes, changes the distance between the fluorophore and the dark quencher, and alters the local environment around the fluorophore and the dark quencher, resulting in the increase or decrease of fluorescence emission. In addition to these changes of probe-derived fluorescence emission, the binding of a plasma component(s) to probe molecules alters their susceptibility to digestion (degradation) by plasma nucleases and often slows the rate of their digestion. Hence “positive results,” indicative of a disease, disorder, or condition can be determined as a difference compared to a healthy or normal subject or to a control or standard. The controls or standards or values for healthy or normal subjects can be empirically determined.

Additionally, while exemplified and described herein, these assays can be performed using any suitable methods/techniques for the detection and/or quantification of intact and/or digested probes without any change of the assay principle and with little need for the modification of assay procedures. Detection and/or quantification methods include, but are not limited to, mass-spectrometry-mediated detection of probes, single-molecule real time sequencing, and detection of probes by fluorometry with fluorometers, including fluorescence plate readers and fluorescence spectrophotometers.

### 2. Cell-Free Nucleases

Exogenous and endogenous cell-free nucleic acids in a body fluid, such as DNA and RNA, can be degraded in blood plasma due to the presence of enzymes that are nucleases, such as DNAses or RNAses, or display nuclease-like activity. For example, the deoxyribonuclease, DNAse I, is responsible for almost 90% of the DNA hydrolytic activity in blood. Acid DNase II, phosphodiesterase I, DNA-hydrolyzing autoantibodies, and lactoferrin also digest DNA. Inhibitors of DNases, such as actin, also can be found in body fluids such as blood. Therefore, extracellular nuclease activity in a body fluid is dependent on the concentrations and composition of nucleases as well as factors influencing the activity of those nucleases.

DNases are enzymes that are able to hydrolyze phosphodiester bonds of DNA molecules. The main classes of DNAses can be divided into two families that differ in biochemical and biological properties, although the ability to hydrolyze DNA is common to both of them: the DNase I and DNase II families. DNases are encoded by several genes and expressed in many tissues. Some of them are secreted, and, therefore, DNases can cleave DNA in both intracellular and extracellular space. In some cases, it has been shown that extracellular DNA is generated first by cleavage inside cells by intracellular DNases, followed by fragmentation with extracellular DNases (Han et al., Am. J. Hum. Genet., 2020, 106:202-214 (2020)). The DNase I family includes members such as DNase I, DNase1L1, DNase1L2 and DNase1L3, while the DNase II family includes DNase II α and DNase II β. Although L-DNase II is considered to be part of the DNase II family, the putative gene is SERPINB1. Other enzymes do not belong to a specific DNAse family but nonetheless have a role in DNA degradation, such as three-prime repair exonucleases (TREX) such as TREX1 and TREX2.

Increased expression of some nucleases is seen in certain cancers. In breast cancer tumor cells, increased expression of various nucleases, namely: EXO1, NEIL3, FEN1, DNA2 and ERCC1 was detected. In addition, the analysis of gene expression data from the European Molecular Biology Laboratories (EMBL-EBI) revealed that 143 out of 160 genes that encode putative nucleases are upregulated in breast cancer cell lines (Kruspe et al., Mol. Ther.—Nucleic Acids, 8:542-557 (2017)).

In Method 2A, Method 2B and certain combination methods as provided herein, cell-free nuclease activity that is modulated by zinc is measured, for example, in the presence of exogenous zinc (Zn²⁺) and/or by using a nucleic acid probe containing at least one zinc finger binding motif as a substrate. Without being bound by theory, in at least some examples, the cell-free nuclease(s) whose activity/activities is/are being assessed are zinc finger proteins (zinc finger nucleases—ZFN) and/or the cell-free nuclease activity is modulated by a zinc finger protein.

Zn fingers (ZNFs) are implicated in disease processes. For example, Zn fingers are involved in tumorigenesis, cancer progression and metastasis formation. Alterations in ZNFs are involved in the development of several of diseases such as neurodegeneration, skin disease and diabetes (see, e.g., Cassandri et al., (2017) Cell Death Discov. 3:17071, doi.org/10.1038/cddiscovery.2017.71).

### 3. Measurement of Nuclease Activity

Methods for measuring nuclease activity include enzyme-linked immunosorbent assays (ELISA), colorimetric assays, radial immunodiffusion (RID) assays, and radial enzyme diffusion (RED) assays. In the methods provided herein, in some examples, digestion of a substrate nucleic acid by a nuclease can be measured by an increase or decrease in a signal associated with a detectable label, such as a chromophore, a radiolabel, a chemiluminescent label or a fluorophore. For example, if the detectable label is a fluorophore, the hydrolysis of fluorescently labeled DNA into fragments can be measured by capillary electrophoresis (Vancevska et al., Laboratory Medicine, 44(2):125-128 (2013)). Several methods of determining nuclease activity are known to those of skill in the art, such as those described, for example, in Laukovi et al., Biomolecules, 10(7):1036 (2020) and references cited therein. For example, the cleavage of nucleic acids by some nucleases is accompanied by an increase of absorption of UV light, which can be measured to determine nuclease activity. Single Radial Enzyme Diffusion (SRED) is based on the digestion of nucleic acid in an agarose gel. The nucleic acid stained with a binding dye, such as, for example for DNA, ethidium bromide, SYBR Green I or other dyes for DNA. Nuclease activity is represented by the size of a dispensed circular well in an agarose gel layer, in which DNA stained by, e.g., ethidium bromide is uniformly distributed. After the incubation, a circular dark zone is formed as the enzyme diffuses from the well radially into the gel and cleaves DNA. The diameter of the dark circle positively correlates with the amount of the enzyme applied to the well. Another assay is a kinetic colorimetric activity assay, based on the degradation of a DNA/methyl green complex. A similar method is based on the ability of PicoGreen dye to enhance its fluorescence when bound to double stranded DNA. In this fluorometric assay, the reaction mixture of DNAse I and a DNA substrate is prepared in a fluorescence microtiter plate. PicoGreen reagent is added to each well at the end of the incubation and the fluorescence intensity is measured. The fluorescence intensity negatively correlates with DNAse activity. Immunochemical microtiter plate-based assays, based on the cleavage of biotinylated and fluorescein-labeled PCR products of different lengths, and on the immunochemical detection of non-digested DNA, can be used. Other methods include microchip electrophoresis for measuring endonuclease activity and a fluorometric assay for measuring exonuclease activity based on the preferential binding of ssDNA over dsDNA to graphene oxide.

Other methods for measuring nuclease activity include a lateral flow immunochemical assay based on a dually labeled dsDNA as the reporter probe. The probe has a biotin-labeled terminal bound to streptavidin immobilized on a lateral flow test strip and a fluorescein-labeled terminal bound to antibody-conjugated gold nanoparticles. Nuclease activity is measured as the test line intensity decreases, caused by the cleavage of the reporter probe. Nuclease activity can readily be measured in replicate analyses using, e.g., a 96-well plate fluorescence reader, or a reader of other detectable signals. For example, DNAse activity can be measured by the degradation of ethidium bromide-double-stranded DNA complexes in a defined time (decrease in fluorescence intensity) using a 96-well plate fluorescence reader.

In examples of the methods provided herein, cell-free nuclease activity is measured using a nucleic acid probe as substrate. Any nucleic acid suitable for use as a substrate for cell-free nucleases can be used, including any of the nucleic acid probes used in Method 1 or other methods that include nucleic acid amplification, such as certain combination methods provided herein. In some examples, at least one nucleic acid probe used to quantitate cell-free nucleic acid, e.g., in Method 1 or combination methods as provided herein has the same sequence as a nucleic acid probe used to measure/determine cell-free nuclease activity in Method 2A, Method 2B or certain combination methods provided herein. In examples, the nucleic acid probes used as substrates can be between about 10 to about 100 nucleotides, between about 10 to about 70 nucleotides, between about 10 to about 50 nucleotides, between about 15 to about 30 nucleotides, generally at least or about 15, 16, 17, 18, 19, 20, 21, 22, 23, 24, 25, 26, 27, 28, 29 or 30 nucleotides in length, or at least about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length.

In some examples, the nucleic acid probes used in Method 2A, Method 2B or other methods provided herein that include measuring nuclease activity, such as the combination methods provided herein, can be associated with a label that can provide a detectable “reporter” signal, such as by covalent linkage or non-covalent interactions, which can emit a signal that is dependent (or inversely dependent) on the amount of cell-free nuclease activity. Any labels that can provide a detectable signal indicative of the amount of cell-free nuclease activity, such as those known to those of skill in the art and/or described herein, can be used. Using distinct labels, more than one nucleic acid probe can be used in the same reaction to analyze the amount of cell-free nuclease activity in a sample of body fluid.

In some examples of Method 2A, Method 2B or other methods provided herein that include measuring nuclease activity, such as the combination methods provided herein, a nucleic acid probe is labelled with a fluorescent reporter molecule and a fluorescence quencher molecule. Examples of fluorescent reporter molecules for nucleic acid include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. Many equivalent detection labels, both proprietary and non-proprietary, are readily available and known to those of skill in the art for use in real-time-qPCR.

Fluorescence quencher labels for labelling nucleic acid probes can include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, BHQ-0, BHQ-1, BHQ-2, BHQ-3, DABSYL (dimethylaminoazobenzenesulfonic acid), Iowa Black FQ/Iowa Black RQ and related fluorescence quenchers such as those described in U.S. Pat. Nos. 7,439,341, 7,803,536, 7,476,735, 7,605,243, 7,645,872, 8,030,460, 8,084,588, 8,114,979, 8,258,276 and 8,916,345, the contents of which are incorporated expressly by reference herein. Many equivalent fluorescence quencher labels, both proprietary and non-proprietary, are readily available and known to those of skill in the art for use in real-time-qPCR. In examples, the fluorescent reporter molecule is at the 5′-end of the nucleic acid probe and the fluorescence quencher molecule is at the 3′-end of the nucleic acid probe. In examples, the fluorescent reporter molecule is FAM and the fluorescence quencher molecule is BHQ-1.

In examples where the nucleic acid probe is labelled with a fluorescent reporter molecule and a fluorescence quencher, measurement of the amount of cell-free nuclease activity can be performed as follows. Before digestion of the nucleic acid probe is initiated, the close proximity of the fluorescent reporter to the quencher prevents detection of its fluorescence. Digestion of the probe breaks the reporter-quencher proximity due to hydrolysis of the nucleic acid probe, thereby permitting unquenched emission of fluorescence, which can be detected by excitation with a laser. An increase in the amount of digestion of the substrate (nucleic acid probe) leads to a proportional increase in fluorescence due to the breakdown of the probe and release of the reporter, which in turn can be used as a measure of cell-free nuclease activity.

The volume of body fluid that is analyzed in each reaction for measuring cell-free nuclease activity can between about 1 μL to about 1 mL, such as 100 μL or less, between about 5 μL to about 50 μL, between about 2 μL to about 25 μL, or between about 1 μL to about 20 μL, or between about 1 μL to about 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL or 10 μL. In some examples, the volume of body fluid analyzed is about or at least about 1 μL, 2 μL, 2.5 μL, 3 μL, 3.5 μL, 4 μL, 4.5 μL, 5 μL, 5.5 μL, 6 μL, 6.5 μL, 7 μL, 7.5 μL, 8 μL, 8.5 μL, 9 μL, 9.5 μL, 10 μL, 15 μL, 20 μL, 25 μL or 30 μL.

The total volume of each reaction for measuring cell-free nuclease activity, in some examples, can be between about 15 μL to about 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL or 100 μL or more, such as at least about 100 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, or 1 mL or more. In some examples, the amount of a nucleic acid probe added to each reaction in Method 2A, Method 2B or other methods that include measuring cell-free nuclease activity as provided herein, e.g., certain combination methods provided herein, can be from at least about or at 0.01 μM to about 2 μM, such as between about 0.05 μM to about 1.5 μM or between about 0.1 μM to about 1 μM. In some examples, the amount of nucleic acid probe added to each reaction in Method 2A, Method 2B or other methods that include measuring cell-free nuclease activity as provided herein, e.g., certain combination methods provided herein, can be about or at least about 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM or 1 μM. In some examples, the amount of nucleic acid probe added to each reaction can be between about 0.5 μM to about 0.8 μM, such as at least or about 0.5 μM, 0.55 μM, 0.6 μM, 0.65 μM, 0.7 μM, 0.75 μM or 0.8 μM.

In some examples, the nucleic acid probe sequence is derived from a target gene of interest. In examples, the target gene from which the nucleic acid probe sequence is derived contains at least one zinc finger binding motif. In examples, the zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats.

In some examples, the nucleic acid probe contains at least one zinc finger binding motif. In examples, the zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats. In examples, x is between about 3 repeats to about 17 repeats. Exemplary sequences containing 3 or more, up to 17 repeats, are shown in Table 23 below:

TABLE 23
Exemplary Nucleic Acid Probe Sequences Containing (CNN) repeats

| Genecards.org | GENE | PROBES | Position | (CNN)X |
|---|---|---|---|---|
| NM_000926.4 | PGR | 5′- | 3143-3168 | (CNN)3 |
| | | CATGTGGCAGATCCCACAGGAGTTTG- | | |
| | | 3′ (SEQ ID NO: 4) | | |
| NM_000125 | ESR1 | 5′-CTGAACCGTCCGCAGCTCAAGATCC- | 307-331 | (CNN)4 |
| | | 3′ (SEQ ID NO: 1) | | |
| NM_001005862 | HER2 | 5′-CCCAGCCCTGGTCACCTACAACAC- | 1280-1303 | (CNN)4 |
| | | 3′ (SEQ ID NO: 7) | | |
| NM_002423.5 | MMP7 | 5′- | 259-294 | (CNN)4 |
| | | CTGGAATGTTAAACTCCCGCGTCATAG | | |
| | | AAATAATGC-3′ (SEQ ID NO: 24) | | |
| NM_001346900 | EGFR | 5′-GTGACTGCTGCCACAACCAG- 3′ | 718-737 | (CNN)5 |
| | | (SEQ ID NO: 20) | | |
| NM_005228.5 | EGFR | 5′- | 2293-2324 | (CNN)10 |
| | | AGCTCCCTCTCCTGCAGCAGCCTCCGC | | |
| | | AGCGT-3′ (SEQ ID NO: 21) | | |
| NM_001193376.3 | TERT | 5′- | 1856-1885 | (CNN)10 |
| | | TTGAAGAGGGTGCAGCTGCGGGAGCT | | |
| | | GTCG-3′ (SEQ ID NO: 27) | | |
| NM_00126114.2 | P53 | 5′- | 423-471 | (CNN)17 |
| | | CCCCTGCACCAGCAGCTCCTACACCGG | | |
| | | CG GCCCCTGCACCAGCCCCCTC-3′ | | |
| | | (SEQ ID NO: 50) | | |

In some examples, the target gene from which the cell-free nucleic acid is derived is selected from among ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9). Exemplary nucleic acid probes for quantitating cell-nucleic acids derived from certain target genes include those selected from among one or more of the following:

SEQ ID NO:1 (complementary to a portion of ESR1), a sequence that has at least 95%, 96%, 97%, 98%, 99% or more identity with SEQ ID NO:1, SEQ ID NO:4 (complementary to PGR), a sequence that has at least 95%, 96%, 97%, 98%, 99% or more identity with SEQ ID NO:4, SEQ ID NO:7 (complementary to a portion of HER2), a sequence that has at least 95%, 96%, 97%, 98%, 99% or more identity with SEQ ID NO:7, SEQ ID NO:10 (complementary to a portion of ARFIP1), a sequence that has at least 95%, 96%, 97%, 98%, 99% or more identity with SEQ ID NO:10, SEQ ID NO:13 (complementary to a portion of COX1), a sequence that has at least 95%, 96%, 97%, 98%, 99% or more identity with SEQ ID NO:13, SEQ ID NO:16 (complementary to a portion of PLIN1), a sequence that has at least 95%, 96%, 97%, 98%, 99% or more identity with SEQ ID NO:16, SEQ ID NO:19 (complementary to a portion of EGFR), a sequence that has at least 95%, 96%, 97%, 98%, 99% or more identity with SEQ ID NO:19, SEQ ID NO:20 (complementary to a portion of EGFR), a sequence that has at least 95%, 96%, 97%, 98%, 99% or more identity with SEQ ID NO:20, SEQ ID NO:21 (complementary to a portion of EGFR), a sequence that has at least 95%, 96%, 97%, 98%, 99% or more identity with SEQ ID NO:21, SEQ ID NO:24 (complementary to a portion of MMP7), a sequence that has at least 95%, 96%, 97%, 98%, 99% or more identity with SEQ ID NO:24, SEQ ID NO:29 (complementary to a portion of MED12), a sequence that has at least 95%, 96%, 97%, 98%, 99% or more identity with SEQ ID NO:29, SEQ ID NO:30 (complementary to a portion of RFX2), a sequence that has at least 95%, 96%, 97%, 98%, 99% or more identity with SEQ ID NO:30, SEQ ID NO:27 (complementary to a portion of TERT), a sequence that has at least 95%, 96%, 97%, 98%, 99% or more identity with SEQ ID NO:27, SEQ ID NO:31 (complementary to a portion of P21), a sequence that has at least 95%, 96%, 97%, 98%, 99% or more identity with SEQ ID NO:31, SEQ ID NO:32 (complementary to a portion of P13 KB), a sequence that has at least 95%, 96%, 97%, 98%, 99% or more identity with SEQ ID NO:32, SEQ ID NO:33 (complementary to a portion of SEPT9), a sequence that has at least 95%, 96%, 97%, 98%, 99% or more identity with SEQ ID NO:33, SEQ ID NO:28 (complementary to a portion of P53), a sequence that has at least 95%, 96%, 97%, 98%, 99% or more identity with SEQ ID NO:28, SEQ ID NO:50 (complementary to a portion of P53), and a sequence that has at least 95%, 96%, 97%, 98%, 99% or more identity with SEQ ID NO:50. It is understood that in some examples, any permutation or any combination of more than one of the nucleic acid probes provided herein, along with any permutation or any combination of more than one of the sets of primers provided herein, can be used in the methods and/or included in the compositions, combinations, kits and articles of manufacture provided herein, e.g., to quantitate cell-free nucleic acid derived from more than one target gene.

In any of the examples of Method 2A, Method 2B or other methods that include measuring nuclease activity as provided herein, e.g., certain combination methods provided herein, the body fluid sample can be selected from among whole blood, serum, plasma, urine, cerebrospinal fluid, pleural fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk and breast fluid discharge. In some examples, the body fluid is plasma.

Reaction conditions for measuring cell-free nuclease activity are known to those of skill in the art; any and all of these methods are incorporated in their entirety by reference herein. Exemplary, but not limiting, conditions can include the following: Each reaction contains a sample of a body fluid, such as plasma, and a suitable reaction buffer such as those described in the working examples, e.g., Tris-HCl and other such buffers, or equivalent thereto as understood by those of skill in the art. A suitable amount of at least one nucleic acid probe, such as in amounts described herein or as known to those of skill in the art, is added. One or more of the following reagents, such as detergents known to those of skill in the art and/or described herein (e.g., NP40 in an amount of about or at least about 0.05%, 0.1%, 0.15%, 0.2%, 0.25%, 0.3% or other such suitable amount as known to those of skill in the art), cofactors such as Mg²⁺ with or without exogenous Zn²⁺, and/or a reducing agent can be added. In some examples, samples can be analyzed in duplicate, triplicate, quadruplicate or higher order, e.g., in plates containing a plurality of wells, such as a 96-well plate. The reactions generally are performed at a temperature of between about 30° C. to about 45° C., such as at least or about 30° C., 31° C., 32° C., 33° C., 34° C., 35° C., 36° C., 37° C., 38° C., 39° C., 40° C., 41° C., 42° C., 43° C. or more, although, depending on the body fluid sample, the nuclease activity to be measured and other conditions, the temperature can be lower, such as between 25° C. or less than 25° C. to about 30° C. The pH of the reactions generally is between about 7 to about 9, although depending on the body fluid sample, the nuclease activity being measured and other such parameters, reactions can be performed at a pH of between about 5 to about 9. In examples, the pH of the reaction is about or at least 7, 7.1, 7.2, 7.3, 7.4. 7.5, 7.6, 7.7, 7.8, 7.9, 8.0, 8.1, 8.2, 8.3, 8.4, 8.5, 8.6, 8.7, 8.8, 8.9 or 9.0. In some examples, the pH is between about 8.0 to about 8.5, such as about 8.0, 8.1, 8.2, 8.3, 8.4 or 8.5. In examples, the pH is 8.3.

The reactions can be performed from between about 25 minutes to about 30, 35, 40, 45 or more minutes, such as at least or about 25 minutes, 30 minutes, 35 minutes, 40 minutes, 45 minutes or more. In examples, the reactions are incubated for about 30 minutes or about 35 minutes at about 33° C., 34° C., 35° C., 36° C., 37° C., 38° C., 39° C., 40° C. or 41° C. In some examples, the reactions can be performed in a multi-well plate, such as a 96-well plate. The plate can be covered with UV-protective sticky film and transferred to a PCR thermocycler, where incubation for a desired length of time can be performed. During incubation of any of the reactions performed according to Method 2A, Method 2B or other methods that include measuring nuclease activity as provided herein, e.g., certain combination methods provided herein, a detectable signal, such as fluorescence, can be recorded during the incubation at regular intervals, such as every 10 seconds, 15 seconds, 20 seconds, 25 seconds, 30 seconds, 35 seconds, 40 seconds, 45 seconds, 50 seconds, 55 seconds, 1 minute, 1.5 minutes, 2 minutes, 2.5 minutes, 3 minutes, 3.5 minutes, 4 minutes, 4.5 minutes, 5 minutes or longer. In some examples, the interval is at least or about 1 minute.

a. Absence of Added Zinc

In some examples, the cell-free nuclease activity in a body fluid is measured in the presence of added Mg²⁺ as a cofactor and in the absence of exogenous Zn²⁺, i.e., the Mg²⁺-dependent nuclease activity is measured. Any salt of Mg²⁺ can be used including, but not limited to, MgCl₂, MgSO₄, Mg(CH₃COO)₂ and others known to those of skill in the art. In some examples, the Mg²⁺ concentration in the reaction is between about 3 mM to about 20 mM. In examples, the Mg²⁺ concentration is about 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM or 18 mM. In some examples, the Mg²⁺ concentration is between about 5 mM to about 7 mM. In examples, the Mg²⁺ concentration is about 6 mM, 6.1 mM, 6.2 mM, 6.3 mM, 6.4 mM or 6.5 mM.

b. Presence of Added Zinc

In some examples, the cell-free nuclease activity in a body fluid is measured in the presence of added Mg²⁺ as a cofactor and in the presence of exogenous Zn²⁺ as a cofactor, i.e., the added influence of exogenous Zn²⁺ as a cofactor on cell-free nuclease activity is measured. Any salt of Zn²⁺ can be used including, but not limited to, ZnCl₂, ZnSO₄, Zn(CH₃COO)₂ and others known to those of skill in the art. In some examples, the Zn²⁺ concentration is between from 10 μm to 15 mM, such as 10-50 μM, 10-25 μM, 15 μM to 30 μM, 20 μM to 100 μM, 1 μM and 10 mM and higher, including 1 mM to 15 mM, such as about 5 mM to about 15 mM. In examples, the Zn²⁺ concentration is about 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM or 15 mM. In examples, the Zn²⁺ concentration is between about 6 mM to about 10 mM. In examples, the Zn²⁺ concentration is about between about 6 mM to about 7 mM. In some examples, the Zn²⁺ concentration is about 9 mM. In practice, the lower concentrations can be used, the range can be 1 μM to 10 mM.

c. Determining a Ratio of Cell-free Nuclease Activities

In some examples, the ratio of cell-free nuclease activities can be determined as a ratio of: (a) the cell-free nuclease activity in a sample of body fluid in the presence of added Mg²⁺ as a cofactor and exogenous Zn²⁺ as a cofactor to (b) the cell-free nuclease activity in the sample of body fluid in the presence of added Mg²⁺ alone and the absence of exogenous Zn²⁺ as a cofactor. If this ratio is at or above a threshold, the subject from whom the sample of body fluid is obtained is identified as having a disease or condition and if this ratio is below a threshold, the subject from whom the sample of body fluid is obtained is identified as not having a disease or condition (Method 2A and certain combination methods as provided herein).

In some examples, the ratio of cell-free nuclease activities can be determined as a ratio of: (a) the cell-free nuclease activity in a sample of body fluid from the subject being tested for the presence or absence of a disease or condition, where the cell-free nuclease activity is measured in the presence of added Mg²⁺ as a cofactor and either the presence or absence of exogenous Zn²⁺ as a cofactor to (b) the cell-free nuclease activity in a control or reference sample measured under identical or analogous conditions as the sample from the subject. If this ratio is at or above a threshold, the subject is identified as having a disease or condition and if this ratio is below a threshold (or differs in a predetermined way from the threshold), the subject is identified as not having a disease or condition (Method 2B and certain combination methods as provided herein).

d. Presence of a Redox Reagent

In some examples, the cell-free nuclease activity in a body fluid is measured in the presence of Mg²⁺ as a cofactor, the presence or absence of exogenous Zn²⁺ as a cofactor, and a redox reagent/reducing agent. In examples, the reducing agent is selected from among TCEP (tris(2-carboxyethyl) phosphine), DTT, DTE, glutathione, N-acetylcysteine, 2-mercaptoethanol, cysteine hydrochloride, sodium thioglycolate, diethyldithiocarbamate, thioglycolic acid and DTBA (dithiobutylamine). In some examples, the reducing agent is DTT.

In examples, the reducing agent is added in an amount that increases the difference between the cell-free nuclease activities and/or ratios of cell-free nuclease activities measured in a subject having the disease or condition and those measured in a subject not having the disease or condition. In some examples, the concentration of the reducing agent is between about 0.1 mM to about 2 mM. In examples, the concentration of the reducing agent is between about 0.1 mM to about 1 mM or 1.5 mM. In some examples, the concentration of the reducing agent is between about 0.1 mM to about 0.6 mM. In examples, the concentration of the reducing agent is about or equal to 0.1 mM, 0.2 mM, 0.3 mM, 0.4 mM or 0.5 mM. In some examples, the concentration of the reducing agent is about or equal to 0.4 mM, or about or equal to 0.5 mM. In some examples, the reducing agent is DTT.

e. Reference Probes

The performance of these assays, Methods 2, can be enhanced by the incorporation of the analysis of a reference probe or probes. Such reference probes are those that do not exhibit appreciable differences in the changes in fluorescence intensity (or other readout) among different subject groups (for example, subjects with a disease of interest and normal or healthy subjects). Such reference probes can be analyzed in parallel, for example in different assay tubes/wells from a probe(s) for the discrimination of different subject groups, or simultaneously, for example in the same assay tubes/wells with the use of multi-color (multiplex) detection (as discussed above for Method 1). Parallel analysis of such reference probes provides for the assessment of overall assay performance in a given reaction. The reference probes can have different nucleotide compositions, such as varying GC contents. The reference probes can be selected so that the nucleotide composition of a reference probe(s) is similar, such as GC content ±20% for the corresponding target, to that for a probe(s) that is used for the discrimination of different subject groups. Similar GC content refers to at least ±25%, ±20%, ±15%, or ±10%, or other pre-determined percentage, of the GC content of the target gene or corresponding segment of the target gene. Generally, probes are considered to have similar nucleotide composition if they have at least ±20% or more of the GC content of the target.

Depending upon the target, and/or disease, disorder, or condition, the reference probe(s) can also include sequences that are related to or not related to potential binding sites for zinc finger proteins.

f. Multiplexing

The performance of the assays of Method 2 can be enhanced by multiplexing, which involves multi-color detection/quantification of probes (substrates). Each fluorophore-quencher pair is used for each probe species. As discussed above, a number of fluorophores are available, and by using appropriate fluorophore-quencher pairs, up to four probe species can be detected and quantified simultaneously. For example, the following four fluorophores can be detected with minimal interference: 6-FAM (peak emission wavelength, 517 nm), Cy3 (569 nm), TEX615 (613 nm), and Cy5 (670 nm). Each of four probe species has one of the four fluorophores (reporter dyes) at one of the termini with a quencher at the other terminus (Black Hole Quencher-1 for 6-FAM, and Black Hole QuencHER2 for the other three fluorophores). Four probe species are mixed with a plasma sample, and the changes in fluorescence intensity, derived from each probe species, are monitored. This provides simultaneous detection and/or quantification of fluorescence emission, derived from each of the four probes. One or more of the probe species can be a reference probe(s), discussed above, that gives no appreciable differences in the changes in fluorescence intensity among different subject groups (for example, subjects with a disease of interest and normal subjects).

### D. COMBINATION METHODS

In some examples of methods provided herein, a combination of modalities can be used to analyze a body fluid from a subject to determine the presence or absence of a disease or condition in the subject. In particular, Methods 1 and Methods 2 can be combined. For example, combination method can include:
1) subjecting a first aliquot of a sample of body fluid previously obtained from the subject to an amplification reaction, where the sample contains cell-free nucleic acid, quantitating the resulting amplified nucleic acid and determining whether the amount of amplified nucleic acid is at or above a threshold level, or below a threshold level;
2) subjecting a second aliquot of the same sample of body fluid to a reaction to determine an enzymatic activity, determining whether the enzymatic activity is at or above a threshold level, or below a threshold level; and
3) if either or both of the following conditions are met: i) the amount of amplified nucleic acid is at or above a threshold level, and/or ii) the enzymatic activity is at or above a threshold level, identifying the subject as having the disease or condition.

The enzymatic activity can be any enzyme whose activity is modulated by the disease or condition. In some examples, the enzymatic activity is reduced in a subject having a disease or condition compared to a subject not having the disease or condition; in that instance, in 3) above, if either or both of the following conditions are met: i) the amount of amplified nucleic acid is at or above a threshold level, and/or ii) the enzymatic activity is below a threshold level, the subject is identified as having the disease or condition. In other embodiments, it can be increased in a subject having disease, disorder, or condition compared to a subject not having the disease, disorder, or condition.

In some examples, the enzyme whose activity is measured can be a kinase. In examples, the enzyme whose activity is measured can be a protease. In examples, the enzyme whose activity is measured can be a nuclease. In examples of the methods provided herein, more than one type of enzyme activity can be measured in a body fluid of the subject being tested for the presence or absence of a disease or condition. In some examples, the cell-free nucleic acid that is amplified is derived from a target gene of interest. In examples, cell-free nucleic acid that is amplified is derived from more than one target gene of interest.

In the methods provided herein, the threshold level of amplified cell-free nucleic acid can be determined based on a level that is measured in at least one subject known to have the disease or condition and/or is based on a level that is measured in at least one control or reference sample, or the threshold level can be determined based on a level that is the mean or the median of levels measured in more than one subject known to have the disease or condition and/or the mean or the median of levels measured in more than one control or reference sample. The threshold level of an enzymatic activity can be determined based on the enzymatic activity that is measured in at least one subject known to have the disease or condition and/or is based on the ratio that is measured in at least one control or reference sample, or can be determined based on an enzymatic activity that is the mean or the median of ratios measured in more than one subject known to have the disease or condition and/or the mean or the median of ratios measured in more than one control or reference sample. In some examples of the combination methods provided herein, determining whether an enzymatic activity is at or above a threshold level is performed by obtaining a ratio of two enzymatic activities, wherein the two enzymatic activities are measured on the same sample of body fluid under two different reaction conditions, and/or the two enzymatic activities are measured in the sample of body fluid from the subject and in a control or reference sample under analogous/identical conditions.

In examples of the combination methods provided herein, quantitation of the amplified cell-free nucleic acid is by real-time-qPCR. In some examples, the quantitation by real-time-qPCR is performed using a nucleic acid probe that hybridizes to the cell-free nucleic acid and measuring a Ct value. The Ct value can be determined by measuring a signal that is proportional to the amount of the amplification product, such as fluorescence, and can be measured as the amplification cycle number at which a signal above a background level is detected or as the amplification cycle number where exponential increase of a signal associated with the amplification product begins. In examples, Ct value is determined using the algorithm provided herein.

In any of the combination methods provided herein, in some examples, the enzymatic activity being determined is a nuclease activity. In examples, the nuclease activity is measured by adding a nucleic acid probe and by measuring digestion of the nucleic acid probe. In some examples, the same nucleic probe that is used to measure nuclease activity in a sample of body fluid from a subject can be used to quantitate cell-free nucleic acid in the sample of body fluid from the subject.

Any of the examples or embodiments of Method 1 can be combined with any of the examples or embodiments of Method 2A and/or 2B and/or additional enzymatic activities to perform the combination methods provided herein. In some examples, multiple (three or more) determinations can be made in a sample of body fluid from a subject, e.g., quantitation of one or more amplified cell-free nucleic acid(s), measurement of nuclease activity/activities, measurement of additional enzymatic activity/activities, and if the threshold level of at least one, at least two, at least three, at least or about a quarter, at least or about a third, at least or about half, at least or about three quarters or more of the parameters measured (amount of cell-free nucleic acid, nuclease activity, other enzymatic activity, etc.) are indicative of the presence of a disease or condition in the subject, the subject is identified as having the disease, disorder, or condition.

### E. DETERMINATION OF A THRESHOLD LEVEL

A “threshold level” generally refers to a predetermined level or amount that differentiates between subjects having a disease or condition and subjects not having a disease or condition. The threshold level can be based on a scoring function (see, e.g., Example 12) that is empirical or is based on prior knowledge regarding assessment of the parameter and/or assays used to assess the parameter, e.g., determination of an enzymatic activity or determination of the amount of cell-free nucleic acid. For example, the threshold level can empirically be determined based on the mean or median of a parameter in subjects having a disease or condition, when compared to the same parameter measured in one or more normal or control or reference sample(s), as measured by one skilled in the art. It is understood that the particular predetermined criteria for setting a threshold level of a parameter for the methods herein are dependent on the assay that is used to measure the parameter, e.g., quantitation of cell-free nucleic acid or an enzymatic activity, such as a nuclease activity. It also is understood that in methods involving comparisons to a predetermined level or amount, or to a control or reference sample, that the references are made with the same type of sample and using the same assay.

For example, when Method 1 and/or combination methods as provided herein that include quantifying cell-free nucleic acid is/are performed, in some examples, the quantitation of cell-free nucleic acid derived from one or more target genes is by real-time quantitative PCR (real-time-qPCR) in samples from one or more subjects having a disease or condition and control or reference samples assayed under the same or analogous conditions. The amount of amplified nucleic acid detected by real-time-qPCR can, in some examples, be quantitated using one or more nucleic acid probes that specifically hybridize to cell-free nucleic acid derived from one or more target genes. Quantitation can be performed by measuring a Ct (cycle threshold) value where if the Ct value is at or below a threshold level, the subject is identified as having the disease or condition and if the Ct value is above a threshold level, the subject is identified as not having the disease or condition. The Ct value can, in some examples, be determined by measuring a signal, such as a fluorescent signal, that is proportional to the amount of the amplification product. In some examples, the Ct value is measured as the amplification cycle number at which a signal above a background level is detected. In some examples, the Ct value is measured as the amplification cycle number where exponential increase of a signal associated with the amplification product begins. In examples, the amplification cycle number where exponential increase of a signal associated with the amplification product begins can be identified using the algorithm provided herein.

The Ct values obtained, e.g., for: (1) a single sample of body fluid using more than one nucleic acid probe, or (2) multiple samples of body fluid from subjects having a disease or condition or not having a disease or condition using the same nucleic acid probe, can be analyzed as a mean or a median of multiple values of Ct, thereby empirically assigning a threshold Ct level at or below which the subject is identified as having the disease or condition and above which the subject is identified as not having a disease or condition. In some examples, a Box Plot analysis can reveal Ct values that are grouped together for subjects known to be normal or reference or control subjects, Ct values that are grouped together for subjects known to have a disease or condition being tested for, and outlier values. The Box Plot analysis can be used to determine the Sensitivity and Specificity of the method for different threshold levels of Ct. In examples, the endpoint signal measurement (e.g., fluorescence measured at the last PCR cycle performed during real-time-qPCR) can be included in the scoring function (in addition to mean or median Ct values).

The threshold Ct value can depend on factors including, but not limited to, the sequence of the cell-free nucleic acid being amplified (e.g., the sequence of the target nucleic acid from which it is derived) and the analysis used to arrive at the Ct value for a sample (e.g., measuring Ct as the cycle at which a fluorescence above a threshold value is measured vs. measuring Ct as the cycle at which an exponential increase in fluorescence begins vs the methods and the method used to make such measurement, such as presetting a threshold fluorescence (e.g., at 10 times over background fluorescence) or measuring Ct as the cycle at which an exponential increase in fluorescence begins using the algorithm provided herein vs other methods.

For example, if the scoring function is based on detecting the Ct (cycle) value in real-time-qPCR at the point where the PCR curve starts its exponential growth, such as by using the algorithm provided herein:

Because the Ct cycles are discreet, linear interpolation between adjacent cycle values can be used to obtain a Ct value. Using the algorithm provided herein, it generally is found that the threshold level has been crossed by the linearly approximated function between 33 and 34 cycles, sometimes a little closer to 33. The threshold value can depend on the cell-free nucleic acid (e.g., the target gene fragment being amplified) but generally is found to be around 33 or 34 cycles. It is understood that the threshold value can vary depending on how Ct is measured/analyzed. In general, a Ct value of less than 35 cycles, such as 34 cycles or less, 33 cycles or less, 32 cycles or less, 31 cycles or less or 30 cycles or less can be indicative of the presence of a disease or condition, such as cancer in the subject whose body fluid is tested and a Ct value that is greater than the threshold value can be indicative of the absence of a disease or condition, such as cancer, in the subject. In some examples, the threshold value is 34 cycles, i.e., a subject is identified as having a disease or condition if the Ct value is 34 cycles or less and the subject is identified as not having a disease or condition if the Ct value is 35 cycles or greater. In some examples, the threshold value is 33 cycles, i.e., a subject is identified as having a disease or condition if the Ct value is 33 cycles or less and the subject is identified as not having a disease or condition if the Ct value is 34 cycles or greater. In some examples, the threshold level can be based on the selection of a value that provides the optimum differentiation between subjects having a disease or condition and subjects not having a disease or condition.

In the methods provided herein, such as Method 1 and certain combination methods that include quantitation of amplified cell-free nucleic acid, it is found that the Sensitivity of the method based on identifying a threshold amount of amplified cell-free nucleic acid is high, of the order of, for example, at least or about 70%, 75%, 80%, 85%, 90%, 95%, 96%, 97%, 98% or 99% or more, up to 100%, and the Specificity of the method is of the order of, for example, at least or about 60%, 65%, 70%, 75%, 80%, 85%, 90% or 95% or more.

For example, when Methods 2A and 2B are performed, in some examples, the quantitation of nuclease activity measured in a sample of body fluid using one or more nucleic acid probes as substrates can be performed by measuring a signal, such as a fluorescent signal, that is proportional to the amount of nuclease activity (i.e., the more the amount of nucleic acid probe that is digested, the greater the amount of measured fluorescence). In some examples, the fluorescence value is measured as one or more of the following: end point fluorescence of the reaction minus the starting point fluorescence of the reaction, end point fluorescence of the reaction divided by the starting point fluorescence of the reaction, initial slope of fluorescence as a function of time, or initial slope of fluorescence as a function of time divided by fluorescence at the starting point of the reaction. Examples of scoring functions to measure fluorescence, as determined at discrete interval throughout the reaction over time (e.g., 1-minute intervals), are as follows:

a. Normalization of endpoints, i.e., all curve endpoint fluorescence values are subtracted by the first point of the curve Y (fluorescence) value.

b. An optional step of removing outliers using the Dixon test: Each measurement is performed more than once, such as for example in triplicate, then each value tested for whether it is an outlier using the Dixon test (W. J. Dixon, Ann. Math. Stat., 21(4): pp. 488-506 (1950)). If the Q-value for this value is equal to or greater than the Q-value threshold (0.970 for CI 0.95), then such value is ignored in further calculations.

c. Averaging 2, 3 or 4 normalized endpoints (function−mean from endpoints). Outliers that might be detected in b. are ignored in this mean value calculation.

d. Division of Resulting Mean Value by “Normal” (Reference) Value Provides the Target K-A3 or K-A3B Coefficient.

In an alternate approach, instead of using the endpoint fluorescence value, the slope of the curve is detected using a linear approximation of the kinetic curve: Y=K*X+B, i.e., the value of K is used in the above analysis instead of the endpoint fluorescence value. Those of skill in the art understand that similar scoring methods can be performed, including scoring by measurement of a signal other than fluorescence (e.g., when alternate detectable labels are used).

The resulting fluorescence measurement(s) can be assessed as a ratio of nuclease activities in one of two ways: In “Method 2A,” the ratio (K-A3) of the nuclease activity (as determined by the amount of fluorescence) in the presence of exogenously added zinc relative to the nuclease activity (as determined by the amount of fluorescence) in the absence of exogenously added zinc and, in “Method 2B,” the ratio (K-A3B) of the nuclease activity measured in the subject relative to the nuclease activity of the control or reference sample. The ratios obtained, e.g., for: (1) a single sample of body fluid using more than one nucleic acid probe, or (2) multiple samples of body fluid from subjects having a disease or condition or not having a disease or condition using the same nucleic acid probe, can be analyzed as a mean or a median of multiple values of K-A3 or K-A3B, thereby empirically assigning a threshold ratio at or above which the subject is identified as having the disease or condition and below which the subject is identified as not having a disease or condition. In some examples, a Box Plot analysis can reveal K-A3 or K-A3B values that are grouped together for subjects known to be normal or reference or control subjects, K-A3 or K-A3B values that are grouped together for subjects known to have a disease or condition being tested for, and outlier values. The Box Plot analysis can be used to determine the Sensitivity and Specificity of the method for different threshold levels of K-A3 or K-A3B.

The Sensitivity and Specificity determined (e.g., based on Box Plot analysis) for different threshold levels of Ct, K-A3 and/or K-A3B values, measured as described above and elsewhere herein, can be used to obtain a receiver/relative operating characteristic curve (ROC curve) created by plotting the Sensitivity (rate of correctly identifying a subject as having a disease or condition) against Specificity (rate of correctly identifying a subject as not having a disease or condition), using various cutoffs. The threshold level typically is selected at the point where a ROC curve plateaus or “at about which” (e.g., within about 1%, 2%, 3%, 4%, 5%, 6%, 7%, 8%, 9% or 10%) of a point at which the ROC curve plateaus, although any point on the ROC curve that provides high sensitivity without significantly compromising specificity can be selected, or that provides the desired combination of sensitivity and specificity can be selected. In some examples, the threshold level can be based on the selection of a value that provides the optimum differentiation between subjects having a disease or condition and subjects not having a disease or condition. In examples, the optimal threshold level can be determined by the cutoff yielding the maximum value of J (Youden's index)=Sensitivity+Specificity−1, and represents the level of biomarker (e.g., amount of cell-free nucleic acid or cell-free nuclease activity) that is optimal in its differentiating ability when equal weight is given to Sensitivity and Specificity.

A receiver operating characteristics curve (ROC curve) is a plot of the true positive rate, generally on they axis (TPR, or Sensitivity, i.e., the rate of correctly identifying a subject as having a disease or condition) against the false positive rate, generally on the x axis (FPR, which is =1−Specificity, the rate of correctly identifying a subject as not having a disease or condition). The area under the curve (AUC) represents the degree or measure of separability, i.e., capability of distinguishing between subjects having a disease or condition and subjects not having a disease or condition. In general, a good model means that the AUC has a value that is as close to 1 as possible, i.e., there is a good measure of separability between the class of subjects having a disease or condition and the class of subjects not having a disease or condition. When the AUC is 0.5, it means that the model has no ability to distinguish between the class of subjects having a disease or condition and the class of subjects not having a disease or condition, and AUC below 0.5 and approaching 0 means that, more often than not, subjects having a disease or condition are identified as not having a disease or condition, and vice versa.

The threshold value can depend on the reactions conditions for measuring nuclease activity, the type of body fluid, the type of nucleic acid probe, the assay used to measure nuclease activities and the like. It is understood that the threshold value can vary depending on how the nuclease activity measured/analyzed and/or variations in reaction conditions as described herein and as known to those of skill in the art. In general, in the methods provided herein, a threshold K-A3 coefficient (in a sample of body fluid, ratio of nuclease activity in the presence of added magnesium and exogenous zinc relative to nuclease activity in the presence of added magnesium alone and no exogenous zinc) or K-A3B coefficient (ratio of nuclease activity in a sample of body fluid from a test subject relative to nuclease activity in a control or reference sample of body fluid), it is found that optimal differentiation between subjects having a disease or condition and not having a disease or condition can be obtained when the threshold level (cutoff ratio) is 1.3; if the ratio is at or greater than 1.3, the subject is identified as having the disease or condition; and if the ratio is less than 1.3, the subject is identified as not having the disease or condition. Depending on the reaction conditions to measure nuclease activity, in some examples, the threshold level for identifying a subject as having the disease or condition is a ratio that is ≥1.2, ≥1.3, ≥1.4 or ≥1.5 and the threshold level for identifying a subject as not having the disease or condition is a ratio that is <1.2, <1.3, <1.4 or <1.5. For example, a subject having a disease or condition can have a ratio (K-A3 or K-A3B coefficient) of at least or about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5 or more and a subject not having a disease or condition can have a ratio (K-A3 or K-A3B coefficient) of less than at least or about 1.6, 1.5, 1.4, 1.3, 1.2, 1.1 or 1.0. In some examples, e.g., in the presence of a reducing agent, the differentiation between subjects having a disease or condition and not having a disease or condition can be more pronounced. For example, in the presence of a reducing agent, a subject not having a disease or condition can have a ratio (K-A3 or K-A3B coefficient) of about or less than 1.0, 1.1, 1.2, 1.3, 1.4, 1.5 or 1.6 and a subject having a disease or condition can have a ratio (K-A3 or K-A3B coefficient) of greater than or equal to at least or about 1.6 to at least or about 20, 21, 22, 23, 24, 25, 26, 27, 28, 29, 30, 31, 32, 33, 34, 35, 36, 37, 38, 39 or 40 or more, such as at least or about 1.8, 1.9, 2.0, 2.5, 3.0, 3.5, 4.0, 4.5, 5.0, 7.5, 10.0, 12.5, 15.0, 17.5, 20, 22.5, 25, 27.5, 30, 32.5, 35, 37.5 or 40 or more.

In the methods provided herein, such as Method 2A, Method 2B and certain combination methods that include the measurement of an enzymatic activity, such as a nuclease activity, it is found that the Sensitivity of the method based on identifying a threshold amount of amplified cell-free nucleic acid is high, of the order of, for example, at least or about 70%, 75%, 80%, 85%, 90%, 95%, 96%, 97%, 98% or 99% or more, up to 100%, and the Specificity of the method is of the order of, for example, at least or about 70%, 75%, 80%, 85%, 90% or 95% or more.

Any of the above determinations can be made manually, or using computers or other devices and/or suitable software or computer program products, e.g., for executing an algorithm to measure the amount of amplified cell-free nucleic acid in a sample of body fluid (e.g., Ct value) or for measuring enzymatic activity or ratios of activities, such as nuclease activity or ratios of nuclease activities, and/or for classifying a subject as having a disease or condition or not having a disease or condition based on the value (e.g., Ct value) of the amount of amplified nucleic acid measured in the body fluid or the measured enzymatic activity or ratio of activities, such as nuclease activity or ratio of nuclease activities.

### F. TARGET GENES

In some examples of the methods provided herein, such as Method 1 and certain combination methods that include the quantitation of cell-free nucleic acids in a body fluid, the cell-free nucleic acid is derived from a target gene of interest. Further, in examples of the methods provided herein, such as Method 2A, Method 2B and certain combination methods that include the measurement of a nuclease activity using a nucleic acid probe as substrate, in some examples, the nucleic acid probe (sequence) is derived from a target gene. The target gene can be any gene of interest, such as, for example, a gene or a product encoded by the gene that is known or found to be associated with the disease or condition whose presence or absence is being analyzed, e.g., the target gene is over-expressed, under-expressed, mutated, has insertions or deletions, is hypermethylated or hypomethylated in a subject having the disease or condition when compared to a control or reference subject. For example, when the disease or condition is caused by a pathogen, the target gene can be a gene that uniquely identifies the pathogen. In some examples, the target gene has a causal link to the disease or condition whose presence or absence is being analyzed such as, for example, an oncogene in cancer.

In some examples, the target gene or fragments thereof is/are known to be present or are identified as being present in cell-free nucleic acid (e.g., for Method 1 and certain combination methods that include the quantitation of cell-free nucleic acids in a body fluid) or are known to contain sequences which, when used as substrates for nucleases (e.g., for Method 2A and Method 2B and certain combination methods that include the measurement of a nuclease activity using a nucleic acid probe as substrate), modulate the activity of the nuclease in a manner that is zinc-dependent, e.g., the nuclease activity is influenced by the introduction of exogenous zinc and/or by using nucleic acid probes that contain at least one zinc finger protein binding motif. In some examples, the target gene is selected based on its presence (or the presence of fragments thereof) in cell-free nucleic acid that is amplified (e.g., by whole genome amplification) in an amount that is at least 5, 6, 7, 8, 9, or 10 or more fold abundant than its presence in a control or reference sample amplified under the same conditions.

In some examples, the disease or condition is cancer. In examples, the target gene(s) is selected from among one or more of ESR1 (estrogen receptor 1), PGR (progesterone receptor), HER2 (human epidermal growth factor receptor 2), ARF family of proteins and regulators, COX1 (cyclooxygenase 1), COX11 (cytochrome c oxidase, mitochondrial), perilipin (PLIN) family, EGFR (epidermal growth factor receptor), MMP7 (matrix metallopeptidase 7), MMP9 (matrix metallopeptidase 9), SOX1 (SRY-Box Transcription Factor 1), TERT (telomerase reverse transcriptase), P53 tumor suppressor protein, MED12 (mediator complex subunit 12), RFX2 (Regulatory Factor X2), P21 (cyclin-dependent kinase inhibitory protein-1), PI3 KB (phosphoinositide 3-kinase beta) and SEPT9 (septin-9).

In examples of the methods provided herein, the target gene sequence includes at least one zinc finger binding motif, i.e., a sequence and/or structural motif to which a zinc finger protein binds. In some examples, the cell-free nucleic derived from the target gene contains at least one zinc finger binding motif. In examples, such as those of Method 1 and certain combination methods that include the quantitation of cell-free nucleic acids in a body fluid, when quantitation is, for example by a PCR method or a qPCR method, one or more of the primer sequences and/or a probe sequence for amplifying/quantitating the cell-free nucleic acid in the body fluid can contain a zinc finger binding motif.

### G. ZINC FINGER PROTEINS

In examples of the methods provided herein, the biomarker being measured in the body fluid, e.g., the amount of amplified cell-free nucleic acid (e.g., for Method 1 and certain combination methods that include the quantitation of cell-free nucleic acids in a body fluid) or the amount of nuclease activity (e.g., for Method 2A and Method 2B and certain combination methods that include the measurement of a nuclease activity using a nucleic acid probe as substrate) is modulated by a zinc finger protein. The modulation by a zinc finger protein can be, for example, due to the addition of exogenous zinc to the body fluid (e.g., for Method 2A and certain combination methods that include the measurement of a nuclease activity in the presence of exogenous zinc), or can be due to endogenous levels of zinc in the body fluid (e.g., for Method 2B and certain combination methods which, in some examples, include the measurement of a nuclease activity in the absence of exogenous zinc), or can be due to the presence of at least one zinc finger binding motif in cell-free nucleic acid derived from a target gene, or primers or nucleic acid probes that hybridize thereto (e.g., for Method 1 and certain combination methods that include the quantitation of cell-free nucleic acids in a body fluid) or at least one zinc finger binding motif in a nucleic acid probe that serves as a substrate for one or more cell-free nucleases activity (e.g., for Method 2A and Method 2B and certain combination methods that include the measurement of a nuclease activity using a nucleic acid probe as substrate).

Zinc finger proteins (ZNFs) are one of the most abundant groups of proteins and have a wide range of molecular functions. Up to 35,000 or more zinc finger proteins have been identified (Cassandri et al., Cell Death Discov., 3:17071 (2017); Iuchi et al., eds., Zinc Finger Proteins, Springer-Verlag, 2007). In general, a zinc finger in a zinc finger protein is a protein motif of between about 27 amino acids to about 35 amino acids, such as about 30 amino acids, characterized by the coordination of one or more zinc ions (Zn²⁺) that can stabilize the fold and bind to a zinc finger binding motif in DNA or RNA. A “Zinc Hand” of a ZNF can contain several zinc fingers, e.g., from between about 2 to about 20 or more fingers, such as about 3, 4, 5, 6, 7, 8, 9, 10, 11, 12, 13, 14, 15, 16 or 17 or more zinc fingers.

There are a wide variety of zinc finger domains, making it possible for ZNFs to interact with nucleic acids, e.g., DNA, RNA, PAR (poly-ADP-ribose) as well as other proteins. ZNFs are implicated in the regulation of several cellular processes including, but not limited to, transcriptional regulation, ubiquitin-mediated protein degradation, signal transduction, actin targeting, DNA repair, cell migration, and numerous other processes. Transcription factors play a central role in regulating gene expression, and therefore coordinate a plethora of biological processes, including differentiation, development, metabolism, apoptosis, autophagy and stemness maintenance. Based on different DNA binding motifs, transcription factors (TF) can be majorly categorized into classical zinc fingers, homeodomains and basic helix-loop-helix. Among these, classical zinc finger containing proteins (ZNFs) form the largest family of sequence-specific DNA binding protein, which are encoded by 2% of human genes. To date, several classes of zinc finger motifs have been reported, including Cys2His2 (C2H2) like, Gag knuckle, Treble clef, Zinc ribbon, Zn2/Cys6, TAZ2 domain like, Zinc binding loops and Metallothionein. Different types of zinc finger motifs can show great diversity in their biological functions. Notably, in addition to DNA binding, studies have recently revealed the RNA, protein and lipids interacting abilities of zinc finger motifs. Therefore, with different combinations of multiple zinc finger motifs, ZNFs can expand their diverse role in gene regulations under different cell contexts or stimuli. Zinc finger proteins have been postulated to be involved in tumorigenesis, cancer progression and metastases (Jen et al., J. Biomed. Sci., 23:53 (2016); Klug, Ann. Rev. Biochem., 79:213-231 (2010)).

Exemplary zinc finger proteins, including zinc finger proteins that are transcription factors (TF), are reproduced in Table 24 below, which is adapted from Cassandri et al., Cell Death Discov., 3:17071 (2017):

| | | Number of | | |
|---|---|---|---|---|
| Zinc Finger | Zinc Finger | Genes in | Number | |
| Type | Structure/Motif | the Family | of TF | Exemplary Members |
| Zinc fingers | C-x-C-x-H-x-H | 720 | 372 | KLF4, KLF5, EGR3, |
| C2H2-type (ZNF) | | | | ZFP637, SLUG, ZNF750, |
| | | | | ZNF281, ZBP89, GLIS1, |
| | | | | GLIS3 |
| Ring finger | C-x-C-x-C-x-H-xxx- | 275 | 12 | MDM2, BRCA1, ZNF179 |
| proteins (RNF) | C-x-C-x-C-x-C | | | |
| PHD finger | C-x-C-x-C-x-C-xxx- | 90 | 0 | KDM2A, PHF1, ING1 |
| proteins (PHF) | H-x-C-x-C-x-C | | | |
| LIM domain | C-x-C-x-H-x-C-x-C- | 53 | 1 | ZNF185, LIMK1, PXN |
| containing | x-C-x-C-x-(C, H, D) | | | |
| Nuclear hormone | C-x-C-x-C-x-C-xxx- | 50 | 47 | VDR, ESR1, NR4A1 |
| receptors (NR) | C-x-C-x-C-x-C | | | |
| Zinc fingers | C-x-C-x-C-x-H | 35 | 2 | RC3H1, HELZ, MBNL1, |
| CCCH-type | | | | ZFP36, ZFP36L1 |
| (ZC3H) | | | | |
| Zinc fingers | C-x-C-x-C-x-C-xxx- | 31 | 0 | EEA1, HGS, PIKFYVE |
| FYVE-type | C-x-C-x-C-x-C | | | |
| (ZFYVE) | | | | |
| Zinc fingers | C-x-C-x-H-x-C | 25 | 2 | CNBP, SF1, LIN28A |
| CCHC-type | | | | |
| (ZCCHC) | | | | |
| Zinc fingers | C-x-C-x-H-x-C-xxx- | 24 | 0 | ZDHHC2, ZDHHC8, |
| DHHC-type | C-x-C-x-H-x-C | | | ZDHHC9 |
| (ZDHHC) | | | | |
| Zinc fingers | C-x-C-x-C-x-C-xxx- | 21 | 4 | PDCD2, RUNX1T1, |
| MYND-type | C-x-C-x-H-x-C | | | SMYD2, SMYD1 |
| (ZMYND) | | | | |
| Zinc fingers | C-x-C-x-C-x-C | 21 | 3 | YAF2, SHARPIN, EWSR1 |
| RANBP2-type | | | | |
| (ZRANB) | | | | |
| Zinc fingers ZZ- | C-x-C-x-C-x-C | 18 | 3 | HERC2, NBR1, CREBBP |
| type (ZZZ) | | | | |
| Zinc fingers | C-x-C-x-H-x-C | 16 | 2 | IKBKG, L3MBTL1, |
| C2HC-type | | | | ZNF746 |
| (ZC2HC) | | | | |
| GATA zinc | C-x-C-x-C-x-C | 15 | 15 | GATA4, GATA6, MTA1 |
| finger domain | | | | |
| containing | | | | |
| (GATAD) | | | | |
| ZF class | C-x-C-x-H-x-H | 15 | 10 | ADNP, ZEB1, ZHX1 |
| homeoboxes and | | | | |
| pseudogenes | | | | |
| THAP domain | C-x-C-x-C-x-H | 12 | 3 | THAP1, THAP4, THAP11 |
| containing | | | | |
| (THAP) | | | | |
| Zinc fingers | C-x-C-x-C-x-C-xxx- | 12 | 2 | CXXC1, CXXC5, |
| CXXC-type | C-x-C-x-C-x-C | | | MBD1, DNMT1 |
| (CXXC) | | | | |
| Zinc fingers | C-x-C-x-C-x-H | 9 | 0 | MAP3K1, ZSWIM5, |
| SWIM-type | | | | ZSWIM6 |
| (ZSWIM) | | | | |
| Zinc fingers | C-x-C-x-C-x-C-xxx- | 8 | 0 | ZFAND3, ZFAND6, |
| AN1-type | C-x-H-x-H-x-C | | | IGHMBP2 |
| (ZFAND) | | | | |
| Zinc fingers | C-x-C-x-H-x-C | 8 | 0 | ZAR1, RTP1, RTP4 |
| 3CxxC-type | | | | |
| (Z3CXXC) | | | | |
| Zinc fingers CW- | C-x-C-x-C-x-C | 7 | 0 | MORC1, |
| type (ZCW) | | | | ZCWPW1, KDM1B |
| Zinc fingers | C-x-C-x-C-x-C | 7 | 0 | TTF2, NEIL3, TOP3A |
| GRF-type | | | | |
| (ZGRF) | | | | |
| Zinc fingers | C-x-C-x-H-x-C | 7 | 1 | PIAS1, PIAS3, PIAS4 |
| MIZ-type (ZMIZ) | | | | |
| Zinc fingers | C-x-C-x-H-x-H | 6 | 2 | ZBED1, ZBED4, ZBED6 |
| BED-type | | | | |
| (ZBED) | | | | |
| Zinc fingers HIT- | C-x-C-x-C-x-C-xxx- | 6 | 0 | ZNHIT3, DDX59, INO80B |
| type (ZNHIT) | C-x-C-x-H-x-C | | | |
| Zinc fingers | C-x-C-x-C-x-C | 6 | 6 | ZMYM2, ZMYM3, ZMYM4 |
| MYM-type | | | | |
| (ZMYM) | | | | |
| Zinc fingers | C-x-C-x-H-x-H | 5 | 0 | ZNF638, ZMAT1, ZMAT3, |
| matrin-type | | | | ZMAT5 |
| (ZMAT) | | | | |
| Zinc fingers | C-x-C-x-H-x-H | 3 | 3 | MYT1, MYT1L, ST18 |
| C2H2C-type | | | | |
| Zinc fingers | C-x-C-x-H-x-H | 3 | 0 | DBF4, DBF4B, ZDBF2 |
| DBF-type | | | | |
| (ZDBF) | | | | |
| Zinc fingers | C-x-C-x-H-x-C | 2 | 1 | LIG3, PARP1 |
| PARP-type | | | | |

The C2H2-type zinc finger motif is the largest group of all zinc finger motif classes, with about 5,926 members in the C2H2-type ZNF family as of 2016 (InterPro database). C2H2-type zinc finger motif is composed of CX2CX3FX5LX2HX3H, and its two cysteine and two histidine residues fold into a finger-like structure of a two-stranded antiparallel β-sheet and an α-helix after interacting with zinc ions. Studies have demonstrated that two to three successive C2H2-type zinc finger motifs are the most suitable unit for DNA binding. In addition, GC-rich or GT-rich sequences serve as C2H2-type ZNF cis-regulatory elements. For example, CTGGCAGCGC has been revealed as SP1 consensus binding element to transcriptionally activate BRK1 expression, while (T/A)(G/A)CAGAA(T/G/C) is the consensus element for ZNF217 to suppress E-cadherin expression. In addition to tandem zinc finger motifs, the C2H2-type ZNF also contains other functional domains, such as BTB (Broad-Complex, Tramtrack, and Bric-a-brac)/POZ (poxvirus and zinc finger), the Kruppel-associated box (KRAB), and SCAN (SRE-ZBP, CTfin51, AW-1 and Number 18 cDNA) domain. These functional domains can control subcellular localization, DNA binding and gene expression by regulating selective binding of the transcription factors with each other or with other cellular component. For instance, zinc finger protein GATA-1 has been reported to interact with different partners, including Fli-1, Sp1, EKLF and PU.1 (see, for example, Jen et al., J. Biomed. Sci., 23:53 (2016) and references cited therein).

Because of their versatility, zinc finger proteins have been engineered as nucleases. For example, hybrid proteins containing a DNA-binding domain such as Cys(2)His(2) and the coding sequence of restriction nuclease Fok I can be used in applications such as genome editing (Bibikova et al., Genetics, 161(3):1169-1175 (2002)). Naturally occurring zinc-dependent nucleases have been found in plants, e.g., nuclease S1, nuclease P1 and Mung bean nuclease. These nucleases require for activity Zn and have specificity towards single-stranded DNA and RNA (Lesniewicz et al., Plant Cell Physiol., 54(7):1064-1078 (2013); Vogt, Eur. J. Biochem., 33:192-200 (1973); Fujimoto et al., Agric. Biol. Chem., 38(4):785-790 (1974); Kowalski et al., Biochemistry, 15(20):4457-4463 (1976)). A mammalian zinc finger nuclease, TZAP, has been found to be involved in the shortening of telomeric DNA during apoptosis; TZAP has 11 zinc fingers and is implicated in the initial telomere trimming process leading to apoptosis (Li et al., Science, 355:638-641 (2017)).

In examples of the methods provided herein, without being bound by theory, it is thought that in some examples, the amount of biomarker, e.g., nuclease activity, that is detected is modulated by zinc finger proteins. The zinc finger protein can be a nuclease, or the zinc finger protein can modulate a nuclease or the quantity of free cell-free nucleic acid (e.g., for Method 1 and certain combination methods that include the quantitation of cell-free nucleic acids in a body fluid) by binding to a target gene, cell-free nucleic acid derived from a target gene, one or more primers or nucleic acid probes containing at least one zinc finger binding motif. For example, it is shown herein that disruption of even a single (CNN) or (GNN) repeat in a nucleic acid probe for measuring nuclease activity can significantly decrease the ability to distinguish between a sample of body fluid from a subject having cancer and a control or reference sample (see, e.g., Example 3 and Example 10).

### H. ZINC FINGER BINDING MOTIFS

In examples of the methods provided herein, one or more of a target gene, cell-free nucleic acid derived from a target gene, one or more primers used to amplify cell-free nucleic acid or one or more nucleic acid probes for quantitating amplified cell-free nucleic acid (e.g., for Method 1 and certain combination methods that include the quantitation of cell-free nucleic acids in a body fluid) or nuclease activity (e.g., for Method 2A and Method 2B and certain combination methods that include the measurement of a nuclease activity using a nucleic acid probe as substrate) contains at least one zinc finger binding motif. As used herein, “zinc finger binding motif” or “zinc finger protein binding motif” refers to any two- or three-dimensional feature of a nucleotide segment to which a zinc finger protein or derivative polypeptide binds with specificity. Included within this definition are nucleotide sequences as well as the three-dimensional aspects of the DNA double helix, such as, but are not limited to, the major and minor grooves and the face of the helix. The motif typically is any sequence of suitable length to which the zinc finger polypeptide can bind. For example, nucleic acid molecules containing sequence motifs that include “GC Box” elements, such as the “GGGCGG” consensus, can be a binding site for zinc finger proteins. In some examples, a zinc finger in a protein can bind to triplet base pairs in the “GC Box,” such as the base pairs formed by a GGG motif, or the base pairs formed by the GCG motif. A three-finger zine finger polypeptide binds to a motif typically having about 9 to about 14 base pairs. Zinc finger proteins also can bind to single-stranded nucleic acids, such as single-stranded DNA or RNA. Generally, to ensure specificity in a genome the size of a human, the recognition sequence is at least about 16 bases or base pairs although nucleic acid probes of smaller size, such as 12, 13, 14 or 15 bases or base pairs, also are contemplated for use herein. Zinc finger binding motifs of any specificity are contemplated and provided herein. The zinc finger binding motif can be any sequence designed empirically or to which the zinc finger protein or derivative thereof binds. The motif can occur in any DNA or RNA sequence, including regulatory sequences, exons, introns, or any non-coding sequence.

In some examples, a zinc finger binding motif includes a sequence of (CNN)x or (GNN)x repeats, where x is the number of CNN or GNN repeats, and the number is at least 2; and N=A, G, C, or T. In examples, x is 3 or more repeats, such as 3, 4, 5, 6, 7, 8, 9, 10, 11, 12, 13, 14, 15, 16 or 17 or more repeats. In some examples, x is between about 3 repeats to about 10, 11, 12, 13, 14, 15, 16 or 17 repeats. In some examples, x is at least 3, 4, 5, 6, 7, 8, 9, 10, 11, 12, 13, 14, 15, 16 or 17 repeats. A “Zinc Neck” refers to an oligonucleotide sequence that is a target for binding a Zinc Hand.

Exemplary nucleic acid probes for quantitating cell-nucleic acids derived from certain target genes include those selected from among one or more of the following: SEQ ID NO:1 (complementary to a portion of ESR1), a sequence that has at least 95%, 96%, 97%, 98%, 99% or more identity with SEQ ID NO:1, SEQ ID NO:4 (complementary to PGR), a sequence that has at least 95%, 96%, 97%, 98%, 99% or more identity with SEQ ID NO:4, SEQ ID NO:7 (complementary to a portion of HER2), a sequence that has at least 95%, 96%, 97%, 98%, 99% or more identity with SEQ ID NO:7, SEQ ID NO:10 (complementary to a portion of ARFIP1), a sequence that has at least 95%, 96%, 97%, 98%, 99% or more identity with SEQ ID NO:10, SEQ ID NO:13 (complementary to a portion of COX1), a sequence that has at least 95%, 96%, 97%, 98%, 99% or more identity with SEQ ID NO:13, SEQ ID NO:16 (complementary to a portion of PLIN1), a sequence that has at least 95%, 96%, 97%, 98%, 99% or more identity with SEQ ID NO:16, SEQ ID NO:19 (complementary to a portion of EGFR), a sequence that has at least 95%, 96%, 97%, 98%, 99% or more identity with SEQ ID NO:19, SEQ ID NO:20 (complementary to a portion of EGFR), a sequence that has at least 95%, 96%, 97%, 98%, 99% or more identity with SEQ ID NO:20, SEQ ID NO:21 (complementary to a portion of EGFR), a sequence that has at least 95%, 96%, 97%, 98%, 99% or more identity with SEQ ID NO:21, SEQ ID NO:24 (complementary to a portion of MMP7), a sequence that has at least 95%, 96%, 97%, 98%, 99% or more identity with SEQ ID NO:24, SEQ ID NO:29 (complementary to a portion of MED12), a sequence that has at least 95%, 96%, 97%, 98%, 99% or more identity with SEQ ID NO:29, SEQ ID NO:30 (complementary to a portion of RFX2), a sequence that has at least 95%, 96%, 97%, 98%, 99% or more identity with SEQ ID NO:30, SEQ ID NO:27 (complementary to a portion of TERT), a sequence that has at least 95%, 96%, 97%, 98%, 99% or more identity with SEQ ID NO:27, SEQ ID NO:31 (complementary to a portion of P21), a sequence that has at least 95%, 96%, 97%, 98%, 99% or more identity with SEQ ID NO:31, SEQ ID NO:32 (complementary to a portion of P13 KB), a sequence that has at least 95%, 96%, 97%, 98%, 99% or more identity with SEQ ID NO:32, SEQ ID NO:33 (complementary to a portion of SEPT9), a sequence that has at least 95%, 96%, 97%, 98%, 99% or more identity with SEQ ID NO:33, SEQ ID NO:28 (complementary to a portion of P53), a sequence that has at least 95%, 96%, 97%, 98%, 99% or more identity with SEQ ID NO:28, SEQ ID NO:50 (complementary to a portion of P53), and a sequence that has at least 95%, 96%, 97%, 98%, 99% or more identity with SEQ ID NO:50. It is understood that in some examples, any permutation or any combination of more than one of the nucleic acid probes provided herein, along with any permutation or any combination of more than one of the sets of primers provided herein, can be used in the methods and/or included in the compositions, combinations, kits and articles of manufacture provided herein, e.g., to quantitate cell-free nucleic acid derived from more than one target gene. In some examples, the disease or condition is cancer, such as any of those provided herein and/or as known to those of skill in the art.

In some examples, the disease or condition is COVID-19, and the nucleic acid probe sequences are selected from among one or more of the following:

(SEQ ID NO: 34)
5′-CCAGATGAGGATGAAGAAGAAGG-3′
(SEQ ID NO: 35)
5′-CATGGAGGAGGTGTTGCAGGAGCCT-3′;
and
(SEQ ID NO: 36)
5′-AAGTTGATGGTGTTGATGTAGAAT-3′

### I. METHODS OF MONITORING AND/OR TREATING A DISEASE OR CONDITION

Any of the methods provided herein, or any combinations thereof, can be used to monitor the progression or regression of a disease or condition in a subject by performing the method(s) at suitable intervals for monitoring such progression or regression. In some examples, the monitoring is performed until the subject is identified as being in remission and/or as no longer having the disease or condition. In examples, prognosis and/or treatment of the subject is based on whether the disease has progressed, has decreased or is in remission or the disease or condition or symptoms thereof no longer exist. Also provided herein are methods of treating a disease or condition in a subject previously identified as having a disease or condition by the method of any of the claims provided herein, or as in PCT/US22/79770, published as International PCT publication No. WO 2023/086970, or any combination thereof, by administering a therapeutically effective amount of a treatment that is suitable for the disease or condition. Exemplary treatments can include, for example, surgery, biologics, anti-cancer agents, small molecule compounds, dispersing agents, anesthetics, checkpoint inhibitors, vasoconstrictors, surgery, radiation, a chemotherapeutic agent, a biological agent, a polypeptide, an antibody, a peptide, a small molecule, a gene therapy vector, a virus, DNA, RNA, such as antisense RNA, a compound that decreases the rate of proliferation of the tumor or neoplastic cells without weakening the immune system (e.g., by administering tumor suppressor compounds or by administering tumor cell-specific compounds) or an angiogenesis-inhibiting compound. In some examples, monitoring the disease or condition can be continued at suitable intervals known to those of skill in the art, based on the diseases or condition and its progression or remission, and the treatment and/or dosages and/or dosage regimens can be adjusted accordingly.

In some examples, the diseases or condition is cancer and a subject previously identified as having cancer can be administered an anti-cancer treatment such as a chemotherapeutic agent for treating cancer, such as a nucleoside analog or other antimetabolite (e.g. gemcitabine or derivative or other nucleoside analog), tumor-targeted taxanes, tumor-targeted taxane and nucleoside analog combination, Acivicins; Avicin; Aclarubicins; Acodazoles; Acronines; Adozelesins; Aldesleukins; Alemtuzumabs; Alitretinoins (9-Cis-Retinoic Acids); Allopurinols; Altretamines; Alvocidibs; Ambazones; Ambomycins; Ametantrones; Amifostines; Aminoglutethimides; Amsacrines; Anastrozoles; Anaxirones; Ancitabines; Anthramycins; Apaziquones; Argimesnas; Arsenic Trioxides; Asparaginases; Asperlins; Atrimustines; Azacitidines; Azetepas; Azotomycins; Banoxantrones; Batabulins; Batimastats; BCG Live; Benaxibines; Bendamustines; Benzodepas; Bexarotenes; Bevacizumab; Bicalutamides; Bietaserpines; Biricodars; Bisantrenes; Bisantrenes; Bisnafide Dimesylates; Bizelesins; Bleomycins; Bortezomibs; Brequinars; Bropirimines; Budotitanes; Busulfans; Cactinomycins; Calusterones; Canertinibs; Capecitabines; Caracemides; Carbetimers; Carboplatins; Carboquones; Carmofurs; Carmustines with Polifeprosans; Carmustines; Carubicins; Carzelesins; Cedefingols; Celecoxibs; Cemadotins; Chlorambucils; Cioteronels; Cirolemycins; Cisplatins; Cladribines; Clanfenurs; Clofarabines; Crisnatols; Cyclophosphamides; Cytarabine liposomals; Cytarabines; Dacarbazines; Dactinomycins; Darbepoetin Alfas; Daunorubicin liposomals; Daunorubicins/Daunomycins; Daunorubicins; Decitabines; Denileukin Diftitoxes; Dexniguldipines; Dexonnaplatins; Dexrazoxanes; Deazaguanines; Diaziquones; Dibrospidiums; Dienogests; Dinalins; Disermolides; Docetaxels; Dofequidars; Doxifluridines; Doxorubicin liposomals; Doxorubicin HCL; Doxorubicin HCL liposome injection; Doxorubicins; Droloxifenes; Dromostanolone Propionates; Duazomycins; Ecomustines; Edatrexates; Edotecarins; Eflornithines; Elacridars; Elinafides; Elliott's B Solutions; Elsamitrucins; Emitefurs; Enloplatins; Enpromates; Enzastaurins; Epipropidines; Epirubicins; Epoetin alfas; Eptaloprosts; Erbulozoles; Esorubicins; Estramustines; Etanidazoles; Etoglucids; Etoposide phosphates; Etoposide VP-16s; Etoposides; Etoprines; Exemestanes; Exisulinds; Fadrozoles; Fazarabines; Fenretinides; Filgrastims; Floxuridines; Fludarabines; Fluorouracils; 5-fluorouracils; Fluoxymesterones; Flurocitabines; Fosquidones; Fostriecins; Fotretamines; Fulvestrants; Galarubicins; Galocitabines; Gemcitabines; Gemtuzumabs/Ozogamicins; Geroquinols; Gimatecans; Gimeracils; Gloxazones; Glufosfamides; Goserelin acetates; Hydroxyureas; Ibritumomabs/Tiuxetans; Idarubicins; Ifosfamides; Ilmofosines; Ilomastats; Imatinib mesylates; Imexons; Improsulfans; Indisulams; Inproquones; Interferon alfa-2as; Interferon alfa-2bs; Interferon Alfas; Interferon Betas; Interferon Gammas; Interferons; Interleukin-2s and other Interleukins (including recombinant Interleukins); Intoplicines; Iobenguanes [131-I]; Iproplatins; Irinotecans; Irsogladines; Ixabepilones; Ketotrexates; L-Alanosines; Lanreotides; Lapatinibs; Ledoxantrones; Letrozoles; Leucovorins; Leuprolides; Leuprorelins (Leuprorelides); Levamisoles; Lexacalcitols; Liarozoles; Lobaplatins; Lometrexols; Lomustines/CCNUs; Lomustines; Lonafarnibs; Losoxantrones; Lurtotecans; Mafosfamides; Mannosulfans; Marimastats; Masoprocols; Maytansines; Mechlorethamines; Mechlorethamines/Nitrogen mustards; Megestrol acetates; Megestrols; Melengestrols; Melphalans; Melphalans (L-PAMs); Menogarils; Mepitiostanes; Mercaptopurines; 6-Mercaptopurine; Mesnas; Metesinds; Methotrexates; Methoxsalens; Metomidates; Metoprines; Meturedepas; Miboplatins; Miproxifenes; Misonidazoles; Mitindomides; Mitocarcins; Mitocromins; Mitoflaxones; Mitogillins; Mitoguazones; Mitomalcins; Mitomycin Cs; Mitomycins; Mitonafides; Mitoquidones; Mitospers; Mitotanes; Mitoxantrones; Mitozolomides; Mivobulins; Mizoribines; Mofarotenes; Mopidamols; Mubritinibs; Mycophenolic Acids; Nandrolone Phenylpropionates; Nedaplatins; Nelzarabines; Nemorubicins; Nitracrines; Nocodazoles; Nofetumomabs; Nogalamycins; Nolatrexeds; Nortopixantrones; Octreotides; Oprelvekins; Ormaplatins; Ortataxels; Oteracils; Oxaliplatins; Oxisurans; Oxophenarsines; Paclitaxels; Pamidronates; Patubilones; Pegademases; Pegaspargases; Pegfilgrastims; Peldesines; Peliomycins; Pelitrexols; Pemetrexeds; Pentamustines; Pentostatins; Peplomycins; Perfosfamides; Perifosines; Picoplatins; Pinafides; Pipobromans; Piposulfans; Pirfenidones; Piroxantrones; Pixantrones; Plevitrexeds; Plicamycid Mithramycins; Plicamycins; Plomestanes; Porfimer sodiums; Porfimers; Porfiromycins; Prednimustines; Procarbazines; Propamidines; Prospidiums; Pumitepas; Puromycins; Pyrazofurins; Quinacrines; Ranimustines; Rasburicases; Riboprines; Ritrosulfans; Rituximabs; Rogletimides; Roquinimexs; Rufocromomycins; Sabarubicins; Safingols; Sargramostims; Satraplatins; Sebriplatins; Semustines; Simtrazenes; Sizofirans; Sobuzoxanes; Sorafenibs; Sparfosates; Sparfosic Acids; Sparsomycins; Spirogermaniums; Spiromustines; Spiroplatins; Spiroplatins; Squalamines; Streptonigrins; Streptovaricins; Streptozocins; Sufosfamides; Sulofenurs; Sunitinib Malate; 6-thioguanine (6-TG); Tacedinalines; Tales; Talisomycins; Tallimustines; Tamoxifens; Tariquidars; Tauromustines; Tecogalans; Tegafurs; Teloxantrones; Temoporfins; Temozolomides; Teniposides/VM-26s; Teniposides; Teroxirones; Testolactones; Thiamiprines; Thioguanines; Thiotepas; Tiamiprines; Tiazofurins; Tilomisoles; Tilorones; Timcodars; Timonacics; Tirapazamines; Topixantrones; Topotecans; Toremifenes; Tositumomabs; Trabectedins (Ecteinascidin 743); Trastuzumabs; Trestolones; Tretinoins/ATRA; Triciribines; Trilostanes; Trimetrexates; Triplatin Tetranitrates; Triptorelins; Trofosfamides; Tubulozoles; Ubenimexs; Uracil Mustards; Uredepas; Valrubicins; Valspodars; Vapreotides; Verteporfins; Vinblastines; Vincristines; Vindesines; Vinepidines; Vinflunines; Vinformides; Vinglycinates; Vinleucinols; Vinleurosines; Vinorelbines; Vinrosidines; Vintriptols; Vinzolidines; Vorozoles; Xanthomycin As (Guamecyclines); Zeniplatins; Zilascorbs [2-H]; Zinostatins; Zoledronate; Zorubicins; and Zosuquidars, for example:

Aldesleukins (e.g. PROLEUKIN®); Alemtuzumabs (e.g. CAMPATH®); Alitretinoins (e.g. PANRETIN®); Allopurinols (e.g. ZYLOPRIM®); Altretamines (e.g. HEXALEN®); Amifostines (e.g. ETHYOL®); Anastrozoles (e.g. ARIMIDEX®); Arsenic Trioxides (e.g. TRISENOX®); Asparaginases (e.g. ELSPAR®); BCG Live (e.g. TICE® BCG); Bexarotenes (e.g. TARGRETIN®); Bevacizumab (AVASTIN®); Bleomycins (e.g. BLENOXANE®); Busulfan intravenous (e.g. BUSULFEX®); Busulfan orals (e.g. MYLERAN®); Calusterones (e.g. METHOSARB®); Capecitabines (e.g. XELODA®); Carboplatins (e.g. PARAPLATIN®); Carmustines (e.g. BCNU™, BiCNU®); Carmustines with Polifeprosans (e.g. GLIADEL® Wafer); Celecoxibs (e.g. CELEBREX®); Chlorambucils (e.g. LEUKERAN®); Cisplatins (e.g. PLATINOL®); Cladribines (e.g. LEUSTATIN®, 2-CdA®); Cyclophosphamides (e.g. CYTOXAN®, NEOSAR®); Cytarabines (e.g. CYTOSAR-U®); Cytarabine liposomals (e.g. DepoCyt®); Dacarbazines (e.g. DTIC-Dome): Dactinomycins (e.g. COSMEGEN®); Darbepoetin Alfas (e.g. ARANESP®); Daunorubicin liposomals (e.g., DAUNOXOME®); Daunorubicins/Daunomycins (e.g. CERUBIDINE®); Denileukin Diftitoxes (e.g. ONTAK®); Dexrazoxanes (e.g. ZINECARD®); Docetaxels (e.g. TAXOTERE®); Doxorubicins (e.g. ADRIAMYCIN®, RUBEX®); Doxorubicin liposomals, including Doxorubicin HCL liposome injections (e.g. DOXIL®); Dromostanolone propionates (e.g. DROMOSTANOLONE™ and MASTERON® Injection); Elliott's B Solutions (e.g. Elliotts B® Solution); Epirubicins (e.g. ELLENCE®); Epoetin alfas (e.g. EPOGEN®); Estramustines (e.g. EMCYT®); Etoposide phosphates (e.g. ETOPOPHOS®); Etoposide VP-16s (e.g. VEPESID®); Exemestanes (e.g. AROMASIN®); Filgrastims (e.g. NEUPOGEN®); Floxuridines (e.g. FUDR®); Fludarabines (e.g. FLUDARA®); Fluorouracils including 5-FUs (e.g. ADRUCIL®); Fulvestrants (e.g. FASLODEX®); Gemcitabines (e.g. GEMZAR®); Gemtuzumabs/Ozogamicins (e.g. MYLOTARG®); Goserelin acetates (e.g. ZOLADEX®); Hydroxyureas (e.g. HYDREA®); Ibritumomabs/Tiuxetans (e.g. ZEVALIN®); Idarubicins (e.g. IDAMYCIN®); Ifosfamides (e.g. IFEX®); Imatinib mesylates (e.g. GLEEVEC®); Interferon alfa-2as (e.g. ROFERON®-A); Interferon alfa-2bs (e.g. INTRON A®); Irinotecans (e.g. CAMPTOSAR®); Letrozoles (e.g. FEMARA®); Leucovorins (e.g. WELLCOVORIN®, LEUCOVORIN®); Levamisoles (e.g. ERGAMISOL®); Lomustines/CCNUs (e.g. CeeNU®); Mechlorethamines/Nitrogen mustards (e.g. MUSTARGEN®); Megestrol acetates (e.g. MEGACE®); Melphalans/L-PAMs (e.g. ALKERAN®); Mercaptopurine, including 6-mercaptopurines (6-MPs; e.g. PURINETHOL®); Mesnas (e.g. MESNEX®); Methotrexates; Methoxsalens (e.g. UVADEX®); Mitomycin Cs (e.g. MUTAMYCIN®, MITOZYTREX®); Mitotanes (e.g. LYSODREN®); Mitoxantrones (e.g. NOVANTRONE®); Nandrolone Phenylpropionates (e.g., DECA-DURABOLIN® 50); Nofetumomabs (e.g. VERLUMA®); Oprelvekins (e.g. NEUMEGA®); Oxaliplatins (e.g. ELOXATIN®); Paclitaxels (e.g. PAXENE®, TAXOL®); Pamidronates (e.g. AREDIA®); Pegademases (e.g. ADAGEN®); Pegaspargases (e.g. ONCASPAR®); Pegfilgrastims (e.g. NEULASTA®); Pentostatins (e.g. NIPENT®); Pipobromans (e.g. VERCYTE®); Plicamycin/Mithramycins (e.g. MITHRACIN®); Porfimer sodiums (e.g. PHOTOFRIN®); Procarbazines (e.g. MATULANE®); Quinacrines (e.g. ATABRINE®); Rasburicases (e.g. ELITEK®); Rituximabs (e.g. RITUXAN®); Sargramostims (e.g. PROKINE®); Streptozocins (e.g. ZANOSAR®); Sunitinib Malates (e.g. SUTENT®); Tales (e.g. SCLEROSOL®); Tamoxifens (e.g. NOLVADEX®); Temozolomides (e.g. TEMODAR®); Teniposides/VM-26s (e.g. VUMON®); Testolactones (e.g. TESLAC®); Thioguanines including, 6-thioguanine (6-TG); Thiotepas (e.g. THIOPLEX®); Topotecans (e.g. HYCAMTIN®); Toremifenes (e.g. FARESTON®); Tositumomabs (e.g. BEXXAR®); Trastuzumabs (e.g. HERCEPTIN®); Tretinoins/ATRA (e.g. VESANOID®); Uracil Mustards; Valrubicins (e.g. VALSTAR®); Vinblastines (e.g. VELBAN®); Vincristines (e.g. ONCOVIN®); Vinorelbines (e.g. NAVELBINE®); Zoledronates (e.g. ZOMETA®), and oncolytic viral therapies, immunotherapies, therapeutic antibodies, CAR-T therapy and other cell therapies, checkpoint inhibitors, radiation, surgery, a peptide, gene therapy, CRISPR-based therapy and others known to those of skill in the art. Anti-cancer agents/treatment regimens for particular cancers and stages of progression as determined by monitoring, including monitoring by the methods provided herein, are known to those of skill in the art. In some examples, a subject is previously identified as having COVID-19 and is administered a treatment for COVID-19, such as the antiviral drug remdesivir, molnupiravir, ritonavir, and anti-Sars-CoV-2 monoclonal antibodies such as bamlanivimab, etesevimab, casirivimab or combinations of two or more antibodies thereof.

### J. TYPES OF DISEASES AND CONDITIONS

Any disease or condition that is characterized by a change in the amount of cell-free nucleic acid in a body and/or by a change in cell-free enzymatic activity, such as a change in kinase, protease or nuclease activity, is contemplated for analysis and use in the methods provided herein, including, but not limited to, cancer, stroke, trauma, including acute trauma, myocardial infarction, cerebral infarction, exercise, transplantation, inflammation, autoimmune diseases and other diseases that increase inflammation such as systemic lupus erythematosus, arthritis, hepatitis, burns, sepsis, infection by pathogens, brain injury and other diseases or conditions. More than one disease or condition, or more than one type or subtype of a disease, such as more than one type of cancer or more than one subtype of breast cancer, can simultaneously be analyzed, monitored and/or treated by the methods provided herein. In some examples, the disease or condition is cancer or a non-cancerous/benign proliferative condition. In examples, the disease is caused by a pathogen.

### 1. Cancers or Non-Cancerous Proliferative Conditions

Exemplary cancers for analysis, monitoring and/or treatment by the methods provided herein, include, but are not limited to, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, fibroadenoma, fibrocystic disease, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt's lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin's lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus.

Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma.

Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma.

Exemplary neoplasias affecting agricultural and pastoral livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

In some examples, exemplary cancers or non-cancerous/benign proliferative conditions for analysis, monitoring and/or treatment by the methods provided herein, include, but are not limited to, ovarian cancer, in situ carcinoma (ISC), squamous cell carcinoma (SCC), prostate cancer, pancreatic cancer, lung cancer, small cell lung cancer, non-small cell lung cancer (NSCLC), adrenal gland tumor, AIDS-associated cancer, alveolar soft part sarcoma, astrocytic tumor, adrenal cancer, bladder cancer, bone cancer, brain cancer, spinal cord cancer, metastatic brain tumor, B-cell cancer, breast cancer, carotid body tumor, cervical cancer, chondrosarcoma, a chordoma, a chromophobe renal cell carcinoma, a clear cell carcinoma, colon cancer, colorectal cancer, cutaneous benign fibrous histiocytoma, fibroadenoma, fibrocystic disease, desmoplastic small round cell tumor, ependymoma, Ewing's tumor, extraskeletal myxoid chondrosarcoma, fibrogenesis imperfecta ossium, fibrous dysplasia of the bone, gallbladder cancer, bile duct cancer, gastric cancer, synovial tissue cancer, gestational trophoblastic disease, germ cell tumor, glioblastoma, hematological malignancy, hepatocellular carcinoma, islet cell tumor, Kaposi's Sarcoma, kidney cancer, leukemia, acute myeloid leukemia, chronic lymphocytic leukemia, chronic myeloid leukemia, hairy cell leukemia, liposarcoma/malignant lipomatous tumor, liver cancer, lymphoma, Burkett's lymphoma, diffuse large B cell lymphoma, follicular lymphoma, mantle cell lymphoma, marginal zone lymphoma, non-Hodgkin's lymphoma, small lymphocytic lymphoma, medulloblastoma, melanoma, meningioma, mesothelioma, pharyngeal cancer, multiple endocrine neoplasia, multiple myeloma, myelodysplastic syndrome, neuroblastoma, neuroendocrine tumors, papillary thyroid carcinoma, parathyroid tumor, pediatric cancer, peripheral nerve sheath tumor, phaeochromocytoma, pituitary tumor, posterior uveal melanoma, renal metastatic cancer, renal cell carcinoma, rhabdoid tumor, rhabdomyosarcoma, sarcoma, skin cancer, small round blue cell tumor of childhood, soft-tissue sarcoma, squamous cell cancer, head and neck cancer, squamous cell cancer of the head and neck (SCCHN), stomach cancer, synovial tissue cancer, synovial sarcoma, testicular cancer, thymic carcinoma, thymoma, a thyroid cancer, thyroid metastatic cancer, uterine cancer, hepatoma, glioma, retinoblastoma, myeloma, lymphoma, and leukemia.

### Breast Cancer

In some examples, the disease or condition that is analyzed, monitored and/or treated by the methods provided herein is breast cancer. Breast cancer is the most common carcinoma diagnosed among women worldwide, accounting for nearly one in three cancers (Harbeck et al., Nat. Rev. Dis. Primers, 5(1):66 (2019)). It also is the second leading cause of cancer-related deaths in women aged between 35 and 75 years after lung cancer (Siegel et al., CA Cancer J. Clin.; 65:5-29 (2015); DeSantis et al., CA Cancer J. Clin., 66:31-42 (2016)). According to the World Health Organization, in 2020, 2,261,419 new cases were detected and 684,996 deaths from breast cancer were recorded. If detected in the early stages, breast cancer is treatable in 70-80% of cases. In addition, early diagnosis reduces the likelihood of death and the appearance of unwanted complications in the terminal stages of the disease. In the later stages (in the presence of metastases), modern approaches to treatment often are ineffective and the disease is considered incurable. Further, as a heterogeneous and dynamic disease, breast cancer exhibits unique acquired somatic mutations and gene expression changes that underpin the two main mortality factors: disease recurrence and drug resistance. Therefore, early diagnosis and predicting and monitoring response to treatment and disease progression is needed due to changes in tumor biology and therapy responsiveness over time.

Cancer diagnosis and metastasis monitoring are performed by tissue biopsy, imaging and/or re-biopsy. For the diagnosis of breast cancer, the so-called triple test is currently used, which includes a clinical examination by a mammologist, mammography and fine-needle aspiration biopsy followed by cytological examination. To detect breast cancer in the early stages, women over 40-50 years of age are advised to undergo regular mammography examinations. The effectiveness of such a mammographic examination depends on age and is most effective for women aged 50-69 years (Lauby-Secretan et al., New Engl. J. Med., 372(24):2353-2358 (2015)). Among the disadvantages of mammography are the presence of false-positive and false-negative results, erroneous medical conclusions (overdiagnosis), radiation exposure, pain during the examination, anxiety and other psychological consequences (Nelson et al., Ann. Intern. Med., 164(4):256-267 (2016)). The probability of an erroneous medical conclusion during mammographic screening can reach as high as 19% (Independent UK Panel on Breast Screening, The Lancet, 380(9855):P1778-P1786 (2012) and the probability of obtaining a false-positive result is 20% (Lauby-Secretan et al., New Engl. J. Med., 372(24):2353-2358 (2015)). False positive results and erroneous medical reports, in turn, can lead to the provision of medical care to patients who do not really need it.

Due to the obvious shortcomings of mammographic examination, because biopsy is an invasive procedure limited only to certain locations and not always feasible in clinical practice, and because imaging often cannot provide enough information on tumor character to direct further treatment, the development of new approaches aimed at early diagnosis of breast cancer are needed. Currently, in clinical practice, there are no generally recognized biomarkers that permit a minimally invasive, accurate, reliable, fast, simple, inexpensive and patient-friendly test for diagnosing breast cancer in the early stages and possessing high specificity and sensitivity. The methods provided herein offer liquid biopsy alternatives using biomarkers, such as the abundance of cell-free nucleic acids derived from certain target genes in a body fluid, and/or certain enzymatic activities, such as nuclease activity, in a body fluid, that permit the diagnosis of cancers such as breast cancer with high specificity and sensitivity. The methods provided herein, including combination methods, e.g., measuring cell-free nucleic acid abundance alone, measuring nuclease activity alone, or measuring cell-free nucleic acid abundance and nuclease activity in a body fluid sample permit the diagnosis, monitoring, and/or treatment of various subtypes of breast cancer, including, but not limited to, Luminal A, Luminal B, triple negative and HER2-enriched breast cancers.

### 2. Pathogenic Infections

Exemplary pathogenic infections that can be analyzed, monitored and/or treated by the methods provided herein, include, but are not limited to, infections caused by viruses, including DNA viruses and retroviruses, fungi, bacteria, parasites and protozoan pathogens. Exemplary pathogens include, but are not limited to, Herpes virus, Ebola virus, West Nile virus, Vaccinia virus, Epstein Barr virus, Hepatitis A Virus (HAV), Hepatitis B Virus (HBV); Hepatitis C Virus (HCV), herpes viruses (e.g. HSV-1, HSV-2, HHV-6, CMV), Human Immunodeficiency Virus (HIV), Vesicular Stomatitis Virus (VSV), coronaviruses (e.g., Sars-CoV-2), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lamblia, trichinella, Dientamoeba fragilis, Trypanosoma brucei, Trypanosoma cruzi, or Leishmania donovani. Exemplary bacteria include, but are not limited to, Bacillus anthracis, Borrelia burgdorferi, Campylobacter jejuni, Chlamydia trachomatis, Clostridium botulinum, Clostridium tetani, Diphtheria, Escherichia coli, Legionella, Helicobacter pylori, Mycobacterium rickettsia, Mycobacterium tuberculosis, Mycoplasma Neisseria, Pertussis, Pseudomonas aeruginosa, Streptococcus pneumoniae, Streptococcus, Staphylococcus, Vibrio cholerae and Yersinia pestis. Exemplary fungi include, but are not limited to, fungi such as Aspergillus fumigatus, Blastomyces dermatitidis, Candida albicans, Coccidioides immitis, Cryptococcus neoformans, Histoplasma capsulatum and Penicillium marneffei. Exemplary protozoa and parasites include, but are not limited to, chlamydia, kokzidiose/coccidiosis, leishmania, malaria, rickettsia, and trypanosoma.

In some examples of the methods provided herein, the disease or condition is caused by a virus. Viral diseases affect millions of people worldwide. Annually, dengue virus disease affects about 50 to 100 million people globally with 9000+ fatalities, rotavirus infects about two million children under five years of age, of whom about 527,000 die, seasonal influenza epidemics cause severe illness in three to five million people and a quarter to a half million deaths, and the most recent SARS-CoV-2 virus has caused over a million deaths within the space of just a few months, not to mention countless cases of serious illness with lingering long-term effects such as organ damage and cardiac disease. Exemplary viral infections that can be analyzed, monitored and/or treated according to the methods provided herein include, but are not limited to, those caused by coronaviruses, adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papovavirus, polio, respiratory syncytial virus, rinderpest, rhinovirus, rotavirus, rubella, SARS virus, smallpox and viral meningitis.

### SARS-CoV-2

In some examples, the disease or condition that is analyzed, monitored and/or treated by the methods provided herein is COVID-19, and the pathogen causing the disease or condition is a SARS-CoV-2. So-called novel coronaviruses (nCoV) are part of a family of beta coronaviruses that can cause relatively mild illnesses such as the common cold, or create more life threatening conditions such as SARS-CoV (Severe Acute Respiratory Syndrome) or MERS-CoV (Middle East Respiratory Syndrome). As a family of viruses, the coronaviruses are transmitted from animals to humans (zoonotic transmission) followed by human-to-human transmission. SARS-CoV was initially spread from civet cats to humans, and MERS-CoV was transmitted from dromedary camels to humans.

SARS-CoV-2 is an enveloped, positive-sense, single-stranded RNA virus that the WHO (World Health Organization) has declared to have caused a global pandemic, causing world-wide distress and economic hardship. COVID-19 disease (SARS-CoV-2) can present with flu like symptoms such as fever, cough, shortness of breath, breathing difficulties, nausea, diarrhea, pneumonia, severe acute respiratory syndrome, coagulation dysfunction, septic shock, kidney failure, and even death. The corona protein spikes situated at the exterior surface of the virus are the binding point for the Angiotensin-converting enzyme 2 (ACE2) receptor in lung tissue. Since patients treated with ACEIs will have increased numbers of ACE2 receptors in their lungs, an increase in ACE2 receptor mediated antigenic stimuli can therefore increase the risk of the corresponding inflammation-related adverse effects associated with COVID-19 infections. Consequently, pneumonia, acute myocardial injury, and/or chronic damage to the cardiovascular system are potential severe complications of this viral disease, and the symptoms of the infection can be more pronounced in patients with hypertension and diabetes. Therefore, affected patients may be at an increased risk of more severe disease outcomes mediated by infection-induced endothelial dysfunction.

Given the highly infectious and deadly nature of SARS-CoV-2, there is a need for a minimally invasive, accurate, reliable, fast, simple, inexpensive and patient-friendly test for diagnosing infections caused by SARS-CoV-2, such as COVID-19, in the early stages that possesses high specificity and sensitivity. The methods provided herein offer liquid biopsy alternatives using biomarkers, such as the abundance of cell-free nucleic acids derived from certain target genes in a body fluid, and/or certain enzymatic activities, such as nuclease activity, in a body fluid, that permit the early diagnosis, monitoring and/or therapeutic intervention/treatment of diseases or conditions caused by SARS-CoV-2.

### K. COMPOSITIONS, COMBINATIONS AND KITS

Provided herein are compositions containing a nucleic acid probe molecule, e.g., for quantitating the amount of cell-free nucleic acid by real-time-qPCR or for measuring an enzymatic activity, such as a nuclease activity, where the nucleic acid probe molecule contains at least one zinc finger binding motif. In examples, the nucleic acid probe is associated with a detectable label (e.g., covalently or non-covalently associated, as described elsewhere herein). Combinations of a nucleic acid probe containing at least one zinc finger binding motif and a detectable label that can covalently or non-covalently be attached to the nucleic acid probe also are provided herein. The combination can further contain one or more primer pairs and/or reactions for performing PCR such as a polymerase and/or deoxynucleotides and/or buffer, e.g., for amplifying, in a body fluid, cell-free nucleic acid derived from a target gene, such as in Method 1 or combination methods as provided herein, or can contain reagents for measuring nuclease activity in Method 2A, Method 2B or combination methods as provided herein, where the reagents can include one or more of Zn²⁺, Mg²⁺, a detergent such as one known to those of skill in the art and/or described herein, exemplary of which is NP40, and a reducing agent such as one known to those of skill in the art and/or described herein, exemplary of which is DTT. In some examples, the combination can include more than one nucleic acid probe molecule and/or one or more reagents for performing more than one method selected from among Method 1, Method 2A, Method 2B and/or combination methods as provided herein.

The combinations also can include treatments to administer to subjects identified as having a disease or condition. For example, a subject previously identified as having COVID-19 can be administered a treatment for COVID-19, such as the antiviral drug remdesivir, molnupiravir, ritonavir, and anti-SARS-CoV-2 monoclonal antibodies such as bamlanivimab, etesevimab, casirivimab or combinations of two or more antibodies thereof. For example, a subject previously identified as having cancer can be administered an anti-cancer treatment such as a chemotherapeutic agent for treating cancer, such as a nucleoside analog or other antimetabolite (e.g. gemcitabine or derivative or other nucleoside analog), tumor-targeted taxanes, tumor-targeted taxane and nucleoside analog combination, Acivicins; Avicin; Aclarubicins; Acodazoles; Acronines; Adozelesins; Aldesleukins; Alemtuzumabs; Alitretinoins (9-Cis-Retinoic Acids); Allopurinols; Altretamines; Alvocidibs; Ambazones; Ambomycins; Ametantrones; Amifostines; Aminoglutethimides; Amsacrines; Anastrozoles; Anaxirones; Ancitabines; Anthramycins; Apaziquones; Argimesnas; Arsenic Trioxides; Asparaginases; Asperlins; Atrimustines; Azacitidines; Azetepas; Azotomycins; Banoxantrones; Batabulins; Batimastats; BCG Live; Benaxibines; Bendamustines; Benzodepas; Bexarotenes; Bevacizumab; Bicalutamides; Bietaserpines; Biricodars; Bisantrenes; Bisantrenes; Bisnafide Dimesylates; Bizelesins; Bleomycins; Bortezomibs; Brequinars; Bropirimines; Budotitanes; Busulfans; Cactinomycins; Calusterones; Canertinibs; Capecitabines; Caracemides; Carbetimers; Carboplatins; Carboquones; Carmofurs; Carmustines with Polifeprosans; Carmustines; Carubicins; Carzelesins; Cedefingols; Celecoxibs; Cemadotins; Chlorambucils; Cioteronels; Cirolemycins; Cisplatins; Cladribines; Clanfenurs; Clofarabines; Crisnatols; Cyclophosphamides; Cytarabine liposomals; Cytarabines; Dacarbazines; Dactinomycins; Darbepoetin Alfas; Daunorubicin liposomals; Daunorubicins/Daunomycins; Daunorubicins; Decitabines; Denileukin Diftitoxes; Dexniguldipines; Dexonnaplatins; Dexrazoxanes; Deazaguanines; Diaziquones; Dibrospidiums; Dienogests; Dinalins; Disermolides; Docetaxels; Dofequidars; Doxifluridines; Doxorubicin liposomals; Doxorubicin HCL; Doxorubicin HCL liposome injection; Doxorubicins; Droloxifenes; Dromostanolone Propionates; Duazomycins; Ecomustines; Edatrexates; Edotecarins; Eflornithines; Elacridars; Elinafides; Elliott's B Solutions; Elsamitrucins; Emitefurs; Enloplatins; Enpromates; Enzastaurins; Epipropidines; Epirubicins; Epoetin alfas; Eptaloprosts; Erbulozoles; Esorubicins; Estramustines; Etanidazoles; Etoglucids; Etoposide phosphates; Etoposide VP-16s; Etoposides; Etoprines; Exemestanes; Exisulinds; Fadrozoles; Fazarabines; Fenretinides; Filgrastims; Floxuridines; Fludarabines; Fluorouracils; 5-fluorouracils; Fluoxymesterones; Flurocitabines; Fosquidones; Fostriecins; Fotretamines; Fulvestrants; Galarubicins; Galocitabines; Gemcitabines; Gemtuzumabs/Ozogamicins; Geroquinols; Gimatecans; Gimeracils; Gloxazones; Glufosfamides; Goserelin acetates; Hydroxyureas; Ibritumomabs/Tiuxetans; Idarubicins; Ifosfamides; Ilmofosines; Ilomastats; Imatinib mesylates; Imexons; Improsulfans; Indisulams; Inproquones; Interferon alfa-2as; Interferon alfa-2bs; Interferon Alfas; Interferon Betas; Interferon Gammas; Interferons; Interleukin-2s and other Interleukins (including recombinant Interleukins); Intoplicines; Iobenguanes [131-I]; Iproplatins; Irinotecans; Irsogladines; Ixabepilones; Ketotrexates; L-Alanosines; Lanreotides; Lapatinibs; Ledoxantrones; Letrozoles; Leucovorins; Leuprolides; Leuprorelins (Leuprorelides); Levamisoles; Lexacalcitols; Liarozoles; Lobaplatins; Lometrexols; Lomustines/CCNUs; Lomustines; Lonafarnibs; Losoxantrones; Lurtotecans; Mafosfamides; Mannosulfans; Marimastats; Masoprocols; Maytansines; Mechlorethamines; Mechlorethamines/Nitrogen mustards; Megestrol acetates; Megestrols; Melengestrols; Melphalans; Melphalans (L-PAMs); Menogarils; Mepitiostanes; Mercaptopurines; 6-Mercaptopurine; Mesnas; Metesinds; Methotrexates; Methoxsalens; Metomidates; Metoprines; Meturedepas; Miboplatins; Miproxifenes; Misonidazoles; Mitindomides; Mitocarcins; Mitocromins; Mitoflaxones; Mitogillins; Mitoguazones; Mitomalcins; Mitomycin Cs; Mitomycins; Mitonafides; Mitoquidones; Mitospers; Mitotanes; Mitoxantrones; Mitozolomides; Mivobulins; Mizoribines; Mofarotenes; Mopidamols; Mubritinibs; Mycophenolic Acids; Nandrolone Phenylpropionates; Nedaplatins; Nelzarabines; Nemorubicins; Nitracrines; Nocodazoles; Nofetumomabs; Nogalamycins; Nolatrexeds; Nortopixantrones; Octreotides; Oprelvekins; Ormaplatins; Ortataxels; Oteracils; Oxaliplatins; Oxisurans; Oxophenarsines; Paclitaxels; Pamidronates; Patubilones; Pegademases; Pegaspargases; Pegfilgrastims; Peldesines; Peliomycins; Pelitrexols; Pemetrexeds; Pentamustines; Pentostatins; Peplomycins; Perfosfamides; Perifosines; Picoplatins; Pinafides; Pipobromans; Piposulfans; Pirfenidones; Piroxantrones; Pixantrones; Plevitrexeds; Plicamycid Mithramycins; Plicamycins; Plomestanes; Porfimer sodiums; Porfimers; Porfiromycins; Prednimustines; Procarbazines; Propamidines; Prospidiums; Pumitepas; Puromycins; Pyrazofurins; Quinacrines; Ranimustines; Rasburicases; Riboprines; Ritrosulfans; Rituximabs; Rogletimides; Roquinimexs; Rufocromomycins; Sabarubicins; Safingols; Sargramostims; Satraplatins; Sebriplatins; Semustines; Simtrazenes; Sizofirans; Sobuzoxanes; Sorafenibs; Sparfosates; Sparfosic Acids; Sparsomycins; Spirogermaniums; Spiromustines; Spiroplatins; Spiroplatins; Squalamines; Streptonigrins; Streptovaricins; Streptozocins; Sufosfamides; Sulofenurs; Sunitinib Malate; 6-thioguanine (6-TG); Tacedinalines; Tales; Talisomycins; Tallimustines; Tamoxifens; Tariquidars; Tauromustines; Tecogalans; Tegafurs; Teloxantrones; Temoporfins; Temozolomides; Teniposides/VM-26s; Teniposides; Teroxirones; Testolactones; Thiamiprines; Thioguanines; Thiotepas; Tiamiprines; Tiazofurins; Tilomisoles; Tilorones; Timcodars; Timonacics; Tirapazamines; Topixantrones; Topotecans; Toremifenes; Tositumomabs; Trabectedins (Ecteinascidin 743); Trastuzumabs; Trestolones; Tretinoins/ATRA; Triciribines; Trilostanes; Trimetrexates; Triplatin Tetranitrates; Triptorelins; Trofosfamides; Tubulozoles; Ubenimexs; Uracil Mustards; Uredepas; Valrubicins; Valspodars; Vapreotides; Verteporfins; Vinblastines; Vincristines; Vindesines; Vinepidines; Vinflunines; Vinformides; Vinglycinates; Vinleucinols; Vinleurosines; Vinorelbines; Vinrosidines; Vintriptols; Vinzolidines; Vorozoles; Xanthomycin As (Guamecyclines); Zeniplatins; Zilascorbs [2-H]; Zinostatins; Zoledronate; Zorubicins; and Zosuquidars, for example: Aldesleukins (e.g. PROLEUKIN®); Alemtuzumabs (e.g. CAMPATH®); Alitretinoins (e.g. PANRETIN®); Allopurinols (e.g. ZYLOPRIM®); Altretamines (e.g. HEXALEN®); Amifostines (e.g. ETHYOL®); Anastrozoles (e.g. ARIMIDEX®); Arsenic Trioxides (e.g. TRISENOX®); Asparaginases (e.g. ELSPAR®); BCG Live (e.g. TICE® BCG); Bexarotenes (e.g. TARGRETIN®); Bevacizumab (AVASTIN®); Bleomycins (e.g. BLENOXANE®); Busulfan intravenous (e.g. BUSULFEX®); Busulfan orals (e.g. MYLERAN®); Calusterones (e.g. METHOSARB®); Capecitabines (e.g. XELODA®); Carboplatins (e.g. PARAPLATIN®); Carmustines (e.g. BCNU™, BiCNU®); Carmustines with Polifeprosans (e.g. GLIADEL® Wafer); Celecoxibs (e.g. CELEBREX®); Chlorambucils (e.g. LEUKERAN®); Cisplatins (e.g. PLATINOL®); Cladribines (e.g. LEUSTATIN®, 2-CdA®); Cyclophosphamides (e.g. CYTOXAN®, NEOSAR®); Cytarabines (e.g. CYTOSAR-U®); Cytarabine liposomals (e.g. DepoCyt®); Dacarbazines (e.g. DTIC-Dome): Dactinomycins (e.g. COSMEGEN®); Darbepoetin Alfas (e.g. ARANESP®); Daunorubicin liposomals (e.g., DAUNOXOME®); Daunorubicins/Daunomycins (e.g. CERUBIDINE®); Denileukin Diftitoxes (e.g. ONTAK®); Dexrazoxanes (e.g. ZINECARD®); Docetaxels (e.g. TAXOTERE®); Doxorubicins (e.g. ADRIAMYCIN®, RUBEX®); Doxorubicin liposomals, including Doxorubicin HCL liposome injections (e.g. DOXIL®); Dromostanolone propionates (e.g. DROMOSTANOLONE™ and MASTERON® Injection); Elliott's B Solutions (e.g. Elliotts B® Solution); Epirubicins (e.g. ELLENCE®); Epoetin alfas (e.g. EPOGEN®); Estramustines (e.g. EMCYT®); Etoposide phosphates (e.g. ETOPOPHOS®); Etoposide VP-16s (e.g. VEPESID®); Exemestanes (e.g. AROMASIN®); Filgrastims (e.g. NEUPOGEN®); Floxuridines (e.g. FUDR®); Fludarabines (e.g. FLUDARA®); Fluorouracils including 5-FUs (e.g. ADRUCIL®); Fulvestrants (e.g. FASLODEX®); Gemcitabines (e.g. GEMZAR®); Gemtuzumabs/Ozogamicins (e.g. MYLOTARG®); Goserelin acetates (e.g. ZOLADEX®); Hydroxyureas (e.g. HYDREA®); Ibritumomabs/Tiuxetans (e.g. ZEVALIN®); Idarubicins (e.g. IDAMYCIN®); Ifosfamides (e.g. IFEX®); Imatinib mesylates (e.g. GLEEVEC®); Interferon alfa-2as (e.g. ROFERON®-A); Interferon alfa-2bs (e.g. INTRON A®); Irinotecans (e.g. CAMPTOSAR®); Letrozoles (e.g. FEMARA®); Leucovorins (e.g. WELLCOVORIN®, LEUCOVORIN®); Levamisoles (e.g. ERGAMISOL®); Lomustines/CCNUs (e.g. CeeNU®); Mechlorethamines/Nitrogen mustards (e.g. MUSTARGEN®); Megestrol acetates (e.g. MEGACE®); Melphalans/L-PAMs (e.g. ALKERAN®); Mercaptopurine, including 6-mercaptopurines (6-MPs; e.g. PURINETHOL®); Mesnas (e.g. MESNEX®); Methotrexates; Methoxsalens (e.g. UVADEX®); Mitomycin Cs (e.g. MUTAMYCIN®, MITOZYTREX®); Mitotanes (e.g. LYSODREN®); Mitoxantrones (e.g. NOVANTRONE®); Nandrolone Phenylpropionates (e.g., DECA-DURABOLIN® 50); Nofetumomabs (e.g. VERLUMA®); Oprelvekins (e.g. NEUMEGA®); Oxaliplatins (e.g. ELOXATIN®); Paclitaxels (e.g. PAXENE®, TAXOL®); Pamidronates (e.g. AREDIA®); Pegademases (e.g. ADAGEN®); Pegaspargases (e.g. ONCASPAR®); Pegfilgrastims (e.g. NEULASTA®); Pentostatins (e.g. NIPENT®); Pipobromans (e.g. VERCYTE®); Plicamycin/Mithramycins (e.g. MITHRACIN®); Porfimer sodiums (e.g. PHOTOFRIN®); Procarbazines (e.g. MATULANE®); Quinacrines (e.g. ATABRINE®); Rasburicases (e.g. ELITEK®); Rituximabs (e.g. RITUXAN®); Sargramostims (e.g. PROKINE®); Streptozocins (e.g. ZANOSAR®); Sunitinib Malates (e.g. SUTENT®); Tales (e.g. SCLEROSOL®); Tamoxifens (e.g. NOLVADEX®); Temozolomides (e.g. TEMODAR®); Teniposides/VM-26s (e.g. VUMON®); Testolactones (e.g. TESLAC®); Thioguanines including, 6-thioguanine (6-TG); Thiotepas (e.g. THIOPLEX®); Topotecans (e.g. HYCAMTIN®); Toremifenes (e.g. FARESTON®); Tositumomabs (e.g. BEXXAR®); Trastuzumabs (e.g. HERCEPTIN®); Tretinoins/ATRA (e.g. VESANOID®); Uracil Mustards; Valrubicins (e.g. VALSTAR®); Vinblastines (e.g. VELBAN®); Vincristines (e.g. ONCOVIN®); Vinorelbines (e.g. NAVELBINE®); Zoledronates (e.g. ZOMETA®), oncolytic viral therapies, immunotherapies, therapeutic antibodies, CAR-T therapy and other cell therapies, and checkpoint inhibitors. Anti-cancer agents/treatment regimens for particular cancers and stages of progression as determined by monitoring, including monitoring by the methods provided herein, are known to those of skill in the art.

The combinations can be packaged as kits, optionally, with instructions for one or more of the following: labeling the reagents (such as nucleic acid probe(s)), using the reagents and/or instructions for performing one or more steps of the method(s). In some examples, the kits also can optionally include one or more software or computer program products, e.g., for executing an algorithm to measure the amount of amplified cell-free nucleic acid in a sample of body fluid (e.g., Ct value) or for measuring enzymatic activity or ratios of activities, such as nuclease activity or ratios of nuclease activities, and/or for classifying a subject as having a disease or condition or not having a disease or condition based on the value (e.g., Ct value) of the amount of amplified nucleic acid measured in the body fluid or the measured enzymatic activity or ratio of activities, such as nuclease activity or ratio of nuclease activities. The kit optionally can include instructions for using the devices and reagents, handling the sample, and analyzing the data. The kits provided herein also can be used with a computer system or software to analyze samples from subjects for selection either to undergo treatment or not undergo treatment or undergo a particular course of treatment for a disease or condition.

The kits can also contain one or more reagents (e.g., solubilization buffers, detergents, washes, or buffers) for processing a sample. Any of the kits provided herein can also include, e.g., buffers, reducing agents, preserving agents such as glycerol or other glycol, reducing agents such as DTT and other reducing agents as provided herein and as known to those of skill in the art, blocking agents, positive control samples, negative control or reference samples, reference standards, software and information such as protocols, guidance and reference data.

Also provided are articles of manufacture containing packaging materials, any composition or combination provided herein, containers such as tubes or wells (such as 96-well plates) for performing reactions using the compositions or combinations, and a label that indicates the contents and/or use. The choice of package depends on the agents, and whether such compositions will be packaged together or separately. In general, the packaging is non-reactive with the compositions contained therein. In some examples, some of the components can be packaged as a mixture. In examples, all components are packaged separately.

### L. EXAMPLES

The following examples demonstrate, among other things, that analyzing the abundance of certain cell-free nucleic acids (Methods 1) and/or Mg²⁺ and/or Zn²⁺-dependent nuclease activities (Methods 2A and Methods 2B) in a body fluid of a subject, with a disease, disorder, or condition, such as cancer patient or subject relative to that of a normal healthy subject, improves the accuracy of disease detection, particularly cancer detection, and determination of a proper treatment strategy. Each of these methods is summarized below and also described throughout the disclosure herein.

In Method 1, the abundance of cell-free nucleic acids in a body fluid, where the cell-free nucleic acids are derived from one or more target genes, is analyzed as indicative of the presence or absence of a disease or condition in the subject. A sample, such as a body fluid, that is obtained from a subject or that has previously been obtained from a subject, is subjected to conditions under which cell-free nucleic acid derived from at least one target gene is specifically amplified. The amplified nucleic acid then is quantified, and: i) if the amount of amplified nucleic acid is at or above a threshold level, the subject is identified as having the disease or condition; and ii) if the amount of amplified nucleic acid is below a threshold level, the subject is identified as not having the disease or condition.

In Method 2A and Method 2B, cell-free nuclease activity in a body fluid from a subject is analyzed and is indicative of the presence or absence of a disease or condition in the subject. A sample of body fluid that is obtained from a subject or has previously been obtained from a subject is subjected to conditions under which cell-free nuclease activity is assessed as a ratio of nuclease activities in one of two ways, designated Method 2A and Method 2B, respectively.

In “Method 2A,” the body fluid sample is exposed to two sets of reaction conditions: one in which the cell-free nuclease activity is measured in the presence of exogenously added zinc (Zn²⁺) and magnesium (Mg²⁺), and another in which the cell-free nuclease activity is measured in the presence of exogenously added magnesium alone, with no exogenously added zinc. If the ratio (designated K-A3) of the nuclease activity in the presence of exogenously added zinc relative to the nuclease activity in the absence of exogenously added zinc is at or above a threshold level, the subject is identified as having the disease or condition, and if this ratio is below a threshold level, the subject is identified as not having the disease or condition.

In “Method 2B,” the body fluid sample of the subject being tested for the presence or absence of the disease or condition is exposed to reaction conditions in which the cell-free nuclease activity of the sample is measured. A reference or control sample is subjected to the same or similar reaction conditions for measuring the nuclease activity, or a predetermined value of nuclease activity from a control or reference sample is obtained. If the ratio (K-A3B) of the nuclease activity measured in the subject being tested relative to the nuclease activity of the control or reference sample is at or above a threshold level, the subject is identified as having the disease or condition and if this ratio is below a threshold level, the subject is identified as not having the disease or condition. The reactions to measure nuclease activity in the sample from the subject and in the control or reference sample both can be performed in the presence of exogenously added magnesium alone, with no exogenously added zinc, or both can be performed in the presence of exogenously added zinc and magnesium.

These methods are less onerous and more cost effective than, for example, imaging methods. The results are analyzed using an algorithmic scoring function and machine learning methods, which can exclude human error. Scoring functions are described in more detail and an example of a scoring function is described and shown in Example 11.

Another aspect of these methods, Methods 1, Methods 2A, Methods 2B, and combinations thereof, is that they use and require small volumes of plasma samples, for example, up to only about 5 μl of plasma per 25-μl reaction. This characteristic derives from the intrinsic sensitivity of these assays, and it offers, among others, advantages, including, but are not limited to the following.
i. Multiple reactions (replicates) can be performed on each sample. This can be used to overcome the generation of false negatives by the reduction of stochastic noise (naturally occurring sampling errors).
ii. The same sample can be analyzed at later times to confirm initial results and/or to enhance the overall accuracy of the results.
iii. The sample can be used for multiple assays, such as Methods 1 with different target genes and Methods 2 with different probes.
iv. Relatively small volumes of blood need to be drawn from subjects, reducing their physical burden.

These assays (Methods 1 and Methods 2) can be used at two different stages in the disease, such as breast cancer, diagnosis and progression. One is for routine screening of subjects to identify subjects for further diagnostic tests, such prior to diagnostic tests by imaging modalities, such as mammography for breast cancer. These assays can distinguish subjects, who should be evaluated further by imaging-based diagnostic tests, from those, who do not need diagnostic imaging. This reduces the total number of diagnostic imaging tests, resulting in the reduction of the number of subjects, who undergo diagnostic imaging tests and are exposed to radiation. The other stage where these assays can be used is after diagnosis, such as after diagnostic imaging. When a given subject is positive for potential development of disease, disorder, or condition, such as breast cancer by diagnostic imaging, the subject is further assessed by Methods 1 and/or Methods 2. Evaluation of two sets of diagnosis results (differential diagnosis), one from diagnostic imaging and the other from the assays of the methods herein, for a given subject offers more accurate diagnosis, such as of breast cancer, and is very useful to determine whether or not the subject should undergo a biopsy, such as a biopsy of breast tissue with pathological evaluation.

The methods provide herein also can be used to monitor subjects for the effectiveness of the treatment the subjects have received (such as surgical resection of tumors and surrounding tissues), the detection of recurrence of a given cancer, the detection of the development of cancer derived from tumors that are not detectable by diagnostic imaging, and the detection of pre-existing or post-treatment metastasis of cancer.

The following examples are included for illustrative purposes only and are not intended to limit the scope of the invention.

### Example 1

Selection of Genes and Design of Primers and Probes for Cancer Detection Blood plasma was collected from 20 donors: 10 healthy subjects and 10 patients with colorectal cancer. Colorectal cancer patients had Stage I or Stage II disease, mean age 68.3 years (49 years-85 years); 5 patients had right-sided colon cancer and 5 patients had left-sided colon cancer. Healthy subjects were not diagnosed with cancer and were of mean age 61.2 years (40 years-80 years). Whole blood was obtained by venipuncture into vacutainer tubes with EDTA and stored at 4° C. before for not more than 2 hours before centrifugation. Tubes were centrifuged twice for 10 minutes at 2600 g to separate plasma from cells.

For each donor plasma sample, DNA was isolated from 200 ul of plasma using the linear polyacrylamide (LPA) precipitation method. Briefly, to each plasma sample in 0.1M salt, 10-20 μg of LPA and 2.5 volumes of ethanol was added. The mixture was vortexed and then centrifuged at 12,000×g in a microcentrifuge. The resulting DNA pellets were resuspended and dissolved in buffer (10 mM Tris/EDTA, pH 8.0).

The DNA was fragmented by ultrasound, while keeping the tubes containing the DNA cool using flowing cold water. Ultrasound was used to create a more uniform size distribution among the tested samples; the sizes of fragmented DNA in the samples were in the range of 300-700 bp, as determined by gel electrophoresis. The resulting sheared fragments were stitched together in rings, using T4 DNA ligase with a DNA concentration of less than 1 μg/mL; ring structures can be amplified effectively in a continuous manner using phi29 DNA polymerase.

Whole Genome Amplification was performed on 200 μg of DNA from each plasma sample, using phi29 DNA polymerase (New England Biolabs (NEB); Catalog #M0269L) in the presence of single-stranded binding protein (SSB) (NEB, SSB protein Catalog #M0249S), trehalose, 3′-S-S(dithio) modified random heptamer primers (Syntol JSC, Moscow, Russia) that are resistant to exonuclease, 8% polyethylene glycol, deoxynucleoside triphosphates (dNTP), magnesium chloride and the plasma nucleic acids processed as described above were mixed in the amounts/concentrations shown in Table 1 below:

TABLE 1
Composition of the mixture for DNA synthesis

| Component | Volume | |
|---|---|---|
| 10 x buffer | 10 | μl |
| 1.35 mM trehalose | 28 | μl |
| 25 mM dNTP | 10 | μl |
| Random primers, 0.5 mM | 3 | μl |
| 10 mg/ml BSA (bovine serum albumin) | 2 | μl |
| 0.1M DTT (dithiothreitol) | 1 | μl |
| 50% polyethylene glycol | 8 | μl |
| Blood plasma | 10 | μl |

| | |
|---|---|
| Protein SSB | 1 μl/20 ng |
| Phi29 DNA polymerase | 5 μl/500 u |
| Water | Up to 100 μl |

For nucleic acid denaturation, the components of Table 1 other than the DNA polymerase and SSB were incubated for 2 minutes at 95° C. The tubes were placed on ice, DNA polymerase with protein SSB was added, and incubation continued for 16 hours at 30° C. After 16 hours of amplification under standard conditions, the amount of DNA increased more than 10,000-fold in each of the 20 samples and 30 μg of DNA was obtained per plasma sample (donor).

Each donor DNA sample (30 μg) was hybridized to a human CpG island chip containing 243,504 fragments (Human CpG Island-Chip 244 K Microarray Kit from Agilent). Separate slides were used for each donor sample. On 4 slides, the donor samples were grouped as 5 samples per slide. The DNA was labeled with Cyanide5 (Cy5) fluorescent dye and scanned on a Scanner 4200; the abundance of individual hybridized fragments was determined by measuring the fluorescence.

The results showed that some gene fragments were detected in equal amounts in the DNA samples from healthy subjects and cancer patients, while for other gene fragments, differences in abundance were observed between and among the “healthy” DNA samples and the “cancer” DNA samples. Fragments that were 5 or more times (generally, 5-8 times) more abundant in cancer DNA compared to healthy DNA were selected for further study, and the corresponding genes identified using the Agilent information file that provides the genes from which each of the fragments on the chip are derived. To ensure that the more abundant genes from subjects with cancer (referred to as cancer DNA genes) were reliably identified, the abundance was measured in fragments on the chip that had adjacent (sequential) numbers, i.e., hybridization signals of more than one fragment derived from the same gene were measured.

272 fragments were initially identified as meeting the above criteria (at least 5 times stronger hybridization signal in cancer DNA). Each of the identified 272 fragments were found to have at least one zinc finger binding motif: (CNN)x, where C is cytosine and N is A, G, C or T. Of the 272 fragments, genes that are implicated in cancers and, in general, are not over-expressed in an organ-specific manner were selected for Method 1 or Method 2A or Method 2B analysis (e.g., EGFR is over-expressed in epithelial cells in many cancers; epithelial cells are present in all organs and, therefore, EGFR can be over-expressed in cancers in many tissues). A subset of the selected genes is listed below:
1. ESR1 (estrogen receptor 1)
2. PGR (progesterone receptor)
3. HER2 (human epidermal growth factor receptor 2)
4. ARF family proteins and regulators, e.g., ARF1 and ARFIP1 (ADP-Ribosylation factor 1) and ARFIP1 (ADP-Ribosylation factor interacting protein 1)
5. COX1 (cyclooxygenase 1)
6. COX11 (cytochrome c oxidase, mitochondrial)
7. Perilipin (PLIN) family (e.g., PLIN1; PLIN3)
8. EGFR (epidermal growth factor receptor)
9. MMP7 (matrix metallopeptidase 7)
10. MMP9 (matrix metallopeptidase 9)
11. SOX1 (SRY-Box Transcription Factor 1)
12. TERT (telomerase reverse transcriptase)
13. P53 tumor suppressor protein
14. MED12 (mediator complex subunit 12)
15. RFX2 (Regulatory Factor X2)
16. P21 (cyclin-dependent kinase inhibitory protein-1; induces tumor growth suppression through P53)
17. PI3 KB (phosphoinositide 3-kinase beta)
18. SEPT9 (septin-9)

In particular, target genes/segments were identified as follows. Cell-free DNA was isolated by a linear polyacrylamide coprecipitation method from plasma, which had been collected from normal subjects and those with colon cancer (10 subjects from each group). The resulting cell-free DNA fragments from each subject (0.2 ng per reaction) were subjected to whole-genome amplification, which involved DNA polymerase from bacteriophage phi29. This resulted in the generation of amplified DNA fragments, the amount of which increased by more than 10,000-fold, providing sufficient amounts of DNA needed for subsequent hybridization analysis. The amplified DNA fragments from each subject were applied to an Agilent microarray (chip) containing 244,000 microarray elements, each of which has a 60-base DNA sequence. The amplified DNA fragments were allowed to hybridize to microarray elements, followed by the detection of hybridization events by fluorescence. Approximately 200 DNA fragments were identified that gave higher fluorescence signals with DNA fragments from subjects with colon cancer by 5-8 times than those from normal subjects. The number of these DNA fragments was reduced to approximately 20, based on additional analysis of the differences in fluorescence intensity in the microarray data between the two subject groups and literature-based assessment of their potential involvement in and relevance to cancers. The resulting 20 species of DNA fragments were analyzed by quantitative PCR. A primer pair and a TaqMan probe were designed for each DNA fragment species and used in real-time quantitative PCR, in which plasma samples from normal subjects and those with breast cancer were analyzed individually for the detection and quantification of each DNA fragment species. This analysis allowed for the identification of the four genes and their segments, based on the differences in the amount of each gene segment between the two subject groups. These gene segments are derived from the coding sequences of estrogen receptor 1 (ESR1), epidermal growth factor receptor 1 (EGFR), epidermal growth factor receptor 2 (HER2), and matrix metallopeptidase 9 (MMP9). All of these gene segments are highly GC-rich, and some of them contain potential binding sites for zinc finger proteins.

In addition to being implicated in cancers, the genes identified and selected for analysis by the methods provided, described, and exemplified herein, have several zinc finger binding sites (motifs). The methods provided herein, as described elsewhere herein and in the first paragraph of this Examples section, are designated Method 1 and Method 2A and Method 2B. To perform the methods, primers and probes for quantitating the relative abundance of these genes in cancer vs. healthy body fluid samples containing cell-free DNA (Method 1), and probes for measuring the relative Mg²⁺ and/or Zn²⁺-dependent nuclease activities in cancer vs. healthy body fluid samples containing cell-free DNA (Methods 2A and 2B), were designed. The probes were designed to target the nucleotide sequence containing the “Zinc Neck” region to which the “Hand” of the zinc finger binding proteins bind. The probes were tagged with a fluorescent label at the 5′-end (e.g., 6-carboxyfluorescein, FAM) and with a fluorescence quencher at the 3′-end (e.g., Black Hole Quencher 1, BHQ1). In the experiments described herein, the probes were labeled with FAM at the 5′-end and BHQ1 at the 3′-end. The primers flank the region that binds to the probe sequence.

For Method 1, for each of one or more genes selected from the above subset of genes, forward and reverse primers and a quantitation probe tagged with a FAM fluorescent label at the 5′-end and BHQ1 fluorescence quencher at the 3′-end were used to obtain a quantitative measure of abundance of each of the genes in body fluid samples from a cancer subject and from a healthy subject, by quantitative PCR (qPCR). In probe-based qPCR, the fluorescence of the quantitation probe remains quenched until the probe hybridizes to its complement in the cell-free DNA and is amplified using a DNA polymerase having 3′→5′ exonuclease activity, which releases the quencher and increases fluorescence in the reaction mix; the amount of fluorescence measured in the reaction mix is a measure of the amount of the gene corresponding to the probe. For Methods 2A and 2B, MgR and/or Zn²⁺-dependent nuclease activities were measured by monitoring nuclease-mediated increase of fluorescence of the label from the probes. The fluorescence remains quenched until the quencher of the probe is released by nuclease activity and increases fluorescence in the reaction mix; the amount of fluorescence measured in the reaction mix at the endpoint of the reaction is a measure of the nuclease activity. Primers and Probes were obtained from Syntol JSC (Moscow, Russia). Exemplary primer sequences (not bolded; “FOR”=forward; “REV”=reverse) for genes analyzed by Method 1 and exemplary probe sequences (in bold; with 5′-FAM and 3′-BHQ1 labels) for genes analyzed by Method 1, Method 2A or Method 2B are listed in Table 2, below.

TABLE 2
Exemplary primer and probe sequences

| Genecards.org* | GENE | PROBES AND PRIMERS (SEQ ID NO) | mRNA |
|---|---|---|---|
| NM_000125 | ESR1 | 5′FAM-CTGAACCGTCCGCAGCTCAAGATCC-3′BHQ1 (SEQ | mRNA_6327 |
| | | ID NO: 1) | |
| NM_000125 | ESR1 | FOR- GGATGGCCCTACTGCATCAGAT (SEQ ID NO: 2) | mRNA_6327 |
| NM_000125 | ESR1 | REV-CTTGCTGCTGTCCAGGTACAC (SEQ ID NO: 3) | mRNA_6327 |
| NM_000926.4 | PGR | 5′FAM-CATGTGGCAGATCCCACAGGAGTTTG-3′BHQ1 | mRNA_13037 |
| | | (SEQ ID NO: 4) | |
| NM_000926.4 | PGR | FOR-CAGCGGATGAAAGAATCATCATTCTATTC (SEQ ID | mRNA_13037 |
| | | NO: 5) | |
| NM_000926.4 | PGR | REV-AGGAACTCTTCTTGGCTAACTTGAAGC (SEQ ID NO: 6) | mRNA_13037 |
| NM_001005862 | HER2 | 5′FAM-CCCAGCCCTGGTCACCTACAACAC-3′BHQ1 (SEQ ID | mRNA_4858 |
| | | NO: 7) | |
| NM_001005862 | HER2 | FOR-GGCCTGCCTCCACTTCAAC (SEQ ID NO: 8) | mRNA_4858 |
| NM_001005862 | HER2 | REV-GGGACAGGCAGTCACACAG (SEQ ID NO: 9) | mRNA_4858 |
| NM_00102559.3 | ARFIP1 | 5′FAM-GTCGCCGGTCCGACTGTCCTCT-3′BHQ1 (SEQ ID | mRNA_3436 |
| | | NO: 10) | |
| NM_00102559.3 | ARFIP1 | FOR-GGCTTCGCTGCTCTTGGTTC (SEQ ID NO: 11) | mRNA_3436 |
| NM_00102559.3 | ARFIP1 | REV-AGCAACTGCAGCTGTCACAAATTC (SEQ ID NO: 12) | mRNA_3436 |
| KY364234.1 | COX1 | 5′FAM-ATTCGGCGCATGAGCTGGAGTC-3′BHQ1 (SEQ ID | |
| | | NO: 13) | |
| KY364234.1 | COX1 | FOR-CAAACCACAAAGACATTGGAACACT (SEQ ID NO: 14) | |
| KY364234.1 | COX1 | REV-CAGCTCGGCTCGAATAAGGA (SEQ ID NO: 15) | |
| NM_001164189 | PLIN1 | 5′FAM-TGTCCTTGGCGCTAGACACCA-3′BHQ1 (SEQ ID | mRNA_2229 |
| | | NO: 16) | |
| NM_001164189 | PLIN1 | FOR-CGGTCACTACGGACTTTGTC (SEQ ID NO: 17) | mRNA_2229 |
| NM_001164189 | PLIN1 | REV-ACCAAGGAGCTTGTGTCGTCTAAG (SEQ ID NO: 18) | mRNA_2229 |
| NM_005228.5 | EGFR | 5′FAM-CACCACGTACCAGATGGATGTGAACC-3′BHQ1 (SEQ | mRNA_9905 |
| | | ID NO: 19) | |
| NM_005228.5 | EGFR | 5′FAM-GTGACTGCTGCCACAACCAG-3′BHQ1 (SEQ ID | mRNA_9905 |
| | | NO: 20) | |
| NM_005228.5 | EGFR | 5′FAM-AGCTCCCTCTCCTGCAGCAGCCTCCGCAGCGT- | mRNA_9905 |
| | | 3′BHQ1 (SEQ ID NO: 21) | |
| NM_005228.5 | EGFR | FOR-TCTGCCGCAAATTCCGAGAC (SEQ ID NO: 22) | mRNA_9905 |
| NM_005228.5 | EGFR | REV-GGACACTTCTTCACGCAGGTG (SEQ ID NO: 23) | mRNA_9905 |
| NM_002423.5 | MMP7 | 5′FAM-CTGGAATGTTAAACTCCCGCGTCATAGAAATAATGC- | mRNA_1119 |
| | | 3′BHQ1 (SEQ ID NO: 24) | |
| NM_002423.5 | MMP7 | FOR-CCAACAGTTTAGAAGCCAAACTCAAG (SEQ ID NO: 25) | mRNA_1119 |
| NM_002423.5 | MMP7 | REV-GCAACATCTGGCACTCCACATC (SEQ ID NO: 26) | mRNA_1119 |

*Genecards.org is a searchable integrative database providing information on all human genes

Additional exemplary probe sequences, for genes analyzed by Method 2A and/or Method 2B in the Examples provided herein, are set forth in Tables 3 and 4, below:

TABLE 3
Additional Exemplary Probe Sequences

| Gene | Probe Sequence (SEQ ID NO) |
|---|---|
| MMP7 | 5′-CTGGAATGTTAAACTCCCGCGTCATAGA |
| (matrix metallo- | AATAATGC-3′ (SEQ ID NO: 24) |
| peptidase 7) | |
| COX1 | 5′-ATTCGGCGCATGAGCTGGAGTC-3′ |
| (Cyclooxygenase | (SEQ ID NO: 13) |
| 1) | |
| PGR | 5′-CATGTGGCAGATCCCACAGGAGTTTG- |
| (progesterone | 3′ (SEQ ID NO: 4) |
| receptor) | |
| TERT | 5′-TTGAAGAGGGTGCAGCTGCGGGAGCTGT |
| (telomerase | CG-3′ (SEQ ID NO: 27) |
| reverse | |
| transcriptase) | |
| P53 | 5′-CCCCTGCACCAGCAGCTCCTACACCGGC |
| (tumor suppressor | GGCCCCTGCACCAGCCCCCTC-3′ |
| protein p53) | (SEQ ID NO: 50) |

TABLE 4
Additional Exemplary Probe Sequences

| Genecards.org | GENE | PROBES | | (CNN)X |
|---|---|---|---|---|
| NM_005120 | MED12 | FAM- | mRNA_ | (CNN)13 |
| | | CAGCAGCAGCAACAGCAACAGCAGCAGC | 6925 | |
| | | AGCAGCAGC-BHQ1 (SEQ ID NO: 29) | | |
| NM_000635.4 | RFX2 | FAM -GTAACAGTCTCCACGGCTACAGTCTC- | mRNA_ | (CNN)4 |
| | | BHQ1 (SEQ ID NO: 30) | 3959 | |
| NM_001193376.3 | TERT | FAM- | mRNA_ | (CNN)10 |
| | | TTGAAGAGGGTGCAGCTGCGGGAGCTGTC | 3850 | |
| | | G-BHQ1 (SEQ ID NO: 27) | | |
| NM_198829.1 | P21 | FAM -ACAGCGGCCCCGGCACCCAG-BHQ1 | mRNA_ | (CNN)6 |
| | | (SEQ ID NO: 31) | 2413 | |
| NM_001256045.1 | PI3KB | FAM-CATGGGGCAGATGCTGGAGCAGT- | mRNA_ | (CNN)7 |
| | | BHQ1 (SEQ ID NO: 32) | 4797 | |
| NM_001113491 | SEPT9 | FAM -CCCTTGGTCAGCCTCCTCCC-BHQ1 | mRNA_ | (CNN)4 |
| | | (SEQ ID NO: 33) | 3244 | |

Bolded C's or G's indicate the start of a (CNN) or (GNN) sequence. The primers and probes were stored as 20 μM stock solutions in TE buffer (10 mM Tris, 0.5 mM EDTA) at −80° C.

### Example 2

### Relative Abundance of Genes in the Cell-Free DNA of Cancer Patients Compared to Healthy Subjects as Indicative of the Presence or Absence of Cancer

The relative abundance of 6 genes in cancer patients, compared to healthy subjects, was analyzed in a total of 32 blood samples from 32 women donors: 24 from breast cancer patients and 8 from healthy subjects. The women ranged in age from 29 years to 79 years.

(1) Isolation of Plasma from the Blood Samples

Plasma was obtained from the blood samples as follows:
1. 10 mL of blood was collected from each donor in PAXgene Blood ccfDNA Tubes (PreAnalytiX GmbH).
2. The blood samples were transported for processing within 1 day.
3. The manufacturer's protocol was followed for isolating plasma from the blood samples: 2 centrifugations for 15 minutes at 1900 G.
4. After the first centrifugation of the PAXgene tubes, for each blood sample, 4 aliquots of 1 mL each of plasma were sequentially collected, starting from the top, and placed in 4 Eppendorf tubes (numbered 1-4).
5. After the second centrifugation of the 4 Eppendorf tubes, for each plasma sample in each Eppendorf tube, 0.2 mL of plasma was sequentially collected, starting from the top, and placed in 4 Eppendorf tubes. Thus, for each donor, 16 tubes with 0.2 mL of plasma in each were obtained.
6. To each tube containing 0.2 mL of plasma was added an equal volume of preserving agent G-75 (80% glycerol, 0.1 M Tris, pH 7.5). The plasma tubes were stored by freezing at −80° C.

### (2) Analyzing the Plasma Samples for the Relative Abundance of the Following 6 Genes: ESR1, HER2, ARFIP1, COX1, PLIN1 and PGR

The relative abundance of 6 genes: ESR1, PGR, HER2, ARFIP1, COX1 and PLIN1, was measured in each of the donor plasma samples as follows:
1. For each of the 32 donors, a 200 μl plasma sample (prepared and stored as described above) was thawed. Each plasma sample was processed and analyzed in subsequent steps as described below.
2. 100 μl preserving agent G-75 (80% glycerol, Tris, pH 7.5) was added to the plasma sample and mixed to homogeneity by pipetting 10 times.
3. To each well of an 8 well strip, 12 μl lysis solution #1 (DNAzol, MRC, Catalog No. DN131) was added. The pH of the solution was adjusted to 7.5 by adding 4 μl of 1M HEPES.
4. To each well of the 8 well strip, 8 μl of the plasma sample from step 2, was added and mixed to homogeneity with the solution #1 by pipetting 5 times.
5. The 8 well strip was incubated for 10 minutes at 22° C., in a thermo shaker.
6. To each well of the 8 well strip, 8 μl proteinase K (10 mg/mL) was added and vortexed gently for 10 seconds.
7. The 8 well strip was subjected to Pre-PCR under the following conditions:

| | | | | # of |
|---|---|---|---|---|
| Stage | Temperature | Time | | cycles |
| 1 | 55° C. | 30 | min | 1 |
| 2 | 95° C. | 3 | min | 3 |
| | 60° C. | 30 | sec | |
| 3 | 95° C. | 2 | min | 1 |

8. During pre-PCR, the following mix was prepared: all solutions were cooled down to 4° C. on ice:

| Solution | Volume |
|---|---|
| H₂O | 680 μl |
| 25 mM MgCl₂ | 82 μl |
| Enzyme Mix | 680 μl |

*Manufactured by Syntol and contains SynTaq DNA-polymerase with inhibiting antibodies 2 u/w in buffer Tris-HCl, pH 8.8, 0.1% Tween-20 and with each of the dNTPs (A, G, C, T) at 250 μM and 2.5 mM MgCl₂.

9. 180 μl of the mix from step 8, was added to each well of the 8 well strip and mixed by pipetting 8 times. The mixing was done rapidly, to avoid renaturation.
10. Using an 8-channel pipette, 47 μl of the mix from each well of the strip was added to a 96 well plate that was held at 4° C. (8 plasma samples in rows A-H; each analyzed in duplicate with the 6 probe-primer mixes added to the 12 columns).
11. To each well of the plate that contained 47 μl of the mix from step 10, was added 3 μl of a solution in TE (Tris-EDTA) buffer containing 0.3 μM forward primer, 0.7 μM reverse primer and 0.4 μM FAM and BHQ1 labelled probe (primer and probe sequences are set forth in Example 1), depending on the gene to be analyzed, as follows:

ESR1:
(SEQ ID NO: 1)
5′FAM-CTGAACCGTCCGCAGCTCAAGATCC-3′BHQ1 (Probe G1)
PGR:
(SEQ ID NO: 4)
5′FAM-CATGTGGCAGATCCCACAGGAGTTTG-3′BHQ1 (Probe G2)
HER2:
(SEQ ID NO: 7)
5′FAM-CCCAGCCCTGGTCACCTACAACAC-3′BHQ1 (Probe G3)
ARFIP1:
(SEQ ID NO: 10)
5′FAM-GTCGCCGGTCCGACTGTCCTCT-3′BHQ1 (Probe G4)
COX1:
(SEQ ID NO: 13)
5′FAM-ATTCGGCGCATGAGCTGGAGTC-3′BHQ1 (Probe G5)
PLIN1:
(SEQ ID NO: 16)
5′FAM-TGTCCTTGGCGCTAGACACCA-3′BHQ1 (Probe G6)

12. The plate was covered with UV-transparent film and put into a real-time PCR (real-time PCR) thermocycler with the following program:

| | | | |
|---|---|---|---|
| Stage | Temperature | Time | # of cycles |

| | | | | | |
|---|---|---|---|---|---|
| 1 | 95° | C. | 5 | min | 1 |
| 2 | 95° | C. | 15 | sec | 45 |
| | 62° | C. | 45 | sec | |
| 3 | 4° | C. | 2 | min | 1 |

13. A scoring function (positive or negative for cancer) was obtained based on Ct (cycle threshold or threshold cycle; the number of cycles to detect a signal) values. Ct values for each reaction were calculated as the first cycle where exponential growth begins, using the algorithm provided herein. An overview of the steps of the algorithm can be described as follows:
   a. Detect baseline angle and adjust the whole curve so the baseline (average between cycles 10 and 20) is flat.
   b. Analyze the change in fluorescence signal (ΔF) recorded by the real-time-PCR thermocycler, starting from the last cycle at the end of the reaction (cycle 46) and moving backwards until you reach the cycle where the value of ΔF changes from a positive value to zero or a negative value. This cycle number is considered to be the Ct value.

For all six probes (G1-G6), Ct values for the cancer samples averaged 34 or less, while Ct values for the healthy samples averaged 35 or greater. A subject was diagnosed as “positive” for cancer (1) if the Ct value of at least two of the six reactions was 34 or lower. A subject was diagnosed as “negative” for cancer, or healthy (0) if the Ct value of zero or one of the six reactions was 34 or lower. In subsequent analyses, probes G4 and G5 were excluded from the scoring as they did not substantially impact sensitivity or specificity of the assay.
14. Endpoint analysis can be added as a second parameter to the scoring function (besides Ct value). The measured endpoints are the amount of fluorescence (Biorads PFU) measured at the endpoint of each real-time-PCR reaction, i.e., cycle 46.
15. A combination of the analyses in steps 13 and 14 was used to diagnose a subject as cancer positive (1) or cancer negative (0). Based on this scoring, of the 24 cancer patients, only 1 was falsely diagnosed as negative for cancer. Of the 8 healthy subjects, all were correctly diagnosed as negative for cancer. Therefore, the Sensitivity of the assay (ability to correctly identify patients having cancer) was determined to be 95.8% ( 23/24 cancer patients correctly identified) and the Specificity of the assay (ability to correctly identify subjects that do not have cancer) was determined to be 100% (8/8 healthy subjects correctly identified). If the Ct parameter alone was used for scoring, the Sensitivity of the assay (ability to correctly identify patients having cancer was determined to be 100% ( 24/24 cancer patients correctly identified) and the Specificity of the assay (ability to correctly identify subjects that do not have cancer) was determined to be 62.5% (⅝ healthy subjects correctly identified).

The results demonstrate that the assay, designated Method 1, has a high degree of sensitivity and specificity and could/can replace more onerous/expensive methods, such as mammography (Sensitivity: 85-87%; Specificity: 91-92%), and/or help confirm/guide the proper course of treatment based on combining these assay results with those obtained by one or more imaging methods.

(3) Sensitivity of the Method 1 Assay is Greater when the DNA is not Extracted from the Body Fluid

In body fluids such as blood plasma, not all genes and not all fragments of DNA are represented in equal amount. For example, some genes are amplified in tumor cells and the plasma is relatively enriched in fragments from these genes. In addition, DNA fragments can be differentially preserved in plasma. For example, DNA fragments enriched in AT nucleotide content decompose faster in the presence of nucleases than DNA fragments enriched in GC nucleotide content. In the process of extraction, 10 to 40 percent of the DNA originally present in the plasma can be lost. For example, when using DNA extraction kits, GC-rich fragments are more firmly bound on silicone columns and are more difficult to elute off the columns. When precipitating DNA with alcohols, some of the shorter fragments can be lost. In general, extraction of DNA from a body fluid for diagnostic assays can compromise the accuracy of the assay by compromising the integrity of the original composition.

As shown in this Example (see above), Method 1 analyzes DNA in a body fluid using real-time-PCR and an algorithm to obtain a Ct value, without extracting the DNA from the body fluid (proteinase K treatment of the body fluid sample without DNA extraction). This approach was compared against performing Method 1 on DNA that had been extracted using commercially available kits: one from Qiagen (DNeasy Blood & Tissue Kit, Catalog No. 69504) and the other from Apostle (MiniMax™ High Efficiency Cell-Free DNA Isolation Kit).

DNA from the plasma of two healthy subjects (S101 and S107) and two breast cancer patients (S69 and S113) was processed in the following three ways: (1) Extracted using the Qiagen kit; (2) Extracted using the Apostle kit; and (3) Not extracted, as described in this Example. Samples (1), (2) and (3) were subjected to real-time-PCR as described in this Example, and the average of Ct values obtained using four primers/probes (ESR1, HER2, EGFR (with probe 5′FAM-CACCACGTACCAGATGGATGTGAACC-3′BHQ1 (SEQ ID NO:19)) and MMP7; see primer and probe sequences in Table 2 in Example 1) were measured. The results are shown in Table 5, below:

TABLE 5
Average Ct Values

| | Ct of Healthy | | Ct of Breast Cancer | | | |
|---|---|---|---|---|---|---|
| | samples | | samples | | Average \|ΔCt\| | |
| Kit | S101 | S107 | S69 | S113 | [Δmin; Δmax] | |
| Qiagen | 34 | 34 | 33 | 35 | 0 | [−1; 1] |
| Apostol | 29 | 29 | 29 | 29 | 0 | [0; 0] |
| Unextracted | 36 | 36 | 34 | 33 | 2.5 | [2; 3] |

The results demonstrate that only the unextracted sample provided a diagnosable difference in the Ct values obtained for the breast cancer samples (Ct≤34) compared to the Ct values obtained for the healthy samples (Ct≥35).

### Example 3

Relative Mg²⁺-dependent and Zn²⁺-dependent nuclease activities in the Body Fluids of Cancer Patients Compared to Healthy Subjects as Indicative of the Presence or Absence of Cancer As discussed above in Example 1, the genes selected for analysis according to the methods provided herein contain zinc finger protein binding motifs. In this example, the ratio of Zn²⁺-dependent nuclease activity to Mg²⁺-dependent nuclease activity (coefficient K-A3) in the plasma of breast cancer patients, compared to healthy subjects, was analyzed according to Method 2A. K-A3 ratios were measured in the plasma of patients having 4 subtypes of breast cancer: Luminal A, Luminal B, HER2-enriched breast cancer and Triple Negative, and compared to the K-A3 ratios measured in healthy subjects.

Plasma from the cancer patients and healthy subjects (donors) was isolated and processed as described in Example 2. For each donor and for each probe, 4 plasma samples were analyzed: 2 in the presence of only magnesium (Mg²⁺-dependent nuclease activity) and 2 in the presence of magnesium and zinc (Zn²⁺-dependent nuclease activity). Plasma samples were thawed, and the reactions were performed as follows:

| | | Per | Per 5 | |
|---|---|---|---|---|
| Step | Reagent | well | Wells | Procedure |
| 1. | *Solution #1 | 4 μl | 20 μl | Added to 8-well strip. To wash the surface of the wells |
| | | | | of the strip with Solution #1, the 8-well strip was |
| | | | | vortexed for 2 seconds |
| 2. | Plasma | 5 μl | 25 μl | Before drawing the thawed plasma, to make the sample |
| | | | | more homogeneous, it was pipetted 10 times with a 1 |
| | | | | ml pipette tip, avoiding formation of air bubbles. |
| | | | | Plasma was drawn immediately after pipetting of each |
| | | | | tube, using a 200 μl pipette tip. The 200 μl pipette tip |
| | | | | was washed 5 times with a 25 μl volume of plasma, |
| | | | | then 25 μl of plasma was drawn and transferred to the |
| | | | | strip containing Solution #1 and then plasma and |
| | | | | Solution #1 mixed by pipetting 3 times. Plasma was |
| | | | | collected one-by-one from the 8 wells of the strip. |
| 3. | — | — | — | The strip was closed with a cap and incubated at 22° C. |
| | | | | for 10 minutes. |
| 4. | Water | 4 μl | 20 μl | Water was added using a repeater pipette. |
| 5 | 50 mM | 3.2 μl | 16 μl | MgCl₂ was added using a repeater pipette and pipetted |
| | MgCl₂ | | | 10 times without air bubbles before transferring the |
| | | | | strip on ice. |
| 6. | | | | Using an 8-channel mμltichannel pipette, 17 μl of the |
| | | | | mix from each well in step 5 was transferred to 4 |
| | | | | vertical columns of a 96-well PCR plate (for analyzing |
| | | | | 4 genes, 64 wells of the plate are used to analyze, 4 |
| | | | | genes, 8 samples, repeated in duplicate (4 × 8 × 2); for 6 |
| | | | | genes, amounts are increased proportionally ) that was |
| | | | | placed in a metal holder at a thermostatic temperature |
| | | | | of 5° C. |
| 7. | Water or | 5 μl | 25 μl | Using an 8-channel pipette, 5 μl of water was added to |
| | ZnSO₄ | | | columns 1 and 2 (to measure magnesium-dependent |
| | | | | nuclease activity) and 5 μl of 45 mM ZnSO₄ was added |
| | | | | to columns 3 and 4 (to measure zinc-dependent |
| | | | | nuclease activity). |
| 8. | Probe | 9 μl | | ** FAM-labeled (BHQ1 Quencher) Probe solution (9 |
| | | | | μl; 20 uM concentration), which was stored at −80° C., |
| | | | | was thawed on ice (5° C.). 18 μl of water was added and |
| | | | | mixed to homogeneity by pipetting 15 times using an |
| | | | | 8-channel pipette. |
| 9. | Diluted | 3 μl | | Keeping the diluted probe solution of step 8 and the |
| | Probe | | | PCR plate at 5°C, 3 μl of the diluted probe solution |
| | Solution | | | was added to each well of the PCR plate using an 8- |
| | | | | channel pipette with 10 μl capacity. |
| 10. | | | | The PCR plate was covered with a UV-protective |
| | | | | sticky film and centrifuged for 10 seconds before |
| | | | | returning it back on ice (5° C.). |
| 11. | | | | The PCR plate was transferred to a real-time PCR |
| | | | | machine when the lid temperature reached 80° C. |
| | | | | Incubation was performed for 35 min at 37° C. |
| | | | | Fluorescence was recorded in real-time to |
| | | | | QuantStudio5. Every 5 minutes, the reading of FAM |
| | | | | fluorescence was performed. For analysis, the end- |
| | | | | point measurements were used. |

*See Example 2 for description of Solution #1
** The following probes were used in the analysis:
ESR1: 5′FAM-CTGAACCGTCCGCAGCTCAAGATCC-3′BHQ1 (SEQ ID NO: 1)
HER2: 5′FAM-CCCAGCCCTGGTCACCTACAACAC-3′BHQ1 (SEQ ID NO: 7)
EGFR: 5′FAM-CACCACGTACCAGATGGATGTGAACC-3′BHQ1 (SEQ ID NO: 19)
MMP7: 5′FAM-CTGGAATGTTAAACTCCCGCGTCATAGAAATAATGC-3′BHQ1 (SEQ ID NO: 24)
COX1: 5′FAM-ATTCGGCGCATGAGCTGGAGTC-3′BHQ1 (SEQ ID NO: 13)
PGR: 5′FAM-CATGTGGCAGATCCCACAGGAGTTTG-3′BHQ1 (SEQ ID NO: 4)
ESR1 Probes containing 1 (ESR1-M1), 2 (ESR1-M2), 3 (ESR1-M3) or 4
(ESR1-M4) mutations in the zinc finger binding motif region:
ESR-M1: 5′FAM- CTGAACCGTACGCAGCTCAAGATCC-3′ BHQ1 (SEQ ID NO: 37)
ESR-M2: 5′FAM- CTGAACCGTACGTAGCTCAAGATCC-3′ BHQ1 (SEQ ID NO: 38)
ESR-M3: 5′FAM- CTGAACCGTACGTAGATCAAGATCC-3′ BHQ1 (SEQ ID NO: 39)
ESR-M4: 5′FAM- CTGAACTGTACGTAGATCAAGATCC-3′ BHQ1 (SEQ ID NO: 40)

The results showed that, in general, the ratio of zinc-dependent nuclease activity to magnesium-dependent nuclease activity is higher in cancer samples compared to the healthy samples, as shown in Table 6, below:

TABLE 6

| Gene | Probe | *Donor | K-A3 |
|---|---|---|---|
| ESR1 | 5′FAM-CTGAACCGTCCGCAGCTCAAGATCC-3′BHQ1 | TNBC | 3.87 |
| | (SEQ ID NO: 1) | | |
| ESR1 | 5′FAM-CTGAACCGTCCGCAGCTCAAGATCC-3′BHQ1 | Lum-A | 1.45 |
| | (SEQ ID NO: 1) | | |
| ESR1 | 5′FAM-CTGAACCGTCCGCAGCTCAAGATCC-3′BHQ1 | Lum-B | 2.05 |
| | (SEQ ID NO: 1) | | |
| ESR1 | 5′FAM-CTGAACCGTCCGCAGCTCAAGATCC-3′BHQ1 | Healthy | 1.21 |
| | (SEQ ID NO: 1) | | |
| HER2 | 5′FAM-CCCAGCCCTGGTCACCTACAACAC-3′BHQ1 | TNBC | 1.97 |
| | (SEQ ID NO: 7) | | |
| HER2 | 5′FAM-CCCAGCCCTGGTCACCTACAACAC-3′BHQ1 | Lum-A | 1.31 |
| | (SEQ ID NO: 7) | | |
| HER2 | 5′FAM-CCCAGCCCTGGTCACCTACAACAC-3′BHQ1 | Lum-B | 2.91 |
| | (SEQ ID NO: 7) | | |
| HER2 | 5′FAM-CCCAGCCCTGGTCACCTACAACAC-3′BHQ1 | Healthy | 0.56 |
| | (SEQ ID NO: 7) | | |
| EGFR | 5′FAM-CACCACGTACCAGATGGATGTGAACC-3′BHQ1 | TNBC | 1.63 |
| | (SEQ ID NO: 19) | | |
| EGFR | 5′FAM-CACCACGTACCAGATGGATGTGAACC-3′BHQ1 | Lum-A | 1.57 |
| | (SEQ ID NO: 19) | | |
| EGFR | 5′FAM-CACCACGTACCAGATGGATGTGAACC-3′BHQ1 | Lum-B | 3.23 |
| | (SEQ ID NO: 19) | | |
| EGFR | 5′FAM-CACCACGTACCAGATGGATGTGAACC-3′BHQ1 | Healthy | 1.05 |
| | (SEQ ID NO: 19) | | |
| MMP7 | 5′FAM- | TNBC | 1.56 |
| | CTGGAATGTTAAACTCCCGCGTCATAGAAATAATGC- | | |
| | 3′BHQ1 (SEQ ID NO: 24) | | |
| MMP7 | 5′FAM- | Lum-A | 1.49 |
| | CTGGAATGTTAAACTCCCGCGTCATAGAAATAATGC- | | |
| | 3′BHQ1 (SEQ ID NO: 24) | | |
| MMP7 | 5′FAM- | Lum-B | 1.14 |
| | CTGGAATGTTAAACTCCCGCGTCATAGAAATAATGC- | | |
| | 3′BHQ1 (SEQ ID NO: 24) | | |
| MMP7 | 5′FAM- | Healthy | 0.93 |
| | CTGGAATGTTAAACTCCCGCGTCATAGAAATAATGC- | | |
| | 3′BHQ1 (SEQ ID NO: 24) | | |
| COX1 | 5′FAM-ATTCGGCGCATGAGCTGGAGTC-3′BHQ1 (SEQ | TNBC | 2.60 |
| | ID NO: 13) | | |
| COX1 | 5′FAM-ATTCGGCGCATGAGCTGGAGTC-3′BHQ1 (SEQ | Lum-A | 1.71 |
| | ID NO: 13) | | |
| COX1 | 5′FAM-ATTCGGCGCATGAGCTGGAGTC-3′BHQ1 (SEQ | Lum-B | 3.34 |
| | ID NO: 13) | | |
| COX1 | 5′FAM-ATTCGGCGCATGAGCTGGAGTC-3′BHQ1 (SEQ | Healthy | 2.27 |
| | ID NO: 13) | | |
| PGR | 5′FAM-CATGTGGCAGATCCCACAGGAGTTTG-3′BHQ1 | TNBC | 3.77 |
| | (SEQ ID NO: 4) | | |
| PGR | 5′FAM-CATGTGGCAGATCCCACAGGAGTTTG-3′BHQ1 | Lum-A | 2.16 |
| | (SEQ ID NO: 4) | | |
| PGR | 5′FAM-CATGTGGCAGATCCCACAGGAGTTTG-3′BHQ1 | Lum-B | 2.72 |
| | (SEQ ID NO: 4) | | |
| PGR | 5′FAM-CATGTGGCAGATCCCACAGGAGTTTG-3′BHQ1 | Healthy | 1.75 |
| | (SEQ ID NO: 4) | | |

*TNBC: triple negative breast cancer; Lum-A: luminal-A breast cancer; Lum-B: luminal-B breast cancer; Healthy: no cancer.

An analysis of 11 replicates of each of the above 6 probes in a healthy sample resulted in K-A3 ratios of between 0.8-1.16. On the other hand, an analysis of 14 replicates of each of the above 6 probes in a Luminal A breast cancer sample resulted in higher K-A3 ratios of between 1.55-2.05.

The presence of one or more mutations in the probe sequence (ESR-M, ESR1-M2, ESR1-M3, ESR1-M4), which could interfere with zinc finger protein binding, was found to disrupt the ability to distinguish between cancer patients and healthy subjects due to a significant decrease in the K-A3 ratio, as shown in Table 7, below:

TABLE 7

| | | | K- |
|---|---|---|---|
| Gene | Probe | *Donor | A3 |
| ESR1 | 5′FAM-CTGAACCGTCCGCAGCTCAAGATCC-3′BHQ1 (SEQ ID | Lum-B | 1.71 |
| | NO: 1) | | |
| ESR1- | 5′FAM- CTGAACCGTACGCAGCTCAAGATCC-3′BHQ1 | Lum-B | 0.87 |
| M1 | (SEQ ID NO: 37) | | |
| ESR1- | 5′FAM- CTGAACCGTACGTAGCTCAAGATCC-3′BHQ1 (SEQ ID | Lum-B | 0.91 |
| M2 | NO: 38) | | |
| ESR1- | 5′FAM- CTGAACCGTACGTAGATCAAGATCC-3′BHQ1 (SEQ ID | Lum-B | 0.83 |
| M3 | NO: 39) | | |
| ESR1- | 5′FAM- CTGAACTGTACGTAGATCAAGATCC-3′BHQ1 (SEQ ID | Lum-B | 0.87 |
| M4 | NO: 40) | | |

*TNBC: triple negative breast cancer; Lum-A: luminal-A breast cancer; Lum-B: luminal-B breast cancer; Healthy: no cancer.

Plasma samples from a cohort of 102 donors, including 31 Healthy subjects, 23 Breast Cancer (BC) patients (unspecified type), 13 HER2-enriched breast cancer patients and 35 triple negative breast cancer patients (TNBC) were analyzed using the method described above and probes for the following genes: ESR1, HER2, EGFR, MMP7. Box plot analysis showed that the K-A3 values for the cancer patients are significantly higher compared to those of the healthy subjects. Based on the Box Plot, the Specificity and Sensitivity is as shown in Table 8, below:

TABLE 8
Box plot analysis of K-A3 values

| | Breast | | |
|---|---|---|---|
| Test | cancer | Healthy | Total |
| Positive | 67 | 6 | 73 |
| Negative | 4 | 25 | 29 |
| Total | 71 | 31 | 102 |

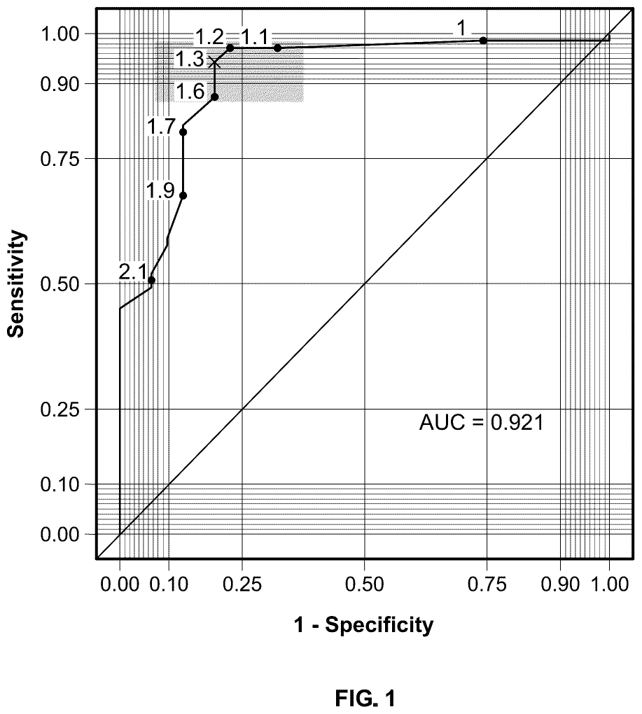

As shown in the ROC curve in FIG. 1, at a K-A3 cutoff of 1.3 (average of K-A3 values obtained from the tested probes), the Sensitivity was found to be 94% (95% CI [86%, 98%]), the Specificity was found to be 81% (95% CI [63%, 93%]), and the ROC curve had AUC=0.921 (see FIG. 1).

The results provided in this Example demonstrate that the ratio of zinc-dependent nuclease activity to magnesium-dependent nuclease activity in a body fluid can be used to differentiate between samples from a cancer patient and samples from a healthy subject.

### Example 4

Differences in Mg²⁺-Dependent Nuclease Activity Between the Body Fluids of Cancer Patients Compared to Healthy Subjects as a Function of Mg²⁺ Concentration

This example demonstrates that the differences between the Mg²⁺-dependent nuclease activities seen in breast cancer patients compared to normal subjects is dependent on the number of zinc finger motifs in the target used to assess nuclease activity, and on the Mg²⁺ or Zn²⁺ concentration.

Plasma samples from a healthy subject (AD4) and a patient with triple-negative breast cancer (AC9) were analyzed as described in Example 3. For studying the effects of Mg²⁺-dependent nuclease activity, no Zn²⁺ was added to any of the samples. The following two probes were used to assay Mg²⁺-dependent nuclease activity in the healthy subject (AD4) and the breast cancer patient (AC9), as described in Example 3 above:

For Substrate PGR (progesterone receptor), the PGR
probe:
(SEQ ID NO: 4)
FAM-5′-CATGTGGCAGATCCCACAGGAGTTTG-3′- BHQ1 (3 zinc
finger binding motifs).
For Substrate ESR1 (estrogen receptor 1) the ESR1
probe:
(SEQ ID NO: 1)
FAM -5′-CTGAACCGTCCGCAGCTCAAGATCC-3′ -BHQ1 (4 zinc
finger binding motifs).

The end-point measurements of FAM fluorescence were used to determine Mg²⁺-dependent nuclease activity. Mg²⁺ concentrations of 4, 6, 8, 10, 12, 14, 16 and 18 mM were tested. The results demonstrate that for the PGR probe (3 zinc finger binding motifs), no difference in Mg²⁺-dependent nuclease activity was seen in the healthy subject compared to the cancer patient. For the ESR1 probe (4 zinc finger binding sites (motifs)), however, at a concentration of 6.0 mM Mg²⁺, the nuclease activity in the plasma of the AC9-breast cancer patient was found to be 2 times higher than that of the AD4-healthy subject. Additional studies in the range of 6-7 mM Mg²⁺ showed that the maximum difference between the plasma of a healthy subject and the plasma of a patient with breast cancer is observed at a concentration of 6.3 mM Mg in the reaction mixture.

### Example 5

Effect of Glycerol on the Measurement of Mg²⁺-Dependent and Zn²⁺-Dependent Nuclease Activities in the Body Fluids of Cancer Patients and Healthy Subjects

The effect of the conserving agent, glycerol, on the coefficient of variation in K-A3 (ratio of Zn²⁺-dependent nuclease activity to Mg²⁺-dependent nuclease activity) between successive plasma draws from the same donor was studied. The donors were as follows:
R11: Invasive Ductal Carcinoma breast cancer
R12: Luminal A breast cancer
R13: Fibroadenoma (non-cancerous breast tumor)
AA40: Healthy subject

For each of the above donors, K-A3 ratios were determined in successive draws of freshly isolated plasma, or plasma that was preserved in 40% (v/v) glycerol, stored at −80° C. and thawed before analysis (see Example 3 for the determination of K-A3 ratios). The results are shown in Table 9, below:

TABLE 9
Variation in K-A3 Values between Plasma
Samples from the same Donor

| | | | | Coefficient |
|---|---|---|---|---|
| | | K-A3 | K-A3 | of Variation |
| Sample Type | Sample # | Draw 1 | Draw 2 | between Draws |
| Plasma | R11 | 1.55 | 1.31 | 12% |
| Plasma | R12 | 1.56 | 1.03 | 29% |
| Plasma | R13 | 1.48 | 1.01 | 27% |
| Plasma | AA40 | 1.64 | 1.21 | 21% |
| Plasma + Glycerol | R11 | 2.41 | 2.12 | 9% |
| Plasma + Glycerol | R12 | 1.93 | 1.95 | 1% |
| Plasma + Glycerol | R13 | 2.30 | 2.22 | 2% |
| Plasma + Glycerol | AA40 | 2.58 | 2.48 | 3% |

The results demonstrate that the coefficient of variation between draws is significantly lower in the plasma samples that have been preserved in glycerol. This is explained by the significant disappearance of protein aggregates when glycerol is added to the plasma samples.

### Example 6

Effect of NP40, DTT, Ca²⁺, Temperature and Proteinase K on the Measurement of Mg²⁺-dependent and Zn²⁺-dependent nuclease activities in the Body Fluids of Cancer Patients and Healthy Subjects

In this experiment, using an ESR1 probe, the healthy subjects, breast cancer patients and patients having non-cancerous conditions of the breast (collectively referred to as donors; details of each provided below) were analyzed for the effect of NP40, DTT, Ca²⁺, temperature and proteinase K on the measurement of Mg²⁺-dependent and Zn²⁺-dependent nuclease activities in cancer patients and healthy subjects. The concentrations of the reagents are as follows: 2 mM DTT, 0.2% NP40, 13 mM CaCl₂, 6.5 mM MgCl₂, 9 mM ZnSO₄.

(1) Differential Effect of NP40 and DTT on Plasma Mg²⁺-Dependent and Zn²⁺-Dependent Nuclease Activities

Plasma samples from the donors were analyzed for Mg²⁺-dependent and Zn²⁺-dependent nuclease activities, as discussed in Example 3 above, using the ESR1 probe. The values of Mg²⁺-dependent and Zn²⁺-dependent nuclease activities in the absence of additives or heat treatment were set as 1; all activity values in the presence of additives and/or heat treatment were normalized relative to activity values in the absence of additives and/or heat treatment. The results (mean values of all the samples tested as depicted in the Table 11, shown in part (2) below), are shown in Table 10, below:

TABLE 10

| | | | | | Prot K 15 | Prot K 15 | 20 min |
|---|---|---|---|---|---|---|---|
| | No | | | | min @ | min @ | @ |
| Activity | Additive | NP40 | DTT | Ca²⁺ | 37° C. | 56° C. | 56° C. |
| Mg²⁺- | 1 | 0.9 | 1.9 | 0.4 | 0.2 | 0.63 | 0.12 |
| dependent | | | | | | | |
| Zn²⁺- | 1 | 2.3 | 0.4 | 0.9 | 1.3 | 0.05 | 0.84 |
| dependent | | | | | | | |

As shown in Table 10, the presence of the NP40 detergent increases Zn²⁺ -dependent nuclease activity by 2.3-fold. DTT, on the other hand, increases Mg²⁺-dependent nuclease activity by 1.9-fold and inhibits Zn²⁺-dependent nuclease activity. Incubation for 15 minutes at 37° C. with proteinase K increases Zn²⁺ -dependent nuclease activity and inhibits Mg²⁺-dependent nuclease activity by 5-fold. Mg²⁺-dependent nuclease activity was greatly decreased, by almost 10-fold, upon heat treatment for 20 minutes at 56° C.; Zn²⁺-dependent nuclease activity, on the other hand, decreased by a much smaller amount. Mg²⁺-dependent nuclease activity was greatly decreased, by almost 10-fold, upon heat treatment for 20 minutes at 56° C.; Zn²⁺-dependent nuclease activity, on the other hand, decreased by a much smaller amount. Similarly, treatment with Ca²⁺-dependent nuclease activity decreased Mg²⁺-dependent nuclease activity by a greater amount than the decrease in Zn²⁺-dependent nuclease activity. The results demonstrate that two distinct types of nuclease activities, Mg²⁺-dependent nuclease activity and Zn²⁺-dependent nuclease activity, are present in body fluids such as plasma.

(2) Differential Effect of NP40 and DTT on Plasma Mg²⁺-Dependent and Zn²⁺-Dependent Nuclease Activities in Normal Subjects and Cancer Patients

Plasma from breast cancer patients, subjects with non-cancerous breast conditions and healthy subjects (donors) was isolated and processed as described in Example 2. Abbreviations in Table 11, below, are as follows:
NOR or “Healthy”: No breast-related condition (cancerous or non-cancerous)
LUM-A: Luminal A breast cancer
LUM-B: Luminal B breast cancer
HER2: HER2-enriched breast cancer
TRIPLE: Triple negative breast cancer
Fib-A: Fibroadenoma (non-cancerous)
FKD: Fibrocystic disease (non-cancerous)

The plasma samples were analyzed for Mg²⁺-dependent and Zn²⁺-dependent nuclease activities, as discussed in Example 3 above and using the ESR1 probe, with no additive (NO) in the presence of NP40 (NP40+) or DTT (DTT+). The values of Mg²⁺-dependent and Zn²⁺-dependent nuclease activities for the various samples are shown in Table 11, below:

TABLE 11
Mg²⁺ - dependent and Zn²⁺ - dependent nuclease activities

| | | | | | Mg²⁺Zn²⁺/ |
|---|---|---|---|---|---|
| Sample# | Diagnosis | Additives | Mg²⁺ | Mg²⁺Zn²⁺ | Mg²⁺ |
| S103 | Healthy | NO | 590 | 508 | 0.86 |
| S104 | Healthy | NO | 498 | 368 | 0.74 |
| S105 | Healthy | NO | 272 | 397 | 0.86 |
| S124 | Lum-B | NO | 218 | 336 | 1.54 |
| S122 | HER2 | NO | 543 | 818 | 1.51 |
| S110 | TRIPLE | NO | 573 | 707 | 2.6 |
| S123 | LUM-A | NO | 119 | 449 | 4.9 |
| S123 | LUM-A | NO | 129 | 462 | 3.6 |
| R5 | Fib-A | NO | 598 | 566 | 0.95 |
| R5 | Fib-A | NO | 1165 | 953 | 0.82 |
| R10 | FKD | NO | 237 | 240 | 1.01 |
| R10 | FKD | NO | 473 | 360 | 0.76 |
| S75 | Fib-A | NO | 621 | 428 | 0.69 |
| S75 | Fib-A | NO | 535 | 515 | 0.96 |
| S105 | NOR | NP40+ | 415 | 1792 | 4.3 |
| S124 | LUM-B | NP40+ | 430 | 1465 | 3.4 |
| S122 | HER2 | NP40+ | 855 | 1477 | 1.7 |
| S110 | TRIPLE | NP40+ | 522 | 1667 | 3.2 |
| S123 | LUM-A | NP40+ | 374 | 1304 | 3.5 |
| S123 | LUM-A | NP40+ | 202 | 1115 | 5.5 |
| R5 | Fib-A | NP40+ | 230 | 1142 | 5 |
| R5 | Fib-A | NP40+ | 260 | 706 | 2.7 |
| S103 | NOR | DTT+ | 645 | 368 | 0.57 |
| S105 | NOR | DTT+ | 528 | 323 | 0.61 |
| S124 | LUM-B | DTT+ | 723 | 264 | 0.37 |
| S122 | HER2 | DTT+ | 616 | 147 | 0.24 |
| S110 | TRIPLE | DTT+ | 1070 | 196 | 0.18 |
| S123 | LUM-A | DTT+ | 735 | 225 | 0.31 |
| S123 | LUM-A | DTT+ | 682 | 228 | 0.33 |
| R5 | Fib-A | DTT+ | 1237 | 318 | 0.26 |
| R5 | Fib-A | DTT+ | 1145 | 248 | 0.22 |
| R10 | FKD | DTT+ | 354 | 235 | 0.66 |
| S75 | Fib-A | DTT+ | 1618 | 378 | 0.23 |

When no additive is present, K-A3 ratios were significantly higher in the samples from the breast cancer patients, with values between 1.51 to 4.9. For subjects that were healthy or had a non-cancerous breast condition, K-A3 ratios were lower, between 0.69 to 1.01. In the presence of DTT, K-A3 values were found to decrease for all the samples tested. The decrease, however, was significantly greater in the samples from the cancer patients and the donor with fibroadenoma. As a result, in the presence of DTT, K-A3 ratios in cancer patients are lower than the K-A3 ratios measured in healthy subjects.

The addition of NP40 at a concentration of 0.1% was found to increase plasma Zn²⁺-dependent nuclease activity in the donors (patients with all forms of breast cancer, fibroadenoma and healthy subject). The Mg²⁺-dependent nuclease activity, on the other hand, was inhibited in plasma from the healthy subject and the subject with fibroadenoma and increased in plasma from cancer patients, e.g., by 2.3-fold, 2.0-fold and 1.6-fold in Luminal A, Luminal B and HER2-enriched breast cancers, respectively.

(3) Differential Effect of Temperature on Plasma Mg²⁺-Dependent and Zn²⁺-Dependent Nuclease Activities

Plasma samples from patient S69 (HER2-enriched breast cancer) were analyzed for Mg²⁺-dependent (Mg) and Zn²⁺-dependent (Zn²⁺, i.e., presence of Mg²⁺ and Zn²⁺) nuclease activities, as discussed in Example 3 above, using the ESR1 probe, by incubation for various times at 56° C. as shown in Table 12, below:

TABLE 12
Mg²⁺-dependent and Zn²⁺-dependent nuclease activities

| Time | Mg | Mg | Mg | Zn+ | Zn+ | Zn+ | K-A3 |
|---|---|---|---|---|---|---|---|
| 0 min | 473 | 286 | 379.5 | 146 | 256 | 201 | 0.53 |
| 1 min | 825 | 318 | 571.5 | 172 | 79 | 125.5 | 0.22 |
| 2.5 min | 320 | 133 | 226.5 | 114 | 144 | 129 | 0.57 |
| 5 min | 281 | 88 | 184.5 | 272 | 354 | 313 | 1.70 |
| 10 min | 70 | 87 | 78.5 | 106 | — | 106 | 1.35 |
| 20 min | 57 | 29 | 43 | 168 | 101 | 134.5 | 3.13 |
| 40 min | 82 | 169 | 125.5 | 195 | 528 | 361.5 | 2.88 |

Table 12 shows that incubation at 56° C. leads to a selective drop in Mg²⁺-dependent nuclease activity, which in turn results in a corresponding increase in the K-A3 ratio from 0.53 to 3.13. Mg²⁺-dependent nuclease activity was found to be more sensitive at this temperature than the Zn²⁺-dependent nuclease activity. After 20 minutes of incubation, the Mg²⁺-dependent nuclease activity decreased by 8 times, leading to a 6-fold increase in the K-A3 ratio.

Incubation of plasma samples from healthy subjects and from cancer patients (Luminal B breast cancer, HER2-enriched breast cancer and Triple Negative breast cancer) for 10 minutes at 94° C. or for 30 minutes at 70° C. led to a complete inactivation of both Zn²⁺-dependent and Mg²⁺-dependent nuclease activities. Incubation for 5 minutes at 94° C. led to a selective drop in Zn²⁺-dependent nuclease activity of all the samples tested. The K-A3 ratio dropped by 7-10-fold in all the breast cancer samples (Luminal B breast cancer, HER2-enriched breast cancer and Triple Negative breast cancer) that were tested.

(4) Differential Effect of Proteinase K Treatment on Plasma Mg²⁺-Dependent and Zn²⁺-Dependent Nuclease Activities

Plasma samples from patient S110 (Triple Negative breast cancer) were analyzed for Mg²⁺-dependent (Mg) and Zn²⁺-dependent (Zn²⁺, i.e., presence of Mg²⁺ and Zn²⁺) nuclease activities, as discussed in Example 3 above, using the ESR1 probe, by incubation with 1 mg/mL proteinase K for various times at 56° C. as shown in Table 13, below:

TABLE 13
Mg²⁺-dependent and Zn²⁺-dependent nuclease activities

| Time | Mg | Mg | Mg | Zn+ | Zn+ | Zn+ | K-A3 |
|---|---|---|---|---|---|---|---|
| 0 min | 295 | 245 | 270 | 668 | 702 | 685 | 2.54 |
| 15 min | 150 | 182 | 166 | 36 | 25 | 30.5 | 0.18 |
| 30 min | 162 | 116 | 139 | 27 | 28 | 27.5 | 0.20 |
| 1 hour | 192 | 155 | 173.5 | 65 | 52 | 58.5 | 0.34 |
| 8 hours | 106 | 124 | 115 | 44 | 40 | 42 | 0.37 |

The results demonstrate that Zn²⁺-dependent nuclease activity is significantly more sensitive to treatment with proteinase K. After 15 minutes of incubation at 56° C., the Zn²⁺-dependent nuclease activity had decreased by 22-fold while the Mg²⁺-dependent nuclease activity decreased by 1.6-fold, resulting in a dramatic fall in the K-A3 ratio.

This example demonstrates the distinct effects of additives, temperature and proteinase K on Mg²⁺-dependent and Zn²⁺-dependent nuclease activities. In the methods provided herein, reaction conditions are fine tuned to distinguish cancer patients from healthy subjects and/or subjects with non-cancerous conditions, based on the ratio of Zn²⁺-dependent nuclease activity (i.e., in the presence of Mg²⁺ +Zn²⁺) to Mg²⁺-dependent nuclease activity (i.e., in the presence of Mg²⁺ alone).

### Example 7

Combined Analysis of: (1) Relative Abundance of Certain Genes in a Body Fluid of a Cancer Patient Compared to a Healthy Subject (Method 1) and (2) Ratio of Zn²⁺-Dependent Nuclease Activity to Mg²⁺-Dependent Nuclease Activity in a Body Fluid of a Cancer Patient Compared to a Healthy Subject (Method 2A), for Improved Sensitivity in Detecting the Presence or Absence of Cancer

Plasma samples from 80 donors (44 healthy subjects, 36 breast cancer patients) from women ages 35-70 years old were analyzed for the relative abundance of cell free DNA (cfDNA) corresponding to ESR1, HER2, EGFR and MMP7 genes (Method 1; see Example 2) and relative Mg²⁺-dependent (Mg) and Zn²⁺-dependent (Zn²⁺, i.e., presence of Mg²⁺ and Zn²⁺) nuclease activities (Method 2A; see Example 3), as discussed above. The breast cancer patients were primarily in the early stages (T1 and T2), and their distribution is as follows:
Luminal A breast cancer: 3 Stage T1, 5 Stage T2, 1 Stage T3
Luminal B breast cancer: 2 Stage T1, 6 Stage T2, 2 Stage T4
HER2-enriched breast cancer: 1 Stage T1, 4 Stage T2, 1 Stage Tx (i.e., exact stage unknown)
Triple negative breast cancer: 2 Stage T1, 3 Stage T2
Breast cancer of unspecified type: 1 Stage T1, 2 Stage T2, 3 Stage T4

A comparison of the analyses using Method 1 and Method 2A, and a combination thereof, showed the following:
Sensitivity (ability to correctly identify true positives for cancer):
   Method 1: 75% (58-88%)
   Method 2A: 91% (71-99%)
   Combined results of Method 1 and Method 2A: 100% (85-100%)
Specificity (ability to correctly identify true negatives for cancer):
   Method 1: 80% (65-90%)
   Method 2A: 88% (70-98%)
   Combined results of Method 1 and Method 2A: 85% (65-96%)
The ratio of the likelihood of a positive result (LR+) was the following:
   Method 1: 3.67 (1.97-6.76), indicating a slight to moderate increase in the probability of disease
   Method 2A: 7.88 (2.70-23.03), indicating a moderate to large increase in the probability of disease
   Combined results of Method 1 and Method 2A: 6.50 (2.64-16.01), indicating a moderate to large increase in the probability of disease.
The ratio of the likelihood of a negative result (LR−) was the following:
   Method 1: 0.31 (0.18-0.56), indicating a moderate decrease in the probability of disease
   Method 2A: 0.10 (0.03-0.39), indicating a large decrease in the probability of disease

Combined results of Method 1 and Method 2A: 0.00, indicating a large decrease in the probability of disease. The results indicate that a combination of the two methods, Method 1 and Method 2A, can increase the accuracy of identifying subjects who are positive or negative for cancer.

### Example 8

Relative Mg²⁺-Dependent and Zn²⁺-Dependent Nuclease Activities in the Body Fluids of COVID-19 Patients Compared to Healthy Subjects as Indicative of the Presence or Absence of COVID-19

In this example, the ratio of Zn²⁺-dependent nuclease activity to Mg²⁺-dependent nuclease activity (coefficient K-A3, determined as shown in Example 3) in the plasma of COVID-19 patients, compared to healthy subjects, was analyzed. K-A3 ratios were measured in the plasma of patients having COVID-19, a month between two analyses (measurements) in a patient with mild COVID-19 symptoms, and in healthy subjects. The probes for the SARS-Cov-2 virus are as shown below:

| Position | Probe sequence |
|---|---|
| 3045 | FAM-CCAGATGAGGATGAAGAAGAAGG-BHQ1 |
| | (SEQ ID NO: 34) |
| 3465 | FAM-CATGGAGGAGGTGTTGCAGGAGCCT-BHQ1 |
| | (SEQ ID NO: 35) |
| 19721 | FAM-AAGTTGATGGTGTTGATGTAGAAT-BHQ1 |
| | (SEQ ID NO: 36) |

The results are shown in Table 14, below:

TABLE 14
Mg²⁺-dependent and Zn²⁺-dependent nuclease activities

| | | | Probe 3045 | | | Probe 3465 | | | Probe 19721 | | |
|---|---|---|---|---|---|---|---|---|---|---|---|
| | Patient | | | Mg + | K-A3 * | | Mg + | | | Mg + | |
| # | ID | Diagnosis | Mg | Zn | 100 | Mg | Zn | K-A3 | Mg | Zn | K-A3 |
| 1 | AU-39 | Healthy | 2 714 522 | 46 701 | 1.72 | 2 484 906 | 33 172 | 1.33 | 2 208 971 | 6 621 | 0.30 |
| 2 | DM-40 | COVID-19 | 5 480 115 | 325 632 | 5.94 | 6 135 433 | 395 392 | 6.44 | 4 860 240 | 211 513 | 4.35 |
| 3 | IA-15 | Healthy | 5 868 845 | 25 870 | 0.44 | 7 688 278 | 39 967 | 0.52 | 5 619 339 | −46 854 | −0.83 |
| 4 | OP-43 | COVID-19 | 3 030 433 | 157 516 | 5.20 | 2 284 379 | 227 423 | 9.96 | 3 106 671 | 175 023 | 5.63 |
| 5 | AM-71 | COVID-19 | 7 192 059 | 470 345 | 6.54 | 7 527 795 | 676 609 | 8.99 | 7 150 328 | 494 617 | 6.92 |
| 6 | AM-71 | Month after | 2 437 438 | 95 297 | 3.91 | 4 403 319 | 94 337 | 2.14 | 4 036 137 | 86 413 | 2.14 |
| | | COVID-19 | | | | | | | | | |

The results demonstrate that for all three probes, K-A3 ratios for the COVID-19 patients were significantly higher than those obtained for the healthy patients. For the patient analyzed a month after recovering from COVID-19, the K-A3 ratio was higher than the ratio in healthy subjects, but lower than the ratio in COVID-19 positive subjects. Thus, the Method 2A assay can be used to diagnose COVID-19 positive patients and subjects who recently have had COVID-19.

### Example 9

### Measurement of Nuclease Activities in the Body Fluids of Cancer Patients Compared to Healthy Subjects as Indicative of the Presence or Absence of Cancer

In a variation of Method 2A (Method 2B), the measurement of nuclease activity (in the presence of Mg²⁺ alone, with no added Zn²⁺) was assessed as an indicator of the presence or absence of cancer. Like Method 2A, probes containing repeat (CNN)x or (GNN)x motifs were used. For Method 2B, the coefficient K-A3B is determined as the ratio of fluorescence of a cancer patient to the fluorescence of a validated healthy donor, as follows:

$${K}_{ARNA - 3} = \frac{( {ES}_{endpoint} - {ES}_{first point} )}{( {RS}_{endpoint} - {RS}_{first point} )} ,$$

where ES is “Examined Sample” (e.g., from a cancer patient) and RS is “Reference Sample” (validated healthy subject). As Reference Sample, a subject previously confirmed as having no breast cancer was used. The First Point represents the first fluorescence value after the plate containing the reaction mixtures for PCR has been set up in PCR machine (usually 2-3 minutes after the start). The Endpoint represents the last point acquired during PCR, which can be an incubation time of anywhere from between about 30 minutes to 2 hours or more, as long as the reaction curve is linear or almost linear.

Using a combination of 3 specific probes (mRNA sequences of the following genes: P53, RFX2 and SEPT9, this method provided a sensitivity for detecting breast cancer at close to 100%, with a specificity close to 85%.

As discussed below, blood plasma of breast cancer positive patients was analyzed in blood collection tubes (BCTs) of various producers, as follows:
PAX gene blood ccfDNA tube (Qiagen/BD, UK)
Streck (cfDNA BCT RU)
MINIMAX cfDNA BCT (Apostle)
K2EDTA BCTs (IMPROVE, China)
CitrateNa (TMPROVE, China)

The best results in terms of differentiation between patients with breast cancer and healthy donors was obtained using vacuum BCTs with K2EDTA.

Exemplary chemically synthesized probes are provided in Table 15, below:

TABLE 15
Exemplary chemically synthesized probes

| Genecards.org | GENE | PROBES | mRNA | (CNN)X |
|---|---|---|---|---|
| NM_005120 | MED12 | FAM- | mRNA_6925 | (CNN)13 |
| | | CAGCAGCAGCAACAGCAACAGCAGCAGCAGCAGCAGC- | | |
| | | BHQ1 (SEQ ID NO: 29) | | |
| NM_198829.1 | P21 | FAM-ACAGCGGCCCCGGCACCCAG-BHQ1 (SEQ ID | mRNA_2413 | (CNN)6 |
| | | NO: 31) | | |
| NM_00126114.2 | P53 | FAM-CCCCTGCACCAGCAGCTCCTACAC-BHQ1 (SEQ | mRNA_2724 | (CNN)8 |
| | | ID NO: 28) | | |
| NM_001256045.1 | PI3KB | FAM-CATGGGGCAGATGCTGGAGCAGT-BHQ1 (SEQ | mRNA_4797 | (GNN)7 |
| | | ID NO: 32) | | |
| NM_000635.4 | RFX2 | FAM-GTAACAGTCTCCACGGCTACAGTCTC-BHQ1 (SEQ | mRNA_3959 | (CNN)4 |
| | | ID NO: 30) | | |
| NM_001113491 | SEPT 9 | FAM-CCCTTGGT CAGCCTCCTCCC-BHQ1 (SEQ ID | mRNA_3244 | (CNN)4 |
| | | NO: 33) | | |
| NM_000125 | ESR1 | 5′FAM-CTGAACCGTCCGCAGCTCAAGATCC-3′ BHQ1 | mRNA_6357 | (CNN)4 |
| | | (SEQ ID NO: 1) | | |
| NM_001005862 | HER2 | 5′ FAM-CCCAGCCCTGGTCACCTACAACAC-3′ BHQ1 | mRNA_4858 | (CNN)4 |
| | | (SEQ ID NO: 7) | | |
| NM_002423.5 | MMP7 | 5′ FAM- | mRNA_1119 | (CNN)4 |
| | | CTGGAATGTTAAACTCCCGCGTCATAGAAATAATGC- | | |
| | | 3′BHQ1 (SEQ ID NO: 24) | | |

Reaction conditions for the 3 probes that were analyzed are provided in Table 16, below:

TABLE 16
Reaction Conditions
Conditions for Method 2B reaction:

| Volume reaction | 25 | ul |
|---|---|---|
| Volume plasma | 3.5 | ul |
| Tris -HCI | 40 | mM |
| pH | 8.3 | |
| MgCl₂ | 16 | mM |
| NaCl | 100 | mM |
| DTT | 0.5 | mM |

| | |
|---|---|
| Probes | 1 ul/20 uM |

Reaction Temperature

| | | |
|---|---|---|
| For probe - RFX2 | 33° | C. |
| For probe - P53 | 39° | C. |
| For probe - SEPT9 | 41° | C. |

The analysis was performed using automated procedures (Opentrons robot) in blinded mode. All blood samples were obtained in vacuum BCTs containing the preservation agent K2EDTA. The following 3 probes have been used which sequences are depicted above: P53, RFX2, SEPT9.

Box Plot analyses of K-A3B values for the above-mentioned 3 probes showed that all 3 probes provide statistically significant results in the differentiation of healthy samples from breast cancer samples (for P53, p=3.9·10⁻⁵, for RFX2, p=3.4·10⁻⁸, for SEPT9, p=1·10⁻⁵). The sum of K-A3B values for each of the above mentioned 3 probes also provide a statistically significant difference between cancer samples and normal samples, p=7.5.108.

Based on the Box Plot, the Specificity and Sensitivity is as shown in Table 17, below:

TABLE 17

| Test Result | Breast Cancer | Healthy | Total |
|---|---|---|---|
| Positive | 21 | 6 | 27 |
| Negative | 1 | 15 | 16 |
| Total | 22 | 21 | 43 |

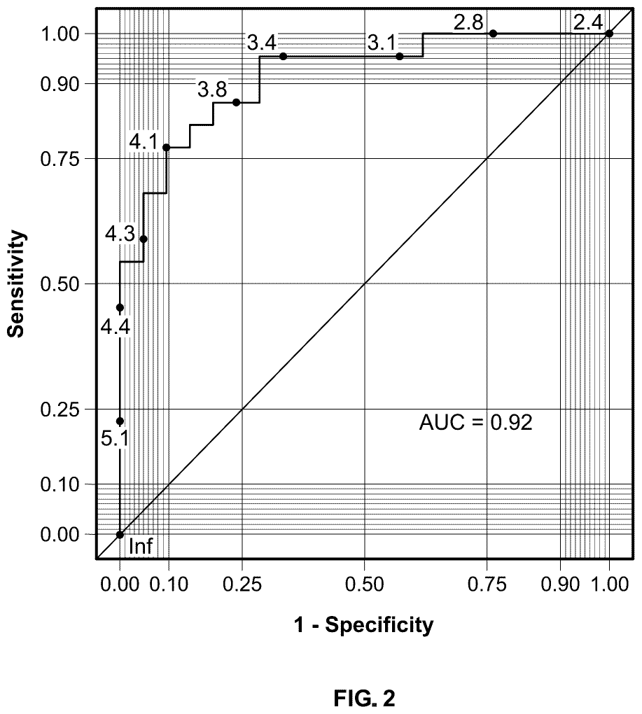

As shown in the ROC curve in FIG. 2, at a K-A3B cutoff of 3.4 (sum of K-A3B values from the 3 probes), the Sensitivity was found to be 95% (95% CI [77%, 100%]), the Specificity was found to be 71% (95% CI [48%, 89%]) and the ROC curve had AUC=0.92 (see FIG. 2).

A second analysis was performed with 135 plasma samples (healthy—42 samples; breast cancer of unknown subtype—31 samples; triple negative breast cancer—45 samples; HER2-enriched breast cancer—16 samples) healthy, in the presence of K2EDTA as a preserving agent, using multichannel pipettes in open mode. Probe sequences used in this experiment are provided in Table 18, below:

TABLE 18

| PROBE | |
|---|---|
| ID | SEQUENCE |
| ESR1- | 5′FAM-CTGAACCGTCCGCAGCTCAAGATCC-3′BHQ1 |
| ZO | (SEQ ID NO: 1) |
| MMP7- | 5′FAM-CTGGAATGTTAAACTCCCGCGTCATAGAAATAA |
| ZO | TGC-3′BHQ1 (SEQ ID NO: 24) |
| COX1- | 5′FAM-ATTCGGCGCATGAGCTGGAGTC-3′BHQ1 |
| ZO | (SEQ ID NO : 13) |
| HER2- | 5′FAM-CCCAGCCCTGGTCACCTACAACAC-3′BHQ1 |
| ZO | (SEQ ID NO: 7) |

Box Plot analyses of K-A3B values for the above-listed 4 probes showed that all 4 probes provide statistically significant results in the differentiation of healthy samples from breast cancer samples. In the box plot graph distribution of K-A3B value (sum of all K-A3B values for genes shown above) by sample group (healthy, HER2, TNBC and unknown−breast cancer without data on molecular subtype), all samples showed strong statistically significant differences from the healthy control group. Wilcoxon test p-values for each subtype are as follows: HER2—1.6·10⁻⁷, TNBC—5.8·10⁻¹³, unknown subtype—1.9·10⁻¹⁰, overall Wilcoxon p-value for comparing healthy vs. breast cancer (all groups) is 3.2·10⁻¹⁶.

Based on the Box Plot, the Specificity and Sensitivity is as shown in Table 19, below:

TABLE 19

| | Breast | | |
|---|---|---|---|
| Test | cancer | Healthy | Total |
| Positive | 89 | 7 | 96 |
| Negative | 3 | 36 | 39 |
| Total | 92 | 43 | 135 |

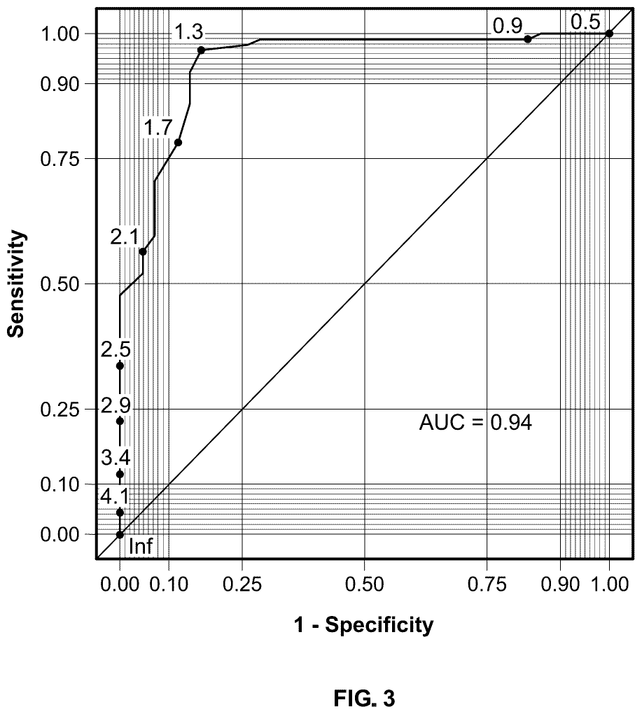

As shown in the ROC curve in FIG. 3, at a K-A3B cutoff of 1.3 (average K-A3B value for a single probe), the Sensitivity was found to be 97% (95% CI [91%, 99%]), the Specificity was found to be 83% (95% CI [48%, 89%]) and the ROC curve had AUC=0.94 (see FIG. 3).

The results provided in this Example demonstrate that the magnesium-dependent nuclease activity in a body fluid can be used to differentiate between samples from a cancer patient and samples from a healthy subject.

### Example 10

### Effects of Integrity of the Probe Motif (CNN)x and DTT Concentration in Measuring Nuclease Activities in the Body Fluids of Cancer Patients Compared to Healthy Subjects

To assess whether the presence or absence of zinc finger binding sites or motif(s) in the probes modulates nuclease activity in the body fluids of samples from healthy subjects or cancer patients, the Method 2B protocol described in Example 9 was tested in probes containing intact “CNN” motifs vs probes in which one or more of the “CNN” motifs are disrupted. Reaction conditions are as shown in Table 20, below:

TABLE 20
Reaction Conditions

| Reaction Volume | 25 | ul |
|---|---|---|
| Plasma Volume | 3.5 | ul |
| Tris -HCl | 40 | mM |
| pH | 8.3 | |
| MgCI₂ | 16 | mM |
| NaCI | 100 | mM |
| DTT | 0.5 mM (added to some samples, | |
| | as described below) | |
| Probes | l ul/20 uM | |
| Temperature for probes | | |
| RFX2 | 34° | C. |
| Reaction Volume | 25 | ul |
| P53 | 39° | C. |
| SEPT9 | 39° | C. |

The plasma was isolated in tubes containing K₂EDTA as a preservative, as described above. The time of reaction was about 45 minutes. The following probes were tested (mutations in the “CNN” motifs are enlarged and marked in bold):

TABLE 21
Exemplary probe sequences

| Probe ID | Probe | Sequence | CNN motifs |
|---|---|---|---|
| P1-O | SEPT9 | FAM-CCCTTGGTCAGCCTCCTCCC-BHQ1 (SEQ | (CNN)4 |
| | | ID NO: 33) | |
| P1-A | SE-T | FAM-CCCTTGGTCAGCCTCCTTCC-BHQ1 (SEQ | (CNN)3 |
| | | ID NO: 41) | |
| P1-B | SE-TA | FAM-CCCTTGGTTAGCCTCCTACC-BHQ1 (SEQ | (CNN)2 |
| | | ID NO: 42) | |
| P1-C | SE-AAA | FAM-CCCTTGGTCAGACTACTACC-BHQ1 (SEQ | (CNN)1 |
| | | ID NO: 43) | |
| P2-O | RFX2 | FAM-GTAACAGTCTCCACGGCTACAGTCTC- | (CNN)4 |
| | | BHQ1 (SEQ ID NO: 30) | |
| P2-A | RF-A | FAM-GTAACAGTCTCCACGGCTAAAGTCTC- | (CNN)3 |
| | | BHQ1 (SEQ ID NO: 44) | |
| P2-B | RF-AT | FAM-GTAACAGTCTCCACGGATATAGTCTC- | (CNN)2 |
| | | BHQ1 (SEQ ID NO: 45) | |
| P2-C | RF-TA | FAM-GTAACAGTCTTCAAGGCTACAGTCTC- | (CNN)2 |
| | | BHQ1 (SEQ ID NO: 46) | |
| P3-O | P53 | FAM-CCCCTGCACCAGCAGCTCCTACAC- | (CNN)8 |
| | | BHQ1 (SEQ ID NO: 28) | |
| P3-A | P53AAA | FAM-CCCCTGCACCAGCAGATCATAAAC- | (CNN)5 |
| | | BHQ1 (SEQ ID NO: 47) | |
| P3-B | PAAA53 | FAM-ACCATGAACCAGCAGCTCCTACAC- | (CNN)5 |
| | | BHQ1 (SEQ ID NO: 48) | |
| P3-C | P5AA3 | FAM-CCCCTGCACAAGAAGCTCCTACAC- | (CNN)3 + 3 |
| | | BHQ1 (SEQ ID NO: 49) | |

| | | 1 | 2 | 3 | 4 | 5 | 6 | 7 | 8 | 9 | 10 | 11 | 12 |
|---|---|---|---|---|---|---|---|---|---|---|---|---|---|
| Row | Column | PN-O | PN-O | PN-O | PN-A | PN-A | PN-A | PN-B | PN-B | PN-B | PN-C | PN-C | PN-C |
| A | S1 | | | | | | | | | | | | |
| B | S2 | | | | | | | | | | | | |
| C | S3 | | | | | | | | | | | | |
| D | S4 | | | | | | | | | | | | |
| E | S1 + DTT | | | | | | | | | | | | |
| F | S2 + DTT | | | | | | | | | | | | |
| G | S3 + DTT | | | | | | | | | | | | |
| H | S4 + DTT | | | | | | | | | | | | |

where:
(i) S1, S2, S3, S4 are the plasma samples (normal, cancer or fibrocystic disease);
(ii) S 1+DTT, S2+DTT, etc. are the plasma samples of (i) with added DTT;
(iii) PN-O represents the original, unmutated, probe N;
(iv) PN-A, PN-B, etc. represent mutant A of probe N, mutant B of probe N, etc.

### Method

1. For each well of the 96-well plate, the endpoint increase (last point fluorescence−first point fluorescence) was measured.
2. Samples were placed in rows as follows:
   a. Row A—sample ID 951-13 (breast cancer)
   b. Row B—sample ID 179-1 (triple negative breast cancer=TNBC)
   c. Row C—sample ID P651-13 (fibrocystic disease=FCD)
   d. Row D—sample ID DON-1 (normal control)
   e. Row E—sample ID 951-13 (breast cancer) with addition of DTT
   f. Row F—sample ID 179-1 (TNBC) with addition of DTT
   g. Row G—sample ID P651-13 (fibrocystic disease) with addition of DTT
   h. Row H—sample ID DON-1 (normal control) with addition of DTT
3. Each measurement was carried out in triplicate, then each value tested for whether it was an outlier using the Dixon test (W. J. Dixon, “Analysis of Extreme Values,” Ann. Math. Stat., vol. 21, no. 4, pp. 488-506, 1950). If the Q-value for this value was equal to or greater than the Q-value threshold (0.970 for CI 0.95) then such value was ignored in further calculations.
4. For each triplicate set of measurements, values that were taken into consideration were averaged and divided by the average normal control value.
5. The resulting K-A3B values are provided in Table 22, below:

TABLE 22
K-A3B Values

| | Without DTT | | | | With DTT | | | |
|---|---|---|---|---|---|---|---|---|
| | BC | TNBC | FCD | NORM | BC | TNBC | FCD | NORM |
| SEPT9 | 1.0 | 1.2 | 0.5 | 1.0 | 3.9 | 9.1 | 5.6 | 1.0 |
| SE-T | 1.3 | 1.5 | 0.6 | 1.0 | 3.1 | 6.3 | 3.3 | 1.0 |
| SE-TA | 1.2 | 1.7 | 1.1 | 1.0 | 2.5 | 3.4 | 2.7 | 1.0 |
| SE-AAA | 0.8 | 1.4 | 1.1 | 1.0 | 1.8 | 2.4 | 2.6 | 1.0 |
| RFX2 | 1.1 | 2.1 | 1.5 | 1.0 | 14.7 | 32.9 | 25.7 | 1.0 |
| RF-A | 1.2 | 2.2 | 1.8 | 1.0 | 2.7 | 5.4 | 4.2 | 1.0 |
| RF-AT | 1.4 | 1.8 | 1.7 | 1.0 | 3.8 | 6.6 | 8.1 | 1.0 |
| RF-TA | 1.0 | 1.9 | 1.9 | 1.0 | 2.4 | 4.4 | 5.6 | 1.0 |
| P53 | 1.1 | 1.6 | 0.6 | 1.0 | 1.8 | 2.7 | 1.5 | 1.0 |
| P53AAA | 1.0 | 1.4 | 0.5 | 1.0 | 2.6 | 3.8 | 2.2 | 1.0 |
| PAAA53 | 1.2 | 1.6 | 1.0 | 1.0 | 3.3 | 4.4 | 3.7 | 1.0 |
| P5AA3 | 0.9 | 1.5 | 1.1 | 1.0 | 1.8 | 2.6 | 2.4 | 1.0 |

### Results

In these experiments, three probes and their mutants were analyzed. The greatest influence of the mutants on probe structure was found for the RFX2 probe, in the presence of DTT. One substitution of C for A is enough for K-A3B to decrease by a factor of 6 in three samples (from 14.7, 32.9 and 25.7 to 2.7, 5.4 and 4.2 respectively, see Table above). For the SEPT9 probe, the introduction of a single mutation (SE-T) resulted in a slight decrease in the K-A3B value, followed by greater decreases with the introduction of each subsequent mutation (SE-TA and SE-AAA). The simultaneous introduction of three substitutions from C to A did not lead to a decrease in K-A3B for the probe for P53. A possible explanation is that this probe has 8 CNN motifs; therefore, disruption of 3 of these still leaves 5 CNN motifs that can interact with zinc fingers. The results demonstrate that the presence of zinc finger binding sites in the probes permit a clearer differentiation based on nuclease activity in cancer samples and FCD samples compared to samples from healthy subjects.

In these experiments, the effect of DTT on the differentiation between breast cancer or FCD samples and samples from healthy donors also was tested. DTT was found to reduce nuclease activity in healthy donors. Less of a decrease in nuclease activity is observed in patients with breast cancer, and virtually no reduction in nuclease activity was observed fibrocystic disease (FCD) positive patients. The results demonstrate that in the presence of DTT, the differentiation between cancer or FCD samples and samples from healthy donors based on K-A3B values is increased.

### Example 11

### Scoring Function

Analysis of the combination of results from the two assays, Methods 1 and Methods 2 provides better discrimination between subjects with disease, disorder, or condition, such as cancer or other particular health condition, and normal or healthy subjects who do not have the disease, disorder, or condition. Scoring functions take quantitative data from the two assays as inputs, and produces discrete or continuous results as output. Application of scoring functions, which take quantitative data from the two assays as inputs, produces discrete or continuous results as outputs can be represented as follows, where in this Example, A2 refers to Method 1, and A3 refers to Method 2:

Output=Fₛ(A2_{ctvalue},A3_{coefficient})
For Methods 1, a single Ct value is used as input for a given sample. This can be the average of Ct values derived from repeated measurements of the same sample.
For Methods 2, a single Method 2 coefficient can be used as input for a given sample. This can be calculated by using the average of data derived from repeated measurements of the same sample.
For Methods 1 and Methods 2A and 2b, discrete values (for example, true/false and positive/negative) can be used based on thresholds that have been set for each of the two assays, and can be set for a particular disease, disorder, or condition.
Output can be calculated as a discrete value, such as true/false and positive/negative, or a probability ratio, which indicates the predicted probability of a positive outcome on a scale of 0% to 100%.

Shown below is a representative example of application of scoring functions to experimental results from the two assays. Eight plasma samples were subjected to repeated analysis by Method land Method 2 assays. The averages of Ct values and A3 coefficients, obtained from Method 2 and Method 2, respectively, are shown below:

| | Average Ct value | Average A3 (Method 2) |
|---|---|---|
| Sample ID | (Method 1 assay) | coefficient (Method 2 assay ) |
| X70 | 32.40 | ND |
| X70 | 33.00 | ND |
| X66 | 33.83 | −3.05 |
| P42 | 32.00 | 12.31 |
| X51 | 33.33 | −12.79 |
| X123 | 34.60 | −2.23 |
| X164 | 32.25 | 2.77 |
| D4 | 33.60 | −2.89 |

ND—Data from Method 2 assay were disqualified based on the quality control of given measurements. These samples are considered negative.

The following scoring functions were applied to the above results:

$$A {2}_{scoring} = \{ \begin{matrix}True , if A {2}_{Ct mean value} \leq 32.5 \\ False , otherwise\end{matrix}$$

$$A {3}_{scoring} = \{ \begin{matrix}True , if A {3}_{coeff} \geq 1.3 \\ False , otherwise\end{matrix}$$

This yielded the following results for each sample:

| | Ct value | A3 coefficient |
|---|---|---|
| Sample ID | (Method 1 assay) | (Method 2 assay) |
| X70 | True | False |
| X70 | False | False |
| X66 | False | False |
| P42 | True | True |
| X51 | False | False |
| X123 | False | False |
| X164 | True | True |
| D4 | False | False |

The Boolean operator “OR” was applied to the above results to draw output, i.e.,

Output=OR(A2_{ctvalue},A3_{coefficient})

This yielded the following output for each sample. The status of each subject is also shown.

| | | A3 | | Subject status | |
|---|---|---|---|---|---|
| | Ct value | coefficient | | (normal or | |
| Sample | (Method 1 | (Method 2 | | breast | Test |
| ID | assay) | assay) | Output | cancer) | correctness |
| X70 | True | False | True | Breast cancer | Correct |
| X70 | False | False | False | Breast cancer | Incorrect |
| X66 | False | False | False | Normal | Correct |
| P42 | True | True | True | Breast cancer | Correct |
| X51 | False | False | False | Normal | Correct |
| X123 | False | False | False | Normal | Correct |
| X164 | True | True | True | Breast cancer | Correct |
| D4 | False | False | False | Normal | Correct |

Since modifications will be apparent to those of skill in this art, it is intended that this invention be limited only by the scope of the appended claims.

What is claimed:

1. A method for analyzing cell-free nucleic acids in a body fluid from a subject as indicative of the presence or absence of a disease or condition in the subject, the method comprising:
a) subjecting a sample of body fluid previously obtained from the subject to an amplification reaction, wherein:
i) the sample comprises cell-free nucleic acid, and cell-free nucleic acid derived from at least one target gene is specifically amplified, and
ii) the cell-free nucleic acid derived from the target gene is present in an amount that is about or less than 5, 4, 3, 2, or 1 molecule(s), or molecule(s) per unit volume, in an analogous control or reference sample, and is present in the sample from the subject with the disease or condition in an amount that is greater than the amount in the control or reference sample by 2 or more molecules or molecules per unit volume; and
b) quantitating the amplified nucleic acid from a), wherein:
i) if the amount of amplified nucleic acid is at or above a threshold level, the subject is identified as having the disease or condition; and
ii) if the amount of amplified nucleic acid is below a threshold level, the subject is identified as not having the disease or condition.

2. The method of claim 1, wherein the control or reference sample is an analogous sample from a normal subject or a subject who does not have the disease or condition.

3. The method of claim 1, wherein the difference between the number of cell-free nucleic acid molecules derived from the target gene that are present in the control or reference sample and the number of cell-free nucleic acid molecules derived from the target gene that are present in the sample from the subject with the disease or condition is up to or about 2, 3, 4, 5, 6, 7, 8, 9 or 10 molecules, or up to or about 2, 3, 4, 5, 6, 7, 8, 9 or 10 molecules per unit volume.

4. The method of claim 1, wherein the threshold level is determined based on a level that is measured in at least one subject known to have the disease or condition and/or is based on a level that is measured in at least one control or reference sample; or the threshold level is determined based on a level that is the mean or the median of levels measured in more than one subject known to have the disease or condition and/or the mean or the median of levels measured in more than one control or reference sample.

5. The method of claim 4, wherein the control or reference sample is an analogous sample from a normal subject or a subject who does not have the disease or condition.

6. The method of claim 1, wherein the quantitating is effected by gel electrophoresis, measuring the amount of dsDNA (Picogreen assay), UV spectrometry, real-time quantitative PCR (realtime-qPCR), droplet digital PCR (ddPCR), beads, emulsion, amplification, magnetics PCRr (BEAMing), or tagged-amplicon deep sequencing (TAm-Seq).

7. The method of claim 1, wherein the quantitating is performed by measuring a signal that is proportional to or inversely proportional to the amount of the amplification product.

8. The method of claim 1, wherein the cell-free nucleic acid in the sample of body fluid is treated with a proteinase prior to analysis.

9. The method of claim 1, wherein the body fluid is whole blood, urine, plasma, serum, cerebrospinal fluid, saliva, sputum, lavage from the lungs, synovial fluid, peritoneal fluid, pericardial fluid, pleural fluid, amniotic fluid, vitreous humor, aqueous humor, spinal fluid, lavage fluid of bronchoalveolar, gastric, peritoneal, ductal, ear or arthroscopic origin, nasal mucous, prostate fluid, lavage, semen, seminal fluid, lymphatic fluid, bile, tears, sweat, breast milk, or breast fluid discharge.

10. The method of claim 1, wherein the amplification reaction is effected on the body fluid sample without extraction or purification of cell-free nucleic acid.

11. The method of claim 10, wherein:
the control or reference sample is an analogous sample from a normal subject or a subject who does not have the disease or condition;
the difference between the number of cell-free nucleic acid molecules derived from the target gene that are present in the control or reference sample and the number of cell-free nucleic acid molecules derived from the target gene that are present in the sample from the subject with the disease or condition is up to or about 2, 3, 4, 5, 6, 7, 8, 9 or 10 molecules, or up or about to 2, 3, 4, 5, 6, 7, 8, 9 or 10 molecules per unit volume;
the threshold level is determined based on a level that is measured in at least one subject known to have the disease or condition and/or is based on a level that is measured in at least one control or reference sample; or the threshold level is determined based on a level that is the mean or the median of levels measured in more than one subject known to have the disease or condition and/or the mean or the median of levels measured in more than one control or reference sample; and
the quantitating is performed by measuring a signal that is proportional to or inversely proportional to the amount of the amplification product.

12. The method of claim 11, wherein the sample of body fluid is treated with a proteinase prior to analysis.

13. A method for analyzing cell-free nuclease activity in a body fluid from a subject as indicative of the presence or absence of a disease or condition in the subject, the method comprising:
a) procuring a first aliquot from the body fluid, wherein the body fluid was previously obtained from the subject;
b) to the first aliquot, adding Zn²⁺, Mg²⁺ and a nucleic acid probe;
c) subjecting b) to reaction conditions under which nuclease activity, if present in the body fluid, is determined by measuring digestion of the nucleic acid probe;
d) procuring a second aliquot from the previously obtained body fluid, wherein the second aliquot is of the same or similar amount as the first aliquot;
e) to the second aliquot, not adding Zn²⁺ and adding Mg²⁺ and the same nucleic acid probe that is used in b);
f) subjecting e) to reaction conditions under which nuclease activity, if present in the body fluid, is determined by measuring digestion of the nucleic acid probe; and
g) obtaining a ratio of the nuclease activity determined in c) to the nuclease activity determined in f), wherein:
i) if the ratio is at or above a threshold level, the subject is identified as having the disease or condition; and
ii) if the ratio is below a threshold level, the subject is identified as not having the disease or condition.

14. The method of claim 13, wherein the threshold level is determined based on the ratio that is measured in at least one subject known to have the disease or condition and/or is based on the ratio that is measured in at least one control or reference sample.

15. A method for analyzing cell-free nuclease activity in a body fluid from a subject as indicative of the presence or absence of a disease or condition in the subject, the method comprising:
a) to a previously obtained sample from the subject, adding Mg²⁺ and a nucleic acid probe comprising at least one zinc finger binding site;
b) subjecting a) to reaction conditions under which nuclease activity, if present in the body fluid, is determined by measuring digestion of the nucleic acid probe;
c) subjecting a reference or control sample to the same or similar reaction conditions as b) under which nuclease activity, if present in the reference or control sample, is determined by measuring digestion of the nucleic acid probe, or obtaining a predetermined value of nuclease activity from a control or reference sample; and
d) obtaining a ratio of the nuclease activity determined in b) to the predetermined nuclease activity or nuclease activity that is determined in c), wherein:
i) if the ratio is at or above a threshold level, the subject is identified as having the disease or condition; and
ii) if the ratio is below a threshold level, the subject is identified as not having the disease or condition.

16. The method of claim 15, wherein, if both the subject and the reference or control are subjected to reaction conditions under which nuclease activity is measured, the reactions are performed sequentially, simultaneously or in any order.

17. The method of claim 15, wherein the threshold level is determined based on the ratio that is measured in at least one subject known to have the disease or condition and/or is based on the ratio that is measured in at least one control or reference sample.

18. The method of claim 17, wherein the control or reference sample is an analogous sample from a normal subject or a subject who does not have the disease or condition.

19. The method of claim 15, wherein the nucleic acid probe comprises a sequence of (CNN)x or (GNN)x repeats, wherein:
a) x is the number of CNN or GNN repeats, and the number is at least 2; and
b) N is A, G, C, or T.

20. The method of claim 19, wherein x is 3 or more repeats.

21. The method of claim 19, wherein x is 3, 4, 5, 6, 7, 8, 9, 10, 11, 12, 13, 14, 15, 16 or 17 or more repeats.

22. The method of claim 15, wherein the reaction conditions further comprise adding a reducing agent.

23. The method of claim 22, wherein the reducing agent is selected from among TCEP (tris(2-carboxyethyl) phosphine), DTT, DTE, glutathione, Na acetylcysteine, 2-mercaptoethanol, cysteine hydrochloride, sodium thioglycolate, diethyldithiocarbamate, thioglycolic acid and DTBA (dithiobutylamine).

24. The method of claim 23, wherein the reducing agent is DTT.

25. A method of analyzing a body fluid from a subject as indicative of the presence or absence of a disease or condition in the subject, the method comprising:
a) subjecting a first aliquot of a sample of body fluid previously obtained from the subject to an amplification reaction, wherein the sample comprises cell-free nucleic acid;
b) quantitating the amplified nucleic acid from a), and determining whether the amount of amplified nucleic acid is at or above a threshold level, or below a threshold level;
c) subjecting a second aliquot of the same sample of body fluid to a reaction to determine an enzymatic activity;
d) determining whether the enzymatic activity in c) is at or above a threshold level, or below a threshold level; and
e) if either or both of the following conditions are met:
i) the amount of amplified nucleic acid is at or above a threshold level, and/or ii) the enzymatic activity is at or above a threshold level, identifying the subject as having the disease or condition.

26. The method of claim 25, wherein the enzymatic activity is a nuclease activity.

27. A method of detecting the presence or absence of a disease, disorder, or condition in a subject, comprising:
a) analyzing a cell-free nucleic acid sample in a body fluid from the subject by the method of claim 1, and detecting the presence of a target gene at a higher level than in a control or reference sample; and,
b) analyzing cell-free nuclease activity to detect activity indicative of the disease or condition,
wherein, when a) and b) are indicative of the disease or condition, identifying the subject as having the disease, disorder, or condition.
